(19) United States
(12) Patent Application Publication
(10) Pub. No.: US 2024/0371510 A1
(43) Pub. Date: Nov. 7, 2024

(54) DIGITAL CONTENT LABELING BASED ON CORRELATED PERSON BEHAVIORS

(71) Applicant: James Andrew Aman, Celebration, FL (US)

(72) Inventor: James Andrew Aman, Celebration, FL (US)

(21) Appl. No.: 18/644,278

(22) Filed: Apr. 24, 2024

Related U.S. Application Data
(60) Provisional application No. 63/530,986, filed on Aug. 6, 2023.
(60) Provisional application No. 63/498,341, filed on Apr. 26, 2023.

(51) Int. Cl. G16H 40/63

(52) U.S. Cl. CPC G16H 40/63

(57) ABSTRACT

A system and method for determining and labeling any one or more components of digital content forming a session work product determined during the session of operation of a computing system by a person/operator. The person provides one or more informational contributions during the session of operation as sensed or determined by one or more input devices in communication with the computing system. The labeling is based at least in part upon correlating any of the informational contributions with any one or more minimal content objects comprising a component of digital content. Informational contributions comprise any one of or any combination of the person's authenticated identity, presence, liveness, and/or behaviors made substantially congruent or concurrent with the receiving of digital information comprising either of minimal content objects or application commands by the computing system.

### REFERENCE TO RELATED APPLICATIONS

The present application is related to PCT application PCT/US22/47511 filed on Oct. 23, 2022, entitled MEASUREMENT AUTHENTICATING WELLNESS DEVICES. The present application is related to PCT application US2021/029186 filed on Apr. 26, 2021, entitled GUEST TRACKING AND ACCESS CONTROL USING HEALTH METRICS.

### FIELD OF INVENTION

The present invention relates to operator authorizing and readiness for asset operation monitoring systems, where operation is authorized, monitored, and limited in accordance with a privately authenticated operator readiness assessment.

### BACKGROUND OF THE INVENTION

The present teachings are related to prior applications from the present inventors including PCT US2021/029186 filed on Apr. 26, 2021, entitled GUEST TRACKING AND ACCESS CONTROL USING HEALTH METRICS, and PCT application PCT/US22/47511 filed on Oct. 23, 2022, entitled MEASUREMENT AUTHENTICATING WELLNESS DEVICES. In general, these prior teachings discuss the further adaptation of personal devices such as smartphones and wearables that are currently limited to traditional uses for determining any of biometrics or health related measurements taken in private and lacking authentication. The referenced art taught adaptations including apparatus and methods for privately authenticating the person substantially before or during the determination of any health-related measurements, where then these “privately authenticated health measurements” are combinable using a “public regiment” to form an “authenticated, publicly trustable” health status. Publicly trustable implies that the state-of-the-health status, such as “passing level 1, level 2, or level 3 public regiments” is assured to be a reliable health representation of the actual person. In this capacity, the publicly trustable health status is usable as a measure of the “readiness” or fitness of a person, where in the prior teachings this readiness and fitness was applied to the domain of the meeting and gathering with people in virtually any location or setting, thus providing a mutual assurance to all individuals for example aggregating on a premises that each individual was sufficiently unlikely to be for example potentially transmissive of an infectious disease.

The further adapted personal devices as taught in the related applications included what was referred to as an “intermediary app” and also and “honest broker app,” where the combination of the personal device (e.g., the smartphone or watch/wearable) executing the intermediary app was referred to as a “primary device.” Primary devices were shown to be capable of communicating with secondary devices, such as other wearables or non-wearables including exercise equipment, breathalyzers, smart scales, etc., where secondary devices determined one or more additional health measurements for provision to and aggregation by the primary device, where the aggregated health measurements increased the reliability of any on-going heath assessment.

Those familiar with the rapid pace of development in biosensors will understand that the types, kinds, and accuracies of physiological measurements are advancing at a significant pace across a multiplicity of device and measurement categories, where the authenticated aggregation of these disparately determined physiological measurements combine to create an ever increasingly accurate predictive tool of a person's health state. Those familiar with psychological measurements will understand that there is a likewise accelerating understanding of means for determining the psychological state of a person, where if sufficiently authenticated these disparate psychological measurements also contribute significantly to “publicly trustable” mental health state regarding a particular person.

Additional teachings were provided to further adapt a “premises” to comprise one or more “self-access points,” where a person using a primary device anonymously interacted with the premises access point to confirm authentication and a sufficient health status. The person was not required to first enroll with the premises or access point, nor to ever share any personal information with the premises or access point in order to gain self-access (where personal information includes biometric information and any of specific health measurements comprising the health status, thus protecting personal privacy). The referenced art also discussed “ticketed access” whereby a person is first provided with a “right-to-access” a premises, where this right is associated with the intermediary app, also referred to as an “honest broker,” where the honest broker executing solely on the primary device registers (or “enrolls”) a person as the sole rights-owner, and then privately confirms the person's authenticity as the enrollee in cooperation with the access point, thus also assuring the premises that a person “has authenticated rights-to-access,” where these rights-to-access combined with a health state provide a powerful new technology for managing at least the aggregation of individuals at a location or event while minimizing the likelihood of health risk exposures, where the locations or events are either restricted (i.e., “ticketed”) or unrestricted. This brief description of the prior teachings is not meant to be limiting, and the interested reader is directed to the prior teachings for a full and complete understanding.

The present teachings further adapt the apparatus and methods of at least these prior teachings to provide for a generalized system for authorizing and monitoring the use (“operation”) of assets comprising vehicles, equipment, structures for occupation such as buildings or homes or otherwise any process influenced or controlled by an operator (thus “the person”). These vehicles, equipment, structures, or processes may be considered as a generalized restricted “premise” comprising an “access point” whereby “entry” through (or into) this access point situates and/or enables the operator to at least in part operate the vehicle, equipment, structure, or process. The access point could be a doorway into a limited space such as a cabin or could be a means by which operation is otherwise restricted, such as a “key” or a “fob” that must be in the operator's possession and properly engaged with the asset prior to any such operation. The access point might also be a control panel for which a password is required prior to accessing, and otherwise any physical and/or digital interface for engaging by a person for the purposes of operating any of an asset such as a vehicle or equipment, or process such as a warehouse assembly line.

For the purposes of clarity, the present teachings are focused on a non-limiting exemplary use case where the operator is a driver, and the vehicle/equipment is an automobile. A careful consideration will show that these teachings extend significantly to include for example all types of transportation as well as industrial equipment and processes, some of which may be associated with the physical transport of the operator, others of which are simply associated with the performance of some combination of restricted mechanical or non-mechanical operations. Uses cases also extend into events such as a sporting event, a surgery, or a job-shift where especially the “ready-performance” of the person is at least in part attributable to the favorable outcome of the event, and where a favorable outcome often includes the safety of the person or other persons.

A common desirable application includes any vehicle, equipment, or process whose operation includes appreciable safety concerns, where the current and on-going “readiness” of the operator significantly effects such concerns, and where this readiness assessment can be based at least in part upon the on-going health status of the operator as determined by the primary device. Many such “desirable application” vehicles, equipment, or processes may be operated at any given time by different operators, where it is valuable to maintain operation-session information per authenticated operator, whereby this session-information is then at least in part usable especially in combination with the operator's on-going health state to create baselines and operation history that informs future vehicle, equipment, or process operation limits.

Session-information comprises both direct and indirect measurements indicative of the operator's on-going readiness and performance. For example, direct measurements of an operator/driver include the use of cameras or other sensors to determine one or more physiological or psychological measurements of the person, where for example physiological measurements include facial gestures such as eye or head movements or body expressions such as heart rate and breathing rate. Indirect measurements include vehicle/equipment movements or otherwise operational changes at least in part responsive to inputs by the operator/driver, where such movements or changes are interpretable as being indicative or non-indicative of an expected “safe” operation or normally desired pattern.

With respect to roadway safter, driver impairment is a leading cause of accidents and death. Impairment is a depressed state of driver readiness that is attributable to one or more physiological or psychological causes.

Readiness for example is greatly affected by fatigue and alcohol or other mind-body altering substances including medications. Current readiness assessment systems are reliant upon direct and indirect measurements determined only during a current driving session, lacking pre-session driver readiness awareness, session-to-session driver readiness and performance continuity of assessment, the combination of which preclude driver readiness baselines and driver categorization by profiles for readiness generalization.

Two other factors significantly affecting driver/operator readiness, in this sense potential “driver impairments,” are environmental conditions and/or route characteristics. Environmental conditions include time of day, ambient lighting, and weather, all projected over the anticipated duration of the time and physical route of operation. The route characteristics include the physical route and the duration of time anticipated for transversal, as well as the routes driving “complexity,” operator familiarity, either specific or by “general route type.” Those familiar with current environmental sensors, GPS routing systems, etc., will understand that data for determining these types of environmental conditions and route characteristics are substantially available in the current marketplace, where then the present teachings incorporate the authentication and application of this information to enhance operator readiness and monitoring.

Significant information regarding an operator's physiological and psychological state, thus their personally measurable readiness for the anticipated operational task, is determinable using today's wellness devices (see prior related applications for more discussion,) with an expectation of increased measurement range and accuracy given the pace of technological advancements. This operator/driver state includes both an on-going (changing) current state as well as a past state history, both of which are predictive. For example, regarding both drowsiness and alcohol impairment, studies have shown a significant range of impairment variation based not just on the individual (thus the benefit of authentication tied with individual baselines and comparative operation histories,) but also based upon the past health state assessment of the individual. For example, a person operating a vehicle or equipment under the duress of a single night of non-ideal sleep or alcohol consumption, is typically less susceptible to impairment than a person with for example several nights of lost sleep (and/or heavier physical activity) or alcohol consumption.

Although currently measurable prior to an operator's engagement by using wearables and other non-vehicle technologies, without authentication any such physiological and psychological measurements lack the necessary public trust for use as operational readiness assessments and subsequent objectified driver operational limits. Likewise, while significant information regarding driver readiness and operation performance is determinable using today's in-vehicle technologies, lacking authentication to a specific driver, any such performance information is not usable as meaningful and transferable history or for establishing individual personal baselines, and is minimally useful for monitoring readiness and setting real-time operational limits.

What is needed is a system for determining the historical and on-going readiness of a vehicle or equipment operator both prior to and during the act of operating. To ensure public trust and the acceptance of individual operators, the system must implement authentication for the data substantially contributing to the personal readiness assessment. The ideal system is generalized rather than vehicle, equipment, or process centric, where generalization also supports operator typing to create predictive categories. All data is not only distributed to the operators for personal encrypted ownership and via this personal data ownership made transferable between sessions across various vehicles, equipment, and processes, but the data in many scenarios also remains private during session assessment and operation, thus providing both a publicly trustable and anonymous system. What is also preferred is that relevant data is anonymized and then centrally aggregated for assessment by third parties, where then using newer pattern assessment technologies such as machine learning and AI that are heavily reliant upon real-world training data, third parties can improve products and offerings, including the manufacturers of the vehicles, equipment, and processes as well as insurers of the individual operators.

### SUMMARY OF THE INVENTION

An object of the present invention is to provide operator readiness assessment that privately authenticated comprising both non-session operation data and session operation data. This readiness assessment data includes the on-going health state of the operator obtained prior, during, and after operation, derived baselines, historical operation tracking, as well as third-party restriction policies based at least in part upon any of the authenticated operation data. An operator is a person that has substantial control over any of a critical operation employing any of an asset including a vehicle, equipment, process, or structure for occupation, especially where the critical operation has personal and/or group and societal safety concerns.

To best accomplish this and other objects, the present invention teaches a system and methods for 1) determining authenticated measurements (“metrics”) of an operator 10 prior, during, or after any vehicle/equipment/process 20 operation session, 2) determining authenticated measurements of a session context prior, during, or after any vehicle/equipment/process 20 operation session, 3.1) aggregating over any two or more operation sessions any of authenticated operator measurements (1) or authenticated context measurements (2), 3.2) determining authenticated assessments such as baselines and histories associated with any of the specific or type of operator 10, vehicle/equipment/process 20, or session, 4.1) providing any of authenticated information (1), (2), or (3) to one or more third parties, 4.2) receiving one or more policy restrictions as determined by a third-party, where restrictions comprise operation limitations based at least in part upon the provided authenticated information, 5.1) determining an operator 10's authenticated “vetted” readiness to conduct a session of operation based at least in part upon any of authenticated information (1), (2), or (3), or policy restrictions (4), 5.2) aggregating authenticated readiness determinations (5.1) with any of authenticated information (1), (2), or (3) for uses associated with function (4) for determining policy restrictions, 6) determining any one or more session authenticated operation limitations to be applied to an upcoming or on-going operation session of an operator 10 based at least in part upon any of policy restrictions (4) or the operator's authenticated readiness (5), 7.1) providing means to an operation rights-allower for determining, enrolling, and/or distributing rights-criteria comprising any of authentication, authorization, and vetting criteria for the allowance, assignment, selection or otherwise assessment of one or more operators 10 desirous of operating a vehicle/equipment/process 20 for one or more sessions of operation, where any of the rights-criteria may be specified in reference to, or are otherwise used at least in part with, any of authenticated information (1), (2), (3), policy restrictions (4), the operator's authenticated readiness (5), or operation limits (6), 7.2) providing for the transfer of operation rights optionally compensated by payment of monies or equivalent, 8) providing generalized means associated with either a specific or a type of vehicle/equipment/process 20 for interacting with a desirous operator 10 to determine sufficient rights-criteria (7.2) and interacting with vehicle/equipment/process 20 for enabling any of entrance, occupation, operation, and/or data recording functions in accordance with the sufficient readiness-vetted rights-criteria, and 9) providing salable authenticated readiness-vetted operationally limitable services in association with any of a vehicle's/equipment/process 20's authenticated operation during any one or more authenticated sessions.

The present teachings are also shown to be extensible to uses where the asset is for example a computer system with various applications being used to generated session work products, where there is a current marketplace desire to ensure that a given work product is composed by a human (thus “organic”) and not what is known as a generative AI algorithm (thus “synthetic”). The present means for assuring the authenticity of an operator (human) of an asset (ranging from a vehicle, to a building, to a computing system and app) is therefore shown to provide a unique solution for assuring that any work products created during an authenticated session of operation of a computing system, such as a digital document comprising virtually any type of digital content, can be labeled as “organic” because their composer is the authenticated asset (computer) operator.

In the following description, numerous specific details are set forth, such as examples of specific component parts and their key configuration as well as alternative variations, arrangements and combinations of these components, exemplary types of usage scenarios for these many arrangements, etc. in order to provide a thorough understanding of the present disclosure. It will be apparent, however, to one skilled in the art that the present disclosure may be practiced without at least some of these specific details and with alternative implementations, some of which are also described herein. In other instances, well-known components or methods have not been described in detail to avoid unnecessarily obscuring the present disclosure. The specific details set forth are exemplary. The specific details may be varied in from or comprise additional related functions and still be contemplated to be within the spirit and scope of the present disclosure.

### BRIEF DESCRIPTION OF THE FIGURES

FIG. 1A is a block diagram showing the major components by function of the present system implemented in the exemplary use case of an operator-vehicle (“driver-car”) authority and readiness assessment system 100.

The exemplary use case system 100 comprises an operator 10 that engages and operates a vehicle (e.g., a car) 20. The depicted system 100 functional components are aggregated into two groups comprising authenticated, authorized, vetted access control group 30 and authenticated, verified, operator “continuous-readiness” assessment group 40. Access control group 30 comprises operator authentication and vetting component 30-2 that works cooperatively with vehicle access control component 30-4. Continuous-readiness group 40 comprises operator seated detection component 40-2 that works cooperatively with operator readiness determination component 40-4 that works cooperatively with vehicle operation limits component 40-6. Any of the components may be configured and operated as either self-compliant 5 or mandatory use 6, and any of the components, or parts of the components, may be deployed on either or both of a device used by the operator 10 or the vehicle 20.

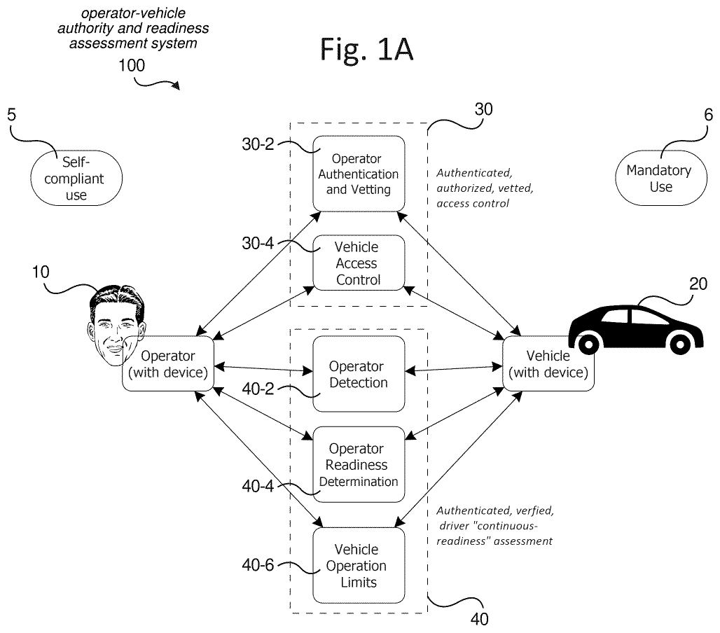

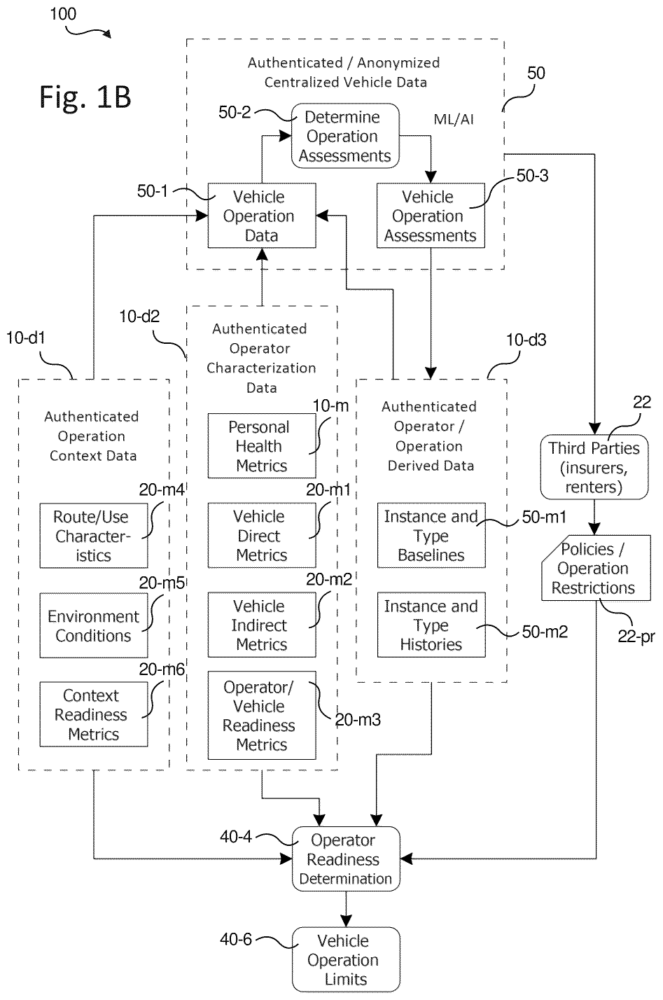

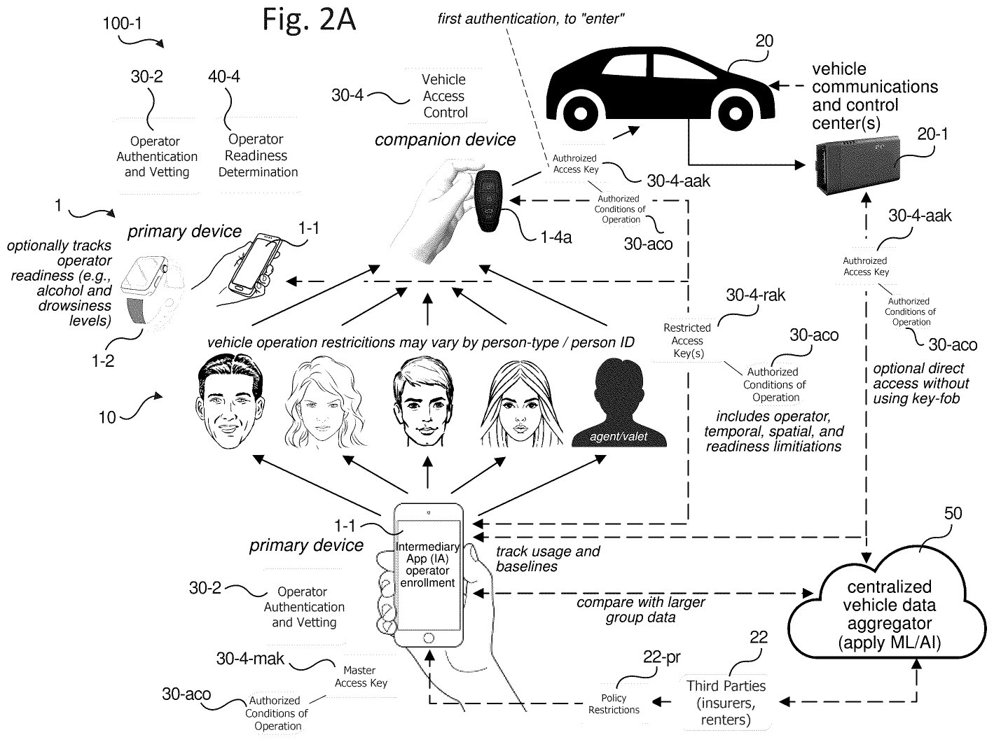

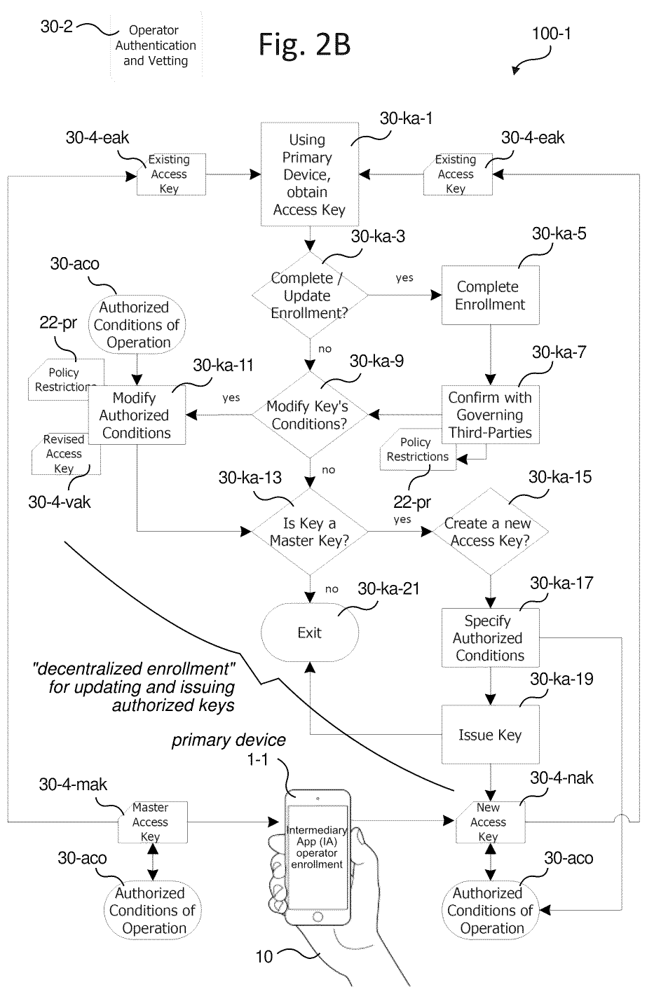

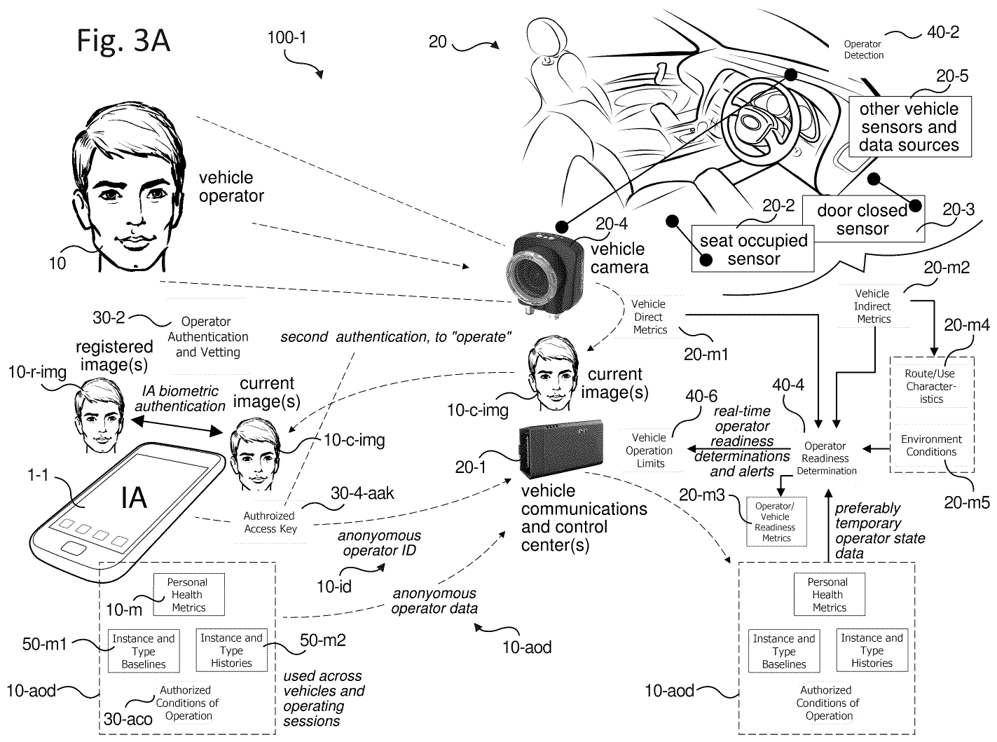

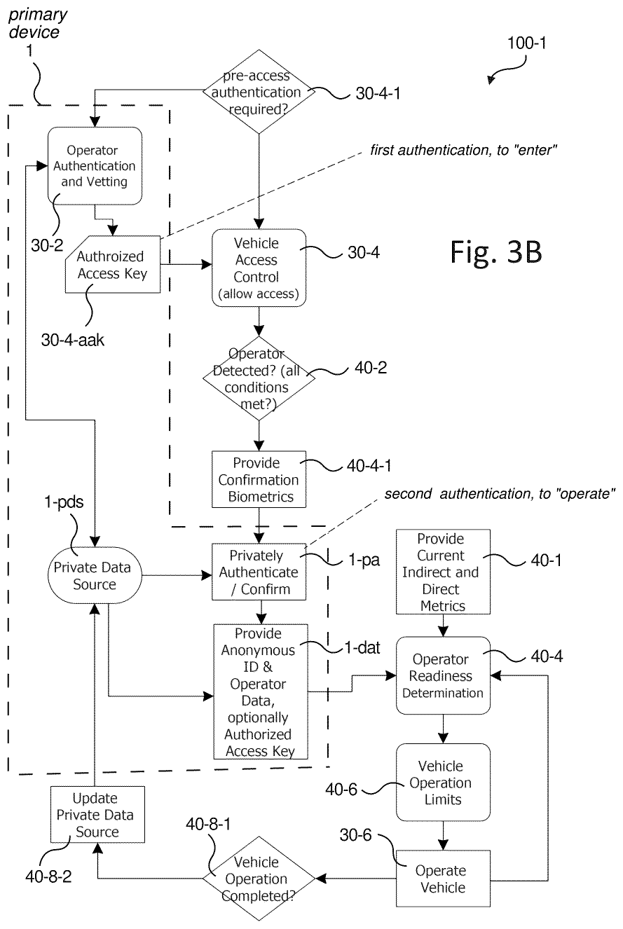

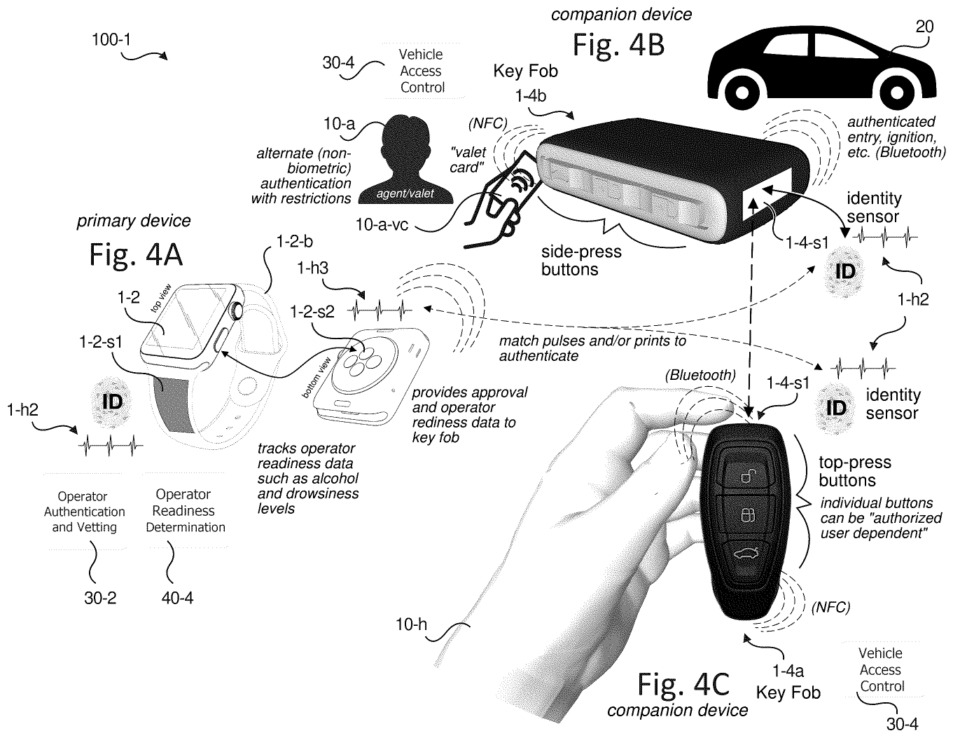

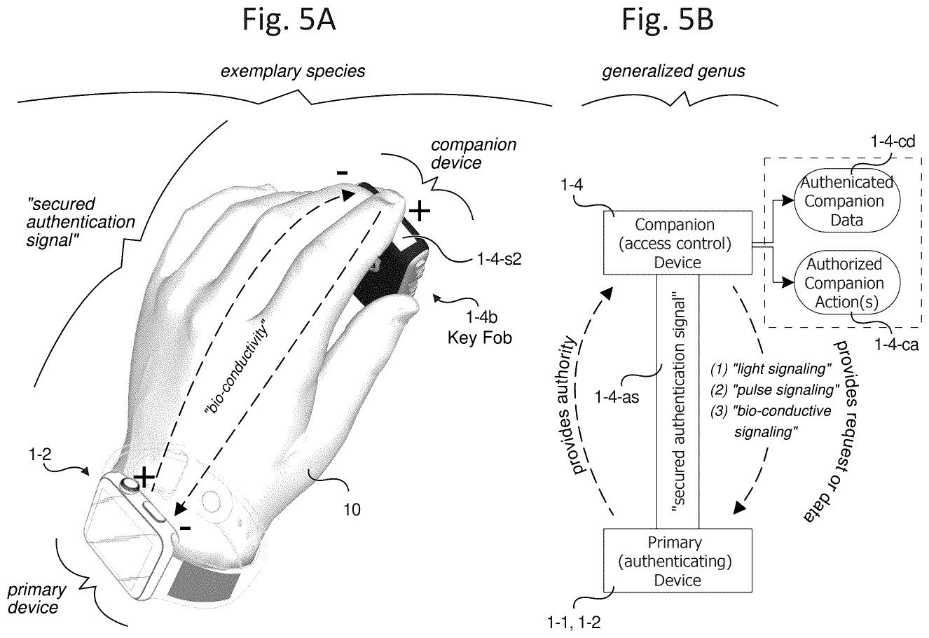

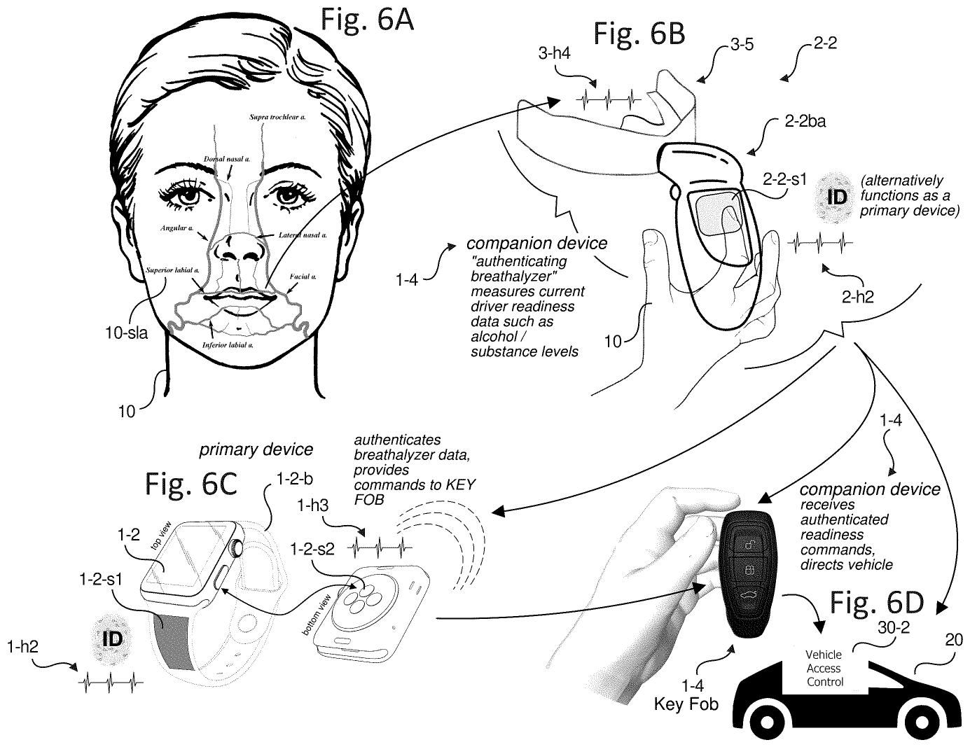

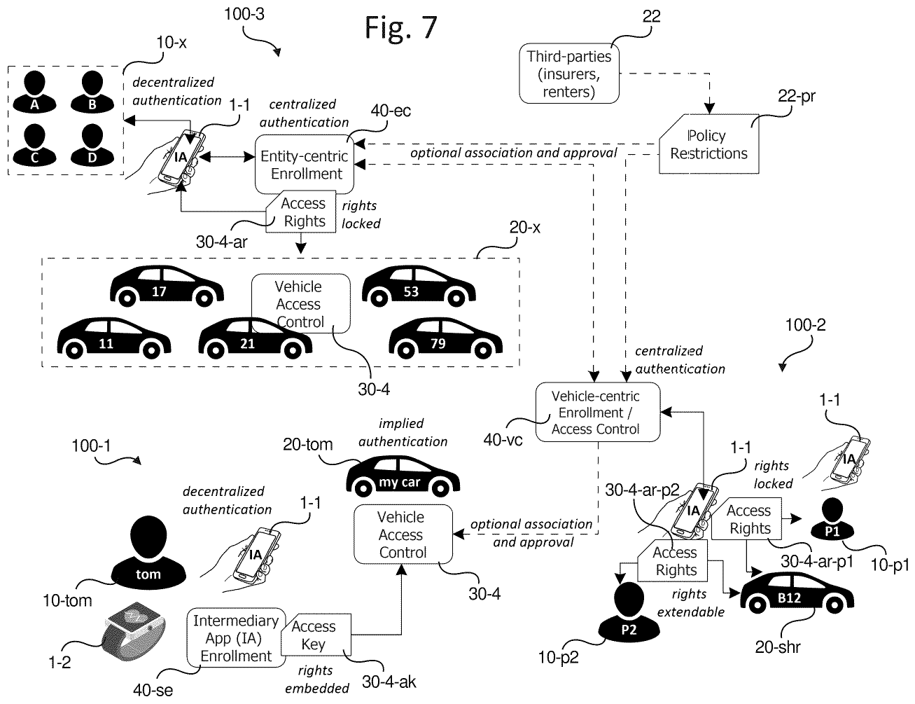

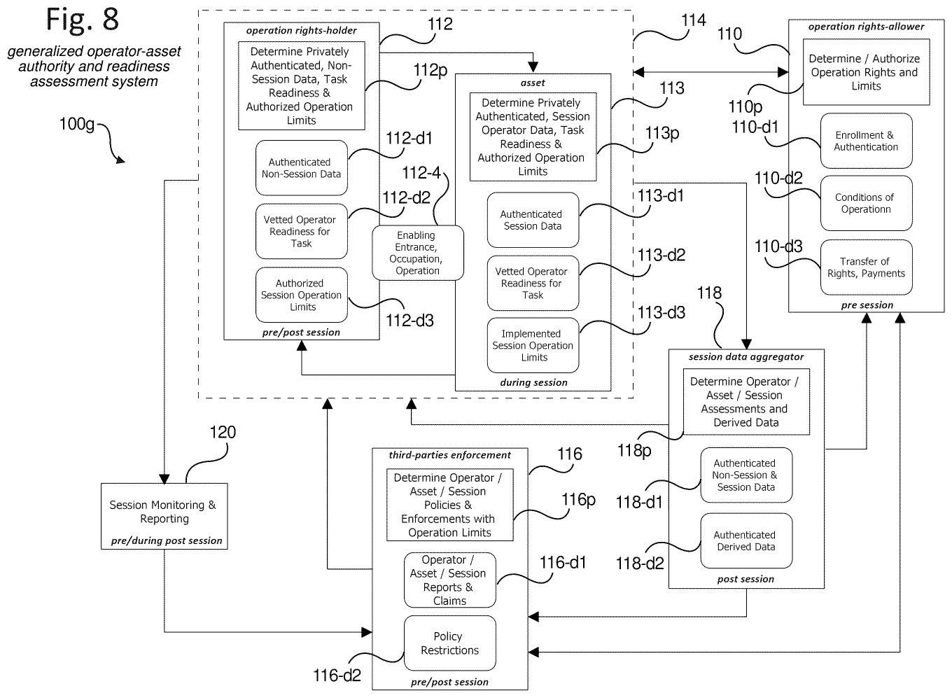

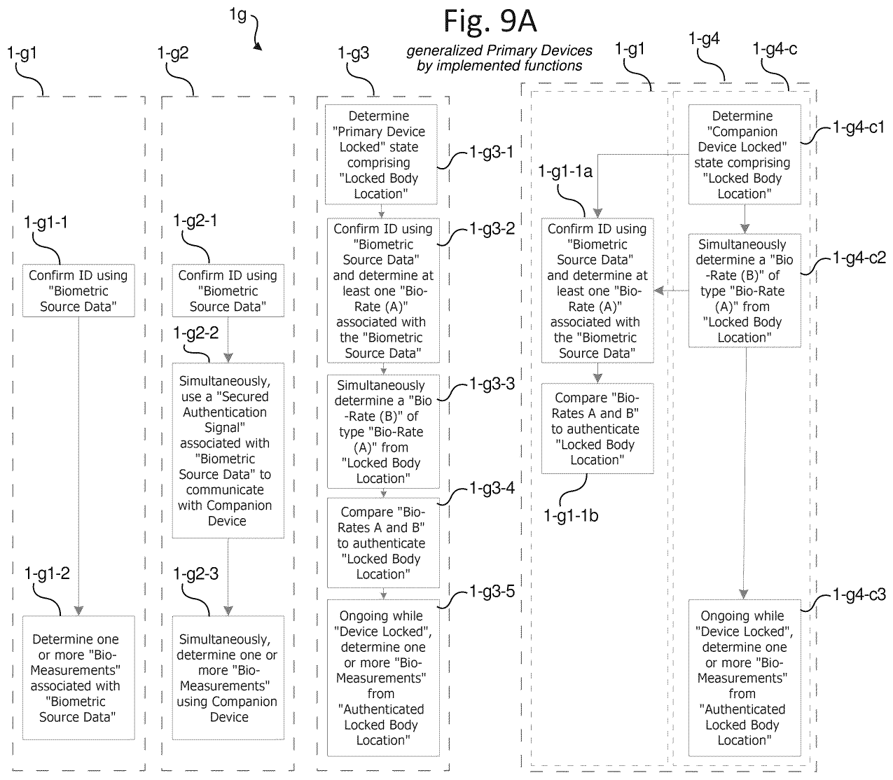

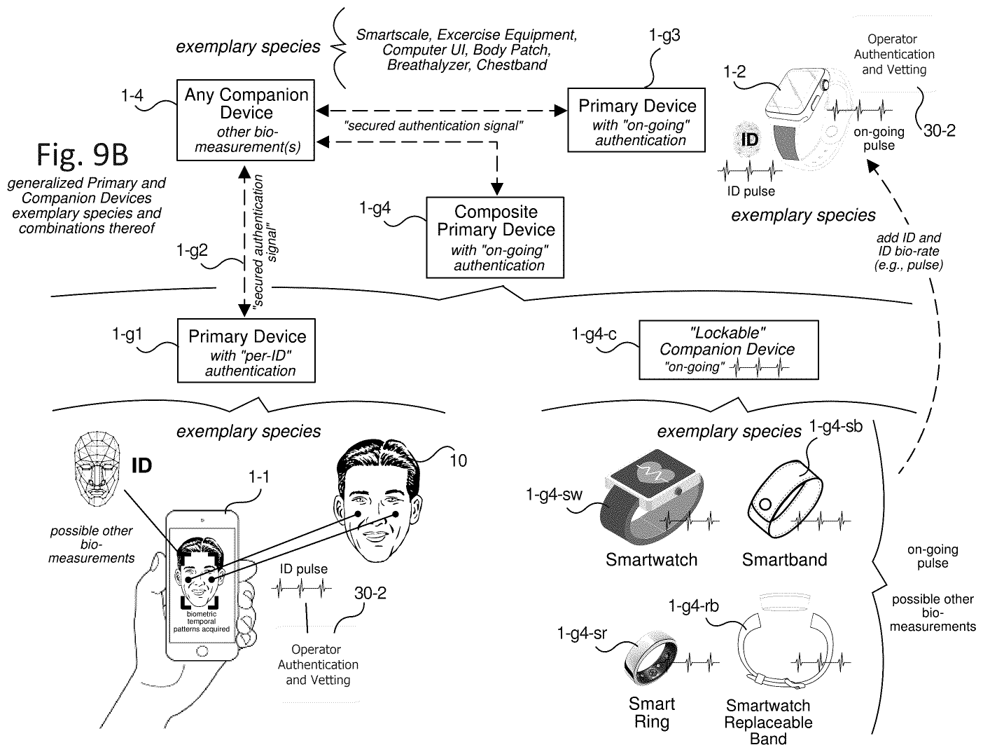

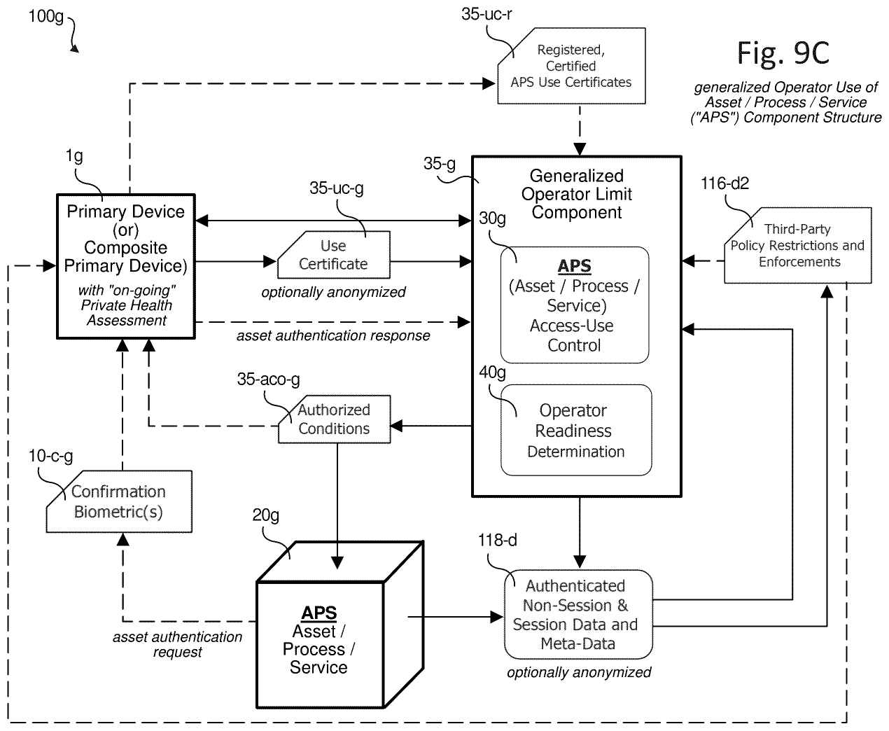

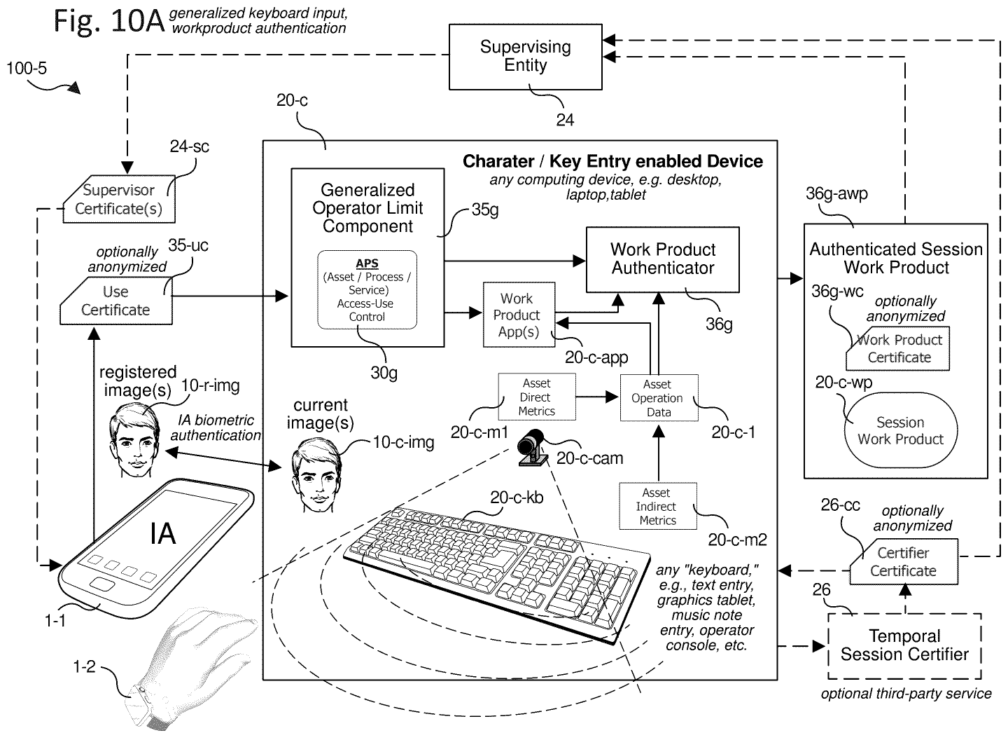

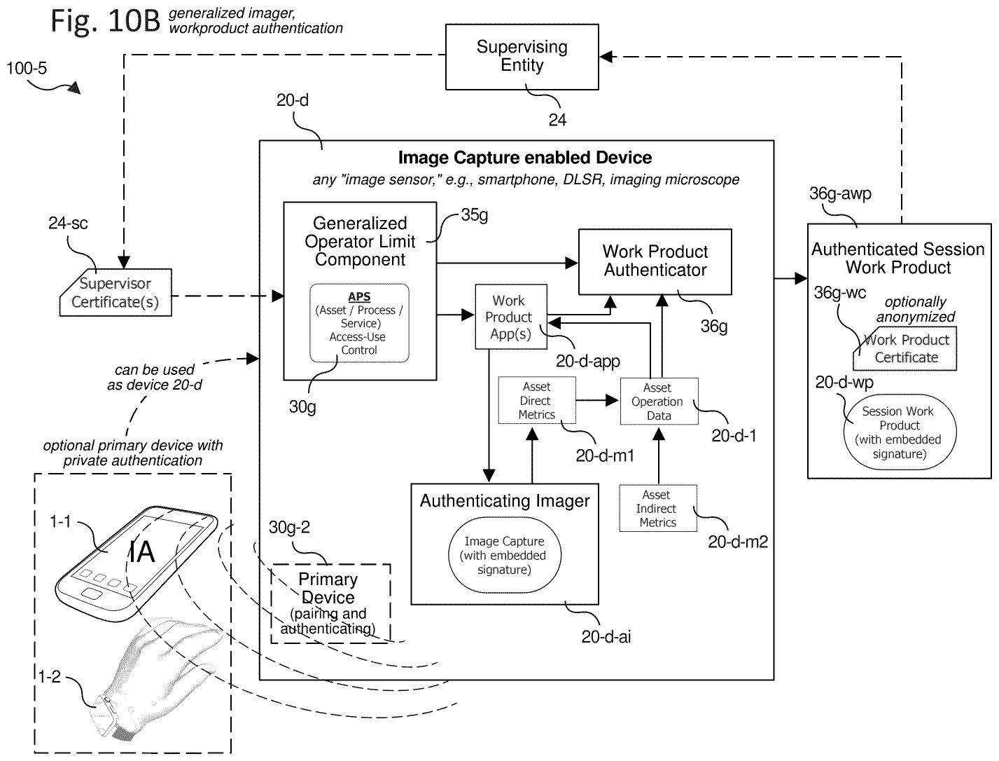

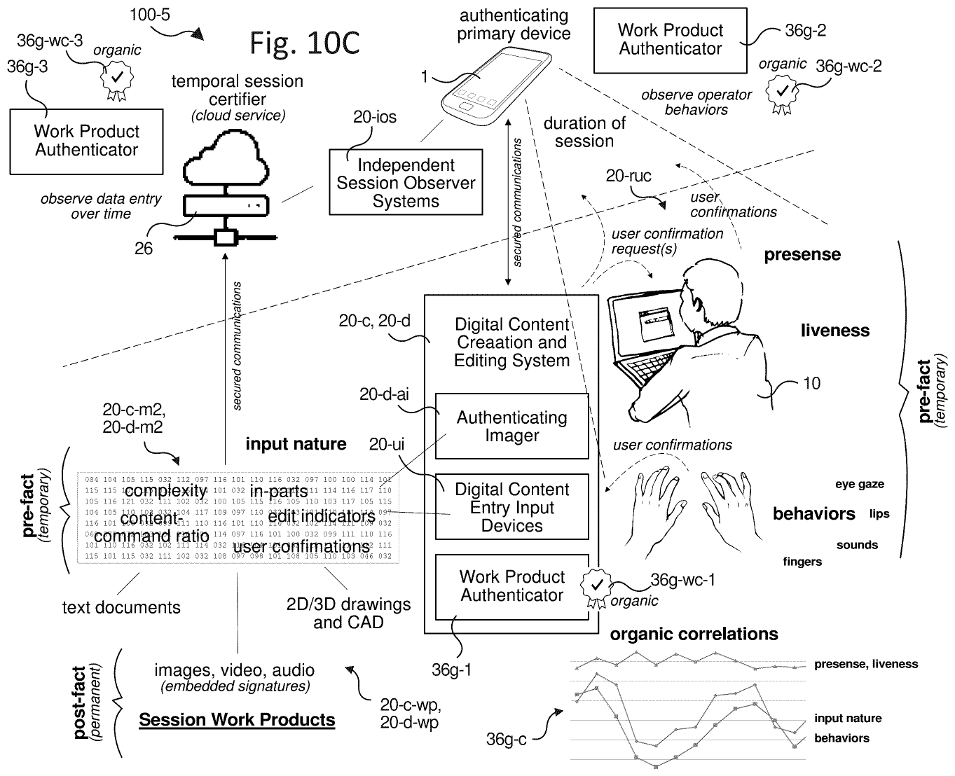

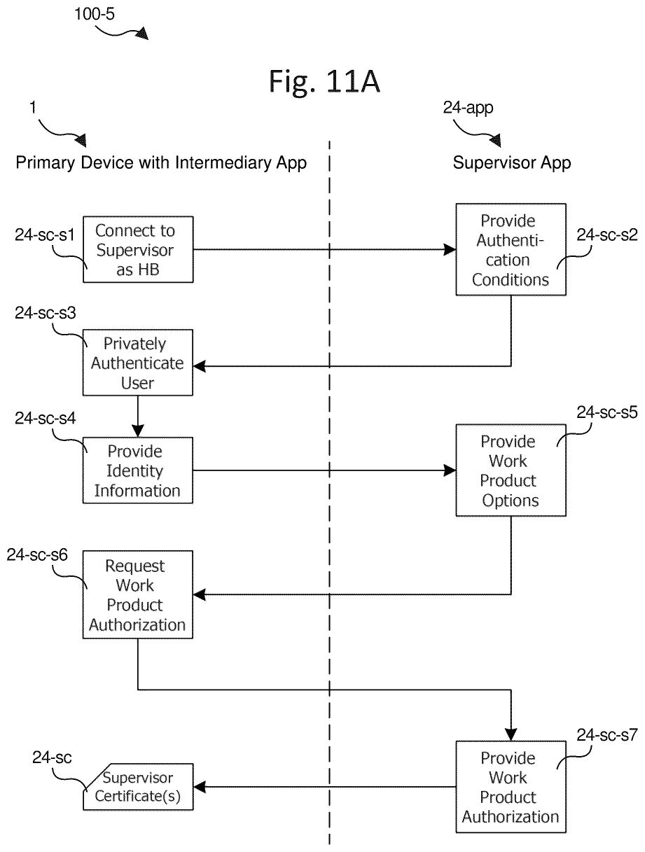

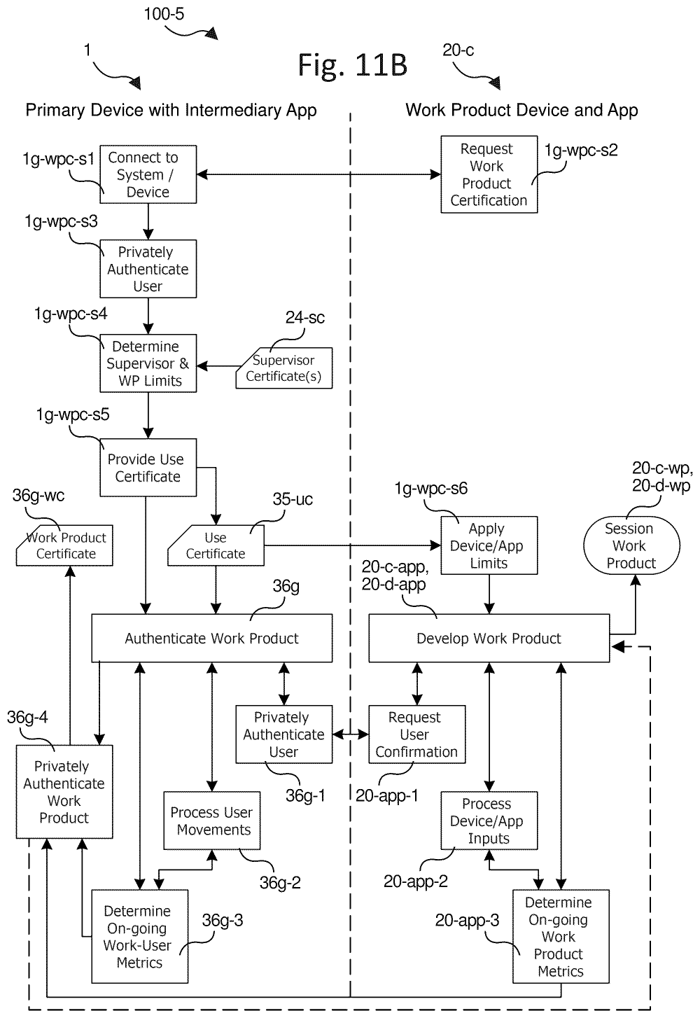

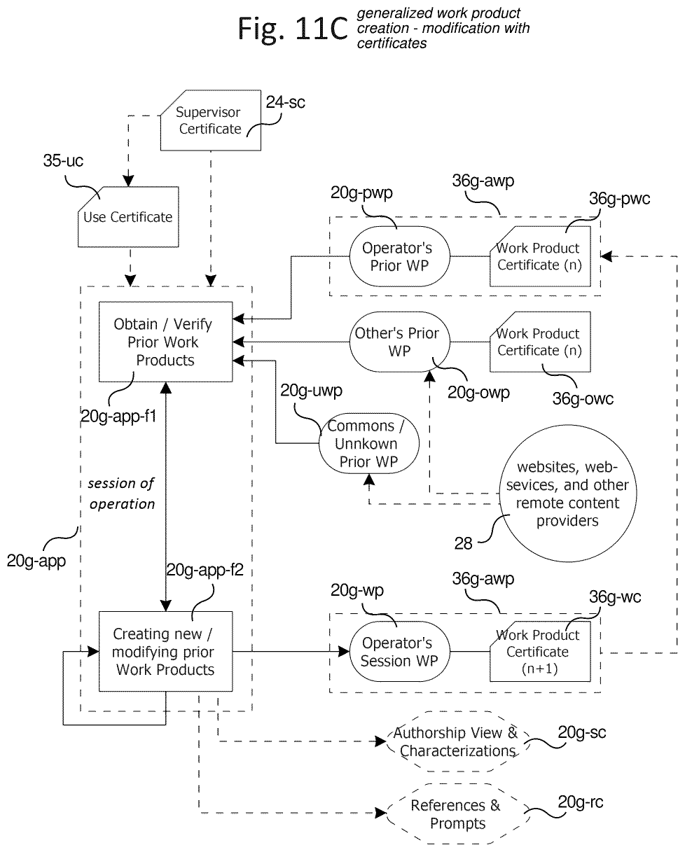

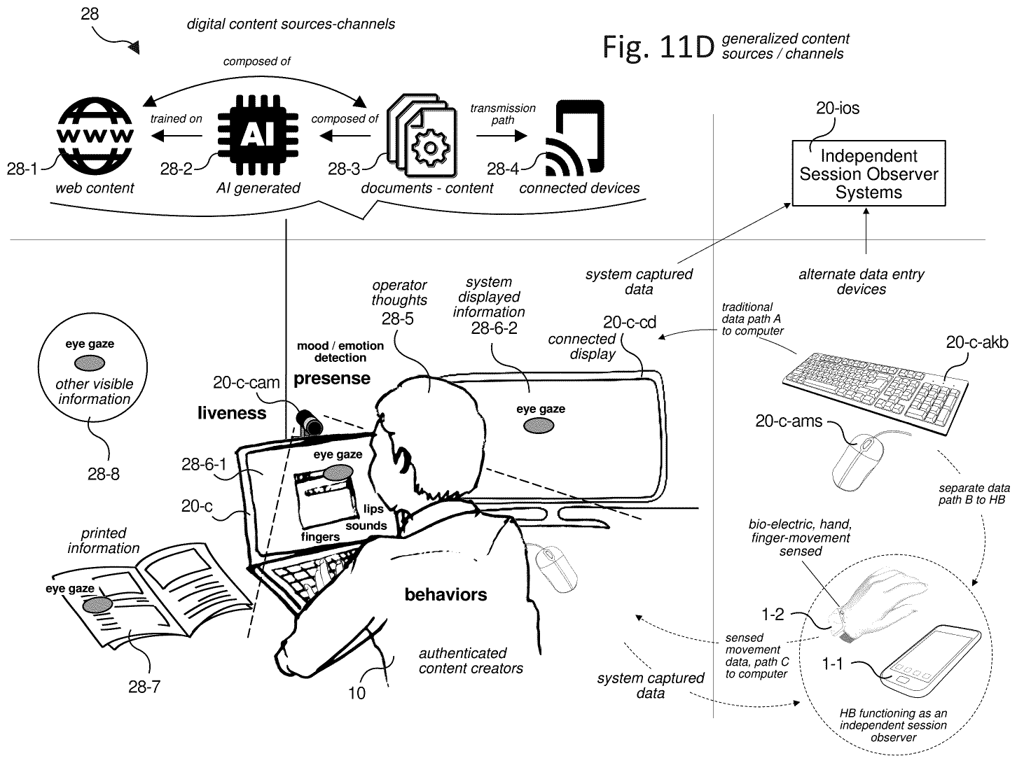

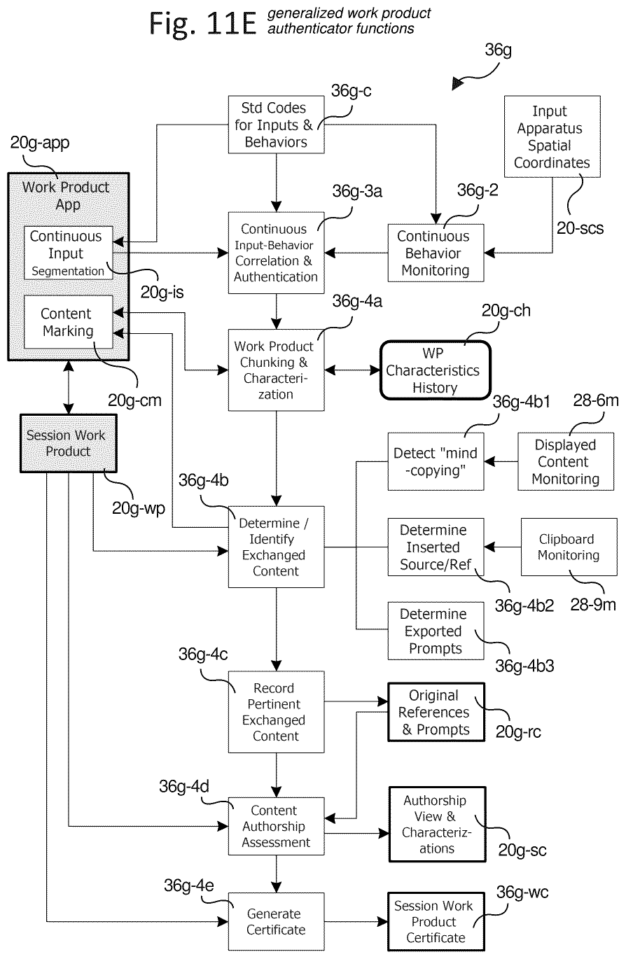

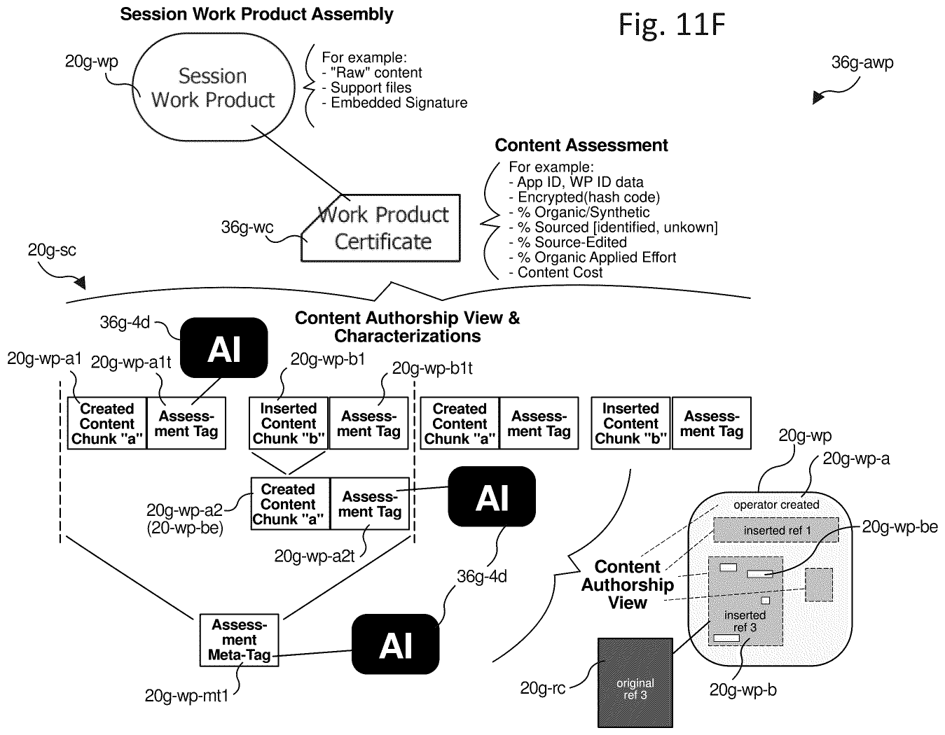

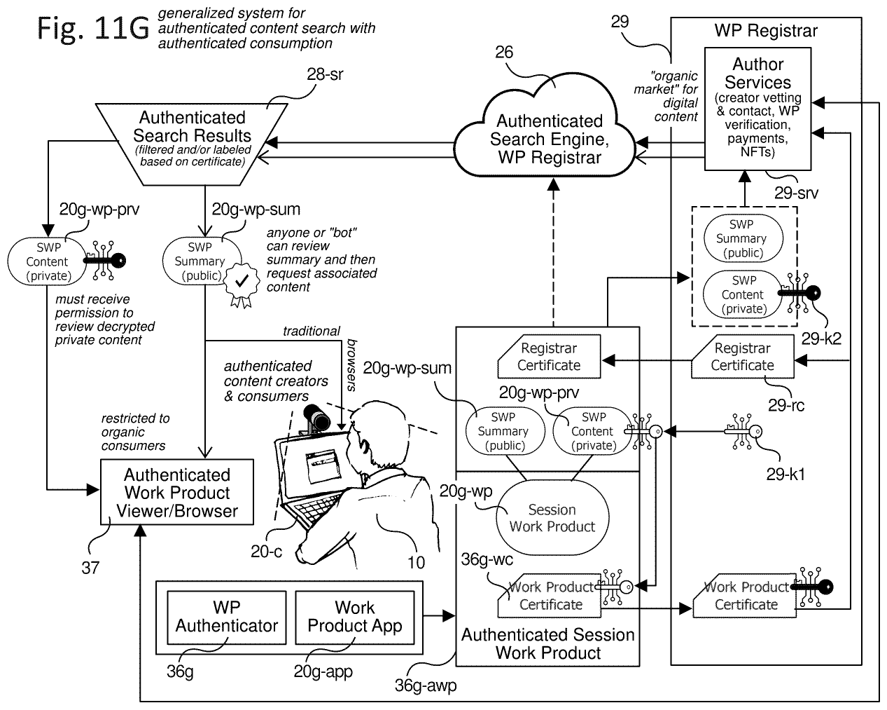

FIG. 1B is a block diagram showing the major components by data of the present system implemented in the exemplary use case of an operator-vehicle (“driver-car”) authority and readiness assessment system 100. The exemplary use case system 100 comprises an operator 10 that engages and operates a vehicle (e.g., a car) 20.

The depicted system 100 data component are aggregated into four main groups comprising authenticated operation context data 10-d1, authenticated operator characterization data 10-d2, authenticated operator/operation derived data 10-d3, and authenticated/anonymized centralized vehicle data 50, where the four data components determine and process vehicle operation data 50-1 related to the operator 10 prior to, during, and after a session of operation of a vehicle 20. Third parties 22 have access to data comprising centralized vehicle data 50 for determining polices/operation restrictions 22-pr. Operator readiness determination function 40-4 has access to any one of or any combination of 10-d1, 10-d2, 10-d3, and restrictions 22-pr for determining vehicle operation limits 40-6.

FIG. 2A is a block diagram including pictorial representations of various components of a preferred decentralized implementation 100-1 of the exemplary use case operator-vehicle system 100 for use in a “private group vehicle sharing” use case. This view is directed to enrollment, authorization, authentication, and readiness vetting enabling controlled vehicle entry. The “private group” (e.g., a family) comprises one or more individuals that are potential vehicle operators 10, some of whom may have pre-known identities, others of whom may be a non-identified “agent/valet.” Implementation 100-1 comprises the use of personal primary devices such as a smartphone 1-1 executing an intermediary app for conducting any of operator authentication and vetting component functions 30-2, where the intermediary app is in communications with devices such as other smartphones 1-1, smartwatches 1-2, key fobs 1-4a, and vehicle 20 “on-board” computing system(s) such as 20-1. Primary devices 1-1 preferably communicate with third-parties such as insurers and renters 22 for conducting contractual transactions including authorization and payment(s) associated with vehicle 20 access and operation, where communication may include the provision of preferably anonymized data to a remote centralized server 50 accessible by third-parties 22. Primary devices such as 1-1 and 1-2 preferably determine and otherwise maintain health state data with respect to a person 10 desirous of accessing or operating a vehicle/equipment 20, where such health state data is usable by operator readiness determination component 40-4 to govern person 10's access and operation in cooperation with vehicle access control component 30-4.

FIG. 2B is a flow diagram describing the major steps and functions associated with the preferred implementation 100-1 of a decentralized “private group vehicle sharing” use case. What is shown is that starting with the intermediary app preferably executing on a device with a larger user interface, like a smartphone 1-1, tablet, laptop or desktop, a person 10 may receive, modify, copy, and generate vehicle access keys for their own use or the use of other members of a group. Functions include an enrollment step 30-ka-5 with an optional step for confirming/obtaining rights, coverage, permissions, etc. from a third-party step 30-ka-7, where step 30-ka-7 optionally includes payments or otherwise some form of a rights-transaction and optionally issues policy restrictions 22-pr for affecting the authorized conditions of operation 30-aco.

Functions also include modifying any of authorized conditions 30-aco in step 30-ka-11 as well as issuing additional keys in steps 30-ka-15 through 30-ka-19 (assuming the authorizing key is a “master key”).

Preferably these “new member” authorization and enrollment steps are a part of operator authentication and vetting component 30-2 as they establish critical information necessary for the preferred vehicle 20 pre-entrance authentication and vetting of an operator 10 such as described in relation to FIG. 2A.

FIG. 3A is a block diagram including pictorial representations of various components of the preferred decentralized implementation 100-1. This view anticipates any type of vehicle 20 entry, traditional (such as a “car key” or number pad) or alternatively authenticated, authorized, and vetted as taught herein (see FIG. 2A,) and is subsequently directed to operator presence detection combined with authenticated, operator readiness vetting, controlled vehicle operation, and vehicle operation data determination. Key teachings of this decentralized approach to vehicle 20 operation include the use of a primary device 1 (such as smartphone 1-1 depicted or watch 1-2 not depicted) with which the potential operator 10 is pre-enrolled including for example having provided a registration biometric such as and preferably an image 10-r-img (see FIG. 2B) and/or a fingerprint. After having entered the vehicle 20, using for example traditional limitations such as a typical vehicle key, vehicle 20 first determines that driver 10 is properly situated using operator detection component 40-4 for example comprising any of seat occupied sensor 20-2, door closed sensor 20-3, or other vehicle sensors and data sources 20-5, preferably whereafter and/or also using vehicle camera 20-4 to determine seat occupation, vehicle 20 then captures or otherwise provides a confirmation biometric such as image 10-c-img of operator 10 for transmission to the primary device 1, whereupon primary device 1 authenticates operator 10 providing in return to vehicle 20 any of an anonymous operator ID 10-id and anonymous operator data 10-aod comprising or otherwise associated with a valid authorized access key 30-4-aak. Vehicle 20 determines and uses any of vehicle direct metrics 20-m1, vehicle indirect metrics 20-m2, route/use characteristics 20-m4, environment conditions 20-m5, or updates to anonymous driver data 10-aod, where uses of such data include determining any of vehicle operation limits 40-6 in conjunction with operator readiness determination 40-4.

FIG. 3B is a flow diagram describing the major steps and functions associated with the preferred decentralized implementation 100-1. What is shown is that the intermediary app executing on a primary device 1 conducts operator authentication and vetting process 30-2 resulting in the provision of an authenticated, authorized and vetted access key 30-4-aak to a vehicle access control process 30-4 for allowing controlled entrance of operator 10 into vehicle 20 and/or controlled operation by operator 10 of vehicle 20. Once situated in vehicle 20, the presence of operator 10 is preferably detected by component 40-2 whereupon vehicle 20 provides confirmation biometrics to the primary device 1 in process-step 40-4-1. After privately authenticating and confirming in process-step 1-pa that the provided confirmation biometrics match registered/authorized biometrics of the operator 10, primary device 1 preferably provides anonymous operator ID and data as well as an authorized access key to operator readiness determination component 40-4 that also receives current indirect and direct metrics 40-1 from vehicle 20, whereafter component 40-4 determines any of vehicle operation limits 40-6 based at least in part upon which the vehicle 20 is operated in process step 30-6. During or upon the completion of vehicle 20 operation, the private data source 1-pds of primary device 1 is preferably updated with session operation data in process-step 40-8-2.

FIG. 4A depicts the primary device smartwatch 1-2 as taught in the copending related applications further adapted to preferably include operator authentication and vetting component 30-2 and optionally include operator readiness component 40-4.

FIGS. 4B and 4C depict two exemplary key fobs 1-4b and 1-4a that have been further adapted to 1) communicate with a primary device, 2) optionally include a scanning means such as an NFC reader for scanning a “valet card” 10-a-c comprising identify information about an agent/valet 10-a desiring entry and access to the vehicle 20, 3) optionally a biometric scanner such as a fingerprint reader 1-4-s1, and 4) internal processing and communications means necessary to accomplish the functions taught herein, including communicating with vehicle 20 for allowing authenticated and restricted entry and operation.

FIG. 5A is a perspective drawing of an exemplary species of bio-conductive authentication signaling depicting an operator 10 simultaneously wearing a primary device such as watch 1-2 while holding and appropriately touching a location on a key fob such as 1-4b, where then the operator 10 forms a bio-connectivity circuit for transmitting signals between the watch 1-2 and the fob 1-4b at least for the purposes of confirming operator 10's authenticity.

FIG. 5B is a block diagram of the generalized genus of secured authentication signaling between a primary device 1 such as a smartphone 1-1 or a wearable 1-2 and a companion device 1-4 (such as but not limited to a key fob 1-4a or 1-4b). The combination of the referenced related pending applications and the present teachings of bio-conductive signaling provide for three types of secured authenticated signaling, namely (1) “light signaling”, (2) “pulse signaling”, and (3) “bio-conductive signaling.” Sufficiently confirmed signaling between primary device 1 and companion device 1-4 results in the provision of operator 10 identity confirmation data by the companion device 1-4 and thereafter the provision of at least authenticated and authorized confirmation data regarding the operator 10. A properly authorized companion device 1-4 provides preferably at least one or both of authenticated companion data 1-4-cd or authorized companion action(s) 1-4-ca to an asset such as a vehicle 20.

FIGS. 6A and 6B depict a person 10 with an overlaid representation of facial veins including the superior labial vein 10-sla using an authenticating breath analyzer 2-2 that is an exemplary companion device 1-4, all as taught in the copending related PCT application PCT/US22/47511 filed on Oct. 23, 2022, entitled MEASUREMENT AUTHENTICATING WELLNESS DEVICES. Breath analyzer 2-2 is able to detect the presence of one or more substances such as alcohol in person 10's breath that might contribute to a measure of potential operation readiness impairment. Analyzer 2-2 as taught in the related application is capable in some configurations of self-authentication while in other configurations cooperative authentication in combination with a primary device 1.

FIGS. 6C and 6D depict a primary device 1 such as a watch 1-2 receiving any of authenticated measurements determined by breath analyzer 2-2, or indications related to such measurements, where then the intermediary app executing on the primary device 1 is able to evaluate the received analyzer 2-2 measurements and/or indications, preferably in light of operation protocols or restrictions/limits (such as insurer policy restrictions 22-pr, see FIG. 2,) to then determine any of access and/or operation request signals to be transmitted directly to a vehicle access control component 30-2 of any of a vehicle (such as 20) or equipment. Either of the breath analyzer 2-2 or a primary device 1 optionally first transmits any of access and/or operation request signals to an access device such as a key fob 1-4, where then fob 1-4 optionally operates to individually or in cooperation determine or otherwise confirm person 10 identity (authentication), and where preferably thereafter communicates access and/or operation request signals to access control component 30-2 of any of a vehicle (such as 20) or equipment. In some configurations, breath analyzer 2-2 is also able to communicate directly with any of a vehicle (such as 20) or equipment to provide any of access and/or operation request signals.

FIG. 7 depicts system 100 in the lower left corner implemented using decentralized authentication variation 100-1, that optionally co-functions with asset/vehicle centralized authentication variation 100-2 (depicted in the lower right,) where then either of variation 100-1 or 100-2 optionally co-function with entity centralized authentication variation 100-3 (depicted in the upper left).

FIG. 8 is a process-data structure diagram of the present operator-asset authority and readiness assessment system 100g that generalizes the exemplary operator-vehicle system 100 across multiple types of assets and operations. Generalized system 100g comprises processes and data associated with each of an operation rights-allower 110, an operation rights-holder 112, an asset for operation 113, an operation session data aggregator 118, third-parties and operation enforcement 116, as well as session monitoring and reporting 120 functions.

FIG. 9A depicts a series of four variations (1-g1, 1-g2, 1-g3, and 1-g4) of a generalized primary device 1-g used for determining and maintaining private and authenticated on-going health measurements for forming a private and authenticated current health status of an operator 10. A purpose of the teachings of these four variations is to help the reader understand the generalized primary device 1-g, implemented in various specie (such as but not limited to 1-1 and 1-2 depicted herein,) for accomplishing the private “authentication” of an operator 10's health state for use in the present readiness assessment system 100.

FIG. 9B is a pictorial-and-block diagram showing the composite primary device 1-g4 comprising a primary device 1-g1 (with “per-ID” authentication) such as a smartphone with intermediary app 1-1 working in combination with a “lockable” companion device 1-g4-c that substantially provides an on-going authentication signal for example a pulse (but does not provide an intermediary app or biometric ID sensor means,) where the provided signal is usable for authenticating health measurements provided by other companion devices after the time of initial authentication of that person. Composite primary device 1-g4 (comprising two separate devices 1-g1 and 1-g4-c) is shown as being similar to a “single-device” primary device 1-g3 with “on-going” authentication. Thus, composite primary device 1-g4 and “single-device” primary device 1-g3 can be seen as substantially implementing the same core functions in a different two-device versus one-device deployment, all as discussed in relation to the prior FIG. 9A.

FIG. 9C is a block diagram of generalized system 100g comprising the two main interacting system objects (1g and 20g) along with a generalized interface 35-g providing for and allowing the two system objects (1g and 20g) to communicate and exchange key data (35-uc-r, 35-uc-g, 35-aco-g, 10-c-g, 118-d, and 116-d2).

System generalized object 1g is any of a primary device 1-g1, 1-g2, 1-g3 or a composite primary device 1-g4 for use by a person/operator 10 to determine any one of or any combination of authentication, authorization, or readiness vetting data prior-to, during, or after a session of operation of the other system object 20g that is any of an asset, process, or service (“APS”) as described or anticipated herein. Generalized interface 35-g is labeled by its main function as an “operator limit component” such that without such an interface 35-g an operator 10 receives substantially (and traditionally) unlimited use of the APS object 20g.

FIG. 10A is a block diagram showing the components of system 100-5 for allowing an operator 10 (not depicted) to use an asset 20-c for the creation of an authenticated session work product 36g-awp, where authentication verifies the “organic” (human directed/human created) nature of the work product 20-c-wp (as opposed to a “synthetic” work product such as created by an “AI generative” process,) and where a work product certificate 36g-wc is generated as being “proof-of-organic.” Asset 20-c is preferably “sedentary” (such as a desktop or laptop computer) providing larger and different user interfaces and supporting the creation and modification by the operator 10 of more complex work products comprising greater amounts of digital content and requiring longer sessions of operation. Operator 10 preferably uses a primary device 1 such as smartphone 1-1 or smartwatch 1-2 to interact with asset 20-c for simultaneously providing measurements of the operator 10's “presence,” “liveness,” or “behaviors correlated with data inputs” that are concurrent and congruent with the data inputs used for determining the creation and modification of a work product 20-c-wp during a session of operation of the asset 20-c. The use of asset 20-c for creating work products 20-c-wp may also be limited according to the requirements of a supervising entity 24, and/or monitored in real-time by a temporal session certifier 26.

FIG. 10B is a block diagram showing an alternative system 100-5 where the asset 20-d is preferably “mobile” such as a smartphone or an “appliance” such as an imaging microscope, but otherwise the asset 20-d either also interacts with a primary device 1 or is the primary device 1. At least some assets 20-d comprise a traditional camera/imaging sensor that is further adapted to be an authenticating imager for embedding one or more signatures into captured “raw” images, where the signatures are usable for proving that the device 20-d produced the image under the direction of the operator 10.

FIG. 10C is a block diagram of system 100-5 with pictorial support for understanding the key concepts for determining work product “organic certification” 36g-1, 36g-2, or 36g-3 pre-fact of created and/or modified post-fact session work product 20-c-wp, 20-d-wp, where “organic certification” is based upon any one of or any combination of (i) determinations with respect to the input nature of the work product 20-c-wp, 20-d-wp as determined by analysis of indirect metrics 20-c-m2, or 20-d-m2, (ii) correlations 36g-c determined between the input nature of the work product 20-c-wp, 20-d-wp and the presence, liveness, and/or behaviors of the operator 10 as determined by analysis of direct data/metrics 20-c-m1, 20-d-m1, and (iii) any of user confirmations made in response to preferably session-random user confirmation requests 20-ruc.

FIG. 11A is a process flow diagram depicting the preferred steps allowing for an authenticated operator to request and receive a supervisor certificate 24-sc, where then the supervisor certificate 24-sc is usable with respect to either of the assets 20-c or 20-d when creating authenticated session work products 36g-awp (see FIGS. 10A and 10B) providing for the restrictions of a supervising entity 24 as comprised within the supervisor certificate 24-sc.

FIG. 11B is a component-process diagram showing the concurrent and congruent operation of a primary device 1 and an asset 20-c or 20-d, where preferably the primary device 1 is executing a component 36g for authenticating the organic nature of a work product 20-c-wp or 20-d-wp as being created or modified responsive to an operator 10 using a work product app 20-c-app or 20-d-app, respectively, where the functions of the asset 20-c or 20-d or app 20-c-app, or 20-d-app can be limited according to a use certificate 35-uc provided by the primary device 1. Authentication of the work product 36g-4 is preferably based upon correlations between tracked “work-user” metrics 36g-3 and tracked “work product” metrics 20-app-3.

FIG. 11C is a block flow diagram depicting the operation of a generalized work product app 20g-app with a focus on “obtaining/verifying prior work products” 20g-app-f1 and “creating new/modifying prior work products” 20g-app-f2, all while still properly vetting and maintaining the associated work product certificate 36g-wc. Thus a work product certificate 36g-wc is first created during an initial session of operation by an authenticated operator 10 using a work product app 20g-app that creates the work product 20g-wp, whereupon closing app 20g-app the work product 20g-wp effectively becomes the operator 10's prior work product 20g-pwp with now a prior work product certificate (n) 36g-pwc. Subsequent sessions of operation by the operator 10 using the work product app 20g-app first confirms a valid prior certificate (n) 36g-pwc and then allow modifications to prior work product 20g-pwp, where upon completion of the session the new modified work product 20g-wp and certificate (n+1) 36g-we are created. Also shown are new optional new artifacts including authorship view and characterizations 20g-sc describing the work product 20g-wp as a possible composition of one or more parts or “chunks of content,” where each chunk has a determined and recorded source and/or reference usable for determining “authorship,” as well as reference and prompts artifact 20g-rc comprising originally inserted content and possible prompts used to at least in part cause the generation or availability of such inserted content.

FIG. 11D is a pictorial representation of the physical environment of an operator 10 using a system 20-c to engage a work product app 20g-app (implied but not depicted) in order to create a work product 20g-wp (implied but not depicted). The physical environment comprises multiple possible sources of information that can be used as component information comprising the final work product 20g-wp, including for example web content 28-1. AI generated (algorithm/“synthetic” content) 28-2, documents (and file) content 28-3, connected device provided content 28-4, operator thoughts 28-5, system displayed information 28-6, printed information 28-7, and other visible information 28-8. The present invention teaches apparatus and methods for best determining any of these possible source of work product 20g-app content during app 20g-app operation (see FIG. 11E). Also depicted are other traditional input apparatus sources that have been further adapted to provide “authentication” support, including authenticating keyboard 20-c-akb and authenticating mouse 20-c-ams, where such authenticating input apparatus additionally provide a unique apparatus ID that can be associated with any of the operator 10, the operated asset such as system 20-c (or device 20-d), a primary device 1 with honest broker being used to authenticate the operator 10, or the work product app 20g-app. Authenticating input apparatus such as keyboard 20-c-akb and mouse 20-c-ams may also provide both a traditional data path “A” to the connected asset (20-c, or 20-d) and a separate data path “B” to a primary device 1 (1-1, or 1-2,) where then these separate data paths A and B can be compared for matching inputs as an additional means for assuring against the insertion of digital content “forged” to mimic operator 10 inputs.

FIG. 11E depicts a flow diagram of the mid-level components (some functions, some data) comprising the preferred work product authenticator 36g operating in combination and coordination with one or more concurrently executing work product apps 20g-wp each creating corresponding session work products 20g-wp. The depicted functions are also used to create session work product certificates 36g-wc, authorship view and characterizations 20g-sc, and original references and prompts 20g-re, all corresponding to a session work product 20g-wp.

FIG. 11F depicts a block diagram of the hierarchy of artifacts comprising the herein taught authenticated work product 36g-awp, including the session work product 20g-wp authenticated by the certificate 36g-wc and described by its distinct compositional components comprising any of created content chunks “a” and inserted content chunks “b,” either chunks “a” or “b” of which have accompanying assessments and linkage or descriptions of any sources/references/and authorship associated with the content comprising the chunk “a” or “b,” as well as linkage to any of original reference content or prompts 20g-rc.

FIG. 11G is a pictorial and block diagram depicting the cyclical flow between an operator 10 for example representing any of a multiplicity of possible creator-modifier (operators) 10 using a herein taught work product app 20g-app and companion work product authenticator 36g to create or update/modify an authenticated work product 36g assembly of artifacts for general distribution over the internet and preferably through the use of author services 29-srv provided by a registrar 29. Authenticated work product 36g-awp is shown to further comprise public summary content 20g-wp-sum verses private content 20g-wp-prv that is preferably encrypted using a registrar key 29-k1. Public summary content 20g-wp-sum of the work product 20g-wp is either made available using traditional means or through any of author services 29-srv or further adapted traditional search engines and web-browsers which then allow for any of filtering, sorting, grouping, labeling, etc., based at least upon work product assessment and characterization information preferably made available in a registrar's receipt 29-rc provided by the registrar and accompanying the public summary 20g-wp-sum. Operators 10 may also be digital content consumers that are first authenticated by the authenticated work product viewer/browser 37 that ensures the content authors 10 and author services 29-srv that the desirous and requestion consumer 10 is a person and not a “bot” or algorithm. The authenticated consumer 10 may only receive and view decrypted private content 20g-wp-prv using the authenticated work product viewer/browser 37, where such access may additionally comprise making a payment via author services 29-srv and receiving proof-of-access/ownership of private content 20g-wp-prv in a form such as a non-fungible token/“nft.”

In the following description, numerous specific details are set forth, such as examples of specific components, examples of various combinations of specific components, types of usage scenarios predicated on certain exemplary combinations, etc. in order to provide a thorough understanding of the present disclosure. It will be apparent, however, to one skilled in the art that the present disclosure may be practiced without these specific details and with alternative components, component combinations, usage methods, all representing many possible implementations or physical deployments of the present teachings, some of which are also described herein. In other instances, well-known components or methods have not been described in detail in order to avoid unnecessarily obscuring the present disclosure. Thus, the specific details set forth are merely exemplary. The specific details may be varied from and still be contemplated to be within the spirit and scope of the present disclosure.

### DESCRIPTION OF THE INVENTION

Referring to FIG. 1A, there is depicted a block diagram showing the major components by function of the present system implemented in the exemplary use case of an operator-vehicle (e.g., “driver-car”) authority and readiness assessment system 100. (The present inventors note that the present system applies to a wide range of vehicles, such as automobiles, aircraft, ships/boats, railway trains, etc., as well as equipment and otherwise processes, some equipment of which may include transportation of the operator while others of which perform any combination of process tasks for accomplishing a physical and/or digital transformation that does not include transportation of the operator, where most interest is focused on such vehicles, equipment, or processes associated with transportation and/or transformation that has one or more safety concerns, either for one or more operators of the vehicle/equipment/process (collectively “assets”,) or for any one or more non-operators that are affected by or could be affected by operation of the vehicle/equipment/process.

Specific use cases are not limited to non-commercial (such as the herein main exemplary use case of private citizens driving automobiles on public roads,) but may also for example include commercial, industrial, military, government, or other applications. Thus, while the present invention is described for example primarily using a specific non-commercial operator-vehicle description, a careful consideration will show that this exemplary “driver-car” use case can be generalization so as to apply to the many other possible uses cases, even where some teachings are limited in some use cases and not limited in others, and as such the present invention should not itself be limited to the present non-commercial operator-vehicle use case description but rather recognized as generally applicable and novel across all manner of vehicles, equipment, and process and types of operation thereof. (See especially FIG. 8 for further discussion on the generalized functions and uses of the present invention.) Discussion will also be provided to describe the technical advantages and benefits of the present invention for use with assets such as office buildings and rental homes where the session of operation is the authenticated, authorized, and vetted occupation of the building/home by any one or more persons/operators.

Still referring to FIG. 1A, at a high-level with respect to exemplary vehicles 20 operated by a driver 10, there are shown the two major system component groups 30 and 40, where component group 30 is purposed for the authentication, authorization, and vetting of the operator 10 at least prior to allowing operation of the vehicle 20, and potentially prior to allowing entry into the vehicle 20. Component group 40 is purposed for the authentication and verification of the operator 10's “continuous readiness” for the safe and proper operating of vehicle 20. At a mid-level, group 30 comprises component 30-2 purposed for the authentication and vetting including validation of authorization and readiness of the operator 10, and component 30-4 purposed for allowing the authenticated and vetted operator 10 to have access and/or control of the vehicle 20. Group 40 comprises component 40-2 purposed to the detection of the operator 10 in a placement with respect to the vehicle 20 to be enabled to operate the vehicle 20, such as being seated in a driver's seat of a vehicle 20.

Component 40-4 is purposed to determine the operator 10's sufficient readiness and even level of readiness to operate/continue operating the vehicle 20, while component 40-6 is purposed to limiting the operation of the vehicle based at least in part upon the determined level of operator 10 readiness, where “limiting” should be understood as determining extents such as minimums and maximums of any given possible operation range, and not necessarily as diminishing a possible operation range, as increasing a “limit” is also anticipated herein. Within system 100, as is taught herein, purposes (such as implemented by components of groups 30 or 40) relating operator 10 authentication, authorization, and vetting of readiness to vehicle operation limits are implemented as any combination of either or both self-compliance 5 and mandatory use 6, where any of the components in groups 30 or 04, or sub-components thereof, may be deployed in any variation for processing in part or in whole on an operator 10's personally controlled device (such as a primary device 1-1, smartphone, or 1-2, smartwatch, see upcoming Fig.'s) or deployed in part or in whole on a processing device embedded or associated with vehicle 20 (such as vehicle communications and control center(s) 20-1, see FIG. 2A,) or some combination of deployment across both devices controlled by operator 10 and embedded within vehicle 20.

Still referring to FIG. 1A and now also the generalized system 100g of FIG. 8, and more specifically referring to the determination of authenticated (and therefore “publicly trustable”) operator readiness, such as implemented in any of operator authentication and vetting component 30-2 (where vetting optionally includes determining a sufficient readiness state of the operator 10) or operator readiness determination component 40-4.

While component 30-2's readiness determination is preferably made prior to a session of operation of an asset such as vehicle 20, thus allowing for readiness information to be used during vehicle 20 access control determinations made by component 30-4, component 40-4's readiness determination is preferably made at the commencement of and during the on-going session of operation of an asset such as vehicle 20, thus allowing for readiness information to be used during vehicle 20 operation to provide for real-time operation limit adjustments.

Regarding “operator readiness,” there is a significant body of growing research and publications discussing what is herein referred to as “common operation impairments” that both are (a) based in everyday choices and behaviors of the operator and that (b) can lead to significant degrading of operational performance, and thus represent a safety risk to the operator 10, a non-operator, and the asset 20. The two main common operation impairments addressed herein result from fatigue and alcohol consumption, both of which are measurable to at least some extent, and barring addictions are considered manageable for the operator 10. Technical goals of the present invention comprise: 1) increasing the measurement data and interpretation accuracy of at least these common operation impairments, where increasing includes providing novel means for integrating authenticated non-session(s) measurement data (see element 112-d1 of FIG. 8) with session(s) measurement data (see element 113-d1 of FIG. 8,) where the session(s) even use different specific assets (e.g., different cars) and even different types of assets (e.g., a car, truck, boat, and aircraft,) thereby substantially improving generalized operation outcome predictability, 2) providing a system that implements operational limitations (see element 113-d3 of FIG. 8) based in part upon the predicted operation outcomes (see “operation assessments” element 50-3 of FIG. 1B) and in part upon any combination of self-compliant 5 or mandatory 6 authorized session operation limits (see element 112-d3 of FIG. 8,) thereby substantially improving group operational governance and liability exposure, 3) providing a system that both maintains the privacy of individual operators 10 while also allowing for the anonymous aggregation of significant operation data (comprising the combination of both non-session and session data, see element 118 of FIG. 8) thereby substantially improving derived assessments for feedback into the system 100, 4) improving overall operational performance across multiple operators 10 and assets 20 by enabling more objective and substantiated policy restrictions (see element 116-d2 of FIG. 8) based upon the operation data and derived assessments, where the policy restrictions are linkable to economic transactions of the operational marketplace (see element 110-d3 of FIG. 8,) thereby increasing marketplace user safety while reducing marketplace partner liability, and 5) several other technical goals as we be apparent by a careful reading of the present teachings.

Still referring to FIG. 1A and now also FIG. 1B, and otherwise in general the teaching provided herein with respect to “operator readiness,” and the two main common measurable and controllable impairments mentioned above, the reader is directed to the article entitled “Fatigue Monitoring Through Wearables: A State-of-the-Art Review,” Martins et. al, published Dec. 15, 2021, in the Frontiers of Physiology. As the article discusses, there are many causes for fatigue including “cognitive overload, physical exertion, sleep deprivation, circadian phase/circadian rhythm disruption, or illness.” A careful consideration of the research will show that these causes typically precede any given “session of operation,” and as such a preferred system such as taught herein privately authenticates and monitors relevant health measurements prior to/leading up to a session. The article states that “fatigue impairs cognitive and/or motor performance, reducing work efficiency, productivity, and product quality, as well as increasing risks for injury and fatality,” for which many statistics are well-known to substantiate this assertion.

The cited research states that “these facts show the important role of continuous monitoring of the level of fatigue in an accurate and unobtrusive manner for early detection and management of fatigue. Among existing technologies, wearable sensors may meet these requirements. Wearables enable continuous, long-term monitoring, paving the way for the development of accurate models for fatigue monitoring in real-time.

Therefore, their application for fatigue monitoring is highly promising.” As taught herein, what is needed is a means for authenticating this continuous “pre-session” monitoring whereby the authenticated pre-session measurements (referred to as personal health metrics 10-m, see FIG. 1B) are then combinable with a different set of authenticated actual performance “session” measurements (referred to as direct metrics 20-m1 and indirect metrics 20-m2,) authenticated “session context” measurements 10-d1, and authenticated operator readiness derived data 10-d3, to create a more complete dataset for operation performance prediction referred to as operator/vehicle readiness metrics 20-m3.

It is not the purpose of the present invention to teach a specific set of health measurements or a particular model or algorithm for best determining a “readiness fatigue score,” as those skilled in the necessary arts will understand that many measurements and algorithms are possible and useful, but rather to show how authenticated pre-session-determined personal health metrics 10-m (already described in the prior related applications) are combinable with other substantially during-session-determined operator characterization data 10-d2 and operation context data 10-d1, as well as other substantially post-session-determined operator/operation derived data 10-d3, to provide for an improvement to any of models and algorithms for best determining “readiness fatigue score.”

The present invention anticipates that operator/operation derived data 10-d3 including instance and type baselines 50-m1 (where a type is a group of individuals 10 with like properties) as well as instance and type history 50-m2 (a specific individual 10) offer a significant technical advantage for determining more accurate and useful readiness scores and otherwise operational outcome predictive assessments. Those familiar with human performance will understand the complexity of creating a generalized formula that accurately spans the range of human bodies and temperaments, where for example it is well-known that sleep requirements can be significantly different between individuals, or that depending upon body conditioning, levels of endurance can also be significantly different.

What is needed is to accumulate large data sets across a broad and varied multiplicity of individuals operating under a broad range of conditions a broad range of assets such that the application of pattern detection algorithms such as machine learning and AI can recognize smaller categories of individuals, conditions, and assets with like proprieties (thus “sub-populations”) that are best-predictive of the individual's future performance. Systems for accumulating this varied set of pre-session and session data, captured even across different vehicle types (such a car versus motorcycle,) and instances within vehicle type (such as “sedan VIN 934 . . . ” verses “SUV VIN 795 . . . ”) are necessarily technically sophisticated and currently not-known, as amongst other considerations personal data privacy is an important consideration.

Additionally beneficial and non-trivial, is the determination of individual (“instance”) performance baselines based in part upon captured histories, where for example an individual's performance can be related to their “normals” (forming their baselines,) which might be significantly different between individuals. Thus, and for example, a younger person might perform better in operational tasks than an older individual even if both individuals have substantially the same objective “rest scores,” (hence the younger individual has similar performance with the a “lesser” rest state assessment,) where then it is also anticipated that the younger person might have a different “average rest state” prior to an “average operation” thus their predictive outcome is best predicated upon their average/baselines for “rest state,” all as will be understood by those familiar with human physiology and its effects on task accomplishment. The operation performance of an asset by an individual may then also be predicted by assigning the individual to a “sub-population” and then using this “sub-populations” baselines as comparatives to the individual (especially with there are no to minimal histories accumulated for the individual).

Those familiar with human performance will also understand that authenticated historical session performance data 50-m2 is highly useful for at least in part determining readiness scores. However, this desirous data 50-m2 remains technically unavailable in traditional systems caused at least by the lack of operator and session authentication (necessary to confirm that a current session's performance data that will become “session history” actually belongs to a given session operator,) but also caused by the lack of technical systems for coordinating the authenticated/anonymized aggregation of underlying operation data 10-d1 and 10-d2 which is then processed for feedback to the operator instance and type histories 50-m2.

The interested reader in fatigue measurement of operators is also directed to: “Smart Alert Systems for Driver's Drowsiness Detection.” Kumar et. al. published Aug. 18, 2022, in the International Journal of Engineering Research & Technology (IJERT); “Managing Transportation Worker Fatigue with Wearables,” published Nov. 14, 2018, in Health and Fitness, and “Wearables That Detect Fatigue,” published Feb. 6, 2018, on unitedrentals.com. A careful understanding of the co-pending cross-referenced applications will show that these various referenced “fatigue measurement” technologies, which are not currently authenticated, can be authenticated using the co-pending and present teachings (see present FIG. 5B for a discussion on “secured authentication signaling” usable for authentication,) where this authentication combined with other present teachings provide for the many significant technical advantages and market benefits described herein.

Regarding the second of the two main common measurable and controllable impairments, namely alcohol levels, the reader is directed to the following articles: “Wearable technology can detect and control drinking habits,” published Jan. 28, 2022, on openaccessgovernment.com, and “Compact Wearable ‘Lab on the Skin’ Continuously Monitors Glucose. Alcohol, and Lactate,” published May 9, 2022, by the University of California, San Diego. As stated above, the co-pending and present invention provides means for privately authenticating data collected by personal wellness devices (herein “primary devices” and “companion devices”,) where for example the collected health measurements (such as the amount of alcohol found even in “imperceptible amounts of sweat” or in the human breath can be detected using either a primary device such as a smartwatch 1-2, see FIG. 6C, or a “paired” companion device such as a breathalyzer 2-2, see FIG. 6B). A careful reading of the former article entitled “Wearable technology can detect and control drinking habits” will show that researchers are aware that a single current measurement of alcohol (such as using an authenticated breathalyzer as taught in the co-pending applications and herein FIG. 6B) taken for example just prior to an operator's “request to operate” an asset (such as a car,) lacks valuable “drinking history,” where history for example includes “how much a person drank, how quickly that drank, and how long alcohol remained in their system,” which can however be determined pre-session using an authenticated wearable such as a smartwatch 1-2, see FIG. 6C. The article also points out that “different levels of alcohol have different outcomes for people,” thus the need for baselines.

A general understanding will also make clear that impairments can have a compound effect, and as such on-going pre-session authenticated measurements for the estimate of drowsiness combined with on-going pre-session authenticated measurements for the levels of alcohol, both with commensurate histories and baselines, provide even greater advantages for predicting asset operation performance. A careful consideration will also show that authenticated operator profiles (usable for determining “sub-populations,” and that can also remain private) such as age, sex, body weight, body mass index, typical activity levels, vision qualifications, etc., all further combine with the private baselines, impairment histories, and session operation histories to improve authenticated personal prediction models, especially as will be understood by those familiar with machine learning and AI algorithms. Blood glucose levels are another important physiological measure that can affect performance and compound with other impairments, where it is known already that glucose levels can be measured using current wearable technologies and at least authenticated using the co-pending taught apparatus and methods.

Thus, the careful reader will see that the “readiness determination” generally described herein with respect to operator vetting in component 30-2 and operator readiness determination in component 40-4 may have many and various implemented algorithms using the same or different physiological or psychological measurements, taken either or both prior-to or during a session of operation, such that what is important to see is that regardless of the specific readiness determination algorithm(s) to be implemented, some of which are at least partially described in the art, some of which will become known, the present invention teaches a means for creating the necessary novel authenticated datasets for supporting any such privately authenticated and readiness determinations and for using such determinations for at least the purposes of asset access and/or limited operation.

Referring again to FIG. 1A, self-compliance 5 implementations of readiness determination comprise a primary device such as a wearable 1-2 (see FIGS. 2A, 4A, 5A, and 6C) being used to maintain an on-going health state of an individual/person/operator 10, all as a careful reading of the co-pending cross-references will further describe. Thus, the operator 10 is privately tracking their own current health state (e.g., over a continuous “14 rolling days”) which, as will be well understood by those familiar with health measurements routinely provided by current wearable technologies, may comprise one or more indications of “impairment,” such as an estimated physical rest levels that is impacted for example by the combination of sleeping patterns and/or physical exertion level patterns, all of which are readily determined by most “fitness trackers” and even “smartwatches,” (The reader is also reminded that the co-pending applications teach how a primary device 1 can pair with and authenticate health measurements communicated by a secondary device, e.g., a chest band, a breathalyzer, etc., or tertiary device, e.g., exercise equipment, smart scale, glucose patch, etc., in order to obtain additional authenticated health measurements not determined by the primary device, thus extending the possible health measurements that can be used for the implementation of a robust and reliable indication of a current health status including a current estimate of “operation impairment” fitted to the anticipated operation of the anticipated asset. (Note that herein secondary and tertiary devices are also collectively referred to as “companion devices.”)

These same primary devices 1 may be indicated as “mandatory use” 6 within authorized conditions of operation 30-aco (see FIG. 2A for initial descriptions,) for example where a parent who is an owner of a vehicle enrolls children for use of the vehicle including restrictions, one of which being that to either or both access and operate the vehicle, the child must be wearing/using a primary device 1 that includes a currently up-to-date health status that is usable for determining a “readiness to operate” (for example combining assessments of impairment including fatigue, alcohol, and glucose levels, all matched to the child's baselines and histories) as associated with operator authentication and vetting component 30-2 and/or operator readiness determination 40-4. Other mandatory use 6 scenarios include law enforcement requiring the use of at least an authenticating breathalyzer (see element 2-2 in FIG. 6B, or using a smartwatch 1-2) prior to access or operation of a given vehicle 20. In still other mandatory use 6 cases, a vehicle or otherwise asset 20 rental company may require authenticated operator 10 readiness assessment prior to allowing access and/or operation of a rented vehicle/asset 20. Large trucking companies can use the present invention to regulate and enforce readiness conditions for their employees/drivers, where such authenticated and determined “sufficiently ready use” can become important documentation for any potential accident litigation.

It is also well-known within the current vehicle insurance industry that devices can be connected to an automobile's communication port (see on-board access port 20-1 of FIG. 2A) for monitoring vehicle motion, such as speed, acceleration, deceleration, measures of turning, time-of-use, duration-of-use, location-of-use, etc., all of which is then combinable into vehicle indirect metrics 20-m2 (see FIG. 3A) used for “scoring” the vehicle's usage and thus indirectly the driver/operator 10, where then this score is applied to potential changes in the insurance rate. This type of use case can be considered as either self-compliant 5 or mandatory use 6, where a careful consideration of the present teaching will show the many possible improvements enabled by the present teachings as compared to capabilities of current devices such as “Snapshot” used by Progressive Insurance.

Still referring to FIG. 1A, each of components 30-2, 30-4, 40-2, 40-4, and 40-6 will be discussed and taught herein as both implementing key functions, and as being deployed in preferred (apparatus/“device”) embodiments, where the careful reader will recognize that the key functions have alternative embodiments (physical “deployments”) while then still implementing the teachings and spirit of the present invention. More specifically, any part or all of, any or all of the depicted components 30-2, 30-4, 40-2, 40-4, and 40-6, can be embodied as apparatus primarily associated with/“worn on”/“carried by”, etc., the operator 10, thus depicted as a “operator (with device)” 10, where these any zero or more devices associated with the person 10 may be used to implement all of or part of the functions described in the present application as performed by the mentioned components 30-2, 30-4, 40-2, 40-4, and 40-6 as well as other components to be detailed herein.

Alternatively, any part or all of, any or all of the depicted components 30-2, 30-4, 40-2, 40-4, and 40-6 can be embodied as apparatus primarily associated with/“built into”/“attached to”, etc., the vehicle 20 (thus depicted as a “vehicle (with device)” 20,) where these any zero or more devices associated with the vehicle 20 may be used to implement all of or part of the functions described in the present application as performed by the mentioned components 30-2, 30-4, 40-2, 40-4, and 40-6 as well as other components to be detailed herein.

FIG. 1B is a block diagram showing the major components by data of the present system implemented in the exemplary use case of an operator-vehicle (“driver-car”) authority and readiness assessment system 100. The depicted system 100 data components are aggregated into three main groups of data, 10-d1, 10-d2, and 10-d3, collectively comprising vehicle operation data 50-1 that is aggregated by centralized vehicle data system 50 for processing by component 50-2 to determine vehicle operation assessments 50-3. Authenticated operator characterization data 10-d2 comprises four types of data including personal health metrics 10-m that are determined at any time prior to, during, or after a session of operation using a “primary device” (see co-pending applications and the remaining present specification) and zero or more “companion devices.” Such determined health metrics 10-m form preferably an “on-going” authenticated health assessment of an operator 10, where authentication includes a process assuring that the health assessment comprises health measurements verified to be “of the operator,” and thus the health assessment is considered to be “publicly trustable” (again, see the co-pending applications). The assessment preferably comprises information indicative of, or otherwise sufficient for determining a “readiness to operate vehicle score,” where for example the scoring algorithm is able to make predictions related to operation impairments such as fatigue levels, alcohol levels, glucose levels, medication levels, etc., for which many such related algorithms are well-known in the art and will become known.

Many current marketplace vehicles 20 comprise apparatus and methods for determining real-time vehicle direct metrics 20-m1 and vehicle indirect metrics 20-m2 substantially during the operation session of a vehicle 20, and thus relatable to the driver/operator 10. Regarding the traditional definition of direct 20-m1 versus indirect 20-m2 metrics, the interested reader is directed to the article entitled “Fatigue detection systems explained,” by Vivek Shah, published Jan. 10, 2021 by CarExpert. (Although the article is discussing the detection/determination of data for the prediction of driver fatigue levels, the present invention is generalizing these concepts beyond fatigue in particular to any “driver impairment” in general or otherwise a predictive assessment of “operator readiness” for the indented “operational task” using the intended “asset.”) The cited article explains that “There are two main types of fatigue detection systems available on cars sold today. More common are indirect systems” (thus determining indirect metrics 10-m2) “that monitor vehicle movement through a road-facing front camera to look for signs associated with driver fatigue. The second type of system uses one or more driver-facing cameras and face tracking technology to look directly for signs of fatigue, such as head movements and how often the driver is blinking” (thus determining direct metrics 10-m1).

While the present invention is incorporating the common marketplace terms of “direct” versus “indirect” systems, the present invention should not be limited to currently available direct versus indirect systems comprising various already known and implemented apparatus and methods, as the teachings provided herein both explicitly and implicitly comprise and extend current apparatus and methods, where it is also known that new technologies already exist but have yet to be implemented for determining any of direct 10-m1 and indirect metrics 10-m2, and still yet other technologies will become known and available. What is important to see is that the present invention provides amongst other technological advantages the ability to authenticate any current and future metrics 10-m1, 10-m2 as “belonging to” the “operator-asset” pair, where then this authentication allows for several additional technical advantages and benefits as taught herein including the derivation of instance and type baselines 50-m1 and instance and type histories 50-m2 comprising authenticated operator/operation derived data 10-d3 that is vehicle operation assessments 50-3 as determined by a process of operation assessment 50-2.

Still referring to FIG. 1B, the aforementioned CarExpert article mentions that indirect systems “look for signs in driving style or the driver's behavior that can commonly be attributed to fatigue,” where a careful reflection will show that “driving style” is not individuated (thus assuming that all person have the same single “style”) and that what is preferred is to determine a “driving style” that is individuated to the operator 10, where this individuation necessarily requires that preferably all gathered information such as 10-d1 and 10-d2 be authenticated to be “of” the operator 10 such that derived information 10-d3 (comprising for example one or more metrics indicative of the “operator's driving style”) is also authenticated to be “of” the operator and thus individualized. It is also noted that using currently available systems, a “driver's behavior” as stated in the article is a “current driver's behavior” assessment based upon any of a vehicle's direct and indirect measurements and is otherwise neither authenticated nor aggregated for processing into any of derived data 10-d3 that includes individualized “specific driver's behavior” data. This “current driver's behavior” limitation is further shown by the article's comment “this initial behavior is sampled and stored as a benchmark for comparison with later parts of the journey,” where initial behavior at the start of each session is current behavior. A careful consideration will show that the driver's prior performance, for example from a prior trip earlier in time, has no bearing using traditional systems on the “current driver's behavior” as this data is essentially lost and not authenticated, stored, and accumulated with respect to the individual driver.

The article points to these current driver limitations that can only consider data determined and the commencement of and during an existing session of operation with comments such as “another flaw is the assumption that the driver is alert and well-rested at the beginning of their journey. It is quite possible someone has commenced their journey already tired (for example, after a sleepless night).” The present teaching overcome these many limitations through both the authentication of current data 20-m1 and 20-m2, the joining of this currently authenticated data with authenticated baselines 50-m1 and histories 50-m2 (thus “historical data”) along with pre-session personal data 10-m regarding the authenticated operator.

Still referring to FIG. 1B, as will be discussed more in relation to FIG. 3A, by authenticating operator 10 (of which several variation methods are provided herein) either prior to allowing vehicle access (thus using authenticated, authorized, vetted access control 30) or upon entry and preferably prior to session operation (thus using authenticated, verified, driver “continuous-readiness” assessment 40,) the present system is then also able to advantageously authenticate vehicle direct metrics 20-m1 measured directly “of” the authenticated operator for determining on-going readiness, and vehicle indirect metrics 20-m2 measured of the vehicle's operation with indirect implications regarding the operator's on-going readiness. Operator readiness determination function 40-4 preferably has access to any and all of personal health metrics 10-m, vehicle direct metrics 20-m1, and vehicle indirect metrics 20-m2, for the processing by some “readiness algorithm” into one or more operator/vehicle readiness metrics 20-m3.

Still referring to FIG. 1B, just as the authentication of operator 10 using either of component groups 30 or 40 supports the authentication of all operator characterization data 10-d2, in a likewise manner operation context data 10-d1 is also authenticated (i.e., as relating to operator 10, and some identifiable and distinct session of operation by operator 10 of some identifiable and distinct asset, such as a particular vehicle, equipment, or process). Those familiar with currently available automobile technologies, and using a careful consideration, will recognize that significant data is available for measurement regarding what is herein referred to as the vehicle's “operation context,” or specifically in terms of a driver-vehicle relationship, any other non-operator, non-vehicle, related data. This other related data for example includes route/use characteristics 20-m4, such as indications of the driver's intended route as can be sufficiently determined by interfacing with any of several variations of well-known “GPS mapping systems,” as well as environment conditions 20-m5, such as any measures of the current and predicted along-route weather or otherwise ambient conditions for which many well-known sensors are available, and where some GPS systems are capable of providing at least some current along-the-route weather conditions and patterns. Route/use characteristics 20-m4 preferably also comprise current traffic patterns along with a measure of the authenticated driver 10's “route familiarity,” where the authenticated driver's instance and type histories 50-m2 for example comprise historical indications of specific routes already driven (thus supporting familiarity,) types of routes driven (e.g., “local city” versus “open road”,) lengths of trips, etc., where then also these same instance (i.e., “specific driver”) histories 50-m2 also include indications of readiness and performance assessments for those histories including specific routes and types of routes, where these indications also comprise histories 50-m2 data.

The present invention further anticipates that one or more “route complexity” algorithms can be readily established such that for example a combination of factors such as time of day, weather conditions, number of turns, length of route, traffic congestion levels, etc., can be translated into for example one or more scalers for scoring the route and accumulating in the authenticated historical records 50-m2, where then analysis of this historical data 50-m2 is useful for determining operator familiarity not just with the specific route or the type of route, but also the complexity of the route, providing great technical advantage for predicting performance/operation success. (Again, it is noted that the specific exemplary use case of a drive-car should not be limiting, as for example “asset-operation-complexity” is generalizable into virtually all the herein anticipated asset classes along with their many and varied types and contexts of operation, where the present system 100's unique technical ability to authenticate this historical data across virtually all operator-asset-operation combinations provides for new and advantageous types of predictive methods.)

Those familiar with current vehicle technologies will recognize that any and all of this operation context data 10-d1 and operator characterization data 10-d2 is currently not available as authenticated data and thus also not aggregated as authenticated vehicle operation data 50-1 which may then be processed into vehicle operation assessments 50-3. Assessments 50-3 are based at least in part upon any of data 10-d1 and 10-d2 (and also data 10-d3 prior determined by system 50,) where any of data 10-d1, 10-d2, and 10-d3 are associated with any one or more operators 10, operating over any one or more sessions of operation, where each session is relatable to any one or more assets (in this example vehicles,) the full combination of which provides greater data for determination of operation assessments process 50-2 comprising for example the use of machine learning and AI algorithms that show significant improvement as data models scale in complexity and training data per model feature increases, all as will be well-known to current algorithm developers.

Data 10-d1 further comprises context readiness metrics 20-m6 while data 10-d2 comprises operator/vehicle readiness metrics 20-m3, where these readiness metrics 20-m6 an 20-m3 are preferably determined by processing any of other respective information 10-d1 and 10-d2, such that the current system provides a means for “scoring” or ranking both the context and the current readiness state of the operator 10 as well as the state of the asset, and in this example vehicle 20, for which many algorithms are possible as will be well understood by those familiar with algorithms and software. Simple readiness metrics can be expressed as a scaler, for example from 1 to 5, where foe example a “1” readiness is the most affirmative and a “5” readiness is the most disapproved, and where such readiness scores are usable for setting authorized conditions of operation (see element 30-aco in FIGS. 2A, 2B, and 3A for more discussion). Each of the context, operator, and asset may have an independent readiness scaler, where then a single scoring/rank/scaler is determined based upon the combination. It is not the purpose of the present invention to teach a preferred algorithm for any of such possible readiness metrics 20-m6 or 20-m3, of which many are possible, but rather to teach a system for aggregating the authenticated data 10-d1, 10-d2, and 10-d3 necessary for determining such readiness metrics with increased accuracy and individualization, all as a careful consideration of the present teachings will show.

Still referring to FIG. 1B, any of vehicle operation data 50-1 and vehicle operation assessments 50-3, as preferably anonymously aggregated by system 50, is then made available for analysis and determinations made by any one or more third parties 22, such as operator and vehicle (asset) insurers and vehicle (asset) renters or providers. It is also not the purpose of the present invention to teach any specific analysis or determinations to be made by third parties 22, as many such analysis and determinations are possible, only that the aggregated information provides a significant technical advantage to such parties 22, where then the present invention anticipates that parties 22 will determine any of policies 22-pr including any operation restrictions based at least in part upon any of the provided assessments 50-3 (comprising any of 10-d3) or operation data 50-1 (comprising any of 10-d1 and 10-d2,) regardless of the state of anonymization.

And finally, FIG. 1B depicts the flow and provision of any one of or any combination of authenticated operation context data 10-d1, authenticated operator characterization data 10-d2, authenticated operator/operation derived data 10-d3, or polices 22-pr for use by operator readiness determination component 40-4, for example to provide any one or more vehicle operation limits 40-6, as will be discussed in more detail especially in relation to upcoming FIG. 3A.

Referring next to FIG. 2A, there is shown a block diagram including pictorial representations of various components of a preferred implementation 100-1 of the present exemplary operator-vehicle system 100 for use in a decentralized “private group sharing” “driver-car” use case. The “private group” (e.g., a family) comprises one or more individuals that are potential operators 10 of a vehicle 20, some of which may have pre-known identities, others of which may be a non-identified “agent/valet.” In a preferred embodiment, a first-member of the group of individuals 10 uses a primary device 1 such as a smartphone 1-1 executing an intermediary app to access a master access key 30-4-mak preferably comprising person 10 enrollment/ID information and vehicle 20 ID information as well as optional authorized conditions of operation 30-aco and optional policy restrictions 22-pr as issued by a third-party 22, where the first member uses the intermediary app to either (1) complete enrollment and/or modify their own authorized conditions, or (2) issues one or more new restricted access keys 30-3-rak to other members of the group 10.

Issuing additional restricted access keys 30-4-rak enrolls or allows enrollment of one or more second-members of the group 10 for authorized operation of the vehicle 20 limited to authorized conditions of operation 30-aco. Enrollment includes assigning, providing, or otherwise determining any of sufficient uniquely identifying personal information regarding or associated with the enrolled individual for whom the key 30-4-rak is issued, where sufficient uniquely identifying personal information comprises any one of or any combination of biometrically substantiated (e.g., a facial image or fingerprint) or non-biometrically substantiated (e.g., a unique pass code) information. The enrolling process is begun and optionally completed by the first-member but may also be completed by the second-member (using their down primary device 1 with intermediary app).

FIG. 2A depicts “decentralized” enrollment (see also the lower left of FIG. 7,) where in decentralized enrollment all personal information preferably remains private to any one or more intermediary apps (executing on a primary device 1) associated with/used by any member within the group of members 10, where remaining private comprises any of well-known data and communication technologies for encrypting, securing, or otherwise keeping private at least any of personally identifying information such that it is accessible to the first-member enrolling themselves, a first-member enrolling or partially enrolling a second-member, or a second-member completing an enrollment process enabled for them by a first-member, but otherwise is not accessible to any other party without provision and authorization by the member 10 for which the personal information applies.

As will be discussed further within the present application, in a decentralized authentication solution 100-1 versus a centralized authentication solution 100-2 or 100-3 (see FIG. 7,) preferably substantially all data remains private including any of enrollment data especially including personally identifying data such as names and biometrics, as well as all operator characteristics data 10-d2, operator/operation derived data 10-d3, and authenticated operation context data 10-d1, where then it is also understood that in some centralized systems such as 50 it is possible to anonymize select personal information and other data 10-d1, 10-d2, and 10-d3 thus combining features of anonymous centralization with privatized decentralization, all as will be well understood by those familiar with the art of software systems and data privacy.

A means for ensuring the maintenance of personal privacy is to have all personal data remain encrypted and stored on the operator's primary device 1, such as a smartphone 1-1 or smartwatch 1-2, where the primary device 1 serves both to authenticate and maintain the operator 10's current health status (see the co-pending related applications, also component 30-2 of FIG. 1A) that is used at least in part to determine any of personal health metrics 10-m, and then to also store and maintain preferably any of other data 10-d1, 10-d2, or 10-d3.

Storing and maintaining any of data 10-d1, 10-d2, and 10-d3 on the operator's primary device 1 provides the additional advantage of “cross-asset” sharing, where for example an individual operator 10 at times operates different vehicles 20 using the same primary device 1, and as such the datasets 10-d1, 10-d2, and 10-d3 comprised on primary device 1 are representative of and shared across all of the different vehicles 20 and the different possible sessions of operations.

As will be discussed in relation to upcoming FIG. 3A, it is also possible to store only some of the data 10-d1, 10-d2, and 10-d3 on the operator's primary device 1, such as the personal data including for example personal health metrics 10-m, operator readiness metrics 20-m3, and any of operator derived data 10-d3, but then to store all other data not stored on the primary device 1 on for example a data storage system (represented as element 20-1) comprised in the vehicle 20, or even in a remote server available for access via any of well-known communications with either of the primary device 1 or the vehicle 20. In relation to FIG. 3A, there is shown that the primary device 1 provides an anonymous operator ID 10-id to the vehicle 20's computing system 20-1 such that any or all of data 10-d1, 10-d2, and 10-d3 can also remain upon the vehicle's system 20-1 and stored in relation to the not-personally identifying anonymous ID 10-id, thus ensuring personal privacy while allowing at least some of the data storage and processing to be conducted on the vehicle's system 20-1. Note that the primary device 1 retains a copy of the anonymous ID 10-id provided to any particular vehicle/asset 20, and thus can always retrieve this information using the same ID 10-id.

Those familiar with means for maintaining data privacy will understand that using a remote server (such as cloud-services provided by Microsoft's Azure, Amazon AWS, or similar,) it is possible to remotely store and maintain substantially all of information 10-d1, 10-d2, and 10-d3 while still maintaining privacy and also allowing cross-asset sharing, where then the primary device 1 and vehicle computing system 20-1 are in preferably secured communications with the remote server for providing and accessing data 10-d1, 10-d2, and 10-d3. Thus, as will be understood by especially those familiar with networked computing systems and data privacy, there are many well-known ways to provide the goals of decentralized system 100-1 that include personal data privacy, and as such any particular implementation portrayed and discussed herein while either preferred or alternative, is otherwise not meant to be limiting of the present invention as many solution architectures are possible without departing from the spirit of the invention.

Still referring to FIG. 2A, associated with enrollment, potential second-member operators 10 may be assigned “authorized conditions” 30-aco for vehicle 20 operation by a first-member, where authorized conditions 30-aco comprise restrictive “a-b” relationships between (a) any one or more of controllable vehicle 20 operation features and settings, thus a limitable feature of vehicle 20's operation along with a description of the limit(s), and (b) any one or more of controlling factors, where controlling factors (b) comprise (b1) operator factors based at least in part upon any of operator characterization data 10-d2, (b2) context factors based at least in part upon any of operation context data 10-d1, and (b3) operation derived factors based at least in part upon any of operation derived data 10-d3. Transferable restricted access key data 30-4-rak preferably comprises or is otherwise associated with authorized conditions of operation data 30-aco describing any of the “a-b” relationship(s) for governing a second member's allowed operation of vehicle 20.

Thus, the process of decentralized enrollment includes a holder of a master access key 30-4-mak (presumably the first-member 10) preferably self-selecting a second member 10 (thus at least partially enrolling the second member) and authorizing this second member for select operational use of vehicle 20, where the holder/first-member is presumably using a first primary device such as 1-1 with access to a master access key 30-4-mak (or a restricted access key 30-4-rak comprising digital information that comprises authorization for making one or more additional restricted access keys 30-4-rak,) and where a result of the enrollment process includes creating a restricted access keys 30-4-rak for a second-member 10. (See FIG. 2B for a more detailed explanation of the preferred flow of steps for issuing access keys to second members and modifying access keys by first or second members.) The careful reader is again reminded that the present use case of an operator 10 being a driver and an asset 20 being a vehicle is exemplary and should not be considered as limiting as the exemplary driver-car use case discussions are generally applicable over a wide range of non-driver-car use cases.

The intended second member 10, presumably using a second primary device 1 such as smartphone and intermediary app 1-1 (typically comprising a larger user interface as compared to alternative primary device 1 that is a wearable/watch and intermediary app 1-2,) then receives the restricted access key 30-4-rak, where enrollment potentially continues for example by requiring the second-member 10 to provide any of personal information for registration such as a name and one or more biometrics such as a face image and/or fingerprint, and then optionally allowing the second-member 10 to modify select authorized conditions of operation 30-aco. This process of enrolling members 10 and assigning limits 30-aco, spanning functions performed by a first-member 10 on a first primary device 1-1 and optionally extending to functions performed by a second-member 10 presumably (but not necessarily) on a second primary device 1-1 are collectively and preferably functions performed as a part of the operator authentication and vetting component 30-2 included with the intermediary app (see especially FIG. 2B,) although as will be understood by those skilled in software and apps, these functions can be provided as one or more other, for example “branded,” apps that are distinct from the core personal health authentication and tracking app (also referred to as “the honest broker app” in the copending related applications).

Still referring to FIG. 2A, a primary device such as 1-1 is able to communicate with other primary devices such as a smartwatch 1-2 executing intermediary app functions and a “fob”/electronic access key such as 1-4a optionally executing intermediary app functions, thus transferring a “final” authorized accessed key 30-4-aak along with any of authentication, authorization, and or vetting/limits information comprising authorized conditions 30-aco for use by vehicle access control component 30-4. The present invention refers to this as the “first authentication, to ‘enter’” the vehicle 20, that is distinguished from the “second authentication, to ‘operate”’ the vehicle 20 (see FIGS. 3A and 3B). As a careful consideration will show, it is possible for a “person 1” to authenticate sufficiently to open a vehicle door providing access, and then to allow a “person 2” to actually enter and then attempt to operate the vehicle 20, where the present invention provides for a double-authentication process to ensure a valid operator 10 both entering and operating, and can also simply implement the “second authentication” such that any person can enter a vehicle, but only a properly authenticated, authorized, and vetted person can then become an operator 10.

As a careful consideration will show, a final key 30-4-aak is referred to a “final” substantially for its place in the herein taught process, thus being used by vehicle access control component 30-4 to provide governed vehicle 20 access and/or operation, and as such it should be understood that any validly enrolled key such as a master key 30-4-mak or restricted key 30-4-rak can serve substantially as-is for a final authorized access key 30-4-aak. It should also be understood that any of devices such as a primary device 1 substantially comprising intermediary app functions such as smartphone 1-1 or a smartwatch 1-2, or a companion device substantially not comprising all or any of intermediary app functions such as key/fob 1-4a (or 1-4b, see FIG. 4B) can communicate with, or even further comprise, parts or all of vehicle access control component 30-4, where component 30-4 using at least in part the provided access key and optionally any authorized conditions 30-aco is responsible for communicating with vehicle 20 (such as through vehicle communications and control component 20-1, see FIG. 3A) for providing governed access/operation to a group member 10.

As a careful reading and consideration of the cross-referenced related teachings will show, a primary device such as smartphone 1-1 or smartwatch 1-2 is anticipated to be determining and otherwise maintaining or comprising an on-going authenticated health state/status of the group member 10 desiring access and operation of an asset such as vehicle 20, where this health state is preferably determined in accordance with a public protocol/regiment (see co-pending related applications,) and where the health state is based at least in part upon one or more authenticated personal health metrics 10-m (FIG. 1B) that are either physiological and/or psychological and preferably determined prior to the attempted access or operation of vehicle 20.

This on-going authenticated health state and/or any of the personal health metrics 10-m are usable at least in part to perform a pre-vehicle-entry “vetting process” (component 30-2) that can be represented as any of operator readiness metrics 20-m3, such as readiness scalers indicative of types and levels of impairment. As prior discussed, exemplary readiness scalers might be indications of alcohol, medications, blood-sugar, or drowsiness impairments, or some combination thereof. Thus, preferably a primary device 1 with an intermediary app implementing component 30-2 determines and provides any of operator readiness metrics 20-m3, but otherwise primary device 1 with intermediary app at least provides one or more authenticated personal health metrics 10-m either directly to a vehicle 20 component such as communications and control component 20-1 comprising vehicle access control 30-4, or to an data transferring device such as key-fob 1-4a, where then amongst other functions key-fob 1-4a provides any of transferred metrics 20-m or 10-m to vehicle control component 20-1. Regardless of the receiving source, in at least one configuration, the vehicle 20 (for example using control component 20-1) processes any received metrics 10-m (using an operator readiness determination component 40-4 to determine any of readiness metrics 20-m3) and/or receives pre-determined readiness metrics 20-m3, where then vehicle 20 comprising vehicle access control component 30-4 selectively allows/denies access by operator 10 to the asset/vehicle 20, and allows/denies other operational functions of the vehicle 20, based at least in part on any of metrics 10-m or 20-m3.

Still referring to FIG. 2A, a companion device such as key-fob 1-4a performs at least one function, that is to authenticate the person (desirous of becoming operator 10) currently pressing a key to request the vehicle 20 to unlock the door or otherwise allow entrance/access (thus key-fob 1-4a is either implementing any or part of vehicle access control component 30-4 or providing information to component 30-4,) where specific apparatus and methods for determining this authentication are discussed further in relation to FIGS. 4A, 4B, 4C, 5A and 5B. Thus, unlike a traditional key-fob for which any (essentially unauthenticated) person can employ a key-fob and gain access to a vehicle 20, the present invention limits vehicle access using a key-fob 1-4a to an authenticated operator 10, and then possibly also a vetted operator 10 with either or both of sufficient personal authenticated health metrics 10-m or authenticated readiness metrics 20-m3. The careful reader will see that in at least one configuration, the primary device 1 (such as a watch 1-2) is currently authenticating the operator 10 and directly provides authentication information such as an anonymous operator ID 10-id (see FIG. 3A) along with optional vetting data to the access control component 30-4 that is for example executing on vehicle communications and control center 20-1, where then the key-fob 1-4a additionally provides to component 30-4 an “authenticated request” for example to unlock (i.e., allow access to) the vehicle 20, where this request preferably comprises an encrypted signal providing an anonymous operator ID 10-id along with an “unlock vehicle” request signal, and where vehicle access component 30-4 compares the two received anonymous ID's 10-id whereupon sufficient match then executes the request signal.

In yet another variation, the operator 10 employs key-fob 1-4a, for example by pressing and “unlock vehicle” button, for which a signal is transmitted by the key-fob 1-4a to a primary device 1 such as a smartphone 1-1, and where this signal includes encrypted information authenticating or otherwise allowing for authentication of the operator (for example by capturing and transmitting a fingerprint, see FIG. 4B). The intermediary app on the primary device 1-1 then confirms the operator by comparing the authentication information provided by the key-fob 1-4a to pre-register authentication information already known to the intermediary app. Upon sufficient match, the primary device 1-1 then directly provides the “authenticated request” for example to unlock (i.e., allow access to) the vehicle 20 to the access control component 30-4 that is for example executing on vehicle communications and control center 20-1, where this request preferably comprises an encrypted signal providing an anonymous operator ID 10-id along with an “unlock vehicle” request signal, but alternately simply provides an authorized “unlock vehicle” signal that does not include any operation authentication information such as anonymous ID 10-id. (Note that an authorized signal for example includes encrypted information identifying the intermediary app's own “proof-of-authenticity”/“honest-broker ID”) that can be validated by the access control component 30-4 such that the access control component 30-4 doesn't “know who the operator is” but “trusts the honest broker,” as a careful consideration will make clear.) Other variations with respect to the coordination of information associated with operator authentication and vetting component 30-2 as well as vehicle access control component 30-4 are possible, and thus the present invention should not be limited to the depicted and our described interactions, but rather seen in the broader sense to include interactions between a primary device 1, a key-fob such as 1-4a or 1-4b, and a vehicle access control component 30-4, where the interactions provide any of operator 10 authorization, or authentication, along with a “request,” such as “unlock” the vehicle 20.

Those familiar with the various deployment architectures for implementing a multi-device software system will understand that any of software functions comprising components 30-2, 30-4, 40-2, 40-4, or 40-6 (see also FIG. 1A) may be distributed as discrete functions implemented essentially on a “person-device” such as 1-1, 1-2, 1-4a or 1-4b, and/or on a “vehicle-device” such 20-1, where those familiar with current automobile technology will recognize that a typical modern car comprises at least one computer processing and data storage element (herein 20-1) that is able to communicate with other non-vehicle 20 devices either wirelessly or through physical ports such as what is known as an “on board diagnostics 2” port. As such, the present invention should not be limited to a specific hardware-device architecture for deploying the herein described software components such as 30-2, 30-4, 40-2, 40-4, or 40-6, as many variations are possible without departing from the present teachings and spirit of the invention. Thus, a potential operator 10 may use any of primary devices 1-1 or 1-2 as well as fob 1-4a or 1-4b (see FIG. 4B,) individually or in combination with a vehicle computing element 20-1, to perform the necessary operator authentication and vetting 30-2 and operator readiness determination 40-4 prior to or as a part of gaining access and/or control of at least some aspects of the vehicle 20 by interacting with vehicle access control 30-4 using authorized access key data 30-4-aak.

Preferably at the completion of a session of operation, vehicle 20 communicates with a primary device such as 1-1 or 1-2 (for example using a vehicle communications and control center 20-1) in order to provide any of vehicle 20 determined vehicle operation data 50-1 (i.e., any of 10-d1, 10-d2, 10-d3) preferably associated with the anonymous driver ID 10-id that is known to and was originally provided by the primary device 1 (see FIG. 3A,) such that this vehicle operation data 50-1 is aggregated at least by the primary device 1 for analysis and processing to effect future sessions of operation, where primary device 1 then optionally provides any of vehicle operation data 50-1 (preferably after anonymizing) to a centralized vehicle data aggregator 50 for further processing. Alternatively, control center 20-1 communicates some or all of this vehicle operation data 50-1 directly to a centralized vehicle data aggregator 50. Third parties 22 preferably have access to the centralized vehicle data aggregator 50 and at least in part use any of vehicle operation data 50-1 or vehicle operation assessments 50-3 (see FIG. 1B) for determining policy (operation) restrictions 22-pr for provision to the primary device 1.

Referring next to FIG. 2B, there is shown a flow diagram regarding the issuing, enrollment, and modification of access keys with associated authorized conditions as implemented in the preferred decentralized system 100-1. Preferably a first-member 10 owning, renting, or otherwise associated with a vehicle/equipment 20, uses a primary device 1 such as the preferred smartphone and intermediary app 1-1 to complete the various steps described in the present figure. Smartphone 1-1 is preferred due to the combination of its mobility, its use for collecting and maintaining a “portable health state/status” (see the cross-referenced art,) and its spatially larger user interface (at least as compared to a smartwatch 1-2,) where those familiar with software systems will understand that there is no particular limitation to a smartphone 1-1, and where for example the functions described in FIG. 2B can also be performed by the taught intermediary app executing on a computing device such as a tablet, laptop or desktop computer, or even a smartwatch 1-2. Those familiar with software systems will also understand that the various functions described throughout the patent as well as in the present figure may be distributed and performed across multiple cooperating computing devices such that the descriptions related to the present figure should not be limited to being performed on a single device, let alone only on a smartphone 1-1.

Still referring to FIG. 2B, person 10 uses computing device such as 1-1 to operate an intermediary app (that for example mediates between the a group member 10, other primary devices 1 operated by other group members or possible group members 10, companion devices such as key-fob 1-4a, 1-4b or breathalyzer 2-2 (see FIG. 6B,) the vehicle/equipment 20, a remote centralized database 50, and any of third parties 22, where the app in a step 30-ka-1 is used by member 10 to acquire an existing digital access key 30-4-cak, where for example the existing access key 30-4-cak is a master access key 30-4-mak for example provided by a car manufacturer, a car rental service, a car sharing service, or otherwise a third party having a relationship to the vehicle 20. Following key acquisition, in step 30-ka-3 member 10 optionally selects to first start, continue, or complete an enrollment of their personally identifying information with respect to the existing access key 30-4-cak (with the presumption enforceable by the intermediary app that the existing key 30-4-cak is not already enrolled to a different member 10).

Completing enrollment step 30-ka-5 is either performed separately or combined with confirming with governing third-parties step 30-ka-7 and comprises one or more various traditional enrollment functions such as but not limited to 1) providing personally identifying information such as a name, address, phone number, etc., and preferably any one of or any combination of biometrically substantiated unique personal information (e.g., a facial image or fingerprint) or non-biometrically substantiated unique personal information (e.g., a unique pass code,) 2) providing contact information, 3) communicating with a third party system for obtaining permissions, confirmations, restrictions, and otherwise access and operation information, 4) providing payments or otherwise engaging in a transaction for enabling vehicle 20 access and/or operation. 5) electronically “pairing” with or otherwise registering association with one or more other primary devices such as a smartwatch 1-2 or other companion devices such as a key fob 1-4a, 1-4b, a companion device such as breathalyzer 2-2 (see FIG. 7B,) or a computing element associated with a vehicle 20 such as an on-board communications and control center 20-1, where various ways of pairing and registration between electronic devices are well known in the art and sufficient for the present teachings, and where pairing or registration provides for current and/or future case of preferably secured communications and exchange of information between the paired devices, and 6) indicating account passwords and permissions especially as it pertains to the storage and processing of personal and vehicle operation data 50-1 such as 10-d1, 10-d2, and/or 10-d3, where storage and processing may be in part or in whole implemented on any one of or any combination of a primary device 1, a vehicle/equipment 20, a remote centralized server 50, or otherwise a third-party computing system.

Still referring to FIG. 2B, step 30-ka-7 optionally comprises the provision by and receiving from a third-party digital data comprising policy restrictions 22-pr, where policy restriction include any indications of conditions of operations 30-aco thus limiting the use and operation of a vehicle 20 by the enrolling member 10. In an optional step 30-ka-9, member 10 choses to enter step 30-ka-11 in order to modify any of authorized conditions 30-aco within the limitations of any provided policy restrictions 22-pr, where any such modifications comprise revised access key 30-4-vak. If the intermediary app determines in a step 30-ka-13 that the existing access key 30-4-cak is encoded as a master key (thus permitting the member 10 to create new access keys 30-4-nak using the master key,) the intermediary app in step 30-ka-13 allows the member 10 in a step 30-ka-15 to choose to create a new access key 30-4-nak. When creating a new access key 30-4-nak, the member 10 optionally performs step 30-ka-17 to specify authorized conditions 30-aco associated with the new access key 30-4-nak, where the conditions are preferably first defaulted to the conditions 30-aco associated with the existing key 30-4-cak.

In either case of accepting default conditions, modifying default conditions, or setting new conditions without default, a member 10 also issues the new access key 30-4-nak in step 30-ka-19, where issuing comprises 1) preferably first selecting a type of member 10, such as an identified person (like a family member) or a non-identified person (like a service agent/valet), 2) for an identified person, optionally starting an enrollment process, for example by providing (such as a name) or capturing (such as an image or fingerprint) any of identifying information, and/or additionally providing or causing the intermediary app to provide an enrollment notification for communication to an intended person (such as via text or email,) where enrollment notification may include links for downloading or otherwise accessing an intermediary app for starting and/or completing enrollment, as well as security information (such as time limits) and codes for ensuring the proper person is completing and/or accepting enrollment including any of requests for return confirmation of provided codes, and 3) for a non-identified person such as a service agent or parking valet, optionally starting an enrollment process, for example by scanning encoded information provided by the agent (such as a barcode, a QR code, or an NFC readable code,) capturing personally identifying information of the agent including ID codes, names, images, fingerprints, etc., and/or additionally providing or causing the intermediary app to provide an enrollment notification for communication to an intended agent (such as via text or email,) where enrollment notification may include links for downloading or otherwise accessing an intermediary app for starting and/or completing enrollment, as well as security information (such as time limits) and codes for ensuring the proper agent is completing and/or accepting enrollment including any of requests for return confirmation of provided codes.

As will be well understood by those skilled in the art of software and user interfaces, there are many variations possible for implementing the teachings with regard to the present FIG. 2B, where some variations include only portions of the stated functions, other variations include functions in a different sequence, and still other variations include additional functions, such that the present flow should be considered as exemplary for the purposes of teaching the core anticipated and preferred system 100 in general, and decentralized system 100-1 specifically. Any such software will have points of exit 30-ka-21 such as either automatically taken by the intermediary app or as indicated by the member 10 using the app.

What a careful consideration will show is that the teachings of the present figure provide a “looping” process where for example a first-member with a master access key 30-4-mak uses the intermediary app to cause a new access key 30-4-nak to be generated and issued to a second-member, where any of a first-member or a second-member use the process to modify, update or even upgrade operational conditions associated with an existing access key 30-4-cak (where upgrading optionally includes step 30-ka-7 for confirming with governing third-parties,) where any of a first-member or a second-member with an existing access key 30-4-cak comprising master access key certification can then issue one or more other new access keys 30-4-nak to one or more other group members 10 including identified and non-identified persons, and where these new access keys 30-4-nak are themselves either authorized or not authorized as master keys 30-4-mak allowing the creation of additional keys.

Referring next to FIG. 3A, there is shown a block diagram including pictorial representations of various components of the preferred decentralized implementation 100-1. This view and teaching anticipates any type of vehicle 20 entry, including (a) traditional (i.e., “open the door and get in” without authentication, authorization, and/or vetting) or alternatively, (b) access enabled based upon some or all of authentication, authorization, and vetting as discussed in relation to FIG. 2A. Traditional entry (a) with the option of alternative entry (b) is herein collectively referred to as “first-optionally-authenticated” vehicle entry and is directed to the physical entrance of the operator 10 into a position (assumed to be a front driver's seat) for then further or presumed beginning to operate the asset/vehicle 20 (such as “turning the vehicle on”). (Again. FIG. 2A was primarily focused on this “first” “getting in the door” function.) The present FIG. 3A presumes that the operator has/will enter vehicle 20, either authenticated, authorized, and vetted or not, (thus “first-optionally-authenticated). The focus of teaching regarding FIG. 3A is referred to herein as “second-authentication” after entry (a) or entry (b), presuming a situating of the operator 10 such as in a vehicle 20 front driver's seat, after which the operator 10 is then authenticated, authorized, and/or vetted before determining any final asset/vehicle 20 operational limits.

In the decentralized system 100-1 depicted in FIG. 3A, driver 10 enters vehicle 20 to be situated in the driver's seat as is traditionally known, thus entering the vehicle 20 possibly without yet being “first” authenticated (identified) or vetted for readiness (hence, presuming “worst-case” traditional systems,) where controlled entry into the vehicle 20 by driver 10 may be accomplished using any of traditional means including but not limited to a vehicle key, a traditional fob (i.e., not required to be a further adapted fob 1-4a,) a vehicle-door access pad code, a wireless key-card, or even an app for issuing a wireless communication to the vehicle 20's traditional door access control system (e.g., similar to a car app as provided by car manufacturer Tesla with at least some of its vehicles,) all examples of which are well-known in the marketplace.

Many current marketplace vehicles 20 comprise seat occupied sensors 20-2 for detecting the situated presence of an operator 10 in the driver's seat, as well as sensors 20-3 for determining that a vehicle door is in the state of opened or closed, where the preferred vehicle also includes a vehicle camera 20-4 of which many varieties are known for capturing images of at least the person situated in the driver's seat, either using visible and/or non-visible light, where non-visible light typically includes infrared light (IR). Any of such traditional sensors 20-2, 20-3, 20-4 or even other sensors 20-5 comprise operator detection component 40-2 for determining that a potential operator 10 is situated in an operator/driver's seat. Prior to vehicle 20 enabling the operation of vehicle 20 at least to some level such as starting the engine, vehicle 20 captures one or more biometrics such as but not limited to current images 10-c-img of the seated driver 10 for example using vehicle camera 20-4, where the one or more images 10-c-img are then communicated via for example a communications component such as 20-1 to a primary device such as 1-1 (or 1-2 not depicted). After communication, the intermediary app of primary device 1 confirms the identity of the driver 10 with respect to an enrolled identity (see FIGS. 2A and 2B) and comparable “registration” biometrics (also see the cross-referenced art for more discussion,) thus performing a “second-authentication” prior to allowing vehicle 20 operation.

Once the operator 10 that is currently detected as present within the driver's seat is properly authenticated and authorized by the primary device 1, primary device 1 communicates preferably both an anonymous operator ID 10-id and anonymous operator data 10-aod to vehicle 20 via a communications component such as 20-1, either as a part of or in combination with an authorized access key 30-4-aak. The computing elements associated with vehicle 20 such as included with or associated with control center 20-1 then act to confirm that for example the received access key 30-4-aak is a valid key according to any of well-known methods. (It is noted and will be clear from a careful consideration, that while the teachings of the present figure and the application as a whole emphasize the value of maintaining operator 10 anonymity, it is well-known that in many situations it is alternatively desirable to provide operator 10 identity information, such as when the operators 10 are working for a municipality, the military, a corporation managing a professional fleet of trucks, etc., and as such the present invention should not be limited to the exemplary anonymous applications as the many novel teachings provided herein also apply to non-anonymous systems.)

At least in part using any one of or any combination of anonymous operator ID 10-id, anonymous operator data 10-aod, and access key 30-4-aak, component 40-4 of vehicle 20 determines any of operator/vehicle readiness metrics 20-m3 while component 40-6 of vehicle 20 determines any of vehicle limitations based at least in part upon any one of or any combination of readiness metrics 20-m3, ID 10-id, data 10-aod, and access key 30-4-aak. Both prior-to and during ongoing vehicle 20 operations, vehicle 20 preferably uses indirect metrics 20-m2 to optionally determine any of route characteristics 20-m4 and environment conditions 20-m5, where then readiness determination component 40-4 uses any of direct metrics 20-m1, indirect metrics 20-m2, route characteristics 20-m4, environment conditions 20-m5, ID 10-id, data 10-aod, and access key 30-4-aak, to further determine current operator/vehicle readiness metrics 20-m3 and to update vehicle operation limits in real-time using component 40-6.

In a variation to the above-described authentication of the operator 10 wherein the final comparison of the operator 10 registered with the intermediary app executing on the operator 10's primary device 1-1 (thus performing operator authentication and vetting function 30-2 on the “operator device,” see FIG. 1A,) it is also possible to shift the authentication and vetting function 30-2 to the vehicle, e.g., to be executed on the vehicle communications and control center 20-1. In this alternative process, once the operator 10 is situated, either vehicle 20 upon such detection communicates with the operator 10's primary device 1-1, or the operator understanding that they are currently situated interacts with the primary device 1-1, such that the intermediary app executing on the primary device 1-1 is caused to transmit the (preferably but not necessarily anonymous) operator ID along with a prior determined biometric such as a registered facial image(s) 10-r-img or a registered fingerprint to the control center 20-1. Intermediary app 1-1 optionally and preferably also communicates the operator 10's authorized access key 30-4-aak along with operator data 10-aod to control center 20-1. The control center 20-1, now executing the authentication and vetting function 30-2 in the vehicle 20 as opposed to the operator 10's primary device 1-1, compares for example the transmitted registered image(s) 10-r-img to current image(s) 10-c-img for determining a sufficient match and thus “authenticating” operator 10. Upon authentication, control center 20-1 executing vetting function 30-2 may then also check the operator 10's access et 30-4-aak for sufficient vehicle 20 access and operation rights and limits, and also check the operators data 10-aod for sufficient “readiness” preferably based upon an impairment scoring algorithm or otherwise any algorithm preferred by the vehicle 20 owner/manager.

As a careful consideration will show, if the intermediary app alternatively or additionally provides a registered fingerprint, then vehicle 20's other vehicle sensors and data sources 20-5 should include a vehicle-operator fingerprint reader for determining a “current fingerprint(s)” similar to current images(s) 10-c-img, for which then a fingerprint comparison for authentication may be performed. It is noted that the present invention allows for “multi-factor authentication” where an operator is authenticated for example using facial images, fingerprints, and even a pass code prompted for and entered by the operator 10 into either the primary device 1-1 or a UI comprised within the vehicle 20 or otherwise in communication with the control center 20-1.

Still referring to FIG. 3A, but now specifically to authorized operator data 10-aod, the preferred data 10-aod comprises personal health metrics 10-m, instance and type baselines 50-m1, instance and type histories 50-m2 as well as authorized conditions of operation 30-aco. The interested reader is especially directed to related PCT application PCT/US22/47511 filed on Oct. 23, 2022, entitled MEASUREMENT AUTHENTICATING WELLNESS DEVICES for teaching as to how the a primary device such as smartphone 1-1 or smartwatch 1-2 can directly determine and privately authenticate personal health metrics as well as electronically pair and interface with a multitude of “secondary” or “tertiary” devices (herein collectively “companion” devices) for determining additional private authenticated health metrics extending beyond the device measurement limits of the primary device 1, all as a careful understanding of the of the related applications will show. As discussed in these related applications and as incorporated herein by reference, such private authenticated health measurements can be “scored” or evaluated against one or more “public health regiments” (also referred to as “protocols”,) where this algorithmic scoring or evaluation has many possible implementations and results in what was referred to as a “current health status” of the operator, where is it important to see that this health status is both “ongoing” (thus day-by-day measurements forming the current health assessment for example by considering the current and prior 14 days) and “non-session” or both “pre-and-post session” (which does not exclude “during-session” additional health measurements by primary device 1,) offering considerable advantage as to the determination of an operator 10's “readiness” just prior to intended asset 20 operation. The present invention has provided additional discussion for including in the scope of health metrics physiological and psychological measurements specifically directed to “operator impairments,” where impairments are generally understood as a diminishing of the anticipated operator's maximumly preferred and expected operation performance.

As prior discussed. “instance” is being used to refer to a particular individual (operator 10) of a “type” of individual, where the individuals of the same type form a group. Those familiar with data science will understand that capturing historical performance data 50-m2 on individuals, and then forming groups based upon features defining types of individuals, allows for determining generalized patterns (50-m1) for the group using significantly more data than any specific individual will accumulate. This is beneficial for example in that accumulated vehicle operation data 50-1 preferably represents thousands to millions of individuals, collectively representing millions to hundreds of millions of session operation data, possible broken into hundreds or less of groups/types. This allows for individuals will relatively few sessions to be predicted based upon their assigned group/type, and also allows “baselines” or averages determined about an individual to be compared to group baselines or averages, all providing a highly valuable technical advantage to the system 100's performance, as a careful consideration will show.

And finally with respect to a summary discussion of authorized operator data 10-aod, it is also preferred that an operator 10 by way of the use of their primary device 1, carries about and transmits to an appropriate asset such as vehicle 20 any of their “authorization to operate” including for example “limits of operation.” As will be apparent to those skilled in the art of information systems, any or all of data 10-ado can be stored, maintained, retrieved, and/or provided to the appropriate asset such as vehicle 20 from either the operator 10's primary device or some other system such as a remote server 50 (see FIG. 1B) or the asset's 20's own information system such as control center 20-1 of the present figure, or even some combination thereof.

Still referring to FIG. 3A, it is important to see that in this decentralized implementation 100-1 (as opposed to asset centralized implementation 100-2 and entity centralized implementation 100-3, see especially FIG. 7) the identity of operator 10 remains private to the operator 10, only being known to the intermediary app executing on the primary device such as 1-1 or 1-2. Thus, from a traditional perspective, the operator 10 neither enrolled with nor ever known to vehicle 20 (and hence any of its “computing devices” such as 20-1,) thus the operation by operator 10 of vehicle 20 remains anonymous while at the same time the operator's identity is confirmed to be valid and authorized for operation by the primary device 1 (such as 1-1, or 1-2)'s intermediary app.

In recap, an operator 10, as described in relation to FIGS. 2A and 2B is enrolled with the primary device 1 intermediary app, where preferably the primary device 1 is under the operator 10's own control and ownership, thus being referred to as a “decentralized,” “private”/“anonymous” system variant of the present invention. As a careful understanding of the cross-referenced art will show, the “intermediary app” (IA) on the primary device is acting as a “trusted honest broker” between both “parties,” where “party-1” is the operator 10 and “party-2” is the operated asset such as vehicle 20 (and by extension its owner, renter, or otherwise provider). As discussed in relation to FIG. 2B, the intermediary app and device 1 is also able to communicate with additional third parties 22 such as but not limited to vehicle/equipment insurers and renters, where this communication can retain the amount of anonymity acceptable to the providing third party 22, many of which will require some of the person 10's identifying information but not necessarily information such as a personally identifying biometric (e.g., facial image, fingerprint), as will be well understood by those familiar especially with 3^{rd} party billing systems.

A careful consideration of the present figure will show that the trusted honest broker/intermediary app (IA) primary device 1 has already acquired “registration” (thus enrollment) biometric information such as facial image(s) 10-r-img of the operator 10, but that the corresponding “confirmation” biometric information 10-c-img is captured by the vehicle 20 upon detecting the proper physical situation of a possible operator 10, and that this cooperative relationship ensures that both the actual operator 10 the vehicle is “seeing” as image 10-c-img and the person 10 that the intermediary app has prior enrolled using an enrollment image 10-r-img are sufficiently determined to be the same individual. (Again, it is noted that this final “sufficient determination”/matching algorithm could alternatively be executed on vehicle 20's control center 20-1 while still ensuring operator 20 anonymity, where the primary device 1 provides to control center 20-1 the registration image(s) 10-r-im along with an anonymous operator ID 10-id, such that the matching algorithm simply confirms the individual being seen by automobile 20 in the driver's position is sufficiently the same as the individual pre-known to the primary device 1. Thereafter, all session data is accumulated by vehicle 20 using the anonymous ID such that the operator 10's ID continues to remain private even though significant and valuable historical driver information is accumulated and even shared with 3^{rd} parties.)

As just stated, in decentralized system 100-1, once the operator 10 is properly and privately authenticated by the primary device 1 (shown as 1-1 but also possibly being wearable 1-2,) the primary device preferably provides an anonymous operator ID 10-id to vehicle 20 for retrieving pre-stored vehicle data 50-1, and then also aggregating new or otherwise updating existing vehicle data 50-1. The present invention anticipates that a vehicle computing element such as 20-1 optionally stores for example any of vehicle operation data 10-d1, 10-d2, 10-d3 (collectively 50-1, see FIG. 1B) at least as determined during any given session of asset/vehicle 20 operation by a given operator 10, where those familiar with data storage systems will understand that it is useful to have a “ID”/unique identifier (such as 10-id) with which to uniquely segregate and associate any stored data. It is also possible that this same session data can be associated not just with the operator 10 (e.g., anonymously using 10-id or non-anonymously using operator identifying information) but also with the asset, in this example vehicle 20, where vehicle 20 could be represented as a non-anonymous ID such as what is commonly known as the vehicle's “VIN” number, or an anonymized version of such a VIN number or otherwise any assigned anonymous vehicle ID.

Considering full anonymity as an exemplary use case, stored vehicle data 50-1 can be accumulated and analyzed for the same anonymous operator 10 operating different anonymous assets/vehicles 20 over different sessions, thus also implying that the same anonymous asset has its own session data accumulated over time in reference to potentially multiple anonymous operators 10. It is further anticipated that the vehicle 20 stores some or all of the saved and maintained vehicle operation data 50-1 on a remote server such as 50 (FIG. 1B,) to be retrieved by using the unique identifier (e.g., 10-id) as needed (and typically also any anonymous asset ID, not depicted) and presumably at the beginning of a new session of operation.

In one example, after receiving from a primary device 1 confirmation of the authenticity of an operator 10, as well as an anonymous ID 10-id preferably combined with a valid authorized access key 30-4-aak, vehicle 20 may take several initial operations such as repositioning the operator's seat, adjusting mirrors, cabin lighting, setting audio preferences and default tunned station(s), many functions of which are currently implemented by either the operator touching a button or providing a biometric such as a fingerprint (see the Mercedes C-class cars).

In another example, after receiving confirmation of authenticity from the primary device 1, the vehicle retrieves any of vehicle operation data 50-1 (such as 10-d1, 10-d2, and 10-d3) from either of both an on-board computing element 20-1 or a remote server 50, and/or receives data 50-1 from the primary device 1 such as exemplary preferred and presently depicted dataset 10-aod. Any retrieved data 10-d1 might for example allow vehicle 20 to present a list of familiar travel routes taken in the past by the operator drawn upon data 20-m4, even using a different vehicle 20 since this information is shown to aggregate “cross-vehicle.” What is also made clear from a careful decision is that vehicle 20, either using its own computing element 20-1 and/or also an external computing element such as any of the primary device 1 or a remote server 50, can determine a “fitness” or “readiness” of both the operator 10 and the vehicle 20 that draws upon any of vehicle operation data 50-1, especially including personal health metrics 10-m (that preferably represent a significant assessment of the operator 10's physiological and psychological state as measured prior to even entering the vehicle 20, thus “pre-session”) as well operation context data 10-d1 such as the route and current traffic, weather, time-of-day, etc, and instance and type baseline and history data 10-d3 (such as the operator 10's familiarity and experience given the proposed operational context and current assessment of the operator 10's state). As a careful consideration will show, the computing elements such as 20-1 or a vehicle guidance system on vehicle 20 might then plan suggested stops along an extended route, such as considering the operator 10's driving habits (speeds, length of trip segments, experience with congested traffic, etc.,) where for example then these suggestions are further linked to vehicle operational limits 40-6 (such as speeds, length of travel time, etc,) enforced by the vehicle 20, based upon the operator 10's pre-authorization as associated with their access key 30-4-aak (e.g., limits on nighttime driving over longer distances or city driving during certain time of the day,) all of which may be further impacted by any determined operator readiness levels 20-m3 such as levels of rest, sobriety, glucose, etc.

A familiarity with and careful consideration of the state-of-the-art in driver assisted car technology will show that some possible vehicle 20 operational limits (such as top speed) can be managed to some degree automatically, where other operational limits such as “city driving during certain times of the day” might otherwise generate notifications (such as texts or emails) to the authorizing authority, where then these notifications become a disincentives to the operator 10, and thus the limit is not strictly enforced and/or otherwise does not “take control” of the vehicle but rather encourages behavioral adaptation to conform with the authorized limits. For example, when the operator 10 is a professional driver using a company truck, the operator 10 is presumably strongly motivated to follow the operational limits set by the workings of system 100 as taught possible herein, even though strictly speaking at least some of these limits could be ignored. In another example, a parent in a family holds a master key 30-4-mak and authorizes restricted access keys 30-4-rak to a child including one or more authorized conditions of operation 30-aco, where some such conditions still allow vehicle operation but also generate notification(s) to the parent, while other conditions disallow the vehicle from being started and operated beyond the limit without an override code provided by the parent back to the child-operator's primary device 1 or the vehicle 20 for example after receiving an email or text notification.

And finally with respect to FIG. 3A, it is again noted that “a-b” operational limits 40-6 (first discussed in relation to FIG. 2A) may comprise the governing of any controllable function of vehicle 20, including determining the amounts of, values of, and/or settings for the controllable function, for example determining maximum velocities, accelerations, distances, consumptions of resources, etc., where these determinations are further relatable to 1) temporal considerations such as time-of-day, day-of-week, season of year, etc., 2) spatial considerations such as a tracked and even changing geo-location, 3) other operational context considerations such as weather, traffic, driving intensity of route, etc., 4) vehicle readiness considerations such as measurements of vehicle fluid levels, recent servicing, mileage, tire pressure, etc., and 5) operator readiness considerations such as rest/sleep normalcy and levels over the past x days, alcohol levels, and even stress levels for example measured by facial analysis conducted by the primary device using images captured by a vehicle camera 20-4.

Referring next to FIG. 3B in combination with 3A, there is shown a process flow chart describing key steps for the operation of the variation 100-1 depicted in FIG. 3A. The primary device 1 such as 1-1 and 1-2 is shown as providing operator authentication and vetting component 30-2 such that these functions remain private to the owner/user of the primary device 1 who is presumably and preferably the vehicle operator (such as 10 depicted in FIG. 3A). The reader is directed to the cross-referenced art for a better understanding of the various means for providing what is herein referred to as “decentralized” “fully-private” authentication using a primary device 1 such as 1-1 and 1-2 (where “fully-private” in brief means that no personal information, especially including a biometric such as a face image or fingerprint, is ever shared by the primary device 1 with any other secondary system, and thus the person is only ever enrolled with/“recognized by” the “honest broker app” herein intermediary app that is executing solely on the primary device 1, where then the honest broker “vouches” for the authenticity of the person 10 to any secondary system including “access points” such as a vehicle door or cabin wherein vehicle controls are situated).

Within the herein taught and many possible variants of a “decentralized systems” of the present invention as described in relation to FIGS. 2A, 2B, 3A, 3B, 4A, 4B, 5A, 5B, and part of FIG. 6, there are two main implementations including 1) pre-confirmed fully-private authentication wherein initial enrollment and subsequent identification confirmation is accomplished on the primary device 1 prior to any interaction with the asset/vehicle 20, and 2) post-confirmed fully-private authentication wherein initial enrollment is accomplished on the primary device 1 prior to any interaction with the vehicle/device 20, and whereafter subsequent identification confirmation is performed in cooperation with the vehicle/equipment 20. FIG. 2A is an example of (1) pre-confirmed fully-private authentication, while FIG. 3A is an example of (2) post-confirmed fully-private authentication.

In pre-confirmed authentication (1) a person 10 desirous of at some point being an operator of an asset such as a vehicle, equipment, processes, etc. (herein any of which being element 20,) wherein especially the operation of element 20 includes safety considerations, the person 10 interacts with a primary device 1 to: (1.1) provide initial enrollment/“registration” biometrics, including any of face images, fingerprints, retinal scans, heartbeat scans, palmprint, and any other of the well-known or to become known biometric identification methods prior to entering or operating asset 20, (1.2) provide subsequent confirmation biometrics under and during controlled circumstances that commence preferably prior to operating asset 20 and possibly also just prior to entering asset 20 (depending for example upon the type of asset 20,) where the confirmation biometrics are compared to the registration biometrics to confirm the person's identity thus authentication during and while the controlled circumstances remain enforced, and (1.3) provide proof-of-identity data to an “access point” of asset 20 after confirmation and before operating 20 and possibly before entering asset 20.

Again, this pre-confirmed authentication method (1) is decentralized and fully-private, where enrollment preferably happens completely between the intermediary app operating on the person 10's presumably personal primary device 1 and no personal data especially including personal biometric(s) are shared with a secondary system such as control center 20-1 of vehicle 20. The intermediary app having enrolled the person 10 is then able to confirm the person 10's identity when device 1 determines that the device 1/operator 10 is within such controlled circumstances, such as being spatially contained within a physical confirmation area or otherwise a geofenced confirmation area or while having a wearable clasped and remaining clasped about a body-part such as the wrist, all of which is described in the cross-referenced art. After confirmation/authentication, the operator 10 still being in the controlled circumstances may then request access via an access point (herein governed by “access control” component 30-4, such as a vehicle door) to the asset, vehicle, equipment, process, 20, wherein the intermediary app of the primary device 1 acts as an honest broker working on behalf of the person and asset 20 to ensure both authentication and authority for access as well as full-privacy.

As will be discussed in greater detail, a primary device 1 as taught in the cross-references also comprises an on-going “publicly trustable” health status determinable based upon the processing of one or more authenticated health measurements of the person 10, where on-going in this case especially includes a duration of time prior to requesting access to and otherwise operation of an asset such as vehicle 20. This on-going health state comprises what is herein referred to as personal health metrics 10-m (see FIG. 3A).

Preferably the primary device 1 is carriable (such as smartphone 1-1) or wearable (such as smartwatch 1-2) to provide convenience to the operator 10. What is shown herein is that this on-going pre-access, pre-operation health status with comprising health metrics 1-m is highly useful for determining at least in part an “operator readiness” using component 40-4, where also any determination of operator readiness is usable by access control component 30-4 to either limit or deny access to or operation of an element 20.

Still referring to FIG. 3B as well as 3A, different from the depictions and teachings of FIGS. 2A and 2B that employ pre-confirmed authentication (1) prior to access/operation of vehicle 20, a person 10 still uses a primary device 1 to maintain on on-going health status, but then alternatively uses primary device 1 to interact with vehicle 20 to accomplish post-access-confirmed fully-private authentication (2). As discussed in relation to FIG. 3A, this interaction comprises the vehicle 20 as at least in part providing the “controlled circumstances” for enabling the step of confirmation including ensuring that the potential operator 10 is spatially contained within a physical confirmation area (such as the front driver's seat) or otherwise a geofenced confirmation area (such as within a close proximity to the vehicle 20,) where the seat area is a physical confirmation area and where a wireless proximity signal active within the “seat volume” is a geofenced confirmation area. From a different perspective, a person 10 detected in a driver's seat (for example using pressure sensors or other traditional means) allows the interpretation of a detection of a closed door to be similar to a clasped wearable, where a subsequently confirmed person 10 remains authenticated at least until the door is opened (hence by comparison “unclasped”). (See the cross-referenced art for a discussion of “clasped” versus “unclasped”.)

For the exemplary use case of vehicle 20 as an asset, the controlled circumstances include a determination by operator detector component 40-4 that the person 10 desirous of operating a vehicle 20 is properly situated in the driver's seat, for which many traditional and well-known methods are available. It is again noted that unlike pre-confirmed authentication (1), a person 10 may enter a vehicle 20 using any of traditional means and is not otherwise restricted to prove either additional identity or authority beyond the practices of these traditional means. (It is also noted that using confirmed authentication (1), it is possible to likewise first allow a person 10 to enter a vehicle 20 without confirmation, where then confirmation is provided strictly by the primary device 1 without any interaction with the vehicle 20 other than the vehicle 20 requesting authentication from device 1 after determination that the person 10 is properly situated in the driver's seat.) Now specifically referring to FIG. 3B, the post-access-confirmed fully-private authentication (2) variation of decentralized system 100-1 commences as preferable vehicle 20 embedded vehicle access control component 30-4 determines in step 30-4-1 if pre-access authentication is required. Such determination may be static in that either pre-access authentication is “always” or “never” required, or dynamic and based upon any number of determinable criteria such as but not limited to a geolocation, time of day, presence of proper wireless signaling, etc. Assuming pre-access authorization is required, vehicle access control 30-4 preferable communicates with primary device 1 to initiate or indicate such requirement, or otherwise provides an indication (e.g., a visible flash or an audible sound) to operator 10 such that operator 10 then initiates operator authentication and vetting process 30-2 executed the primary device 1. Process 30-2, as prior discussed, preferably accepts one or more confirmation biometrics of operator 10 for matching with stored registration biometrics, where upon sufficient match process 30-2 communicates at least authorization access key 30-4-aak optionally including the anonymous operator ID 10-id to vehicle access control process 30-4 (and possibly also communicates any of anonymous operator data 10-aod). (Again, it is noted that operator 10 might be allowed to by component 30-4 to enter vehicle 20 without authentication, only to the be authenticated after entering and situating in the vehicle 20, see steps 40-2, 40-4-1, and 1-pa.) After entering vehicle 20 and properly situating in the driver's seat, vehicle access control component 40-2 preferably detects the seating of operator 10 by using any of well known means some of which have been prior discussed in relation to FIG. 3A. Once the operator 10 is sufficiently detected by component 40-2, component 40-2 causes one or more biometrics such as confirmation image(s) 10-c-img to be capture and provided to primary device 1 whereupon a private authentication/confirmation 1-pa of operator 10 is performed, for example by comparing the provided confirmation biometrics with prior determined registration biometrics (such as registration image(s) 10-r-img) as all prior discussed. Authentication/confirmation 1-pa processing on primary device 1 has access to a preferably private and encrypted data source 1-pds maintained by the primary device 1, for example at least including operator 10 personal data such as the registration biometric image(s) 10-r-img. Once confirmed, primary device 1 provides of vehicle 20's operator readiness determination component 40-4 a preferably anonymous operator ID 10-id and other anonymous operator data 10-aod, and may then also provide the authorized access key 30-4-aak (for example in the case that the pre-access authentication 30-4-1 was determined to not be required).

Operator readiness component 40-4 preferably has access to vehicle 20 determined and provided indirect and direct metrics 40-1, some of which may have been prior captured and maintained “on-vehicle” by control center 20-1 of “off-vehicle” for example by server 50, where in either case metrics 40-1 are preferably associated with the anonymous operator ID 10-id and thus retrievable for use by operator readiness determination component 40-4 using the operator ID 10-id provided by the primary device 1, for example as a part or associated with key 30-4-aak or with data 1-dat. As prior discussed, it is also assumed that vehicle 20 continues to determine and provide indirect and direct metrics 40-1 to component 40-4 during the subsequent operation of the vehicle 20 by authenticated operator 10. (As depicted in FIG. 3A, metrics can include any of 20-m1, 20-m2, 20-m3, 20-m4, or 20-m5.) Thus, preferably just prior to operation as well as during operation, operator readiness determination component 40-4 determines vehicle operation limits 40-6, after which operator 10 may then proceed to operate vehicle 30-6 in accordance with the prescribed limits 40-6.

Upon completion of a session of operation, e.g., the end of a driving trip in vehicle 20, preferably the vehicle 20 determines that vehicle operation has completed in step 40-8-1 and then subsequently and preferably initiates a transfer of any of vehicle operation data 50-1 to the primary device 1 for accumulation and storage.

Preferably transferred operations data 50-1 comprises any of vehicle direct metrics 20-m1, vehicle indirect metrics 20-m2, operator/vehicle readiness metrics 20-m3, route/use characteristics 20-m4, environment conditions 20-m5, and context readiness metrics 20-m6, all associated with the current and just ended session of operation, where primary device 1 stores, accumulates and maintains such data in private data source 1-pds. As a careful reading of the present teachings will show, and as those familiar with data architectures will appreciate, many viable options are possible for the storage of such information as data 50-1, including any combination of on primary device 1, on vehicle 20 such as stored in control center(s) 20-1, or on a remote server such as 50 (see FIG. 1B). Furthermore different portions of data 50-1 may advantageously be stored in any of 1, 20, or 50, and/or redundantly on any combinations of 1, 20, or 50. What is important to see is that system 100 is teaching the integration of pre and post session data, all relevant to the determination of operator readiness as described herein, to be maintained privately or shared publicly under an anonymous ID or using some personal information, all as the particular needs of the final desired application may be, some applications of which are described herein, but many more of which will be apparent to those familiar with a particular asset class.

And finally with respect to FIG. 3B, and to summarize, authentication and vetting 30-2 potentially issues authorized access key 30-4-aak to vehicle access control 30-4 as a part and means for gaining access to the vehicle 20, otherwise access is physically granted without authorization to any person 10 after which operation is limited to the subsequent person 10 authentication and confirmation while properly situated in the operators location (e.g., front driver's seat). Component 30-2 is shown to further comprise or otherwise function with: 1) intermediary app algorithms 1-pa for privately authenticating/confirming the identity of the scatted operator 10 which draws upon a private data source 1-pds preferably maintained on the primary device 1, and 2) intermediary app algorithms and data 1-dat optionally including an original or updated authorized access key 30-4-aak to be provided to the vehicle 20 for the authenticated and verified operator 10. Vehicle 20 functions include but are not limited to vehicle access control 30-4 that works cooperatively with operator authentication and vetting 30-2, operator seated function 40-2 for determining when the operator 10 is properly seated, provide confirmation biometrics function 40-4-1 for capturing and transferring at least one confirmation biometric to be privately authenticated by primary device 1 function 1-pa, operator readiness component 40-4 for preferably receiving current indirect and direct metrics from vehicle 20 function 40-1 and anonymous operator ID and other data from primary device 1 function/data 1-dat and then cooperating with vehicle component 40-6 to determine any of vehicle operation limits used to control the vehicle operation in function 30-6. Vehicle operation 30-6 preferably includes a determination of the completion of a session of vehicle operation in a function 40-8-1, where then thereafter, or otherwise, select vehicle operation data is provided to update the private data source in a function 40-8-2.

Referring next to FIG. 4A, there is depicted the primary device smartwatch 1-2 as taught in the copending related applications, where the “intermediary app” and alternatively named “honest broker” app of the prior applications is the intermediary app of the present application and further comprises preferably some of all of the functions of either or both of operator authentication and vetting component 30-2 and optionally operator readiness determination component 40-4. A primary device such as 1-2 thus comprises functions for the authentication, authorization, and vetting of an operator 10 to first access and/or subsequently operate a vehicle 20 (such as but not limited to an automobile,) as well as functions for gathering and assessing at least physiological and optionally psychological data about the operator 10 prior to or even during a session of operation in order to maintain a preferably on-going assessment of operator 10's readiness for initial and continued operation of vehicle 20. Readiness for operation of a vehicle 20 for example includes any determination indicative of potential impairment (all as prior discussed herein and for which significant literature exists in the public domain,) such as drowsiness brought on by sleep, physical and mental activity patterns, alcohol consumption, prescription medication, etc., all determinations that can be made using currently available and soon anticipated wellness devices and algorithms for processing measurement information.

It is noted that smartphone primary device 1-1 is also usable for executing components 30-2 or 40-2, either separately or in combination with smartwatch 1-2, where, by acting in combination with smartwatch 1-2, smartphone 1-1 serves to at least off-load processing from smartwatch 1-2 that also reduces available watch 1-2 battery power while also providing a larger screen interface for the operator 10.

Referring next to FIGS. 4B and 4C, there is depicted two exemplary key fobs 1-4b and 1-4a, respectively, each capable of communication with a primary device 1 such as smartphone 1-1 or smartwatch 1-2 for exchanging information related to the at least the functions taught herein for governing access to and operation of a vehicle 20, as well as communication with the vehicle 20 for exchanging information comprising at least in part access rights and optionally conditions-of-operation. Key fobs 1-4a and 1-4b preferably further include any of traditional means such as buttons for accepting operator inputs, as well as preferably shortrange wireless communication means such as an NFC reader for receiving alternative information associated with authentication and restrictions of an agent 10-a requesting or otherwise being granted authorized access and operation of the vehicle or equipment potentially without biometric identity confirmation.

Fobs 1-4a and 1-4b preferably further include any of personal biometric identity sensors 1-4-s1, for example used to capture either or both of a fingerprint or pulse 1-h2 of the holder 10-h of the key fob 1-4a/1-4b, where then any such captured identity information is transmitted by the fob to the primary device 1 as a means of confirming substantially in real-time the holder 10-h of the key fob 1-4a/1-4b's identity. Assuming primary device 1 is able to authenticate the operator using pre-registered biometrics for comparison with the identity information captured and transmitted by the fob 1-4a/1-4b, it is then preferred that the primary device 1 communicate an “operator authenticated” signal in return to the fob 1-4a/1-4b, where upon receiving the signal the fob then sends a “request to grant entry” to the vehicle 20's access control component 30-4 in this example operating on vehicle 20. It is also possible that the primary device 1 transmits an authorized access key 30-4-aak along with or instead of an “operator authenticated” signal, as the key 30-4-aak works as a confirmation of authentication and then can also include additional access rights and even authorized conditions of operation 30-aco.

While a careful consideration will show that it is possible (and anticipated herein as a variation) for the primary device 1, e.g., either 1-1 or 1-2, to communicate directly with the vehicle access control 30-4 component, one benefit of separating the functions of operator authentication and vetting 30-2 (performed on the primary device 1) from access request signally (performed on the fob 1-4a/1-4b and possibly using encoded/encrypted signals,) is that both devices 1 and either of 1-4a or 1-4b must be present and working together to allow access providing another level of security. For example, a rental car company may have fobs 1-4a or 1-4b for each of its rental cars, where only the fob has the ability to issues a “request to entry” and is thus individually paired to a particular car, where as the fob is also able to communicate with any primary device 1 executing an operator authentication and vetting 30-2 component for example as provided by the rental company to the operator 10 as a part of the rental process. As a careful consideration will show, the rental provided component 30-2 can then include accompanying authorized conditions of operation 30-aco which for example include times day and time limits such that the vehicle 20's access and operation is highly controllable and more difficult to “hack” because of the reliance upon the intermediary fob 1-4a and 1-4b. Still referring to FIGS. 4B and 4C, the preferred fob 1-4a or 1-4b includes wireless reader means such as an NFC reader for reading “valet 10-a identify” information for example from a “valet card,” where then the fob 1-4a or 1-4b preferably transmits this same or sufficiently similar scanned valet 10-a identity information to the primary device 1 such that the presumed owner of vehicle 20 then has critical information when providing a valet/agent 10-a temporary access to their vehicle 20 (such as at a parking lot or a automobile service station). In a preferred operation, the operator 10 (presumed owner of vehicle 20) then uses their primary app 1 to indicate restrictions such as the maximum duration of time (e.g. “3 mins” or “5 mins”) after using the fob 1-4 or 1-4b to scan the valet card 10-a-vc that the fob 1-4a or 1-4b should allow the agent 10-a to access and operate the vehicle 20 using the fob, or even other limits such as total miles of driving, amounts of gasoline or energy used, maximum speeds, use of an audio system or other vehicle features, etc. It is also possible and preferred for the primary app 1 to allow the operator/owner 10 to indicate a maximum duration of time that the fob 1-4a or 1-4b will allow entry and access to the vehicle 20 without “reauthorization” (e.g., “5 hours” or “3 days”).

As a careful consideration will show, the “maximum duration of time between valet card 10-a-ve swipe and vehicle entry/operation” function requires the valet to have the same valid “valet card” that was first scanned into the fob 1-4a or 1-4b (presumably in the presence of operator/owner 10) and subsequently registered and authorized on primary device 1 whenever subsequent access and operation of the vehicle 20 is desired, for example at some later time when moving vehicle 20 becomes necessary. As a more detailed example, upon authorization of the valet 10-a during the drop-off of vehicle 20 by the operator/owner 10, it is presumed that after authorization the agent 10-a may then enter and drive off with the vehicle 20, all for example within 5 mins of first authorization (where 5 mins has been selected by the operator as the aforementioned “maximum duration of time between swipe and entry” and where of course upon initial drop off if the maximum duration is exceeded before the valet 10-a initially moves vehicle 20, then valet 10-a can simply re-swipe their valet card 10-a-vc and proceed to enter and operate the vehicle 20). Presumably, after moving and parking the vehicle 20 for the first time, the valet 10-a will need to move the vehicle 20 again (e.g., perhaps hours later,) and even upon multiple occasions, at which time the valet 10-a must have both the fob 1-4a or 1-4b and their valet card. This provides at least two advantages. First, as a careful consideration will show, this fob-valet card combination defeats a criminal from stealing a parked vehicle 20 in an open lot by first stealing the fob from a valet station. Second, each time the valet card 10-a-ve is swiped, this provides a time log and that can be store on the fob even while not in communication with the primary device 1 and used to show legal responsibility for accessing and operating vehicle 20.

It is also important to see that a key fob such as herein taught that can pair and communicate with a primary device 1, control authorized access and entry, and record usage times and duration of operation for each vehicle usage, provides valuable information to the vehicle's owner, where otherwise collecting such information would require a modification to the vehicle 20's onboard computing processes, something that might easily preclude the benefits of such a system for vehicle's already existing in the marketplace as they may not be easily upgraded to include such functions and otherwise creates an additional burden on the automobile manufacturers that the may be unwilling to accept.

Still referring to FIGS. 4B and 4C, in yet another valet 10-a control of vehicle variation, it is possible that the valet 10-a be required to additionally provide a fingerprint using fob fingerprint reader 1-4-s1, either in place of the valet card 10-a-ve or in addition to the valet card. Such biometric factor authorization is more secure than the valet card factor authorization, where then a dual factor authorization using both the fingerprint and the card is also possible, further increasing security. It will also be clear that by requiring the valet 10-a's fingerprint, the vehicle 20 can only be accessed and operated by that individual valet 10-a, as opposed to some other individual valet 10-a (or criminal) using the originally authorized valet 10-a's valet card.

Referring next to FIG. 5A, there is shown a perspective drawing of an exemplary new species/individual of the more general genus/class of “secured authentication signals” first taught in the related applications.

These signals provided means for privately authenticating health metrics captured or determined by a companion device and included (1) light signaling and (2) pulse signaling (as depicted in FIG. 5B). The new signaling method (3) taught herein for authenticating companion device activations and data collections is referred to herein as “bio-conductive signaling” and uses well-known technology for pulsing electrical signals through the body. Bio-conductive signally is currently used for other purposes such as with “smart scales” and other body fat measurement devices and is referred to in the art as “bioelectric impedance analysis” (“BIA”). While not relevant to the present invention, different types of body components (e.g., fat, muscle, water, bone) each conduct electricity in measurably different ways such that by sending weak electrical pulses through the body it is possible to determine the relative amounts of the various body components by percentage.

Still referring to FIG. 4A, for the present purposes, what is important to see is that when sending (via a companion device such as key fob 1-4b) a detectable electrical signal from a first body point location (such as a fingertip) to be detected at a second body point location (such as the wrist,) the body is then only transport mechanism for the pulsed signal, the understanding of which ensures that the receiving of the signal (at the wrist via the already authenticated smartwatch 1-2) is sufficient to confirm that the first location and second location are of the same body, thus authenticating the first location. While the minus electrode is depicted as a connection between fob 1-4b and the middle finger, and the plus electrode is depicted as a connection between fob 1-4b and the pointer finger, this is merely exemplary as many variations are possible for establishing the electrical circuit path between one or more fingers (that are taking an action with a companion device such as pressing a button or touching a surface, both as depicted) and a second location associated with a prior authenticated wearable primary device such as smartwatch 1-2.

Regarding the companion device key fob 1-4b, the surface 1-4-s2 being touched by one or more fingers of operator 10 could be for example any of a) a mechanically activated button-press surface, b) a touch-sensitive activated button-press surface, and/or c) a surface comprising a fingerprint reader (such as 1-4-s1 shown in FIGS. 4B and 4C). As a careful consideration will show, the implementation (c) comprising a fingerprint reader allows the first body location (defined herein as the location originating the weak electrical signal) to become a self-authenticating location (similar to a primary device 1, which can then work in combination with another primary device such as 1-2, or with a companion device (defined as not comprising a primary biometric identification means such as a fingerprint reader,) thus many configurations of the present teachings are possible. As a careful consideration will also show, it is possible that the primary device 1-2 be the first body location generating the weak electrical pulse that is then detected by the companion device such as fob 1-4b, where then the companion device such as fob 1-4b and the primary device such as 1-2 are in preferably wireless communication (such as Bluetooth) for confirming the pulse was both sent and received or similar.

Thus, the present depiction should be considered exemplary rather than limiting, as many variations are possible for implementing bio-conductive signaling as a means for authenticating that two disparate devices are coming into substantially simultaneous contact with the body of an individual such as operator 10, where preferably but not necessarily, one of the devices is a prior authenticated device such that the second device is then authenticated by assurance of the authentication of the first device, all as will be well understood by a careful consideration of the teachings of the present and related applications.

Referring next to FIG. 5B, the primary device 1 is shown as either of smartphone 1-1 or smartwatch 1-2 that is seeking to authenticate a companion device 1-4 (such as fob 1-4b or many other types of companion devices especially as discussed in the related applications) using any of taught methods (1) light signaling, (2) pulse signaling, or (3) bio-conductive signaling. In the preferred configuration, the companion device 1-4 provides any of a request (for example as indicated by a surface touch or press) or data (such as a fingerprint, pulse, or other bio-measurement) to the primary device 1, where using a secured authentication signal 1-4—as such as (3) bio-conductive signaling the primary device 1 is able to authenticate that the companion device 1-4 is being used by the same individual/operator 10 that is currently using the primary device 1, and whereupon operator 10 identity confirmation primary device 1 then provides an authorization signal to companion device 1-4 and/or accepts data determined and collected by device 1-4.

Once authenticated, companion device 1-4 may for example provide authenticated companion data 1-4-cd to the primary device, where such data is many and varied and should not be limited to any particular example as provided herein, especially considering the teachings of the related applications where companion data was shown to have a significant range of possibilities. Authenticated companion device 1-4 may then also use any authorization signal provided by the primary device 1 to then enable or cause secondary authorization signaling from the companion device 1-4 to a connected operational device (such as an asset/vehicle/equipment/process 20,) where then for example a “traditional operator indication” (such as a surface touch or button press) for activating an associated asset/vehicle/equipment/process 20 without any of authentication of the operator and/or vetting of the readiness of the operator to indicate/initiate the asset 20 action/activity, becomes an “authenticated and readiness vetted operator indication,” the significant advantage and many benefits of which will be apparent for use in many use cases involving operators 10 and assets 20.

In one exemplary use case the operator 10 is a driver of a vehicle 20 and the companion device 1-4 is a key fob 1-4b capable of sending an “unlock door request” to the vehicle 20's vehicle access control component 30-4, where for a traditional vehicle key fob any person pressing the appropriate key fob button can cause the unlock request to be sent and thus also gain access to the vehicle 20, whereas the present invention teaches further adapted key fob 1-4a or 1-4b optionally using bio-conductivity (or some other secured authentication signal 1-4-as) to first authenticate the operator 10 and possibly also ensure the operator 10's readiness prior to sending to unlock request, which may then be denied if either or both of the operator's identity or readiness is insufficient. It is noted that in this system, only the key fob 1-4 must be further adapted to work in combination with the primary device 1, and as such the vehicle 20 does not need to be further adapted as it is already capable of responding to the unlock request, where a careful consideration will show the advantage of this solution considering that many vehicles already exist in the market for use with key fobs and could not reasonably and/or cost effectively be further adapted to work directly with the primary device 1.

What will also be clear is that while it is also anticipated and possible that the authenticated and readiness vetted operator 10 using a primary device 1 could initiate the “authenticated and readiness vetted unlock request” directly from the primary device 1, this also requires that the primary device 1 be further adapted to learn the vehicles unique unlock code (similar to a universal remote needing to be trained to operate a specific type of home theater device such as a tv, DVD player, sound bar, etc.), whereas the preferred alternative is that a primary device 1 is further adapted to “universally connect” to any key fob 1-4 (thus able to open any vehicle 20 with ever “knowing” that vehicle 20's unique “unlock code”) where each particular key fob 1-4 has already embedded the necessary unlock code(s) for a given and specific vehicle 20, in total providing a more cost effective marketplace solution to implement. (As a careful consideration will show, a traditional universal remote typically provides “operation instruction commands” based upon a “manufacture-model” and not upon a “serial number,” and thus the universal remote by analogy could send an “unlock request” to any Ford Mustang, which would be problematic and allow one operator to open any car for example of the same MFG-Model. However, in the case of at least vehicles, a key fob is traditionally programmed to open only one vehicle, thus tied to the “serial number” at least conceptually. Understanding this reveals one of the significant benefits of allowing the primary device 1 to “universally” operate any key fob 1-4, or perhaps any of a certain type such as MFG-Model, but then it is the key fob 1-4 that is specifically programmed/enabled to issue the appropriate “serialized” “unlock” or other command request.)

In yet another example, the companion device 1-4 is the “start button” now found in many modern vehicles, where traditionally any operator pressing this button (including a passenger or a child) could cause the vehicle 20 to be started. Using the teachings of the present invention, the traditional vehicle start button (that neither authenticates the operator nor the operator's readiness) is further adapted to include a secured authentication signal 1-4-as (such as but not limited to bio-conductivity (3)) for substantially simultaneously communicating with a primary device 1 during the button press action, such that merely pressing the further adapted start button is insufficient for causing a start signal to be transmitted to the vehicle 20's control systems, but rather the “start engine” signal is only transmitted if the operator 10 is authenticated by the primary device 1 and optionally also readiness vetted. As a careful consideration will show, further adapting the a traditional start button according to the teachings herein is potentially less costly than adapting the rest of the vehicle's ignition system, thus facilitating a process of upgrading an existing market vehicle 20 by simply replacing the traditional start button with a herein further adapted button.

In another vehicle 20 example, the traditional steering wheel is further adapted to either include a contact surface 1-4-s2 for determining that the hands now being placed on the steering wheel are of an operator 10 wearing a primary device 1-2 or using a primary device 1-1, where then the steering wheel upon authentication and/or readiness vetting via the primary device 1, and possible in combination with primary device 1 transmits any number and types of signals to the vehicle 20's control center 20-1. Such authenticated signals might include directives to set/reset driver seat positions, cabin lighting or temperature, etc., as well as virtually any possible vehicle 20 operational limit. The present application anticipates that this further adaptation to the traditional steering wheel can be accomplished as an applied material such as a leather steering wheel wrap, where the applied material includes the necessary contact surface(s) 1-4-s2 and associated electronics. Using such an approach, it is possible that adapting existing marketplace vehicles 20 is an “aftermarket” solution (e.g., like the start button and key fob above,) where the material is applied and presumably a software upgrade is implemented in the vehicle 20's control center 20-1.

Thus the reader will see that many useful variations of an “authenticated and readiness verified operator-asset user interface” are possible for use in virtually any type of asset 20 wherein an operator uses a physical interface to provide activation signals or otherwise indications, and thus the present invention should not be limited to the current examples, as many other examples for all types of assets 20 (and not exclusively vehicles 20) are possible. It will also be clear that the present teachings have use beyond the operator 10 of an asset 20, for example where a passenger in a back seat may have a similar “authenticated and readiness verified operator-asset user interface” for indicating the setting of one or more vehicle 20 limits. In general, many assets 20 can be considered to have multiple “operators 10,” each operator 10 having their own primary device 1 and access to their own asset user interface, such that a system 100 should not be limited to a “single operator” use case. As will also be clear by careful consideration, the operator-asset user interface may also be non-physical. For example, a camera situated in asset 20 (see 20-4 in FIG. 3A) might be imaging the operator 10 and communicating with primary device 1 as prior discussed to authenticate operator 10 and possibly confirm operator 10's readiness for operation. While imaging and confirming the identify operator 10, operator 10 might for example provide any of gestures such as head or hand movement commands or voice commands, where then the commands are treatable as authenticated and possibly readiness vetted, all as taught herein. Thus, the present invention should not be limited to the use of physical interfaces for providing authenticated and readiness vetted indication by an operator 10 to an asset 20, as non-physical interfaces or even some combination thereof are also possible.

Referring next to FIGS. 6A and 6B, there is shown figures from the copending related PCT application PCT/US22/47511 filed on Oct. 23, 2022, entitled MEASUREMENT AUTHENTICATING WELLNESS DEVICES. FIG. 6A depicts a person/operator 10 showing an overlaid representation of facial veins including the superior labial vein 10-sla, while FIG. 6B shows traditional hand-held breath analyzer 2-2ba further adapted to comprise either or both of a fingerprint reader 2-2-s1 with optional pulse 2-h2 reader and/or a mouth insert 3-5 comprising a lip pulse 3-h4 detector (not depicted but see element 3-5-s1 of FIG. 4D in the related application). The related application taught the use of such further adapted breath analyzer 2-2ba with a primary device 1 such as a smartwatch 1-2 shown in FIG. 6C. The present invention teaches the further adaptation of analyzer 2-2ba to communicate directly with either of key fob 1-4 or vehicle 20 via any of vehicle 20's components such as vehicle access control 30-2 (as depicted) and/or operator readiness determination component 40-4 or control center 20-1.

What is important to see is that the breath analyzer can (1) self-authenticate its measurements, e.g., by using a fingerprint reader 2-2-s1, (2) be authenticated by a primary device 1 using any of a secured authentication signal 1-4-as including (1) light signaling, (2) pulse signaling where the pulse is detected as a finger pulse 2-h2 using detector 2-2-s1 or a lip pulse using detector 3-5-s1 (not depicted but depicted in the related application,) or (3) bio-conductive signaling using any portion of the analyzer 2-2ba as the “first” or “second” “body point” forming the conductive circuit with a primary device such as 1-2. Once authenticated, any companion data 1-4-cd such as the detected level of alcohol in the operator 10's breath, can be considered as authenticated and transmitted by the breath analyzer 2-2ba to the vehicle 20 along any or several communication paths (see also FIG. 6D) such as a) to primary device 1-2 which communicates with fob 1-4 as prior discussed, where the fob 1-4 then issues one or more authenticated and readiness vetted commands to the vehicle 20 such as an “unlock door” or even “start vehicle” request, b) to fob 1-4 providing either or both of an operator 10 authentication (presuming the fingerprint ID reader 2-2-s1, thus acting as a primary device 1) and an indication of readiness such as a blood alcohol score, where fob 1-4 then issues one or more authenticated and readiness vetted commands to the vehicle 20 such as an “unlock door” or even “start vehicle” request, or c) to a vehicle 20 component such as vehicle access control 30-2, operator readiness determination 40-4, or control center 20-1, providing either or both of an operator 10 authentication (presuming the fingerprint ID reader 2-2-s1, thus acting as a primary device 1) and an indication of readiness such as a blood alcohol score.

As prior discussed, there area many technologies currently in the market other than a hand held breath analyzer for determining a measure of blood alcohol, such as wearable devices and patches, where it is known that breath analysis typically provides the most reliable estimates but also is less convenient than a wearable.

The present invention teaches the determination of any number of “operator readiness” factors, including but not limited to alcohol levels, including but not limited to a measurement taken just prior to entering or operating vehicle 20. Thus, the example breath analyzer with further adaptations 2-2 depicted in FIG. 6B and its many possible communication paths such as shown in related FIGS. 6C and 6D, should be considered as exemplary rather than as a limited solution for vetting the alcohol or otherwise breath analysis readiness determination of the present invention, as many other technologies are possible and will become possible for sufficient use with the present teachings. For example, in a preferred solution, the primary device 1-2 comprises a blood alcohol sensor and thus is able to take multiple pre-session and even during session readings of the operator 10's alcohol levels for use in limiting pre-session vehicle 20 access and/or operation, or post entry/during session operation. As previously stated, it is also desirable that the determination of operator 10 readiness be based upon multiple factors and not just alcohol levels, such as also including a measures of drowsiness, glucose levels, and other foreign substances, all of which have wearable technologies already present in the marketplace or currently under laboratory development.

Referring next to FIG. 7, there is depicted system 100 deployed in three different configurations for the executing of the many and various system components described herein, where the configurations provide different tradeoffs such as between operator anonymity and operator-vehicle (asset) relationship complexity.

In brief, in the lower left corner system 100 is implemented using decentralized authentication variation 100-1, that optionally co-functions with asset/vehicle centralized authentication variation 100-2 (depicted in the lower right,) where then either of variation 100-1 or 100-2 optionally co-function with entity centralized authentication variation 100-3 (depicted in the upper left).

In decentralized authentication systems 100-1, an operator such as 10-tom enrolls with a privately controlled primary device 1 executing an intermediary app, such as a smartphone 1-1 or watch 1-2, where then preferably the primary device 1 provides data such as a preferably anonymized access key 30-4-ak to the vehicle access control component 30-4 associated with a vehicle such as 20-tom that the person 10-tom is desirous of entering and operating.

In asset centralized system 100-2, enrollment is at least in part hosted or otherwise conducted or maintained by an asset such as vehicle 20 (e.g., a shared vehicle 20-shr,) where then each authorized person 10-p1 and 10-p2 may then use a personal primary device 1 such as smartphone 1-1 or smartwatch 1-2 that includes issued access rights 30-4-ar-p1 or 30-4-ar-p2, respectively, to gain access to for example a shared vehicle 20-shr, where at least in some cases operator anonymity is desirable (e.g., a car shared in a family or group where the individuals are known) and in other cases operator identity is desirable (e.g., in a car sharing/rental situation where the individual car registers its uses and maintain records for liability purposes).

In entity centralized system 100-3, enrollment is at least in part hosted or otherwise conducted or maintained by an entity 40-ec (such as a vehicle rental service) comprising a fleet of vehicles 20-x (such as “11”, “17”, “21”, “53” or “79”,) where a possible person(s) 10-x (such as “A”, “B”, “C”, or “D”) is provided access rights 30-4-ar associating the specific person(s) such as “A” with a specific vehicle such as “21”. Authorized person(s) such as “A” then preferably use a primary device 1 such as smartphone 1-1 or smartwatch 1-2 to gain preferably non-anonymous access to and otherwise receive operation of the vehicle and may or may not have optional rights to authorize additional operators using either of systems 100-2 or 100-1.

As a careful reading of the present invention will show, especially the components described in relation to FIG. 1A, namely operator authentication and vetting 30-2, vehicle access control 30-4, operator detection 40-2, operator readiness determination 40-4, and vehicle operation limits 40-6, can be deployed in different system architectures, where processing of the components resides on a computing element 1) under the control of the operator 10, for example a primary device 1, 2) under the control of the asset 20, for example a compute and control center such as 20-1 of a vehicle 20, or 3) under control of a remote server that is neither (1) or (2). It is important to see that both the computing element for executing a particular component as well as the computing element for the storage of the data processed by a particular component do not have to be the same, as will be well understood by those familiar with computer systems architecture and networking. It is also important to see that the choice of a deployment architecture for the processing and data storage is an independent consideration from the anonymity of the data with respect to for example the operator 10, the asset 20, and the session of operation. Thus, the deployment examples portrayed and discussed in the present invention, especially with respect to the present figure, should be understood as representative of types (decentralized, asset-centralized, and entity-centralized) where those familiar with computing systems will understand that these general types have many variations.

There are many reasons for preferring a decentralized system 100-1, including the use case where the vehicle is owned by a person who is the operator 10, where the person (self-compliant use 5) or the guardian of the person (mandatory use 6) wishes to implement authentication and readiness restrictions for the perceived benefit of the person and the asset. In such cases, it is reasonable to assume that the access and operation of the asset (e.g., a vehicle) can be anonymous, and that what is most important is that the session of operation is properly authenticated (i.e., the person/operator 10 is validly identified for example to the intermediary app 1 using one or more biometrics,) properly authorized (e.g., that the authenticated operator 10 is allowed to operate the asset 20 at the place and time being requested, or even for example the route being requested in the case of a vehicle,) and properly readiness vetted (e.g., that the authenticated and authorized operator 10 is compliant with a health regiment that includes measures of impairment, thus “ready”). It is also noted that in a decentralized system, it is possible to record data regarding a session of operation that is either shared with an authorizing guardian or remains private to the operator 10, where session operation data is understood to be distinct from operation limits, such that for example a guardian understands that the asset is being used in accordance to the set and authorized operation limits, especially considering operator readiness, but is otherwise unaware of any other details of any particular session of operation.

There are many reason for preferring a vehicle-centric (generalized to be asset-centric) system 100-2, including the use case where the vehicle is owned by an individual or entity that allows authenticated, authorized, readiness-vetting sharing of the vehicle, where each session of sharing/operation can be recorded for the owner, and optionally include a payment-for-use transaction prior to or after the session of operation (see FIG. 2B, steps 30-ka-5 and 30-ka-7). For example, an asset can be a privately owned house (or a vehicle) that is shared on a rental basis, similar to the well-known “Airbnb” type service, where the present system teaches that the individual house/vehicle owner can enroll a guest “operator” for use of the house under certain conditions including readiness before entering the house and while in the house. Hence, the system components for enrollment, access control, operator detection, readiness determination and operation limits can all be deployed to a computing element essentially under the control of the asset owner for the control of the use of for example a single asset.

In both the use case of a house and a vehicle, it is further noted that the “other vehicle sensors and data sources” 20-5 (see FIG. 3A) can be generalized as “other asset sensors and data sources” and include “occupancy sensors,” of which many well-known solutions are available in the market. An occupancy sensor in the use case of a house detects the number of current occupants for example by monitoring all accessways, although other “room-by-room” sensors are also available. An occupancy sensor in the use case of a vehicle detects the number of current passengers and even their respective seat locations and possibly their identities especially if working with a primary device 1. In either case, current occupancy can be measured and is anticipated as a limitable value of operation 40-6 for which certain types of assets 20 such as buildings and vehicles can be limited or controlled using the present system 100 in any of its depicted and alternative variations. It is further noted that asset 20 access control 30-4 for a building or vehicle can be controlled as described herein for the operator 10 (which may then also be considered the “legally responsible” or “contracting” individual) as well as any other “non-operator” occupants, such as friends or co-workers in a building or friends or family in a vehicle. Thus, an operational limit as taught herein may be applied to the operator 10, the asset 20 and any of its operational features, or occupants, where occupants can be allowed without identification and for example up to a quantity limit or might only be allowed with identification and then also matching a specific enrolled individual.

There are many reason for preferring an entity-centric system 100-3, including the use case of a rental service such as Enterprise, Hertz, and many other services, where the service maintains a “fleet” of assets, each with possibly different features and benefits of operation as well as costs and liabilities. Such entities prefer to keep full and accurate records of each asset 20 rental, including information such as the identity of the operator 20.

Thus, a traditional asset rental agency performs the functions of enrollment (that is at least a part of component 30-2) on an entity controlled computing element, such as a server 50. In a traditional service, after a person who desires operation of a rented asset such as a vehicle 20 presents identification and thus both “enrolls” and “authenticates” themselves with the entity, they are typically provided with “unlimited” access control means, traditionally in the form of keys to a vehicle 20 or some other means for “self-service” and “unsupervised” access control, for which many access control means are possible including key cards and codes, or the ability to present biometrics to an access control system governing access to the asset (such as a building). At this juncture, after the operator is enrolled and authenticated to the rental agency, and they have received their asset control means, the actual entering and use of the asset is not traditionally monitored or tracked, thus being a disadvantage of the traditional systems.

The present teachings anticipate that the operator 10 during or prior to enrollment secures from the entity an intermediary app for use on for example their smartphone, thus in combination becoming a primary device 1.

Enrollment including registration of personal biometrics may then proceed on the primary device 1 with notice being provided by the intermediary app 1 to the entity server 50 upon sufficient completion of enrollment and authentication, where personal identity including biometrics may then optionally remain private as information known to the intermediary app 1 but not otherwise shared with the entity (where the entity may otherwise be provided with a unique operator ID for tracking and associating data purposes). It is expected however, that a traditional service that can benefit from the system 100-3 as taught herein, will prefer to receive identifying information including contact information about the person to be granted access and operation to the rented asset 20. As will be clear from a careful consideration of the teachings herein, and especially to those familiar with software systems and networks, any of enrollment and initial registration may be performed wholly or in part upon either or both of the primary device or entity server 50, where regardless the necessary and required personal information is ultimately made available to server 50, even if entered and also maintained on the primary device 1.

Still referring to FIG. 7, what will be apparent upon a careful reading and consideration of the present teachings is that the present system 100-3 may then associate a given asset 20 (such as “vehicle 17” in group of vehicles 20-x) for exclusive and limited operation by an enrolled and authenticated individual (such as “person C” in a group of persons 10-x,) and thus the person C is now “authorized” for operational use of vehicle 17. As prior described, this operation use may include re-authentication and readiness vetting (thus combined with decentralized system 100-1) for each session of operation as dictated by the entity in policy restrictions 22-pr and as desired by person C. Where then any or all of vehicle operation data 50-1 (see FIG. 1B) is then preferably captured and made available to the entity's server 50 providing significant benefits as will be obvious to those familiar with such possible entities. What will also be clear is that two or more persons typically traveling together in a traditional rental service scenario may only enroll and register a single individual for operation, after which the additional other person(s) at some later time might unbeknownst to the rental service also take over operation of the asset/vehicle 20, for which a careful understanding of the present invention will show that this additional sharing can be stopped by requiring authentication for each session operation. Combining entity-centric system 100-3 with vehicle centric 100-2, it is then possible that multiple individuals might rent a single asset/vehicle 20 for joint or shared operation, where then vehicle 20 has access to the enrollment information and serves at least in part to authenticate and authorize an individual for vehicle operation.

In still yet another example of the benefits of the present system, entities using system 100-3 can accumulate and “score” the operational usage of each enrolled operator 20, where then some or all of vehicle operation data 50-1, such as context data 10-d1, operator characterization data 10-d2, and operator/operation derived data 10-d3 are accumulated for determining operation assessments 50-2. Some insurance providers such as Progressive use data collector devices (such as “Snapshot” provided by Progressive) to be plugged into a modern vehicles control center 20-1 for receiving any of at least vehicle indirect metrics 20-m2 and transmitting the information back to an entity server 50 where it is scored for use in determining policy discounts and pricing. A careful consideration will show that multiple drivers sharing the same vehicle and “Snapshot” device will not be differentiated, thus the “session data” 20-m2 is not authenticated to be “of” or “in relation to” a specific identified person who is associated with the policy and its discount or pricing. The present system 100 not only teaches systems and methods for authenticating asset operation data 50-1, but also teaches how this data 50-1 includes both non-session data (e.g., pre-session personal health metrics 10-m) and session data (such as vehicle direct metrics 20-m1 and indirect metrics 20-m2 as well as any of context data 10-d1). The present invention also teaches a feed-back loop where any of (asset) vehicle operation data 50-1 becomes (asset) vehicle operation assessments 50-3, and where any and all of the accumulated operation data 50-1 including subsequent operation assessments 50-3 are accessible to both third parties 22 for updating polices and operation restrictions 22-pr based upon individual operator session performance and analysis and to the operator readiness determination component 40-4 for setting vehicle operation limits 40-6 on a session-by-session and even during session basis (also including the consideration of any updated policies 22-pr).

Many other benefits and advantages will become apparent upon careful consideration, where some benefits and advantages are different for each of variations 100-1, 100-2, and 100-3 of system 100. The reader is again reminded that system 100 is anticipated to be generalizable across virtually any asset including buildings, vehicles, equipment, industrial processes, etc., especially were control is localized to a given operator and the asset or operation of the asset carries significant value and/or liabilities, and thus should not be limited to the many driver-vehicle exemplary depictions and discussions.

Referring next to FIG. 8, there is shown a process-data structure diagram of the present operator-asset authority and readiness assessment system 100g that generalizes the exemplary operator-vehicle system 100 across multiple types of assets and operations. Generalized system 100g applies to any operation of any asset, where an asset comprises a vehicle or vessel, equipment and machinery or a process involving equipment and machinery, a structure for occupation such as a building or house, where operation by an operator is desirous and can be limited, and preferably for which operation is understood to include safety or otherwise concern for any of the asset, operator or other persons. Generalized system 100g process-data structure comprises operator rights-holder and asset management 114, operation rights-allower management 110, session data aggregator 118, third-parties, enforcement interface 116, and session monitoring and reporting 120.

The present diagram does not depict any particular deployment of processes and data using specific computing elements such as a primary device 1 that is “operator-controlled,” an asset-embedded computing element such as control center(s) 20-1 that is “asset-controlled,” or a computing element such as server 50 that is “entity-controlled” (where an entity has either an interest in the asset and/or the operation of the asset, but is otherwise not considered to be the asset operator for a session, where entity examples include an asset manufacturer, distributer, owner, controller, manager, provider, renter, insurer, operation licensor, operation rules enforcer, etc., where one example of which is preferably at least the issuer of the initial asset use rights to an operator, and where the issued use rights may be exclusively limited to the issued operator or available for transfer and re-issue by the initially receiving operator, see FIG. 2B) as many deployments are possible and some of which as described herein are preferred depending upon the use case and desired benefits, while other deployments that are not discussed will be obvious to those familiar with computing architectures and systems networking. However, for the purposes of teaching, the following discussion relating to the processes and data depicted without deployment in the present figure will be described with a preferred deployment being any of operator-controlled, asset-controlled, or entity-controlled.

Operator rights-holder and asset management 114 comprises operation rights-holder management 112 that cooperates with asset management 113. A core function of operation rights-holder management 112 is to determine privately authenticated, non-session data, task readiness and authorized operation limits 112p, all as prior discussed herein. A novel technical advantage of the present teachings includes the pre/post session nature of this operator 10 monitoring and data determination, where a careful understanding of traditional asset and process management will show that operator assessment is currently unable to provide objective physiological and/or psychological qualification and quantification of an operator absent a second or third party human evaluation which is situationally impractical and unachievable let alone significantly cost prohibitive, where these 112 component objective assessments are based upon privately authenticated wellness device measurements processed through a first-party (operator 10) primary device 1 into an on-going assessment, as opposed to a second or third party determined “spot assessment,” Determination component 112p is preferably operator-controlled and executed on a primary device 1, such as a smartphone 1-1 or smartwatch 1-2, or some combination thereof including companion devices, and provides authenticated non-session data 112-d1, from which at least in part is derived vetted operator readiness for task 112-d2, from which at least in part is derived authorized session operation limits 112-d3, all determined advantageously pre and post session operation.

A core function of asset management 113 is to determine privately authenticated, session operation data, task readiness and authorized operation limits 113p, all as prior discussed herein. A novel technical advantage of the present teachings includes the means for authenticating operation measurements captured during a session, which heretofore have been at least in part captured but otherwise non-authenticated (for example, a modern vehicle may measure driving performance but cannot assign such measurements to a particular identified driver/operator, and thus the measurements are not authenticated and then further cannot be accumulated into an operator history and further into operator type histories, all supporting significant derived assessments (such as baselines and normals). Determination component 113p is preferably asset-controlled and executed on the asset 20 or an associated server 50 in communication with asset 20 (where for example communications and control center(s) 20-1 can accommodate both configurations was well as communicating with a primary device 1) and provides authenticated session data 113-d1, from which at least in part is derived vetted operator readiness for task 113-d2, from which at least in part are implemented session operation limits 113-d3, all determined by necessity during session operation.

Each of core functions 112 and 113 cooperate to enable asset entrance (if applicable), occupation (if applicable), and operation 112-4. Such cooperation as taught herein provides a novel technical advantage for decreasing overall performance risks by providing objective and cost-effective means for limiting asset operation to authenticated, authorized, and readiness vetted operators 10. Regarding operator readiness, the present invention further anticipates that readiness can be a confirmation of sufficient training or competency with respect to the asset in consideration for operation. For example, many vehicles require an operator's license which certifies an operator as to being sufficiently trained, where also licenses may be for different levels or classes of a similar type of asset or operation. In another example, rented assets or equipment often have some complexity or even new features for which the renter/rights-allower might be desirous of best ensuring that select operation information including advisory notices and usage guides are provided, received, and/or accepted prior to any allowed session operation. In yet another example, proof-of-readiness might include an objective measure of prior experience operating the same or a similar asset, and/or for the same or similar operational use, and/or for the same or similar contexts.

It is well-known that operator licenses can be represented as certified digital information, where the present system anticipates that for example, the intermediary app running on the primary device 1 can be used by a person to acquire sufficient digital certification prior to requesting operation of an asset such that the primary device augments readiness to include proof of proper asset operation licensing. It is also well-known that advisory notices, training guides, feature update descriptions, etc., can be provided digitally as well, where the present system anticipates that for example, the intermediary app running on the primary device 1 or in combination with a networked computing system can be used by a person to receive, review, and/or accept any such notices, guides, and descriptions such that the primary device augments readiness to include proof of receipt of renter/rights-allower communications and information, or otherwise asset and/or operation related communications and information. As a careful review of the present invention will show, another novel technical advantage of the present teachings is that the prior experience of the operator operating the same or a similar asset, and/or for the same or similar operational use, is objectively measured and accumulated such that the present system 100g for example provides such information as additional vehicle operation assessments 50-3 and further augments readiness to include sufficient operator experience.

Thus the reader will see that operator readiness as herein taught comprises any one of or any combination of physiological assessment, psychological assessment, use-license assessment, notification confirmation, training assessment, experience assessment, as well as other assessments that will be clear and obvious variations of those exemplified and discussed herein. (It is also important to see that each of the aforementioned assessments are substantially non-session assessments, although not exclusively as at least physiological and psychological assessment may continue during a session of operation, and thus formed primary using pre-session data and measurements which are traditionally not available for asset operation regulation.) The careful reader will also see that in addition to operator assessment, the present invention also anticipates asset readiness assessment that can include asset self-measurements (e.g., a vehicle indicating that “all systems are go”,) asset third-party certification (e.g., a maintenance crew certifies that a vehicle has been inspected,) asset age and wear assessment and thus “predictive” or “preventative” assessment, as well as many other possible types of asset self-assessment and third-party assessment, where using such asset assessment in combination with operator assessment to determine an overall session operation readiness is another novel technical advantage of the present invention.

Still referring to FIG. 8, another novel technical advantage of the present system is operator rights-holder and asset management 114 comprising a core function to determine, distribute and authorize operation rights criteria 110p, all a prior discussed herein. Determination component 110p is preferably executed pre-session by an entity-controlled computing element such as server 50 (which may or may not also be the same computing element as for performing the aggregation of session related data 118). A novel technical advantage of the present teachings includes the pre-session nature for establishing multi-use or single-use rights criteria for an asset preferably linked to a specific operator 10 and either transferable or non-transferable, where the established criteria are “live” and both measurable and enforceable during asset 20 entrance/access (where applicable) and during asset 20 operation, and where the criteria are updatable post-session forming a feedback loop for adjusting criteria based upon changing and objective operator and operation-results data, where a careful consideration of traditional asset and process management will show that use-criteria including any of enrollment, authentication, authorization, and readiness vetting are not used for setting operator, asset, and/or session-of-operation individualized conditions of operation 110-d2 or operation policy restrictions 116-d2 that become enforceable as session operation limits 112-d3, 113-d3.

Determination component 110p at least in part does any one of or any combination of initiating, determining, establishing, maintaining, and otherwise managing enrollment and authentication data 110-d1 (data of which may be completed at a later time by an operator using a operator-controlled primary device 1 or a asset-controlled device 20-1,) from which at least in part is derived conditions of operation 110-d2 (data of which may be overridden or altered at a later time by an operator using a operator-controlled primary device 1 or a asset-controlled device 20-1,) from which at least in part is derived transfer of rights, payments 110-d3 (payments of which may be made in any of well-known methods including preferably e-commerce/electronic payments). It is important to see that the present invention teaches technical means for limiting a “transferred right” to be less than the “full operational use” of an asset, and thus some asset functions or features may be limited, and further for limiting any particular operation-use-limitations to an assessment of operator authenticity, authorization, or readiness, where this then allows for the transfer and payment of a “conditional-right” that is determinable at the time of use, where it is further anticipated that payment or additional debits and credits to the initial transfer payment are determined and processed after the per session determination of the final conditional-rights.

Still referring to FIG. 8, yet another novel technical advantage of the present system is session data aggregator 118 comprising a core function to determine operator/asset/session assessments and derived data 118p, all as prior discussed herein. Determination component 118p is preferably executed post session by an entity-controlled computing element such as server 50 (which may or may not also be the same computing element as for operation rights-allower management 110). A novel technical advantage of the present teachings includes the post-session evaluation and determination of any one of or any combination of specific operator, operator type, specific asset, asset type, specific operation, operation type, context related, operator or asset characterization related, and other possible assessments including algorithmic functions computing scores, scaler representations, rankings, and otherwise qualification and quantifications all based upon aggregated authenticated non-session and session data 118-d1, where a careful consideration of the of traditional operator-asset evaluation will show no generalized solutions for use in a broad consumer market comprising a multiplicity of potential operators and a multiplicity of potential assets, and furthermore will show limited to no ability to determine or combine authenticated pre, during, and post session relevant data as well as no capacity for providing this range of data using any of an anonymized operator, an anonymized asset, or an anonymized session of operation.

Determination component 118p at least in part does any of or any combination of receiving, storing, maintaining, and processing session related data as authenticated non-session and session data 118-d1 (alternatively described as authenticated asset [vehicle] operation data 50-1, see FIG. 1B) provided by one or more instances of operator rights-holder and asset management components 114 (or any other component of the system 100g,) from which at least in part is determined authenticated derived data 118-d2 (alternately described as authenticated operator/operation derived data 10-d3, see FIG. 1B). A novel technical advantage of the present teachings includes aggregating (especially but not limited to a broad consumer market) authenticated non-session and session data including operator, asset, and operation data, where also the privacy rights of individual operators, and asset “owners” (of any type herein mentioned) are optionally protected via via any of well-known anonymization methods or as otherwise taught herein.

Still referring to FIG. 8, yet another technical advantage of the present system is third-parties enforcement management 116 comprising a core function to determine operation/asset/session policies and enforcements with operation limits 116p, the functions of which are described as follows. It is well understood that in any given marketplace comprising a multiplicity of independent persons wishing to be operators of a multiplicity of possible assets over a multiplicity of different contexts and thus a multiplicity of different sessions of operations, that the market is likely to include operation insurers (as prior discussed) and operation regulation and rules enforcers (e.g., police with respect to drivers and vehicles used on public roads). Thus third parties at least includes insurers and enforcers that have a significant interest is obtaining for example session related data from aggregator 118 (as well as potential other system 100g determined data). While any such system 100g data, for example specifically including some or all of session data from aggregator 118, may be made available to such third-parties, the present system anticipates anonymizing any of this data and/or encrypting and privatizing its access to specific third-parties under specific situations.

Another means for providing useful system information to such third-parties is through component 116p's processing of such data into operator/asset/session reports and claims 116-d1, where such reports should be generally understood to include any of well-known means for retrieving and supplying in any paper or digital form system data typically related to a “query” or topic of interest.

With respect to claims, a careful consideration of the present invention will show significant benefits provided by the many novel technical advantages taught herein especially for determining pre, during, and post session authenticated data, where for example a traditional accident claim filed with an insurer might include some presumably authentic digital images, possible accurate written statements, and even enforcer (e.g., police) assessment statements, but lack the many authenticated data discussed and described herein applying to any one of or any combination of the operator, the asset, or the session of operation. This data can for example reveal causes and reasons for a given accident either in support of or questioning a given filed claim. This provision of information 116-d1 is akin to “recovering the black box/flight data recorder” associated with attempting to understand an airplane accident where then by comparison of airplane to automobile operation, the airplane is akin to the vehicle/car, the pilot(s) are akin to the driver, and the flight is akin to a road trip. A careful consideration will also show the same or similar interest in system 100g's data authenticated non-session and session data by third-parties responsible for enforcement and protection of the public's interests (such as but not limited to law enforcement). There are no such traditional systems for achieving these third-party enforcement 116 goals and needs thus highlighting another significant advantage of the present system 100g in any and all of its variations.

Still referring to FIG. 8 and third-parties enforcement component 116, as will be understood by those familiar with computing systems, any of non-session and session data determined at least by operator rights-holder and asset management component 114 may be made available as session monitoring and reporting 120 substantially in real-time to any of the interested third-parties, at least as legal agreements and governing laws and rules provide or anticipate. For example, if a driver/operator 10 has been arrested for “driving under the influence” (“DUI”) the enforcement party may require operator 10 to for example use any of the system taught or anticipated impairment/readiness determination apparatus and methods, such as a breathalyzer 2-2 discussed in relation to FIGS. 6A, 6B, 6C, and 6D or the use of a primary device 1 for tracking readiness including an on-going state of impairment, where then at the commencement of a session or during a session any of non-session or session related data can be automatically transmitted as data 120 via any of well-known electronic communications to the enforcement party, where such data 120 might include the current geographical location of the asset (thus “context data”) and where the processing of such received real-time data might cause feedback to the asset or operator changing the limits of operation essentially in real-time. And finally with respect to FIG. 8 and the general system 100g as well as its many taught an anticipated variations, the careful reader should understand, and as those familiar with data processing will know, data classified herein for clarity as “pre,” “during,” or “after” type session should not be unnecessarily limited to the described classification, as such data might also be determinable or at least provided as some other type “pre,” “during,” or “after” than described herein. With respect to the various system process and data components 114, 110, 118, 116, and 120 depicted in FIG. 8, there are shown arrows connecting these components and even arrows connecting the sub-components 112 and 113 of system component 114, where it should be understood that these arrows represent a preferred and primary direction of flow but otherwise should not be limited to only the depicted flow directions, as any and all components my “exchange information” thus requiring a depiction of an arrow head in both directions.

Referring next to FIG. 9, there is depicted a series of four variations (1-g1, 1-g2, 1-g3, and 1-g4) of a generalized primary device 1-g used for determining and maintaining private and authenticated on-going health measurements for forming a private and authenticated current health status of an operator 10. A purpose of the teachings of these four variations is to help the reader understand the generalized primary device 1-g, implemented in various specie (such as but not limited to 1-1 and 1-2 depicted herein,) for accomplishing the private “authentication” of an operator 10's health state for use in the present readiness assessment system 100. Just as a “driver-car” specie of the present readiness assessment system 100 has been shown to generalize (e.g., see FIG. 8) over virtually any type of asset 20 including for example vehicles, equipment, processes, and even services (which can be seen as either physical or digital/virtual assets or processes provided on a per-use basis to an operator,) similarly the primary device 1 generalizes as 1-g into core components and functions which have many possibly deployment variations, some exemplary species having been discussed (such as 1-1 and 1-2,) while others as anticipated herein, and while yet still others to be clear from a careful understanding of the generalized “primary device pattern.” Within this generalized pattern, it is also noted that all of variations 1-g1, 1-g2, 1-g3, and 1-g4 can interact with any one or more companion devices 4 to gather and authentic additional health measurements (see especially upcoming FIG. 9B for more discussion).

Starting with leftmost generalized primary device 1-g variation 1-g1, the core implemented functions are 1-g1-1 for confirming the operator's ID using biometric sense data and 1-g1-2 for determining one or more bio-measurements (such as heart rate, breathing rate, emotional state, etc.,) where it is noted that the determining of a rate (changing pattern over time) necessitates that the “biometric sense data” such as an image also continue to sample the operator over time, thus being a video or at least a series of two or more images. At least one example of device 1-g taught in the related patents was the use of a smartphone using what is referred to in the art as “remote photoplethysmography” (“rPPG”) to capture a video clip of a person (the clip then being the “biometric sense data”) from which can be determined both the identity of the person (e.g., by using any of well-known facial recognition algorithms and comparing to a “pre-registered” image(s) of the same person) and one or more bio-measurements as mentioned. In the present teachings, smartphone 1-1 is an example specie of generalized primary device 1-g1 and substantially functions as described in the present paragraph and as discussed in greater detail in the referenced patents. (For the interested reader, there is at least one company, Binah, headquartered in Israel, that is currently offering a smartphone-based app that determines at least pulse rate of a person using rPPG.)

In another specie example of the generalized primary device 1-g1, key fobs 1-4a and 1-4b are shown to alternatively comprise identity sensor 1-4-s1 for sensing a fingerprint (the “biometric sense data”) sufficient for determining identity, while then substantially simultaneously determining a health measurement that is a biometric rate being the pulse. This pulse data is typically determined using well-known photoplethysmography (“PPG”) using LED(s) in close proximity to the person's skin (thus different from “remote” PPG). This PPG determined pulse data may be different from the “biometric sense” data sufficient from determining the fingerprint (for which many sensing technologies are known,) where then the reader should understand that the present invention considers all bio data collected substantially simultaneously and from substantially the same body surface (for example a fingertip or a face,) albeit with the same or different sensor technologies, to be generally “biometric sensed data” usable for determining both the operator identity and the one or more bio-measurements. In yet another variation, as a careful consideration will show, the traditional “start button” in a more modern automobile can be further adapted into include one or more biosensors such as a fingerprint reader, such that enabling the start function comprises being properly identified along with pressing the button, where then the contact surface of the start button can further include other sensors, such as for measuring what is sometimes referred to as subcutaneous sweat useful for determining levels of blood alcohol.

Other variation species of generalized primary device 1-g1 are possible. What is important to see as a core function is that bio-data is gathered at substantially the same time or over substantially the same duration of time of substantially the same surface area (e.g., the face or the fingertip) of a person using one or more sensors (e.g., a camera or LEDs,) and that this one or more sensor data is the herein referred to “biometric sensed data” from which at least can be determined (a) a form of person identification (to any sufficient level of accuracy,) and (b) at least one health measurement. It is also important to see that a device 1-g1 (as well as upcoming device 1-g2) determines the one or more health measurements per each sampled biometric sense data, and as such a new person identification must be accomplished for each next health measurement over time. Note that this is different as compared to generalized primary devices 1-g3 and 1-g4 (where 1-g4 comprises 1-g1,) each of which is capable of capturing and authenticating “on-going” health measurements without needing to re-identify the person about which the health measurements are being determined, all as will be discussed further. It is also important to see that device 1-g1 determines its one or more health measurements using sensors comprising the device 1-g1, whereas device 1-g2 to be discussed next determines at least one of the one or more health measurements using sensors comprising a “companion” device 1-4.

Still referring to FIG. 9A, next is discussed generalized primary device 1-g2 comprising core implemented functions 1-g2-1 for confirming the person's ID using biometric source data, 1-g2-2 for substantially simultaneously using a “secured authentication signal” (see especially FIG. 5B and the related patents) associated with the biometric source data to substantially simultaneously communicate with a companion device, and 1-g2-3 for substantially simultaneously determining one or more bio-health measurements using the companion device. Function 1-g2-1 is like function 1-g1-1, and may for example be accomplished by a smartphone using a camera to capture one or more images of a person to be identified and “health measured.”

Function 1-g2-2 comprises any of a secured authentication signal, such as any of “light signaling” or “pulse signaling” as first discussed in the related applications, or “bio-conductive signaling” as first taught herein, to authenticate a companion device 1-4. The reader is directed to the referenced art and to FIG. 5A herein for a further understanding. What is important to see is the secured authenticated signal serves to authenticate the companion device, where then any one or more health measurements determined by the companion device 1-4 substantially simultaneously with the provision of the authentication signal, are considered to thereby by authenticated health measurements of the person, even though these measurements are for example determined with sensors within the companion device and even though these sensors may be measuring a surface area of the person that is not substantially the same as the surface area used in function 1-g2-1.

The reader is directed to the related applications for exemplary species of generalized device 1-g2-1 comprising for example the combination of a mobile device, such as a smartphone capable of sensing the person for example using a camera, and a companion device such as a digital thermometer (using light signaling) or a smart scale (using pulse signaling). What is important to see as a core function is that bio-data is gathered at substantially the same time or over substantially the same duration of time but now possibly related to different surface areas of the person. Like generalized device 1-g1, device 1-g2 does not determine “on-going” health measurements and as such must re-identify the person (function 1-g2-1) about which the health measurements are being determined for each “instance of determination” (thus separated by a substantial amount of time that is beyond the duration of the capturing of the bio-data, for example minutes, hours or days).

Still referring to FIG. 9A, next is discussed generalized primary device 1-g3 comprising core implemented functions 1-g3-1 for determining that the device is in a “locked state” necessarily comprising a “locked body location” (or alternatively “locked body surface area for sensing one or more bio-data such as the back of the wrist for a watch or a portion of the finger for a ring,) 1-g3-2 for confirming the person's ID using biometric source data and determining at least one “bio-rate (A)” (or alternatively any “bio-rhythm” such as a heartbeat/pulse or breathing) associated with the biometric source data, 1-g3-3 for substantially simultaneously determining from the “locked body position” a “bio-rate (B)” of the same type (e.g., heartbeat vs. breathing) as “bio-rate (A),” 1-g3-4 for comparing bio-rates (A) and (B) as a means for authenticating the locked body location, and 1-g3-5 for substantially determining one or more bio-measurements from the now authenticated locked body location “on-going” while the device remains in a locked state. In the present teachings, smartwatch 1-2 is an exemplary species of generalized primary device 1-g3 and substantially functions as described in the present paragraph and as discussed in greater detail in the referenced patents.

In another specie example of the generalized primary device 1-g3, it is possible to further adapt the existing marketplace device known as the Oura Ring (or similar competing “wellness device” rings) to further include an external facing fingerprint ID/pulse sensor such as sensor 1-2-s1 depicted on smartwatch 1-2 (see FIG. 4A) or sensor 1-4-s1 depicted on fob's 1-4a and 1-4b (see FIG. 4B). As a careful consideration of the workings of smartwatch primary device 1-2 will show (see especially the referenced patents,) this further adaptation of a “wellness ring” such as the Oura Ring allow the ring to (a) determine the fingerprint and pulse (i.e., “bio-rate (A)”) of a person touching this external facing sensor 1-4-s1 while then substantially simultaneously determining the finger pulse (i.e., “bio-rate (B)”) of the person upon which the inside surface of the ring is currently placed, where if bio-rate (B) sufficiently matches bio-rate (A), then the ring is “authenticated” as being currently worn by the person who has provided the fingerprint ID. As a careful consideration will show, the ring itself can determine for example using any of a skin contact sensor or the monitoring of a pulse if it remains on the now authenticated finger, and if remaining, then over any duration of time any of additional “on-going” bio measurements may then be taken of the also now authenticated finger surface area (i.e., the “locked body location”) substantially in contact or close proximity with the inside of the ring.

Still another example of a generalized primary device 1-g3 would be what is often referred to as a “body patch,” such as a glucose monitoring device that has been further adapted to include a similar ID/bio-rate A sensor such as 1-2-s1 or 1-4-s1, where then the patch is also further adapted to determine a similar rate B (such as a pulse) from its body contact surface, for which many body contact surface pulse measuring technologies are well-known in the art. The related patents also discussed a chest band sufficiently adapted to “clasp” and detected its own “locked” or “open state” along with bio-measurement sensors and a similar fingerprint/pulse sensor such as 1-2-s1 or 1-4-s1, where in general these devices were referred to in the related patents as “secondary devices” (because they did not also contain the full “intermediary app”/“honest broker” for determining the on-going health status but rather off-loaded their authenticated bio-measurements to preferably a paired smartphone running the intermediary app). It is important to see that device 1-g3, unlike 1-g2, can determine its health measurements on an on-going basis once having accomplished the authentication of the locked body location and while this locked body location is determined to still be substantially in contact or in close proximity to the body location, for which many technologies are well-known and have been discussed herein as well as the related patents. Like devices 1-g1 and 1-g2, device 1-g3 can be further adapted (through the use of a secured authentication signal) to communicate with and authenticate bio-measurements determined by any number of companion devices 1-4.

Still referring to FIG. 9A, next is discussed generalized primary device 1-g4 that is referred to herein as a “composite primary device” 1-g4 (see upcoming FIG. 9B) and provides several critical technical and marketplace advantages, such as allowing for an “all-software” solution (to be explained further shortly) for providing privately determined, authenticated, on-going bio-measurements leading to an privately authenticated and on-going health status, such as is discussed for use with the herein taught readiness assessment system 100. Another advantage is that the composite primary device 1-g4 can be implemented using existing marketplace devices (such as a smartphone combined with a traditional “fitness tracker,” a traditional smartwatch (thus not smartwatch 1-2 which has been further adapted over the state-of-the-art,) or well-ness “ring” such as the Oura Ring.

As depicted, generalized primary device 1-g4 comprises the generalized device 1-g1 (such as a smartphone running the intermediary app, thus 1-1) working in combination with a companion device (again, and for example, devices like the well-known Fitbit, Apple Watch, or the Oura Ring,) where the companion device does not require any further physical adaptations (such as adding fingerprint/pulse sensor 1-2-s1 or 1-4-s1,) and where the two devices 1-g1 a and the companion remain physically separate as traditionally found in the marketplace but in conception working together provide for the “composite” primary device 1-g4. Primary generalized device 1-g4 uses generalized device 1-g1 to implement core functions 1-g1-1a for confirming the person's ID using biometric source data and determining at least one “bio-rate (A)” (or alternatively stated, a bio-rhythm such as a heartbeat/pulse or breathing) associated with the biometric source data (that is like core function 1-g3-2,) and 1-g1-1b for comparing bio-rates (A) and (B) (where rate (B) is provided by the companion device in function 1-g4-c2 to be discussed shortly) as a means for authenticating the locked body location associated with the companion device (that is like core function 1-g3-4).

Composite companion device 1-g4-c comprises core implemented functions 1-g4-c1 for determining that the device is in a “locked state” necessarily comprising a “locked body location” (or alternatively “locked body surface area for sensing one or more bio-data such as the back of the wrist for a watch or a portion of the finger for a ring) that is like core function 1-g3-1, 1-g4-c2 for substantially simultaneously determining from the “locked body position” a “bio-rate (B)” of the same type (e.g., heartbeat vs. breathing) as “bio-rate (A)” that is like core function 1-g3-3, and 1-g4-c3 for substantially determining one or more bio-measurements from the now authenticated locked body location “on-going” while the device remains in a locked state that is like core function 1-g3-5. As a careful consideration will show, the core functions of generalized primary device 1-g3 have been redistributed to be implemented on two separate devices (namely 1-g1 and a companion device 1-g4-c,) rather than being implemented on a single device (such as a smartwatch 1-2). A careful consideration will also show to those familiar with such devices, that a smartphone has considerably more processing power, data storage, and battery life than a typical smartwatch that might be further adapted becoming primary device 1-2. Thus, by off-loading key functions that include the intermediary app and simply using the companion device 1-g4-c (such as a traditional fitness tracker, smartwatch, or wellness ring) for authenticated on-going data collection, generalized composite primary device 1-g4 is shown to be an “all-software” implementation in the respect that it uses traditional and currently available devices/hardware and that all other adaptations are implemented in software providing considerable technical and marketplace advantages.

A careful consideration will also show that a composite device 1-g4 can comprise the generalized device 1-g1 along with one or more companion devices, such that generalized device 1-g4 is not limited to only two cooperating physical devices. For example, device 1-g4 might include the use of both a Fitbit and an Oura Ring, or any of the other companion devices known in the marketplace and discussed in the related patents. It is important to see that any companion device 1-g4-c must include a means for providing a secured authentication signal, for example the detection of a body pulse (thus “bio-rate B”) substantially simultaneous to the determination of any one or more health measurements provided by the companion device, where those familiar with a Fitbit (or in general most “fitness trackers”,) an Apple Watch (or in general most smartwatches adapted to also determine health measurements,) and an Oura Ring (or in general most wellness rings adapted to also determine health measurements) will appreciate that these existing marketplace devices do already determine the wearer's heartrate/pulse/“bio-rate B” that is useable as an authentication signal (see the related patents).

What can also be understood by a careful consideration is that the combination of the generalized device 1-g1 and a first companion 1-g4-c for forming composite device 1-g4 as just described, provides for an additional technical advantage in that the composite device 1-g4 can now itself determine an “on-going” authenticated bio-rate A (after the initial authentication provided by the composite functions shown in the present figure) on an as-needed basis by using the prior authenticated locked body location of the companion device 1-g4-c to determine a “current” bio-rate (such as the body pulse) similar to that first used in device function 1-g4-c2 but now, since the composite device 1-g4 has already been authenticated, using this “current bio-rate” as rate-A for comparison with yet another and different companion device 1-4, where the different companion device 1-4 is then providing the current bio-rate B associated with its substantially simultaneously sampled health measurements.

Thus, composite primary device 1-g4 now functions similar to generalized device 1-g3 without requiring any “hardware adaptations” to any of the companion devices 1-4 used as 1-g4-c or the device 1-g1 being used in the composite device 1-g4. What can be further understood is that like device 1-g3, device 1-g4 can now authenticate bio measurements from a “non-lockable” companion device 1-4 (such as a smart scale, exercise equipment, or even a wearable that could determine a locked state except that it is not so further adapted, such as an exercise chest band,) where the “non-lockable” companion device 1-4 is not first “locked” to a specific body location but rather uses any of the authentication signaling methods such as preferably providing a “current bio-rate B” pulse that coincides with the companion device 1-4's health measurement(s). Hence and for example, the present teachings show the advantageous combination a smartphone running the intermediary app working in combination with a first lockable wearable such as a fitness tracker, smartwatch, or wellness ring (together forming a composite primary device 1-g4) and then as the composite device 1-g4 capturing on-going health measurements from any number of other companion devices that are either of lockable or non-lockable.

Referring next to FIG. 9B, there is depicted a pictorial-and-block diagram showing the composite primary device 1-g4 comprising a primary device 1-g1 (with “per-ID” authentication) such as a smartphone with intermediary app 1-1 working in combination with a “lockable” companion device 1-g4-c that substantially provides an on-going authentication signal for example a pulse (but does not provide an intermediary app or biometric ID sensor means,) where the provided signal is usable for authenticating health measurements provided by other companion devices after the time of initial authentication of that person. Composite primary device 1-g4 (comprising two separate devices 1-g1 and 1-g4-c) is shown as being similar to a “single-device”primary device 1-g3 with “on-going” authentication. Thus, composite primary device 1-g4 and “single-device” primary device 1-g3 can be seen as substantially implementing the same core functions in a different two-device versus one-device deployment, all as discussed in relation to the prior FIG. 9A.

In total, the present FIG. 9B depicts three different generalized species of the genus primary device 1g, where in general a primary device 1g is intended for “private” use and is responsible for (1) authenticating a person using a currently captured biometric for comparison with a prior captured/“registered” or “pre-registered” biometric, (2) determining one or more health measurements of the person is such a way as to create assurance that the one or more privately determined health measurements are “authenticate,” or “of the person,” and (3) preferably also determining a therefore authenticated health status of the person using any one or more of the one or more authenticated health measurements. These three different species include a primary device 1-g1 operating with “per-ID” authentication, a primary device 1-g3 operating with “on-going” authentication, and composite primary device 1-g4 also operating with “on-going” authentication.

“Per-ID” authentication means that each subsequent one or more health measurements determined at a time that is substantially outside of (i.e., timewise beyond) the duration of time of the initial determination of bio-metric data that was used at least in part to initially determine the person's identity (see core function 1-g1-1 in FIG. 9A) requires a new subsequent determination of temporally distinct bio-metric “ID” data captured substantially during a subsequent time overlapping the determining of the one or more subsequent health measurements. For example, a smartphone with intermediary app 1-1 is an exemplary specie of the generalized specie 1-g1, where at least in one variation the device 1-1 is taught to capture one or more images of the person, where these captured images serve as the bio-metric “ID” data for identifying the person (such as by using any of the well-known facial recognition algorithms,) and where the capturing happens over some finite duration of time (e.g., 3 to 5 seconds). As discussed especially in the related art, it is possible to also derive one or more health measurements using the same bio-metric “ID” data, such as a pulse determined by small color variations over time around the eyes where the skin in thinner, or in the checks of the person (both referred to in the art as “remote Photoplethysmography” (“rPPG”),) as well as for example an estimated body temperature, breathing rate, and through facial analysis an emotional state.

It is also possible “per-ID” to use secured authentication signaling (see the related patents and especially FIG. 5B of the present application) to gather one or more additional health measurements as sensed by a companion device 1-4, thus 1-g1 is functioning as 1-g2. In either case 1-g1 or 1-g2, the careful reader will see that each health measurement requires a bio-metric measurement sufficient for determining the ID of the person of whom the health measurement is currently being determined. For example, if two distinct measurements of a heart rate are determined by 1-g1 two minutes apart (i.e., enough time between to exceed the duration of initial ID core function 1-g1-1,) then each distinct measurement requires its own “re-authentication” with it own distinct set of biometric ID data. It is desirable to perform this core person authentication once and then to possibly determine one or more health measurements at times substantially after the authentication of the person without having to “re-authenticate,” thus referred to in the present application as the “on-going” authentication of any subsequent health measurements.

Still referring to FIG. 9B, generalized primary device 1-g3 is capable of such “on-going” authentication of subsequent health measurement(s), where no additional biometric data for authenticating the person is required during the duration of time that each of these subsequent health measurements are being determined, and where these subsequent health measurement(s) are determined either directly by primary device 1-g3 or in combination through secured authentication signaling with a companion device 1-4. Smartwatch 1-2 as discussed in the related patents and herein is an exemplary species of the device 1-g3.

As a careful reading of the related patents and present application will show, smartwatch 1-2, that is an example of device 1-g3, can (1) be clasped/“locked” to a person's wrist, (2) determine a bio-ID (in this example a fingerprint) with a matching “bio-rate A” (in this example a fingertip-pulse) using a sensor for example located on the watch band, (3) determine a wrist-pulse (“bio-rate B”) using the sensors located substantially on the underside of the watch or band and thus in contact or close proximity to the person's wrist, and (4) confirm that bio-rate B substantially matches bio-rate A thus enabling the biometric of the fingerprint to authenticate the wearer of the smartwatch 1-2. As will also be clear, any additional health measurements determined by sensors comprising smartwatch 1-2 can then be considered to also be authenticated as belonging to the person matching the provided biometric data (i.e., e.g., the fingerprint ID). A companion device 1-4 may then communicate with the smartwatch 1-2 at some later point after person authentication in order to provide subsequent health measurement(s) along for example an “authentication signal” such as a pulse (thus “bio-rate B”) that can then be compared by the smartwatch 1-2 to a substantially concurrently resampled wrist pulse (thus and now acting as “bio-rate A”,) where upon determination of sufficient A-B match the companion 1-4 provided health measurements are said to be “authenticated” and “of the person” without any requirement for person “re-identification” (and thus providing “on-going” operation). As a careful consideration will show, an on-going primary device 1-g3 provides technical advantages over a per-ID primary device 1-g1/1-g2, but then also requires a further adaptation of an existing marketplace device, such as a smartwatch or fitness tracker that traditionally do not comprise person ID and associated bio-rate A apparatus (e.g., a fingerprint reader that also detects the finger pulse). Generalized composite primary device 1-g4 provides an important alternative to devices 1-g1 and 1-g3 in that like 1-g1 but unlike 1-g3 device 1-g4 does not require any further adaptations to existing marketplace wearables (such as smartwatches, fitness trackers, wellness rings, fitness chest bands, etc.) but unlike 1-g1 and like 1-g3 can determine on-going health measurements without requiring the person to subsequently re-identify themselves to the composite device 1-g4 for each on-going health measurement. Thus, as a careful consideration will show, composite primary device 1-g4 can be deployed as an “all-software” solution where for example the intermediary app running on a smartphone 1-1 performs the normal 1-g1 functions of capturing an ID biometric (such as a facial image) for identifying the person along with a bio-rate A (such as the pulse detected in the facial image) while then substantially simultaneously communicating with a “lockable” companion device 1-g4-c to determine a current bio-rate B, and where if the intermediary app running on smartphone 1-1 determines a sufficient match between bio-rates A and B than the “lockable” companion device 1-g4-c can be considered as authenticated and therefore as being currently worn by the biometrically identified person up until the point where the device 1-g4-c determines that it has been removed from (i.e., “unlocked,” “unclasped,” etc.) the person (for which at least contact sensors and pulse detectors have been shown to be sufficient and for which at least some existing marketplace wearables are currently equipped thus not requiring further adaptations).

Exemplary species of the generalized “lockable” companion device 1-g4-c include a traditional smartwatch 1-g4-sw (such as the Apple watch) or a fitness tracker (also 1-g4-sw, such as a Fitbit,) both comprising the apparats to sense a wrist pulse, or a traditional a fitness-ring/smart ring 1-g4-sr (such as the Oura Ring) comprising apparatus to detect a finger pulse. New market devices are also anticipated such as a smartband 1-g4-sb that comprises means for communication with the primary devices, detecting a bio-rate B such as a wrist pulse, and any necessary computing and power apparatus, all as will be understood by those familiar with wearables. Smartband 1-g4-sb may for example draw a small amount of power as compared to a traditional smartwatch or fitness tracker, may be much smaller and thinner in size as compared to a traditional smartwatch or fitness tracker (thus the current depiction is not limiting,) may be better water-proofed, may be significantly lower in cost to manufacture, and in general may provide for a significantly longer-term wearable as compared to other traditional solutions (such as 1-g4-sw and 1-g4-sr) that require at least removal for battery charging. Such a smartband 1-g4-sb may further comprise additional apparatus for self-recharging of an embedded battery, where several technologies including “RF harvesting,” “thermoelectric devices,” “mechanical conversion to electric” devices, etc., are all well-known, and with other technologies available and anticipated such that the smartband 1-g4-sb, by virtue of needing minimized power and including self-recharging offers a composite primary device 1-g4 with “continuous authentication” (being the same as “on-going” but then also for a considerably longer period of time between recharging).

The careful reader will also see that it is possible to use a composite device 1-g4 comprising for example a smartband 1-g4-sb to allow for on-going “instant authentication” of another wearable, such as a smartwatch 1-g4-sw and/or smart ring 1-g4-sr. Instant authentication means that after using a smartphone 1-1 (that determines a first bio-rate A) to initially authenticate the smartband 1-g4-sb (that determines a similar type first bio-rate B for comparison,) smartphone 1-1 may then at some later time and for example in response to a communication from another wearable such as smartwatch 1-g4-sw or smart ring 1-g4-sr communicate with smartband 1-g4-sb to receive a second bio-rate from 1-g4-sb (this time serving as rate-A) and then communicating with the smartwatch 1-g4-sw or smart ring 1-g4-sr to receive a bio-rate from this device to be used as second bio-rate B, where upon sufficient A-B matching the communicating smartwatch 1-g4-sw or smart ring 1-g4-sr is determined to be authenticated.

In yet another possible variation of the smartband 1-g4-sb (or any of lockable devices 1-g4-c) the smartband 1-g4-sb is further adapted to include a biometric identification means such as a fingerprint reader (similar to 1-4-s1 or 1-2-s1) but lacking the concurrent bio-rate (in this case finger pulse) reader. In operation, a person 10 places wearable 1-g4-sb (or any of so further adapted devices 1-g4-c) onto the respective body location (such as a wrist for smartband 1-g4-sb) whereupon using any of several well-known technologies the wearable 1-g4-c determines its “locked” state. After entering this locked/“currently being worn” state and being in communication with primary device 1-g1 (such as a smartphone with intermediary app 1-1,) wearable 1-g4-c provides to device 1-g1 the captured biometric identification such as a fingerprint. Upon receiving the fingerprint (with no associated finger pulse/bio-rate “B”,) the primary device 1-g1 then compares the received fingerprint to a registered or pre-registered fingerprint and thereby determines/authenticates the identity of the person, and thus authenticates the wearable 1-g4-c, in this example smartband 1-g4-sb. The careful reader will see that the primary device 1-g1 (in this example 1-1) is then not required to determine a bio-rate A (such as by using rPPG technology,) does not also receive a bio-rate B from the smartband 1-g4-sb and then also does not subsequently authenticate the smartband 1-g4-sb/wearable 1-g4-c by sufficiently matching bio-rates A and B, thus obviating the “bio-rate” portion of core functions 1-g1-1a and substantially eliminating the need for core functions 1-g4-c2 and 1-g1-1b.

In a careful consideration, any wearable 1-g4-c such as smartband 1-g4-sb that has been further adapted to determine biometric identification data such as an image of the person's face or a scan of the person's fingerprint can be self-authenticated via wearable-initiated and “user transparent” communication with the primary device 1-1, where “user transparent” includes not requiring that the person putting on the wearable 1-4g-c to be authenticated then must also operate the primary device 1-g (such as by using for example a primary device 1-1 to capture one or more facial images along with any of a secure authentication signal, such as a bio-rate A/face pulse,) as the intermediary app on the primary device can perform this authentication without requiring any input through a user-interface, but could then for example communicate to the person via an digital notification that the wearable 1-g4-c has been properly authenticated (or not). It will also be apparent to the careful reader that a so further adapted and alternatively authenticated wearable 1-g4-c will then still comprise a “locked body surface area” (where “surface area” is also herein referred to as “location”) which is then also authenticated, such that core function 1-g4-sb for “on-going” use while the wearable 1-g4-c remains locked to determine one or more bio-measurements from the “authenticated locked body location” is implemented. Furthermore, the so further adapted and authenticated wearable 1-g4-c may be used in combination with the primary device 1-g1 for authenticating additional health measurements from any of “lockable” or “non-lockable” companion devices, as a previously described for wearables 1-g4-c that do not so comprise the necessary apparatus for determining biometric identification data (such as a face image or fingerprint).

In a deeper consideration, it will be apparent that primary device 1-g1 (in this example smartphone 1-1) must comprise a registered or pre-registered and authenticated biometric identification data of the person for comparison with any such biometric identification data determined by a wearable 1-g4-c further adapted with the necessary biometric sensors. In the case of a smartphone 1-1 as a primary device 1-g1, biometric information that is a facial image(s) is easily captured and authenticated, whereas biometric information that is a fingerprint may be captured but upon consideration is not easily authenticated. This is apparent when considering a first person may be having their image captured by the smartphone 1-1 while a second person is having their fingerprint detected by the same smartphone, where the smartphone 1-1 is unable to verify that the fingerprint is biometric information gathered from the same first person being imaged by the smartphone 1-1's cameras. In one solution, the smartphone 1-1's fingerprint reader can be further adapted to include a finger pulse reader (thus being like sensors 1-2-s1 and 1-4-s1,) but this requires further adaptations to the traditional smartphone and at the least is not workable for exiting smartphones without such a combined fingerprint plus finger-pulse reader, were also some smartphones do not even comprise a fingerprint reader.

In the related PCT application US2021/029186 filed on Apr. 26, 2021, entitled GUEST TRACKING AND ACCESS CONTROL USING HEALTH METRICS (see especially FIGS. 15A and 15B) there was taught a separate fingertip device (referred to in the related application as “fingerprint device 3-1-d9”) that communicated via wire or wirelessly with smartphone 1-1 (labeled 2a in the related application) and used secured light signaling to authenticate that the fingerprint being captured by related device 3-1-d9 was authenticated to be of the same person as facial images (see label 84-img in the related figures) being substantially concurrently captured by smartphone 1-1. As a careful consideration will show, such a separate device 3-1-d9 as taught in the related application can be manufactured at relatively low cost and then be made available for use with virtually any currently existing or future anticipated smartphone 1-1 (since all smartphones are reasonably anticipated to always include wireless communications and most will also include a “wired port,” for example what is referred to as USB-C). As will also be apparent, such as device as 3-1-d9 that includes a fingerprint reader (see 3-1-d9-fpr) could alternatively include a fingerprint plus finger-pulse reader (such as 1-2-s1 and 1-4-s1) thus alternatively providing for secured authentication signaling using the bio-rate pulse, rather than light.

While these solutions are possible and anticipated, any such further adapted fingerprint reader 3-1-d9 of the related application is then also serving a key function of the herein described companion device 1-g4-c, albeit not being anticipated to remain “worn” or locked and thus not allowing for a core function such as 1-g4-c3 or use in authenticating “on-going” health measurements taken either by device 1-g4-c or by other “locked” or “not-locked” companion devices 1-4. Given this consideration, it is preferable to use a herein described companion device 1-g4-c to also serve the function of the related application's “fingerprint device 3-1-d9” (by including a secured authentication signal of either the bio-rate B, such as a finger-pulse, or a light signal such as described and taught regarding related application device 3-1-d9). Thus the reader will see that many variations are possible, and that it is important that the authenticating (“A”) device 1-g1 responsible within composite primary device 1-g4 for authenticating the companion (“B”) device 1-g4-c receive any of information that is “matchable” to existing registered, pre-registered, or substantially currently captured data, where then the match operation confirms authentication. In some examples, the “any of information” can be biometric information such as a fingertip pulse or even a facial image captured by a camera comprising the companion device 1-g4-c, or alternatively can be a “light signal” provided by an LED comprising the companion device 1-g4-c and responsive to a control signal provided by the contributing device 1-g1, all as taught in the related application as “authentication using light signaling.”

And lastly, it is noted that any companion device 1-g4-c that is adapted to capture a biometric such as a fingerprint but not also a corresponding fingerprint pulse, but is then also adapted with an LED or similar to provide for light signaling, can then serve as an initial means in cooperation with the authenticating (“A”) device 1-g1 such as a smartphone 1-1 (that otherwise cannot obtain an authenticated fingerprint using its own embedded sensors) to provide the smartphone 1-1 with a captured fingerprint that the smartphone 1-1 then receives and authenticates “one-time” using light signaling as described in the related patent. After this first “one-time” receiving and authenticating, since the smartphone 1-1 (device 1-g1) now has what is effectively a “registered” and authenticated fingerprint, when a person 10 then at some future time again uses a wearable 1-g4-c comprising the fingerprint reader (but no pulse reader,) it is no longer necessary to use any of secured authentication signaling as the “at some future time” determined wearable 1-g4-c fingerprint is sufficient for authentication of the wearable 1-g4-c by the smartphone 1-1 via comparison to the “registered” (and prior authenticated) fingerprint.

In yet another possible new device for a lockable companion device 1-g4-c, the present invention anticipates a replaceable watch band 1-g4-rb that otherwise shares the same apparatus and functions as just described for the smartband 1-g4-sb but alternatively can be attached to virtually any existing marketplace watch that might for example not already include either the necessary communication, computing, or bio-rate sensing means as taught herein for smartband 1-g4-sb. It is also noted that any of the lockable companion devices 1-g4-sw, 1-g4-sr, 1-g4-sb, and 1-g4-rb may additionally comprise means for determining one or more other bio-measurements and thus should not simply be limited to providing a bio-rate for use by primary device 1-1 but also seen as further adaptable to provide one or more health measurements. The careful reader will see that many variations are possible not just for implementing the per-ID primary device 1-g1 or on-going primary device 1-g3, but also for implementing composite primary device 1-g4 (and thus also lockable companion devices 1-g4-c) such that the present teachings should be considered as exemplary rather than limiting. As a careful consideration will also show, by further adapting any of lockable companion devices 1-g4-c, such as 1-g4-sw, 1-g4-sr, 1-g4-sb, and 1-g4-rb, to also comprise means for determining bio-metric ID data along with a bio-rate A, each of the further adapted devices 1-g4-c then become primary devices 1-g3.

Referring next to FIG. 9C, there is shown a block diagram of generalized system 100g comprising the two main interacting system objects (1g and 20g) along with a generalized interface 35-g providing for and allowing the two system objects (1g and 20g) to communicate and exchange key data (35-uc-r. 35-uc-g, 35-aco-g, 10-c-g, 118-d, and 116-d2). System generalized object 1g is any of a primary device 1-g1, 1-g2, 1-g3 or a composite primary device 1-g4 for use by a person/operator 10 to determine any one of or any combination of authentication, authorization, or readiness vetting data prior-to, during, or after a session of operation of the other system object 20g that is any of an asset, process, or service (“APS”) as described or anticipated herein. Generalized interface 35-g is labeled by its main function as an “operator limit component” such that without such an interface 35-g an operator 10 receives substantially (and traditionally) unlimited use of the APS object 20g.

It is important to see that without operator limit component 35-g that provides any one of or any combination of the functions of APS access-use control 30-g and operator readiness determination 40g, the accumulation of authenticated non-session and session data and meta-data 118-d is not accomplished (due to the lack of an operator ID for differentiating the accumulated data by operator, regardless of the operator ID being either of anonymous or non-anonymous, all as prior taught herein,) where “meta-data” as described herein refers to any system data that is not any of “non-session data” and not any of “session data,” but is derived at least in part from any of “non-session data” and/or any of “session data.” It is also important to understand that the present depiction is not intended to limit the deployment of the generalized operator limit component 35-g, which as described herein may be executed on any one of or any combination of the main objects (1g and 20g) or some other computing system such as a remote system 50 (see for example FIGS. 1B and 2A) that is in communications with objects 1g and/or 20g.

Still referring to FIG. 9C, in the preferred generalized operation of system 100g, the operator limit component 35-g automatically detects the proximity of a primary device object 1g and upon detection provides an electronic “use certification request” for the operator's authentication and authorization to access/operate object 1g. In response to such a request, or otherwise prompted by the operator 10's use of the primary device object 1g rather than in response to a request by 35-g, object 1g performs any of the necessary and described functions taught herein for authenticating operator 10 and then subsequently providing a generalized “use certificate” 35-uc-g to operator limit component 35-g. Exemplary use certificates 35-uc-g as described herein include what was referred to as an “authorized access key” 30-4-aak (see for example FIG. 2A) but otherwise generally include any information necessary to provide any one of or any combination of (i) proof of authentication of the operator, (ii) proof of the operator's authorized rights with respect to any of access and/or operation of the APS object 20g, or (iii) proof of the operator's “fitness” or “readiness” for operation of the APS object 20g, where fitness or readiness broadly and generally includes (iii-a) any information regarding the physiological and/or psychological state of the operator, (iii-b) any information regarding the relevant certified or otherwise training or operational familiarity and/or relevant past experience of the operator, and (iii-c) otherwise any information related to the operator and relevant to the operation of the APS object 20g.

Generalized system 100g optionally comprises the use of third-parties (such as an internet based digital/web service) for acting as intendent “registrars” or “certifiers” of an operator's “registered use certificate(s)” 35-uc-r related to an given APS 20g, where registered use certificates 35-uc-r comprise any one of or any combination of (a) any of the operator 10's personal information (such as name, contact information, biometrics, etc.), (b) any information related to the “intermediary application” executing on the operator 10's primary device 1g (such as a hash code, encryption key, or identifying “app ID”), and (c) any of the operator 10's one or more existing use certificates 35-uc-g applicable to an APS object 20g (or any information indicative of such use certificate(s)). As presently depicted, generalized primary device object 1g may provide any of a use certificate 35-uc-g directly to the operator limit component 35g or otherwise at some prior time provide the same or similar (and possibly additional) sufficient information to a third-part registrar/certification service as “registered, certified APS use certificates” 35-uc-r, where then in response to a use certification request by operator limit component 35-g generalized primary device 1g may then provide a “full” use certificate 35-uc-g directly to component 35-g (where “full” implies comprising sufficient information) or provide a “less-than-full” certificate such as comprising simply the “app ID” of the primary device 1g and/or an anonymous ID related to the operator 10, where upon receiving of such less-than-full certificate 35-uc-g, component 35-g uses any of the information provided in less-than-full certificate 35-uc-g to communicate with a third-party registrar/certifier to then retrieve or otherwise receive sufficient “full” use certificate information comprising registered use certificates 35-uc-r.

Still referring to FIG. 9C, after component 35-g determines or otherwise confirms the sufficiency of use certificate 35-uc-g, provided either “in full” or “less-than-full” by primary device 1g, component 35-g then determines based at least in part upon certificate 35-uc-g any of generalized authorized conditions 35-aco-g after which component 35-g then provides or otherwise makes accessible to APS object 20g and optionally primary device 1g the authorized conditions 35-aco-g. As prior discussed, authorized conditions should be broadly interpreted and provide of any limit on any of access and/or operation of the APS object 20g, where the limit should not be construed as merely a “reduction of full capacity operation,” but rather as any possible change to an APS object 20g's normative operation that is otherwise expected and allowed absent system 100g. Also as prior discussed, in some configurations, APS object 20g may determine and provide any of “confirmation biometric(s)” 10-c-g (such as images captured of the operator's face while the operator 10 is currently physically or virtually “positioned” with respect to APS object 20 to begin control of the APS object 20) to primary device 1g for confirmation, where upon receipt of such confirmation biometric(s) 10-c-g primary device 1g at least in part uses confirmation biometric(s) 10-c-g (for example by comparing such confirmation biometric(s) to prior registered or pre-registered biometrics of the operation 10) to confirm that the operator 10 that is currently set and presumed ready to operate APS object 20g is authenticated by the intermediary app/“honest broker” executing on the primary device 1g to be the person purported in the use certificate 35-uc-g or otherwise a “valid” operator.

Generalized primary device 1g preferably provides an “operator ID” that is any of anonymous or non-anonymous either commensurate with the provision of the use certificate 35-uc-g or after additional processing of any confirmation biometric(s) 10g, where then APS object 20g uses such operator ID as data for associating with any and preferably all operation related data (with many variations as specified herein) that comprises any of non-session data, session data, or meta-data derived at least in part from such data. Those familiar with data aggregation will understand that ideally data to be associated has at least one organizing “key” or “identifier,” where the present system prefers the use of an identifier that crosses over multiple possible sessions of operation, where such an identifier might be the “operator ID” or also some other ID for example related to the primary device 1g and then not specifically to the operator 10, and where crossing over multiple possible sessions means that two or more sessions use the same organizing key/identifier. However, it is also possible that the primary device 1g provides instead or in combination with a “cross-over” ID a “per-session”/“transaction” ID that is intended to be unique to a single session of operation (thus not “crossing overing multiple sessions”).

In any case, system 100g in general, and then preferably APS object 20g at least in part, is responsible for determining some or all of session related data and preferably storing and maintaining any and all of non-session and session related data either “locally” on a computing element comprising object 20g, and/or remotely on an associated server 50, all as will be well understood by a careful reading of the present teachings and by those familiar with computing systems architectures. As prior discussed, at any point during capture and determination or afterwards, any of non-session and session related data may be further processed into meta-data such as assessments and ratings of the session as well as the many other types of meta-data described and anticipated herein. In the preferred system, some or all of authenticated non-session and session data and meta-data 118-d is made available, either anonymized or non-anonymized, to any of third-parties for use at least in part for determining any of third-party policy restrictions and enforcement 116-d2, where then such restrictions and enforcements 116-d2 in portion or in whole are provided to either of operator limit component 35-g and/or generalized primary device 1g.

And finally, with respect to FIG. 9C, the reader is directed to see the many advantageous feed-back loops allowing for the dynamic alterations, “evolution,” and otherwise “best-fitting” of operation limitations with respect to specific operators 10 and types of operators 10 as well as specific APS objects 20 and types of APS objects 20, thus forming a powerful mechanism for improving the safe and useful operation of APS objects 20 by operators 10, all as will be understood especially by those familiar with what are in general referred to as “learning systems” that often include the use of technologies such as “AI” (artificial intelligence) and “ML” (machine learning).

Referring next to the combination of FIGS. 10A and 10B, the present system is shown for use when the generalized asset 20g is more specifically an implementation of a computing system such as computer 20-c of FIG. 10A or device 20-d of FIG. 10B, where as will be appreciated by those skilled in the art of computer systems, 20-c and 20-d are fundamentally the same in that they both except user inputs, process these inputs with respect to a computer program (the “work product app” 20-c-app and 20-d-app of FIGS. 10A and 10B, respectively,) and then create a “session work product” 20-c-wp and 20-d-wp, respectively. In this sense, the distinction between a computer 20-c verses a device 20-d should not be considered as limiting, but rather as a general marketplace distinction mostly related to form and function, where again both 20-c and 20-d comprise traditional computing elements such as a CPU (or data “processor” of some kind,) memory, data storage, input and output means, communication means, etc.

Regarding form and function, computer 20-c tends to be larger in size and is more “general purpose,” “sedentary,” and for “extended duration use,” where typical implementations include a desktop, laptop, and even a tablet computer (although the tablet moves closer to a device 20-d, and thus could be considered as functioning as either 20-c or 20-d). Regarding form and function, a device 20-d tends to be smaller in size and is more “limited purpose,” “mobile,” and for “quick duration use,” where typical implementations include a smartphone, DLSR camera, or even specialized marketplace devices such as an imaging microscope, and where a common feature of 20-d as taught herein is the inclusion of an embedded imager 20-d-ai for capturing images. While computer 20-c is also depicted as comprising an imager such as camera 20-c-cam, either embedded or as an attached peripheral, as will be appreciated the uses of camera 20-c-cam on computer 20-c within a larger form factor desktop or even a laptop are traditionally different than the uses of an imager 20-d-ai 20-ai within a smaller mobile form factor smartphone.

What is common with respect to the session operation of the assets 20-c and 20-d as compared to the assets prior discussed herein, is that assets 20-c and 20-d are typically used in operations that produce digital output, such as a text document, spreadsheet, computer program, music score, email, text message, tweet, 2D/3D vector-based drawing or model, vector or bitmap digitally created image, pixel based captured photograph or video, etc., whereas prior discussed assets 20 such as a vehicle or industrial equipment were typically used for providing physical output, such as transportation, manufacturing of products, housing, etc. As will be shown, the creation of digital work products 20-c-wp and 20-d-wp by assets such as 20-c and 20-d, respectively, can be described by a set of operational choices expressed as biological behaviors made over time by an operator/user 10, where the operational biological behaviors expressed by the operator are transduced into digital information through the use of input devices such as a keyboard, drawing tablet, digital (music) keyboard, mouse, touch interface, buttons, etc., and where there is a significant marketplace need and advantage to authenticating the operator/user 10 making these operational behaviors and showing their sufficient correlation to resulting computer 20-c or device 20-d inputs, and thus providing “proof-of-authorship” of the work products 20-c-wp and 20-d-wp, respectively. This “authentication of a work product” is representable as digital information, herein referred to as work product certificate 36g-we determined by a work product authenticator 36g that is a computational component executing on any one of or any combination of computer 20-c, device 20-d, primary device 1 (such as smartphone 1-1 or smart watch 1-2,) or otherwise a separate system in communication with any of computer 20-c, device 20-d, or primary device 1, such as a remote server executing a temporal session certifier service 26. The combination of the work product 20-c-wp or 20-d-wp and a work product certificate 36g-wc is herein referred to as an authenticated session work product 36g-awp, where it will be obvious to those skilled in the art of information systems that these two digital “documents”/“files” can be combined into one or alternatively embodied as more than two documents/files.

With the advent of new computer technologies, such as generative AI or large language learning models such as Chat GBT, it is now possible that a computer algorithm can create virtually all manner of digital output such as a text document, spreadsheet, computer program, music score, email, tweet, CAD model, digital image, captured photograph or video, etc., where this machine/“synthetic” work product is often indistinguishable in content from a human/“organic” generated work product. There are currently some technologies for algorithmically assessing a work product “post-fact” (i.e., after being created) in order to detect or determine its synthetic versus organic origin, although these algorithms are becoming less and less effective as AI technologies continue to advance. One of the purposes of the present teachings is to provide an alternative “pre-fact” versus “post-fact” approach for verifying an organic work product that instead relies on authenticating that a person is the operator of the asset (such as 20-c or 20-d) substantially during a session of operation (such as “typing up a term paper”) that produced the session work product (hence the term paper itself) in response or correlated to the asset inputs (e.g., keystrokes and mouse movements) verified as being input by the person during the session of operation. Thus, the present invention teaches systems and methods for establishing what is herein referred to as “the authenticity of work products.”

As will be shown, using a primary device with intermediary app 1, such as smartphone 1-1 or smartwatch 1-2, where the primary device 1 is functioning as an “honest broker” to authenticate a person 10 as being the operator during a session of operation of an asset (e.g., 20-c, 20-d) in which digital content/work products are created or modified, accomplishes the authentication of a session work product 36g-awp comprising the work product 20-c-wp or 20-d-wp, respectively, along with a work product certificate 36g-wc. As shown in both FIG. 10A and FIG. 10B, the present teachings further anticipate a supervising entity 24 for issuing supervisor certificates 24-sc, where a supervising certificate 24-sc is preferably obtained using a primary device 1 acting as an honest broker (see FIG. 11A,) and where the supervisor certificate 24-sc then provides for unique instructions and data for use at least in part when encoding either of the work product certificate 36g-wc or the work product 20-c-wp or 20-d-wp (all to be further discussed shortly) as well as for use at least in part to limit the functions of the asset 20-c or 20-d.

Referring next primarily to FIG. 10A, alternative operator-content-creation system 100-5 for use in creating authenticated digital work products 36g-awp during a session of asset 20-c operation comprises asset/computer 20-c for use by an operator 10 (implied, but not depicted) under the optional limitations as specified by a supervising entity 24, where the operator 10 uses a primary device 1 such as smartphone and intermediary app 1-1 or smart watch and intermediary app 1-2. For the purposes of illustration, the present figure will be described in reference to an educational use-case where the operator 10 is a student, the optional supervising entity 24 is a digital service or similar used by a school, a teacher, or otherwise educational party for whom the student is required to produce an authenticated work product 36g-awp, for example a term paper. As will be appreciated by those familiar with the challenges to educational systems presented by AI in general, or more categorically some form of “generative AI,” or more specifically systems such as Chat GPT, it is possible that a student uses such AI or similar automated tools to “synthetically” produce a credible term paper/session work product that is substantially indistinguishable from an “organic” work product 20-c-wp.

Current means for “post-fact” receiving by a supervising entity 24 of a synthetic work product to then be manually or programmatically analyzed as a means for determining the likelihood of being synthetic versus organic are limited and anticipated to be decreasing in accuracy as AI increases in ability. It is further noted that any such expenditure of time and energy for such “post-fact” work product analysis, whether manual or programmatic, represents a significant additional cost and burden on the supervising entity 24.

In yet another educational example the work product 20-c-wp is some form an on-line exam or test, where a student working in isolation (such as at home during the recent pandemic, or simply taking a remote class) is expected by the “honor system” to be taking the test themselves, and possibly to not be using prohibited resources, such as AI, on-line forums, communications with friends, web-searches, etc. In still another example, a student in a computer programming class is assigned a project to develop a software algorithm, where now using generative AI it is possible to create significant portions if not the entire algorithm using these synthetic approaches, were also generating programs as assignments are typically done in isolation at home/remote and thus not directly monitored by the supervisor 24. In each of the term paper, test, and computer program exemplary use cases, the present teaching provide for the pre-session and real-time (i.e., “during session”) authentication of the student 10 as being the operator of the asset (20-c, or 20-d,) where this operator 10 authentication is accomplished using the honest broker functions provided by primary device 1 as taught in the prior related applications and herein.

As will be clear, by authenticating the student 10 as being the current operator of an asset 20-c, it is then reasonable to assess that any digital content created in response to inputs into the asset 20-c, especially where these inputs are shown to further correlate to directed detected and tracked biological behaviors of the student 10, is likely of organic workmanship. Those familiar with computing technology will also appreciate that the student 10 may use the asset 20-c to connect with another computing device and as such “download” or otherwise receive other work products, organic or synthetic, not necessarily created by the student 10, where the system prefers that work product apps 20-c-app (and 20-d-app of FIG. 10B) are further adapted to identify such portions of the final authenticated work product 36g-awp, where it is also possible to provide additional work product certificates corresponding to these “downloaded” or otherwise received other work products, and even to assess whether only some portion or all of the other work product is being used as a portion or all of the authenticated work product 36g-awp. Thus, and for example, cutting and pasting from “on-line” documents and information sources such as Wikipedia, or inserting pictures from the internet, etc., are objectively determinable by the further adapted apps 20-c-app (or 20-d-app) by analyzing the authenticated operator/student 10's directives as translated into asset 20-c or 20-d inputs. Those familiar with the education marketplace will recognize the many uses cases similar to those cited above that can also benefit from this “pre-fact” (i.e., during session of operation) authentication, all to be discussed in further detail shortly. The present teachings however should not be limited to the education market, as many other markets including corporations and the government have an interest in assuring that individuals as operators 10 are performing tasks as assigned (thus “as dictated by the supervisor” 24).

As will also be clear from a careful consideration, the vast majority of the content that has heretofore been made available on the internet has simply been understood to obviously have been created by human effort (thus “organic”,) where synthetic documents simply did not exist to any great extent or sufficient quality to be misconstrued as organic. The present invention anticipates for example that in the upcoming era of comingled organic and synthetic content, appropriate labeling of content will be extremely beneficial, just as earlier efforts were made to begin labeling food products as organic with the implication that all other products include at least some minimal synthetic/man-made chemicals treatments (“pesticides”) or even genetic modifications (“GMOs”). It is currently reasonable to establish organic internet content simply by “creation dates” (or similar) such as “prior to 2023,” with an understanding that going forward in time (such as “2023 or after”) synthetic AI generated content will co-mingle with organic human generated content and may be essentially indistinguishable, thus the urgent need for a global digital content labeling system and methods.

Additional concerns are related to the idea that AI generated content relies upon organic content for what is known as training, such that at least some sources (e.g., individuals or companies) of organic content will wish to forbid their organic content from being used as AI training data, or at least require a license payment for such training use, where then the “organic content” labeling is critical. Training AI on “raw organic” human generated content is anticipated to produce different pattern recognition (thus different synthetic content) than training on mixed organic-synthetic or only synthetic content, where again the organic label or even “1^{st} generation synthetic” (thus synthetic derived directly and only from training on organic content) will become advantageous. The present teachings provide novel technical means for achieving such positive labeling of organic (human created) digital content, where the assumption that anything not so labeled as authentically organic carries at least a probability of being some degree of synthetic. It is noted and preferred, that using a similar work product certificate 36g-wc, future AI generative and similar systems could also positively label their outputs as synthetic, even providing a generation representing the degree or amount of organic versus synthetic content used during training. And thus all digital content could be positively labeled and then otherwise assumed as potentially synthetic.

Still referring to FIG. 10A, and using the example of a student (the operator/user 10) creating authenticated digital content that is a term paper (authenticated session work product 36g-awp) using their computing system (asset 20-c) over one or more durations of time (sessions of operation) and under the restrictions provided by their teacher (supervising entity 24,) the reader is now also directed to FIG. 11A wherein the student using their primary device 1 first receives from the supervisor 24 a supervisor certificate 24-sc, a preferred but optional step.

FIG. 11A depicts a preferred sequence of 7 steps conducted on a combination of a primary device 1 and a supervisor app 24-app that ultimately provides supervisor certificate 24-sc to the primary device 1 and therefor the student/operator 10. The primary device 1 is preferably in the configuration of a smartphone with intermediary app 1-1, but as the extensive prior teaching in the related applications and herein describes, this configuration is not mandatory as other device deployments such as a smart watch 1-2 can be used to implement the intermediary app and to comprise sufficient sensors for use in authenticating the student/operator 10. The supervisor app 24-app should be understood as any digital service (preferably an internet/web-service) that supports communication with the primary device 1. Where a careful consideration and reading will show that while the primary device 1 functions in real-time interaction with the student operator 10 (the presence of the operator being necessary for operator authentication,) the supervisor app 24-app docs not require the physical presence of a supervisor 24, e.g., the teacher, but rather as mentioned could be an automated digital service made available over the internet.

Those familiar with secured internet transactions will understand that significant teachings and commercially available solutions exist for preferably ensuring that the digital communication and transaction (thus the receiving of the supervisor certificate 24-sc by the primary device 1) remain private and secure to the student/operator 10, where it is even possible that the student/operator 10 remain anonymous to the supervisor by for example using an anonymous operator ID (such as 10-id in FIG. 3A). Those familiar with types of encryption systems in general, and in particular with what is known as asymmetric encryption, will also understand the benefits of using public keys exchanged by two parties for helping to ensure that digital documents (such as authenticated work products 36g-awp) communicated between the two parties remain private to the two parties, where for example the supervisor's public key is provided along with or contained within the supervisor certificate 24-sc and usable for encrypting some portion or all of the authenticated work product 36g-awp, and where optionally a student/operator 10's public key is provided either during the process steps described in FIG. 11A, or within authenticated work product 36g-awp.

It is not the purpose of the present invention to teach encryption methods, but rather to describe the primary device 1 as exchanging sufficient encryption information with the supervisor app 24-app such that the future transmission of an authenticated session work product 36g-awp can optionally include encryption of some or all of 36g-awp, where then only the supervising entity 24 can decrypt some or all of 36g-awp. For example, session work product 20-c-wp or 20-d-wp comprising authenticated work product 36g-awp might be a traditional digital file that is typically not encrypted (although it might be transmitted via a secured communication method,) where work product certificate 36g-wc is encrypted prior to transmitting to the supervising entity 24. What can be understood is that the operator 10, in this example a student, may prepare to provide authenticated work products 36g-wp to a supervising entity 24, for example using an internet service provided by an educational system, by first obtaining through a sequence of steps a supervisor certificate 24-sc that preferably comprises encryption information such as a supervising entity 24 public key, where the primary device 1 of student 10 may acquire this certificate 24-sc while exchanging personal identification information (thus not anonymously) or while withholding personal identification information (thus anonymously).

In “step 1” 24-sc-s1 of the acquisition of a supervisor certificate 24-sc by the student 10 using a primary device 1, the student 10 first uses device 1 to communicate preferably via a secured internet connection with the supervisor app 24-app, where primary device 1 acts as an “honest broker” (“HB”) in the transaction on behalf of both the student 10 and the supervising entity 24. During the connection process of “step 1” 24-sc-s1, as discussed in the related applications, the primary device 1 may supply an “honest broker certificate” or otherwise identifying digital information for use by the supervisor app 24-app in authenticating the primary device and its intermediary app 1. After authenticating the primary device 1, in “step 2” the supervisor app 24-app preferably provides one or more authentication conditions to the primary device 1, where for example an authentication condition indicates one or more personally identifying information the student 10 must at least provide to the honest broker/intermediary app 1 such as a current facial image, fingerprint, access code, password, etc.

As discussed especially in the related applications, the student 10 is presumed to have prior registered with the honest broker executing on the primary device 1, where such registration or “private enrollment” with device 1 includes providing one or more initial proofs of authentication such as but not limited to registration facial images, fingerprints, access codes, passwords, etc. Thus, authentication in “step 3” 24-sc-s3 as performed by the intermediary app executing on device 1 includes accepting personally identifying confirmation information through device 1 to be sufficiently matched in comparison to prior personally identifying registered information. Upon sufficient match, at least the primary device intermediary app 1 “recognizes” the authenticity of the student 10, upon which in “step 4” 24-sc-s4 the primary device communicates a positive confirmation of authenticity along with the required personally identifying information or anonymous ID as dictated by the authentication conditions provided in “step 2” 24-sc-s2. After receiving at least a confirmation of student 10 authenticity in “step 4” 24-sc-s4 as well as possible anonymous or non-anonymous identification information, supervisor app 24-app optionally provides work product options in “step 5” 24-sc-s5, or otherwise proceeds directly to “step 7” 24-sc-s7 and provides to the primary device 1 a supervisor certificate 24-sc, thereby authorizing the student to generate an organic work product 20-c-wp (or correspondingly 20-d-wp). If necessary and executed, optional “step 5” 24-sc-s5 allows the supervisor app 24-app to provide a list of two or more types of work products (for example, a term paper, a homework assignment, a quiz, and a survey) for which each type of work product may then have its own corresponding supervisor certificate 24-sc, such that in a “step 6” the student 10 using the primary device 1 selects the desired type of work product providing a request for the provision in “step 7” 24-sc-s7 of the corresponding supervisor certificate 24-sc. As will be appreciated by a careful consideration, these multiple possible certificates for multiple possible work product types may also be for multiple possible supervisors, such as different teachers, classes, or curriculum in the education example or different managers, divisions, work groups in a corporation example, with virtually limitless possibilities and combinations across the many varied marketplaces.

Thus the reader will see that the desirous operator 10 of a computing assets 20-c or device asset 20-d used in the generation of a digital work product 20-c-wp or 20-d-wp, respectively, may first acquire a supervisor certificate 24-sc for governing and limiting the asset 20-c or 20-d operation and/or for providing information on the codification of either the work product 20-c-wp or 20-d-wp or the corresponding work product certificate 36g-wc.

As a careful consideration of FIG. 10A will show, and as will be discussed in more detail shortly, this “supervisor authorization” such as detailed in FIG. 11A is preferred but optional and not strictly necessary for the generation of an authenticated session work product 36g-awp. Thus it is anticipated that a person 10 using a primary device 1 and an asset such as 20-c or 20-d can generate authenticated work products 36g-awp absent “supervision,” such as a free-lance writer generating copy to be submitted to various media outlets or an individual 10 capturing images with their smartphone 1 to share on Instagram.

As a careful consideration of FIG. 11A will show, and as will be discussed in more detail in relation to upcoming FIG. 10B, this “supervisor authorization” may alternatively be provided directly to the asset, such as 20-c or 20-d, thus bypassing the operator 10 using the primary device 1, such that the asset 20-c or 20-d is then itself authorized to generate the proper authenticated session work products 36g-awp with work product certificates 36g-wc independent of a particular operator 10. It is anticipated that a non-identified person 10, still using a primary device 1 for private authentication purposes, may use any of a supervised asset 20-c or 20-d without ever performing the same or similar functions as described in FIG. 11A, where then the supervised asset 20-c or 20-d preferably still requires person 10 authentication for enabling a session of authenticated operation. Furthermore, it is also anticipated that the person 10, regardless of how or whether a supervisor certificate 24-sc was acquired for providing supervising information to an asset 20-c or 20-d, may interact with the generalized operator limit component 35g for initiating a session of operation of asset 20-c or 20-d, either providing personally identifying information (presumably for encoding with work product certificate 36g-wc) or omit any such information (in which case the work product certificate 36g-wc preferably and simply confirms an “organic” creator,) or still alternatively provides an anonymous ID (which also can be encoded into certificate 36g-wc,) such that the resulting work product 36g-awp is always certified “organic” but otherwise may or may not be directly associated with a specific person or anonymous ID.

Referring now to FIG. 10A and assuming the operator/student 10 has acquired a supervisor certificate 24-sc using their primary device 1, it is preferred that the primary device 1 stores and holds the certificate 24-sc. In this preferred arrangement, it is then possible for the operator/student 10 to engage any number of assets 20-c or 20-d conveniently using the same supervisor certificate 24-sc without requiring the student to transfer this same certificate to each and every asset 20-c or 20-d being used to create associated digital content/work products. What is further anticipated and preferred is that the intermediary app running on the primary device 1, which then has access to the supervisor certificate 24-sc uses information from this certificate at least in part to derive the operator 10's use certificate 35-uc, where the reader is directed especially to FIG. 9C and the discussion of generalized use certificate 35-uc-g for more teaching. What can be seen is that both a supervising entity 24 and the intermediary app executing on the primary device 1 (acting as an honest broker on behalf of both the supervisor 24 and the operator 10) each have input into establishing the operational limits associated with both the operation of the asset 20 (in this case 20-c of FIG. 10A and 20-d of FIG. 10B) and now also the operation of software executing on the asset 20 (in this case work product app(s) 20-c-app of FIG. 10A and 20-d-app of FIG. 10B) that are used by the operator/student 10 during a session of operation to at least in part cause the generation and/or determination of digital content/session work product 20-c-wp (and 20-d-wp of FIG. 10B).

Referring now to FIGS. 10A and 10B, as prior mentioned, it is desirable to provide to assets such as 20-c and 20-d with public supervisor 24 keys for use in optionally encrypting any one of or any combination of the session work product 20-c-wp or 20-d-wp, respectively, the work product certificate 36g-wc, or the combination of the two that together comprise the authenticated session work product 36g-awp (where it is also understood that certificate 36g-we could alternatively be embedded in work product 20-c-wp or 20-d-wp while still achieving the spirit of the invention). If as preferred, the operator 10 uses a process such as described in FIG. 11A to authenticate themselves while then also obtaining a supervisor certificate 24-sc optionally comprising a supervising entity 24 public key based upon the level of desired security, any so provided supervising entity 24 public key may then be incorporated into the use certificate 35-us by the primary device 1 along with an optional operator 10 public key, where then the use certificate 35-us is provided to generalized operator limit component 35g (see also FIG. 9C) preferably executing on either or both assets 20-c and 20-d.

In one operation of the present teachings, during the execution of the steps described in FIG. 11A, preferable using a secured internet transaction, the supervisor app 24-app provides a supervisor 24 public key to the primary app 1 and the primary 1 provides an operator 10 public key to the supervisor app 24-app. Later, during the operation of either asset 20-c or 20-d, some or all of authenticated session work product 36g-awp is first encrypted by either the asset 20-c or 20-d or the primary device 1 using the operator 10's private key, after which this first encryption in then followed by a second encryption using the supervisor 24's public key, also performed by either the asset 20-c or 20-d or the primary device 1. Upon receiving of the work product 36g-awp, only a supervisor 24 or entity with a matching supervisor 24 private key can perform a first decryption of the second encryption, which may then expose some or all of the information comprising work product 36g-awp, and then furthermore only a supervisor 24 or entity with a matching operator 10 public key can perform a second decryption of the first encryption exposing some or all of the information comprising work product 36g-awp. As will be understood by those familiar with asymmetric encryption, this “double encryption” method provides the receiving supervisor 24 or entity with proper matching keys additional assurance that the work product 36g-awp was provided by the operator 10 whose public key is able to perform the second decryption of the first encryption.

Referring still to FIGS. 10A and 10B, generalized operator limit component 35g serves to limit any traditionally available asset 20-c (or 20-d) functions, ranging from “operating limits” to “application limits,” where “limit” should not be construed simply as reducing, blocking, restricting, etc., but rather should also be seen as “setting values and ranges of values” or “setting options,” etc., where in at least some circumstances a limit may otherwise appear to “increase” or “enable” an asset 20-c or 20-d function that might otherwise not be available to an operator 10.

Operating limits for example include: (1) limiting which specific assets 20-c and 20-d can be used to by the operator to create an authenticated work product 36g-awp, where the limit can be applied by a number of means known and sufficient for identifying individual assets 20-c and 20-d, and where for example a teacher limits a student to taking a test on one or more computing systems with specific IDs pre-known to be located in a specific campus building, or alternatively on their personal asset 20-c or 20-d that includes a specific ID pre-registered with either the intermediary app and/or the supervising entity 24, (2) limiting dates, times, durations of time, etc., of any one or more sessions of operation for creating an authenticated work product 36g-awp, (3) limiting the number of displays/monitors or otherwise peripherals connected to the asset 20-c or 20-d, for example only one display and specifically using a camera built into the asset, (4) limiting any connected computing services such as services that provides web/internet access, and otherwise 5) limiting any traditionally or currently available operation of the asset 20-c or 20-d that is not associated with a specific work product app 20-c-app or 20-d-app, respectively.

Application limits are applied to any such specific work product apps 20-c-app or 20-d-app and for example include: (1) limiting session work product 20-c-wp or 20-d-wp data formats, additionally specifying any embedded signatures (to be discussed in greater detail in relation to 20-d-wp of FIG. 10B,) (2) limiting authenticated session work product 36g-awp encryption methods, either in regards to the session work product 20-c-wp or 20-d-wp or the work product certificate 36g-wc, (3) limiting the occurrences, timing, and types of user confirmation requests (to be discussed in greater detail in relation to 20-app-1 of FIG. 11B) that are useful for confirming user/operator 10 current presence at and engagement with the asset 20-c or 20-d. (4) designating which if any device/app inputs and ongoing work metrics should be processed for use in work product authentication (to be discussed in greater detail in relation to 20-c-app, 20-app-2, and 20-app-3 of FIG. 11B), (5) designating which if any user movements and work-use metrics should be processed for use in work product authentication (to be discussed in greater detail in relation to 36g, 36g-2, and 36g-3 of FIG. 11B), (6) designating the types of correlation (e.g., hand movements to keyboard inputs), the formulas (e.g., hand motion X>Y within time T of keystroke) and sufficiency levels (e.g., 95%<1 ms T) of correlation between any on-going work product metrics and any on-going work-use metrics should be implemented to privately authenticate the work product (to be discussed in greater detail in relation to 36g-4 of FIG. 11B), and otherwise (7) limiting any traditionally or currently available operation of the work product app 20-c-app or 20-d-app.

Typical and exemplary work product apps 20-c-app traditionally executing on a computer asset 20-c include word processors, spreadsheets, presentation software, 2D and 3D digital image and model creation or editing software, video creation and editing software, animation software, music composition software, video gaming software, mathematical simulation software, computer programming environments and tools, etc. Typical and exemplary work product apps 20-d-app traditionally executing on a device asset 20-d include photo and video capture apps, communication and social media apps including email, texts, photo-sharing, etc., voice communication and recording apps, game apps, etc.

What will be clear from a careful consideration of the present teachings, is that virtually any app/software executing on a computing asset such as 20-c or 20-d intended for use by an operator-person 10 to create a digital work product 20-c-wp or 20-d-wp, regardless of the content type and extent of the digital work product, can be further adapted for ensuring and/or cooperating with other applications for ensuring that the resulting traditional work product 20-c-wp or 20-d-wp is determined to be an authenticated work product 36g-awp, where authentication requires the objective and evidentiary substantiation that the creation and modification of the traditional digital work product 20-c-wp or 20-d-wp is substantially concurrent and congruent with operator-person 10 behavior, where sufficient objective evidence comprises the detection of operator-person 10 “presence,” “liveness,” and “behavior” motions in correlation to transduced app inputs themselves correlated to work product 20-c-wp or 20-d-wp creation and modification. As will also be clear to those familiar with synthetic work product 20-c-wp or 20-d-wp creation and modification, such concurrent and congruent operator presence, liveness, and motion neither exists nor is simple to synthetically fabricate.

What will also be clear is that some apps/software such as video games that are not traditionally considered to be used for creating digital work product (although the streaming of video games is a traditional example of a video game digital work product) could still otherwise be activated or receive inputs from a synthetic sources such as an “automaton”/AI/“bot”, etc., (a practice typically considered to be cheating,) where then using the present teachings it is now possible to ensure that activation or inputs being provided to such non-traditional work product apps as video games are organic (thus provided by a person and not an “automaton”/AI/“bot”, etc., hence synthetic). It is also important to see that in any case, the determination of a concurrent and congruent operator-person 10 includes a range of specificity of optional personally identifying information from fully anonymous to identifying an individual person.

Referring to FIGS. 10A and 10B, generalized operator limit component 35g comprises asset/process/service access-use control 30g (see also FIG. 9C) that is preferably responsible for assigning such aforementioned operation and application limits to either of the asset 20-c, 20-d general control components (such as what is traditionally referred to as the “operating system”) or to any one or more specific work product apps 20-c-app or 20-d-app executing on the asset 20-c or 20-d, respectively, where control 30g may use any information provided by any or either of a supervisor certificate 24-se or use certificate 35-uc in determining any of such limits. Operator limit component 35g preferably also provides input to the work product authenticator 36g executing preferably on the asset 20-c or 20-d, but also alternatively on the primary device 1, or some combination thereof (see also upcoming FIG. 11B for more detail). Work product authenticator 36g preferably also receives input from any one of or any combination of the work product app(s) 20-c-app (or 20-d-app of FIG. 10B) or the asset 20-c (or 20-d)'s operating system comprising indications of asset operation data 20-c-1, 20-d-1, respectively, where it is understood that at least some portion of concurrent and congruent asset operation data 20-c-1, 20-d-1 are indicative of the organic nature of the session of operation and the resulting created work products 20-c-wp (or 20-d-wp,) and where the work product authenticator 36g at least in part uses any such indications of asset operation data 20-c-1, 20-d-1 for use in determining work product metrics (see FIG. 11B).

As a careful consideration will show, for an asset 20-c, 20-d at least some of the various direct and indirect input devices generating asset operation data 20-c-1 or 20-d-1, respectively, are necessary for allowing an operator 10 to “operate” the asset 20-c, 20-d during a session of operation resulting in the creation or modification of work products 20-c-wp or 20-d-wp, respectively. By analogy, such various inputs resulting in operation data 20-c-1 or 20-d-1 are equivalent to input means (such as a steering wheel, gas pedal, brake pedal, etc.) for at least in part causing the operation of the prior mentioned vehicle asset 20 during a session of operation. As with the vehicle 20 asset, direct metrics are determined by sensing the operator 10, such as with a camera, whereas indirect metrics are determined by sensing the asset including any of its various input devices. As will be shown, both the direct and indirect metrics can be at least temporally correlated (e.g., the operator 10 “moves” in some way as determined by direct sensing of the operator 10 while substantially at the same time a various input is received,) and often also spatially correlated (e.g., the operator 10's “right index finger touches the ‘h’ key on a keyboard” while substantially at the same time a various input being the encoding of an ‘h’ key is received).

As a careful consideration will also show, like the vehicle asset 20, there is a range of possible implementations for determining the various possible operator 10 inputs used during a session of operation of an asset 20-c, or 20-d. This range of possible implementations includes interfacing with any of the one or more input devices, interfacing with the asset 20-c, or 20-d's operating system that itself is interfacing with any of the one or more input devices, and/or interfacing with a product app 20-c-app, or 20-d-app, respectively, that itself is either or both of interfacing with the operating system or the one or more input devices. What is important is that the work product authenticator 36g is able to receive from the input devices, the operating system, and/or an app at least some indications indicative of the current and on-going various inputs at least in part causing the creation or modification of work product 20-c-wp, or 20-d-wp, where again, such current and on-going indications are desirable for the determination of a correlation of these various inputs with indications of operator-person 10 behavior such as looking, speaking, moving hands, etc. (see again FIG. 11B for more discussion,) thus authenticating these inputs and the organic causation of work product 20-c-wp, or 20-d-wp.

Various input devices related to assets 20-c, 20-d for determining asset direct metrics 20-c-m1, 20-d-m1, respectively, traditionally include a camera and microphone, where again a direct metric is indicative of an operator behavior such as a movement of the head, eyes, lips, or fingers. Various input devices related to assets 20-c, 20-d for determining asset indirect metrics 20-c-m2, 20-d-m2, respectively, traditionally include a keyboard, mouse, digital tablet with pen or finger input, digital music keyboard, etc., but may also include a camera or microphone, where again an indirect metric is indicative of information received by the asset 20-c. 20-d for processing, where the traditional assumption is that the information received was caused by an operator behavior such as looking, speaking, or most often the movement of hands.

As prior discussed and now reiterated, there are several alternative ways for the work product authenticator 36g to receive or determine asset operation data 20-c-1, 20-d-1 including through direct interaction with various input devices such as cameras, microphones, keyboards, mice, digital tablets, digital music keyboards, etc., direct interaction with the asset 20-c (or 20-d)'s operating system, or direct interaction with the work product app(s) 20-c-app (or 20-d-app, respectively). As such, the present FIGS. 10A and 10B should be considered as illustrative of concepts and otherwise non-limiting of implementation and deployment as many variations are possible without departing from the spirit of the invention. What is important to see is that the work product authenticator 36g is ultimately receiving at least some (preferably and substantially real-time) indications of both operator 10's behaviors (thus “direct metrics” of the operator 10) as well as any asset 20-c (or 20-d) inputs (thus “indirect metrics” responsive at least in part to the operator 10's behaviors) that are being used at least in part by a work product app 20-c-app (or 20-d-app, respectively) to determine the creation and/or modification of a session work product 20-c-wp (or 20-d-wp).

Still referring to FIGS. 10A and 10B, as will be discussed further in relation to FIG. 11B, and as a careful consideration will show, by sufficiently correlating real-time direct metrics with real-time indirect metrics it is possible to substantiate (based upon varying degrees of correlation) the “authenticity of inputs” to an asset 20-c (or 20-d), where authenticity of inputs means that the verified causal agent of the inputs is a person at least known to the primary device intermediary app 1. Those familiar with synthetic generation of digital content will understand that while the synthetic digital content may “post-fact” appear to be substantially equivalent to organically created digital content, the “pre-fact” generational process of synthetic digital content comprises two highly distinguishable features being (1) that the synthetic content is generated in a much more shortened time duration as compared to organic content generated over a more lengthy time duration (for example it may only require an AI process seconds to generate a term paper that would take the student hours to generate similar digital content), and (2) that the synthetic content is generated “complete” and “final” without any exposed iteration of the digital content as compared to organic content that is generated “partial” and “iteratively” (for example an AI process provides the finished term paper as a single process output, whereas the student provides and also modifies an on-going stream of characters working towards a finished term paper).

This real-time correlating of operator 10's input-causing behavior with asset 20-c (or 20-d) work-product-causing-inputs can be monitored and correlated using a work product authenticator 36g that executes on any or both the asset 20-c (or 20-d) or the primary device 1. As depicted in FIG. 10A, it is also possible to establish as real-time communications link to a temporal session certifier 26 that receives any combination of asset operation data 20-c-1 (or 20-d-1) indicating preferably real-time session operation activity. Thus, at least in use cases where an operator 10 (such as a student) is using an asset 20-c to develop a “complex”/“intricate” work product 20-c-wp over typically an extended duration of time (such as a term paper created in one or more extended sessions of operation,) it is possible to additionally or alternatively engage a temporal session certifier 26 in real-time communication with the asset 20-c to verify that the work product 20-c-wp is being generated both (1) “over a more lengthy time duration,” and (2) in a “partial” and “iterative” manner, both of which are anticipated to be sufficient for determining an organic causal agent. Temporal session certifier 26 is preferably a web-service which then provides a certifier certificate 26-cc for each “one or more extended sessions of operation” or at least associated with a completed work product 20-c-wp (thus serving in addition to or in replacement of the work product certificate 36g-wc,) where the certifier certificate 26-cc is provided directly to a supervising entity 24 and/or back to the asset 20-c for addition to or replacement of the work product certificate 36g-wc generated by the work product authenticator 36g.

Still with respect to FIGS. 10A and 10B, what is important to see is that several systems and methods have been shown to be sufficient for (a) determining the correlation using a work product authenticator 36g of operator 10's behaviors with inputs received and processed by work product app 20-c-app (or 20-d-app)'s (the received inputs of which are themselves correlated to the generated work product 20-c-wp (or 20-d-wp)), or (b) tracking the computing activity comprising on-going changes to asset operation data 20-c-1 (or 20-d-1) and/or work product 20-c-wp (or 20-d-wp) as communicated preferably by the work product app 20-c-app (or 20-d-app) to a temporal session certifier 26, where either or both of (a) or (b) are usable at least in part to determine and confirm that a work product 20-c-wp was created or modified with sufficient (1) duration of session time, and/or (2) iteration of partial content to be sufficiently indicative of organic versus synthetic digital content generation.

What is also important to see is that the level of sufficiency for providing “pre-fact” “proof-of-organic” digital content creation is adjustable to fit various marketplace use cases, and may result in simple “proof” being that the type, complexity, and “digital size” of the work product was generated in a human-like duration of time and/or iterative process without any direct authentication of the operator 10 (such as by using a primary device 1,) or stringent “proof” being that a specific and authenticated person 10 was determined through direct measurement to have provided the behaviors that tightly correlate to the inputs that were processed at least in part to cause the work product, regardless of whether or not the authenticated person 10 known to the primary device 1/“honest broker” remains anonymous or is shared via personally identifying information along with the generated work product.

A careful understanding of the present and prior related teachings will show that the “honest broker” (also herein “intermediary app”/“IA”) may be executing on either the primary device 1 or the asset 20-c, 20-d, or alternatively a primary device 1 with camera such as smartphone 1-1 may be in communications with the asset 20-c, 20-d to serve as a means for determining direct metrics 20-c-m1, 20-d-m1, respectively (e.g., as opposed to camera such as 20-c-cam,) where the communicating primary device 1-1 for example is mounted on the asset, such as affixed atop of 20-c's display, and maintains a view of the operator 10's face and preferably also their hands engaging the various input means such as a keyboard and/or mouse. (Note also that a smartphone 1-1 is an exemplary form of an asset 20-d, and thus could be both capturing direct metrics 20-d-m1 and indirect metrics 20-d-m2.)

Thus and for example, a student 10 taking a test remotely or writing a term paper in a school library or at home may use their own primary device 1-1 smartphone with honest broker to essentially watch themselves (using a camera of the smartphone 1-1) take the test or enter the term paper over time and in partial iteration, where then the student's primary device 1-1 can receive information from the work product app 20-c-app (e.g., the software administering the test or the word processor creating the term paper) indicative of the inputs received by the app 20-c-app for any of temporal or spatial correlation by the primary device 1-1 with the students behaviors as determined by the honest broker analyzing images taken by the device 1-1 camera, the solution of which then assures the student of complete privacy as only the honest broker executing on their own smartphone 1-1 ever determines information directly related to themselves and may or may not share such information with a supervising entity 24, all within the control and agreement of the operator/student 10, where many variations are possible as will be clear from a careful consideration.

In at least one example variation, a student 10 interacts with their personal laptop 20-c to take an on-line test or enter a term paper (both are exemplary work products 20-c-wp). As the student 10 initiates the appropriate app 20-c-app (i.e., the software providing the on-line test or a work processor,) the app 20-c-app at least in part causes the laptop 20-c to take a picture of the student 10 for providing as a current image 10-c-img, where then the student 10's smartphone acting as the primary device 1-1 and honest broker receives the current image 10-c-img for comparison to a pre-registered image 10-r-img of the student, where upon sufficient matching of images 10-c-img and 10-r-img, the primary device 1-1 provides an indication of both the authentication of the student 10 and the current presence of the student 10 with respect to the laptop 20-c.

It is then possible that the laptop 20-c subsequently captures additional current images 10-c-img repeating the authentication and “presence detection” of the student during the on-going session of operation that is creating the work product 20-c-wp. It is alternatively possible that once initially authenticated by the primary device 1-1, the laptop 20-c continues to capture additional images 10-c-img of the student and then simply self-assures via image analysis that these additional images 10-c-img substantially match the originally captured and authenticated current image 10-c-img (thus obviating the need to further interact with the primary device 1-1 to re-confirm student 10 authentication).

Thus, once a student 10 is initially authenticated as commencing a session of operation with asset 20-c for the creation or modification of a work product 20-c-wp using an app 20-c-app, it is then possible that the asset 20-c continues to self-monitor the presence of the student 10 with respect to the asset 20-c, and only needs to “re-authenticate” using the primary device 1-1 when this “on-going student presence” is “broken” or “lost,” The determination of student/operator 10 presence over substantially the entire duration of a session of operation is minimally supportive of the organic nature of the work product 20-c-wp output as a result of the session of operation, especially where the session is of an extended time duration and the on-going work product output is partial and iterative (which can be assessed by using asset indirect metrics 20-c-m2 without needing additional asset direct metrics 20-c-m1,) where additional support includes then correlating other operator 10's detected behaviors (thus additionally requiring asset direct metrics 20-c-m1,) such as eye and hand movements (determined for example using on-going image analysis of current images captured by camera 20-c-cam) with the on-going changes in app inputs (asset indirect metrics 20-c-m2,) all as prior discussed, where again, these behaviors may be detected by the honest broker executing on the operator/student 10's primary device and/or detected directly or indirectly by the asset 20-c.

Referring to FIG. 10B, there is shown a computing device 20-d for use by an operator 10 to create digital content/work product 20-d-wp during one or more sessions of operation, where different from computing system 20-c, the form of device 20-d tends to be smaller to support “mobility,” which in turns causes practical limits on the types of work products 20-d-wp that can be created, as well as the duration of a typical session of operation for creating the work product 20-d-wp. Good examples of device 20-d include general purpose computing devices such as smartphones (e.g., a primary device 1-1 being also the asset 20-d) and tablets, to more specific purpose/“appliance” computing devices such as a DSLR camera, or an imaging microscope used in a research lab. Each of the examples just listed, i.e., the smartphone, tablet, DSLR, and imaging microscope were chosen because they traditionally include image sensors for capturing light and transducing this light into photographic images, but the careful reader will understand that while this limitation is useful for then understanding how the present invention can be applied to the organic labeling of photographic images, the present teaching have significant value even for computing devices 20-d (or system 20-c) that do not traditionally include imaging sensors.

In comparison to the teachings related to FIG. 10A and computing system 20-c, it is significant to first consider that the primary device 1-1 (shown as a separate from the computing system in FIG. 10A) is (or may be) the computing device 20-d depicted in FIG. 10B, and thus the intermediary app performing honest broker functions is executing on the device 20-d that is creating or modifying the work product 20-d-wp that is to be labeled as organic when proper conditions are met. As a careful consideration will show, another critical difference between a device 20-d and a system 20-c is that the smaller form factor of device 20-d in turn means limited surface area for accepting the operator 10's hand movement behavior typically used for text entry (as well as pen input, musical key input, etc.) as opposed to system 20-c that typically includes a larger keyboard (or connects to a graphics tablet or has a larger display for pen or touch input, connects to a digital music keyboard, etc.). This limited surface area as input means then tends to preclude the creating of longer session duration-based content creation and modification, where for example a student 10 is unlikely to create a multi-page term paper using device 20-d versus 20-c. This shorter duration hand-movement entry of device 20-d, such as reduced text entry, also tends to decrease the complexity of thought expression and therefore entry movement (e.g., keyed characters or pen strokes,) which then also reduces the “partial” and “iterative” nature of the typical work product 20-d-wp.

A careful consideration will show that a person 10 could memorize a short text originally generated synthetically, which they could then enter via a system 20-c or device 20-d posing this text as organic by reentering it with hand behavior rather than for example using a well-known “cut-n-paste” command for the insertion of a sequence of text characters (where this cut-n-paste command is detectable by work product authenticator 36g or work product apps 20-d-app and 20-c-app using analysis of asset indirect metrics 20-d-m2, since the cut-n-paste character commands are well-known). However, what is also important to see is that regardless of the original synthetic verses organic generation of a shorter text such as a text message or tweet, the present invention teaches a solution for confirming that this text was “provided through” or in the “operational presence” of a person 10, where then this provision or presence, regardless of the content being actually synthetic, additionally supports the determination of legal responsibility.

Studies of the typical function of a human mind have established that the average person can remember between 5 to 7 words, which is also considered by some to be the average number of words in a text message (although text messages are limited to 160 characters, longer texts can be generated and may be split by the message carrier into multiple messages). Another short-form work product 20-d-wp is a tweet that is currently restricted to 280 characters, where the typical word is considered by some to be 5 characters long, thus a 160 character text might represent roughly 25 words with spaces in between, while a 280 character tweet might represent roughly 45 words, where then the shorter form work product 20-d-wp of a text message and a tweet are still reasonably beyond the ability of a typical person 10 to memorize from a synthetic source and simply reenter with hand behavior to pose the entry as organically created. Thus, even on a “short-content” device 20-d, as opposed to a “long content” device 20-c, correlating operator 10 behavior with app 20-d-app inputs is sufficient for showing the resulting work product 20-d-wp's organic nature.

As a careful consideration will show, using either of a device 20-d or system 20-c, it is possible that a person 10 wishing to pose more “complex” synthetic text-based digital content (e.g., a character based text longer than 7 words, regardless of being a text, tweet, email, or traditional document) as their own organic content could set up a second device or system, or a printed material, showing the synthetic content which the person 10 then uses to prompt their hand behavior/entry into the device 20-d or system 20-c, thus removing the practical issue of memorization, where the present system prefers that either of device 20-d or system 20-c includes a camera for at least viewing the person 10's face and eyes during entry as a means for providing a greater authentication of work product 20-d-wp or 20-c-wp, respectively, where the well-known image processing technique often referred to as “gaze tracking” allows the intermediary app 1-1 acting as an honest broker to detect the necessarily back-and-forth motion of the eye behavior of the person 10 and to correlate this back-and-forth eye motion with the temporal start-and-stop detection of hand-behavior inputs. For example, while a person is gazing away at the source (e.g., a second screen or piece of paper) of the synthetic material they are typically not also moving their hands and fingers and thus pausing the current text entry stream detectable with indirect metrics 20-d-m2.

As will be discussed further with respect to FIG. 10C, individual operators 10 are anticipated to exhibit typical behaviors with respect to the use of assets 20-c and 20-d for establishing particular types of digital content, where these typical behaviors become quantifiable baselines for individualizing the assessment of the likelihood of organic composition. For example, with respect to traditional text entry by a given operator using a keyboard, mouse, and one or more displays on a “sedentary” asset 20-c, there are at least three basic “input patterns,” all discernable using operator 10 behavior tracking. In a typical input pattern, an operator is substantially gazing at a main one of one or more displays, while touch-typing the words of their thoughts, all at a certain “speed,” such as the typical 40 words-per-minute. In a different pattern, an operator may be gazing away from the main display that is showing the words being typed by the operator 10 in order to view a source material from which to copy text (such as a second display or a printed material,) where then the operator 10 is able to type words while not gazing at the main display. Often the speed of this “copy-to-enter” style of entry exceeds the normal “think-to-enter” style, but in any case the gaze of the operator with respect to the main display showing currently entered words is detectably different. In yet a third style, some operators 10 are less efficient typists and often transfer their gaze at the keyboard to help find the desired keys, also detectable using currently available gaze tracking software (see for example “Gazerecorder.com”).

What is important to see is that by determining operator 10 behaviors concurrent and congruent with specific types of digital content entry, types of assets 20-c and 20-d, types of asset input means (such as keyboards, mice, touch pads, touch screens, digital tablets, voice dictation, etc.,) and by associating these behaviors with either the specific individual or a specific anonymous ID (see 10-id in FIG. 3A) it is possible to accumulate historical asset operation data comprising any of 20-c-1, 20-d-1, respectively, where then this accumulated data may be used to determine specific baseline input and otherwise operation behavior baselines (norms) that are highly correlated to a specific identified or anonymous operator. Especially when considering the input by an operator 10 of complex digital content of any type, where the digital content typically requires extended and/or multiple sessions of duration before arriving at a finished digital content ready for distribution, and where then each session of operation also typically exhibits higher degrees of partial content entry intermittently mixed with input entry pauses and then repositioning inside the digital content for editing a prior content entry or positioning to provide input to the content entry app 20-c-app, 20-d-app for enabling functionality, there is a significant difference in objectively measurable input patterns even between individual operators, let alone between any operator 10 and a synthetic algorithm that creates “single-shot” (i.e., fully “complete”) digital content in significantly shorter and single sessions of duration with substantially no observable partial content, pausing, repositioning, and content editing.

It is also helpful to understand that it is possible and typical for an operator 10 developing content of virtually any type, to insert fully completed portions of content (which may be either synthetic or organic in nature) (e.g., a “cut-and-pasting” of text or an insertion in of an image,) where then a careful consideration of the present teachings will show that it is possible to detect such fully complete insertions and even to objectively quantify their effective total portion of a finished digital content (e.g., 5% of a term paper versus 80%,) where this quantification is anticipated to be an optional parameter comprising the work product certificate 36g-wc.

It will also be clear that as more digital content is labeled, ultimately not just as from an organic source verses synthetic, but additional with an estimated percentage organic versus synthetic, that fully complete insert operations using such prior labeled digital content can then use this content labeling as a means for helping to determine the new digital contents own mix of organic and synthetic. A careful consideration will also show that a synthetic algorithm being trained on data or even responding to a prompt after being trained, may use this same prior digital content labeling to estimate its “trained on” content source (i.e., percent organic versus synthetic) and its response-output mix (again, percent organic versus synthetic).

Still referring to FIG. 10B and the discussion related to a “mobile” “quick duration use” device 20-d, but also as related to FIG. 10A and a “sedentary” “extended duration use” system 20-c, especially when using newer AI or machine learning based technology, over the last roughly 10 years speech-to-text translations has become significantly improved leading to another form of possible digital content entry (at least text entry,) where this entry uses an asset 20-c, 20-d microphone rather than keyboard. Those familiar with voice recognition will also understand that beyond the translation into text, using recorded voice it is possible to determine to a high degree of accuracy an operator 10's personal voice patterns, where then these patterns become a voice-signature, especially as operator 10 recorded/historical voice baselines are determined.

Those familiar with state-of-the-art AI “text-to-voice generators,” will also understand that AI systems are capable of creating near to completely undetectable forgeries of an operator 10's voice, and thus it is possible for an operator 10 to first generate synthetic text content (such as a term paper) and then to use a text-to-voice generator to “enter” this same synthetic text into an asset 20-c, 20-d in an “organic” manner, hence, voice-over-time.

Especially with reference to upcoming FIGS. 11B and then 10C, the present invention will show how combinations of behavior detection modalities (such as simultaneous voice recognition, face recognition, lip movement detection, and hand movement detection,) optionally combined with or otherwise separately used randomly generated operator 10 “challenges” to determine a real-time app 20-c-app, 20-d-app prompted live operator 10 challenge-response input, it is possible to authenticate to a high degree of accuracy that a work product (such as text) is organic as opposed to originally being synthetic and then entered via “synthetic-copies-of-organic “deep fakes” of the operator 10's face and voice (for more detail see FIG. 11B request for confirmation 20-app-1). As will also be clear from a careful understanding of the present teachings, even using any such sophisticated attempts, the original synthetic content is still “fully complete” and thus to simulate organic entry the synthetic content must be input with both normal “partiality” and “editing,” even matching the operator 10's baselines, which if not available can be substituted with normalized operator baselines preferably draw from a similar operator type, where realistic partial entry and editing of digital content are then two additional signs of organic creation that make the synthetic forgeries even more difficult to accomplish.

Now referring more probabilistically to the use of a sedentary/extended duration session system 20-c as opposed to the mobile/quick duration device 20-d, another means for an operator 10 to pass-off synthetic content as organic is by using headphones to listen for example to synthetic generated text being read while then otherwise appearing to enter this text organically into system 20-c. As a careful consideration will show, this solution is extremely problematic for the operator especially for larger and more significant texts that would require significant sessions of listening and typing. If the text-to-speech system is being operated on the system 20-c, then it is possible for the text entry app 20-c-app to interface with the operating system of the system 20-c to determine that such a text-to-speech system may be in operation, whereby it is further possible to convert this same text-to-speech in real-time back into text, which may then be compared to the text being entered into the text entry app 20-c-app showing that the source of the text is coming from the text-to-speech source and not from the operator 10's own origination. It is also noted that this synthetic-text-to-synthetic-speech then being reversed from synthetic-speech back into entered text via an operator 10's behaviors, all in an attempt to pass the original synthetic speech off as organic, will still exhibit the “full complete” and “non-partial”/“non-edited” nature of synthetic “single-shot” digital content.

Hence, even without determining where the operator 10 sourced the original content (e.g., from a second external source that may be synthetic or from a creative process in their own minds and hence organic,) simply by determining the nature of the entered digital content (again and for example. “single-shot” indicating synthetic whereas “partial-edited” indicating organic) it is possible for the work product authenticator 36g to definitively detect and certify a work product 20-c-wp (or 20-d-wp) as synthetic versus organic by only analyzing the asset indirect metrics 20-c-m2 (or 20-d-m2) (e.g., including keyboard and mouse movements over time) and not also analyzing asset direct metrics 20-c-m1 (or 20-d-m1) (e.g., including concurrent and congruent operator 10 behaviors such as head movements, lip movements, eye gaze, and hand and finger movements all correlated to the indirect metrics,) which only then further add to the assurance of the synthetic versus organic certification.

While it is possible to conceive of a content “scrambler” that transforms (at least text based) single-shot synthetic content into partial content with edits, presenting this scrambled content to an operator 10 wishing to present the second source synthetic content off as organic, this content scrambler solution creates a significant difficulty for the operator 10 who then must be prompted by this scrambled content to understand when to pause entry, to “go back” and edit, etc., all to make the entry appear organic, where then the total effort required of the operation 10, even if possible, is pragmatically infeasible and at least otherwise defeats the advantage of synthetic systems to quickly create and distribute single-shot content. It is further noted that any person or organization desirous for any reason of creating such synthetic content to be mislabeled by a work product authenticator 36g as organic must then incur a much more significant work product 20-c-wp (or 20-d-wp) creation cost as compared to paying for what might be mere seconds of computer AI algorithm time. When considering for example other types of digital content such as 2D and 3D drawings, the challenge of creating realistic scrambled content of either text or drawings is significantly beyond the current capabilities of AI technology, as well as being outside the intentions of mainstream AI developers with the budget and resources for creating any such content scrambler.

While it is further conceivable that an industrious person creates synthetic scrambled text with a matching “deep fake” video-audio of themselves reading this synthetic scrambled text, there are still the problems that this scrambled-content with face-voice entry solution does not include the necessary concurrent and congruent hand behaviors, where the hand behaviors may also be detected via cameras such as 20-c-cam making any use of robotics non-pragmatic at best. Furthermore, the present invention still teaches the random prompt-challenge that can be used by either of the work product app 20-c-app (or 20-d-app) or work product authenticator 36g, where the random prompt-challenge forces the entry method (synthetic or organic) to recognize the challenge prompt, pause, and then properly respond to the challenge (see request user confirmation 20-app-1 in FIG. 11B) before continuing with scrambled content entry, where these prompt challenges can be made in such a way to be impractical for any currently existing AI to properly respond (as demonstrated by current “captcha” technology). It is also noted that while 2D printed images of an operator 10's face have been shown sufficient to fool face recognition algorithms, such algorithms have been improving (often using both visible and non-visible light,) but otherwise newer low-cost 3D cameras are nearing commercialization such that any deep-fake video of a person is then easily detectable by determining its flat 2D nature, all as will be well understood by those familiar with 2D and 3D facial recognition.

What is important to see is that single-shot synthetic content of any content type is substantially detectable at the pre-fact time of creation by a work product authenticator 36g (at least because it lacks the organic style of extended duration, partiality, and/or edits,) where the authenticator 36g is working as a part of or in combination with a work product app 20-c-app (or 20-d-app,) and where the authenticator 36g and/or app 20-c-app (or 20-d-app) simply analyzes asset 20-c (or 20-d, respectively) inputs and/or associated indirect metrics 20-c-m2 (or 20-d-m2, respectively) to determine sufficient organic style duration, partiality, and edits, possibly even with respect to operator 10 baselines. It is noted that these asset inputs or associated metrics 20-c-m2 (or 20-d-m2) for example must by their nature include the same commands (as opposed to digital content) that are interpretable by the app 20-c-app (or 20-d-app) to properly accept the single-shot content for inclusion into the work product 20-c-wp (or 20-d-wp, respectively,) and thus are inherently interpretable as indicative of single-shot (such as “copy-and-paste” commands) verses partial entry with edits over time. It is also important to see that attempts to then scramble otherwise single-shot synthetic content to appear as longer duration, partial, and edited still require organic operator 10 input, the proof of which can be verified by the work product authenticator 36g at least by using asset direct metrics 20-d-m1 or by direct processing of images input from cameras such as 20-c-cam (FIG. 10A) or 20-d-ai (FIG. 10B). As prior discussed, the creation of synthetic entry means to pass off as organic content entry, such as deep-fake videos and audios are problematic and realistically infeasible if not also cost prohibitive.

Referring now to FIG. 10B and device 20-d, in some examples device 20-d is mobile and meant to be carried or worn by the operator (such as a smartphone or smartwatch,) where these mobile solutions may then also themselves by a primary device (such as 1-1 or 1-2, respectively,) in other examples devices 20-d might be a more stationary data entry “appliance,” such as an imaging microscope found in a research lab or university.

The present invention anticipates that a significant portion of short-duration content creating devices 20-d (such as 1-1, 1-2, or an imaging microscope) might include cameras (in FIG. 10B referred to more fundamentally as an “imager,”) where it is further desirable to label images captured by the authenticating imager 20-d-ai as “sensor captured” and then also as “organically directed.” The label “sensor captured” is compared to current new methods of generative AI that are able to create images and videos often referred to as “deep fakes” that are virtually indistinguishable “post-fact” from original sensor captured images. The label “organically directed” as compared to robotically or synthetically directed indicates that the sensor capturing was positioned and initiated by a person/operator 10.

Regarding “sensor captured,” as will be discussed in greater detail with respect to FIG. 10D, in summary a camera sensor captures (analog) light photons that are transduced into (analog) electrical potential, which is then itself transduced from an analog signal into digital data using what is known as an “A-to-D” (analog to digital) converter chip. Virtually all camera sensors come with their own embedded A-to-D chip, where then the resulting digital information, often referred to as the “raw” image comprising for a typical sensor what is known as a “Bayer pattern” of red-green-blue (“RGB”) pixel values, all as will be well understood by those familiar with commercial camera sensors. Often, this raw RGB Bayer pattern is also translated into a matrix of RGB values per pixel, were each R, G, and B value is often referred to as a sub-pixel value, where this process is traditional referred to as demosaicing, and where this second demosaicing step may be performed and thus embedded on the image sensor or may otherwise be performed by a computing element not embedded in the sensor that receives the first original raw RGB Bayer pattern.

What is important to see is that the preferred authenticating imager 20-d-ai at least provides a first raw Bayer pattern digital output ensured to be a non-synthetic transduction of physical light into electrical signals, where this digital output being generated on the imager 20-d-ai is non-addressable by an external computing element and therefore cannot be “hacked” or intentionally altered. The present invention teaches a preferred solution where the authenticating imager 20-d-ai's A-to-D chip is further adapted to embed a secured and hidden signature within the raw RGB data (or within any demosaiced format if this second digital output is also provided by the sensor and thus non-addressable to an external computing element). In the preferred embodiment, a first encrypting algorithm uses one or more of what are generally referred to as “algorithm seeds” to process into a randomly unique set of multiple encoding parameters describing an encryption strategy for each image captured by the sensor, where the preferable parameters comprise (1) spatial neighborhood characteristics, (2) neighborhood sequence, and (3) neighborhood encoding method.

Preferred seeds comprise (a) a sensor ID, sensor private key, or otherwise some sensor related datum preferably embedded into the authenticating imager 20-d-ai by the sensor manufacturer at the time of sensor manufacturing and uniquely representative of the sensor and substantially only known by the sensor manufacturer, optionally (b) the operator ID, operator-sensor public key, or otherwise some operator related datum as provided to the authenticating imager 20-d-ai by a primary device 1 and substantially only known by the primary device 1, optionally (c) a supervisor ID, supervisor-sensor public key, supervisor-operator-sensor public key, or otherwise some supervisor related datum as provided to the authenticating imager 20-d-ai either directly by a supervising app 24-app or by the supervising app 24-app in communication with the authenticating imager 20-d-ai via a primary device 1 or an asset 20-d (or 20-c,) and substantially only known by the supervising entity 24, and optionally (d) an image ID, or otherwise some per-image related datum generated by the authenticating imager 20-d-ai uniquely representative of each distinct captured image.

What is preferred is that any combination of the seeds may be used to generate the encoding parameters in a repeatable manner such that at least the authenticating imager 20-d-ai is able to generate the encoding parameters and then some other computing element (such as the sensor manufacturer, a primary device 1, an asset 20-d (or 20-c,) or a supervisor app 24-app in receipt of a unique image captured and encoded by the authenticating imager 20-d-ai) is also able to generate the identical encoding parameters by obtaining the same original seeds. What will be clear to those familiar with data encryption is that the vulnerability of the preferred system is the regeneration of the encoding parameters which will then provide the information for decrypting the “image signature” embedded within preferably each unique image captured by the authenticating imager 20-d-ai, where then by using multiple seeds the present invention will show that this vulnerability is minimized.

As will be discussed in greater detail with respect to FIG. 10D, the present system teaches an image sensor comprising authenticating imager 20-d-ai that is further adapted to embed one or more unique digital signatures into each unique image captured by the authenticating imager 20-d-ai, where for example a first digital signature is only decipherable by the sensor manufacturer and can be used by any concerned third-party to verify that the uniquely captured image is organic (thus being a transduction by the sensor of photons of light energy) verses synthetic (this being a algorithmically generated image). A second and different digital signature is only decipherable by the primary device 1/honest broker working on behalf of a registered operator 10 and can be used by the operator 10 to provide proof to a third-party that a given organic image was captured in response to behaviors provided by the operator 10. A third and still yet different digital signature is only decipherable by the supervisor 24 (or for example the supervisor-app 2-app) and can be used by the supervisor 24 to ensure that a received unique image was captured for the supervisor 24, or for the operator 10 acting under the direction of the supervisor 24.

As will also be discussed, a preferred first, second, or third embedded signature is predicated on the unique encoding parameters generated by use of the appropriate seeds (a, b, or c combined with d) matching the first, second, or third embedded signatures, where a+d seeds are used to encode the first sensor manufacturer signature, b+d encode the second operator signature, and c+d encode the supervisor or supervisor-operator signature (thus a single image can include one to three unique signatures). Regarding the encoding parameters, the spatial neighborhood characteristics (1) preferably describe at least a pixel neighborhood size, e.g., 4×4, 8×8, or 16×16, while the neighborhood sequence (2) preferably describes a sequence or neighborhood addresses for sequencing a multiplicity of neighborhoods to form the embedded signature, and while the neighborhood encoding method (3) preferably describes how to set the value of one or more encoding pixels/sub-pixels either residing in the neighborhood or residing in a determinable location with respect to the neighborhood, where the setting of the pixel/sub-pixel value is preferably based upon a mathematical combination of neighborhood pixel/sub-pixel values, for example a green encoding sub-pixel is set to the “average value+5” or the “average value−1” of all green sub-pixels in the neighborhood.

As will be clear to those familiar with image representation, a small change to an encoding pixel/sub-pixel that is spatially within or near the neighborhood of pixels (especially of the same color, red, green, or blue) is substantially undetectable by a human observer and will be undiscernible to an algorithm attempting to find the signature because the encoded pixel value can be kept within a typical sensor “noise” range meaning that sub-pixels of the same color in the same neighborhood of an image can typically vary within some sensor noise limited amount and thus any encoded pixel will appear to be “normal” for a given neighborhood. For example, and green encoding sub-pixel that is set to +5 or −1 with respect to the average of 4 neighboring green sub-pixels can appear to be within a normal expected deviation, whereas being set to +100 or −50 would be more discernable to an algorithm looking for the encoding pixels/sub-pixels. It is also noted that both the addresses and sequencing of the neighborhoods (2) used to encode the signature preferably vary image by image, where potentially each image comprises hundreds of millions of possible neighborhoods each sequenced in a different manner, where then each sequenced neighborhood may use a different encoded pixel/sub-pixel value, thus making the signature virtually undetectable without knowing the appropriate seeds.

Referring next to FIG. 11B, there is shown a preferred sequence and arrangement of interacting processes to accomplish work product authentication resulting in a session work product 20-c-wp, or 20-d-wp along with a work product certificate 36g-wc, the combination forming an authenticated session work product 36g-awp. The processes represent preferred functional components of system 100-5 comprising the use by an operator 10 of a primary device 1 (categorically any primary 1g, see FIGS. 9A, 9B, and 9C) interacting with an asset such as computer system 20-c or computing device 20-d, where in at least some implementations the primary device 1 is the computing device 20-d, or yet in other examples the traditionally less-mobile computer system 20-c further comprises some or all of the functions of the intermediary app so as to also function as the primary device 1. Thus, the reader will see that while the processes are shown in the present figure as executing on separate computing platforms, those familiar with computing systems will also understand that other deployment arrangements are possible without departing from the scope, spirit, and teachings of the present invention, where then these different possible deployments might then necessitate the omission of some processes described in the present figure, the combination of others, as well as the further adaptation and/or inclusion of other processes.

In the exemplary deployment as depicted in the present FIG. 11B, an operator 10 uses a primary device 1 to interact with either of a substantially “sedentary” and assumed “extended duration use” system 20-c or a substantially “mobile” and assumed “quick duration use” device 20-d to create an authenticated “organic” session work product 20-c-wp or 20-d-wp, respectively, where the primary device 1, such as a smartphone 1-1 or smartwatch 1-2 executes the intermediary app that functions as the “honest broker” working on behalf of both the operator 10 and the “public trust” to ensure an authenticated “organic” session work product 36g-awp, and where the primary device 1 preferably executes an authenticate work product 36g process in coordination with the creation, on-going development of, or otherwise modification to a work product 20-c-wp, 20-d-wp by one or more apps 20-c-app, 20-d-app, respectively, executing a develop work product process on system 20-c or device 20-d, respectively. The combination of these processes is also referred to herein as collectively “work product certification” abbreviated in the element labeling as “wpc.”

Still referring to FIG. 11B, in a “step 1” 1g-wpc-s1 of the exemplary deployment of system 100-5, the operator 10 uses the primary device 1 to connect to the system 20-c/device 20-d establishing electronic communications, often involving a step referred to in the art as “pairing.” With the primary device 1 sufficiently paired to system 20-c, device 20-d (or, alternatively being device 20-d,) the operator 10 desirous of creating an authenticated session work product 36g-awp then presumably and preferably commences an app 20-c-app, 20-d-app, respectively, wherein the app 20-c-app, 20-d-app is further adapted to execute “step 2” 1g-wpc-s2 to request work product certification, where the careful reader will understand that any app 20-c-app, 20-d-app may still create non-authenticated session work products, and where it is possible further adapt any app 20-c-app, 20-d-app to always create authenticated session work products 36g-awp such that step 2 1g-wpc-s2 is automatically executed without any operator 10 input.

In response to either of an automatic or an operator 10 caused request for work product certification as preferably issued by the any app 20-c-app, 20-d-app, in “step 3” 1g-wpc-s3 the primary device 1 privately authenticates the user/operator 10 of the primary device 1 and then also presumably the user/operator 10 of the app 20-c-app, 20-d-app. Authentication has been discussed extensively in the related applications and herein, suffice to say that the primary device 1 preferably accepts one or more biometrics (such as a facial image or a fingerprint) to use as confirmation of the user/operator 10's identity. It is also noted when using primary device 1 capable of “on-going” authentication, such as a primary device 1-g3 or composite primary device 1-g4 (see FIG. 9B,) it is possible that the user/operator 10 is already authenticated such that step 3 1g-wpc-s3 is complete without requiring further interaction with the user/operator 10, all as will be clear from a careful reading and understanding of the present application.

Given a properly authenticated user/operator 10, in “step 4” 1g-wpc-s4 the primary device preferably (but optionally) determines one or more supervisor and work product limits, such as limits specified in a supervisor certificate 24-cc and otherwise limits associated with any one of or any combination of an associated supervisor 24, the operator 10, a type of work product 20-c-wp, 20-d-wp, features and aspects of the asset 20-c, 20-d, respectively, or the app 20-c-app, 20-d-app, respectively, being used to generate the work product, and otherwise any technologically controllable function related to the system, or the taught and alternative components of system 100-5. Any such determined work product limits are preferably included in a use certificate 35-uc for providing to an operator limit component 35g, where component 35g is preferably being executed on the asset 20-c, or 20-d (see FIG. 10A, 10B, as well as 9C,) and where component 35g processes any such use certificate 35-uc (or similar, including for example processing any directly provided supervisor certificate 24-cc) to apply device/app limits in “step 6” 1g-wpc-s6.

Use certificate 35-uc may also be optionally provided to the work product authenticator component 36g that conducts the process of authenticating and “certifying” the session work product 20-c-wp, 20-d-wp, where then the limits preferably specify the type and extent of authentication to be implemented by component 36g, and where types of authentication for example include (1) authenticating the presence of the operator 10, such as by confirming that a current image (such as 10-c-img) captured in real-time of the operator 10 sufficiently matches a prior registration image (such as 10-r-img) of the operator 10 known to the primary device 1 prior to the session of operation or otherwise matches a prior authenticated current image (such as 10-c-img) captured substantially during the session of operation, (2) authenticating the “liveness” of the operator 10 beyond the presence, where liveness includes any indication of biological movement over time such as but not limited to head movements, eye movements, lip movement, voice sounds, hand movements, etc., or even the detection of the pulsing of blood for example through portion of the face of the operator 10 or through a body surface such as a finger or wrist of the operator 10 (see FIGS. 9A and 9B, as well as the related art,) (3) authenticating the behavioral correlation between operator 10 and asset 20-c, 20-d inputs for processing especially by app 20-c-app, 20-d-app, respectively, where correlations include detected operator 10 movements, such as eye gaze changes, head turns, lip changes, and hand or finger movements, etc., that are contemporaneous or congruent with inputs such as mouse movements, keystrokes on a keyboard, touches on a touch surface, changes in audio, etc., and (4) a succession of asset 20-c, 20-d inputs for processing especially by app 20-c-app, 20-d-app indicative of the complexity of the inputs (e.g., mouse movements interspersed with keyboard keystrokes,) the extended duration and time statistics of the inputs (e.g., the total duration from the first to last detected input as well as the average time between inputs,) the size of content inputs entered (e.g., the total number of inputs translated into digital content versus indications of application function control such as button clicks on a UI,) the “in-part” nature of the inputs (e.g., single keystrokes thus character input verses extended content insertion such as a cut and paste an entire page of words or the insertion of a picture,) and any editing of the inputs (for example mouse movements redirecting the selection of prior entered input content,) etc. In summary, these types of authentication as anticipated herein include (1) operator presence, (2) operator liveness, (3) operator correlated behaviors, and (4) input nature.

Still referring to FIG. 11B and now examples of the “extent of authentication” that might be specified in a use certificate 35-uc, example extent specifications include (a) limitations for determining any of authentication types (1), (2), (3), or (4) in any combination and at any one or more times preferably in relation to the commencement or duration a session of operation, and (b) limitations on the personal information used in determining the work product certificate 36g-wc, for example including full anonymity, an anonymous ID (such as 10-id of FIG. 3A,) personally identifying information (such as a name or operator ID, or personally identifying information and biometric information (such as a face image, finger print, voice print, etc.,) and (c) limitations on the supervisor information used in determining the work product certificate 36g-wc.

As prior discussed in relation to FIGS. 10A and 10B, use certificate limitations may also apply to “operating limits” and “application limits,” where operation limits on session operation in summary include restricting to (1) specific useable assets 20-c, 20-d, (2) dates, times, durations, (3) usable asset (“hard”) peripherals (such as displays, keyboards, mice, etc,), (4) usable app (“soft,” “connected”) services (such as web-services and APIs), (5) otherwise any traditionally available use option, and where application limits on session operation in summary include (1) data formats and the use and types of an embedded signatures in work product 20-c-wp or 20-d-wp, (2) encoding and encryption types and means for use with either or both of work product 20-c-wp or 20-d-wp, or work product certificate 36g-wc, (3) occurrence, timing, and types of user/operator 10 confirmation requests 20-app-1, (4) mapping inputs 20-app-2 into ongoing work product metrics 20-app-3 for use in authentication 36g-4, (5) mapping user/operator 10 detected behaviors 36g-2 into ongoing work-use metrics 36g-3 for use in authentication 36g-4, (6) type, formulas, and sufficiency levels for use in determining authentication 36g-4 when analyzing the correlation between work product metrics 20-app-3 and work-use metrics 36g-3, and (7) otherwise limiting any traditionally available use work product app 20-c-app or 20-d-app operation.

Those familiar with computing systems will also appreciate that the classifications of “types of authentication” from “extents of authentication” and “operating limits” from “application limits” is helpful for teaching the invention but otherwise may be implemented in any number of various data arrangements within the use certificate 35-uc, or even not implemented as a use certificate 35-uc, where even many limits may be omitted while yet other non-specified limits may be added. What is important to see is the any one of or any combination of one or more supervisors 24 and operator 10, both preferably but not necessarily acting through an honest broker (i.e., the primary device with intermediary app 1,) may communicate “limits” of various sorts to any of the asset 20-c, or 20-d, a work product app 20-c-app, or 20-d-app, respectively, or a work product authenticator 36g, such that the processing of the operator 10's behaviors into digital input and ultimately digital content/work products 20-c-wp, 20-d-wp, respectively, can be best verified or authenticated as being of human origin and thus organic as opposed to of artificial origin and thus synthetic.

Referring still to FIG. 11B, and now particularly to the bottom half of the figure depicting the side-by-side component processes of the work product authenticator 36g (preferably executing on a primary device 1) working in cooperation with a work product app 20-c-app, or 20-d-app (preferably executing on an asset 20-c, or 20-d) that is being used by an operator 10 to develop a work product 20-c-wp, or 20-d-wp, respectively.

Both side by side components, thus authenticator 36g and work product app 20-c-wp, or 20-d-wp, are portrayed as comprising three interacting sub-processes coordinated and similarly depicted by like purpose which are invoked by the components in the performance of the component's stated goals. Those familiar with software architectures will understand that the portrayal of FIG. 11B is for teaching purposes and may be implemented in virtually limitless ways in functioning software while still achieving the purposes and technical advantages taught herein, thus the present depiction should be considered as exemplary and for illustration purposes, rather than limiting to a specific software implementation.

The present invention teaches the advantage of having “parallel” components, where the first component is the authenticator 36g executing on the primary device 1 essentially monitoring the user/operator 10's behaviors, where authenticator 36g and device 1 are separately securable from the second parallel processing component of the asset 20-c, or 20-d that is processing the digital inputs responsive to the operator 10's behaviors, where then as will become more clear with further discussion and upon a careful consideration, this separation of cooperating but different components, each with their own secure data paths, then significantly increases the difficulty for an adversary to create a “deep fake” synthetic in an attempt to pose a synthetic work product as an organic work product, as this deep fake must run through the separated parallel paths providing tightly correlated but substantially different types of “deep fakes,” the first being imagery (currently limited to 2D) posing as a live operator attempting to be captured and processed by the primary device 1 to match the imagery of a real (3D) operator 10 interacting with the asset 20-c, 20-d input peripherals, and the second being posed digital inputs attempting to be injected into the asset 20-c, 20-d via a communications path so as to be processed by the app 20-c-app, 20-d-app, respectively, to match real human style inputs, where then this posed imagery along with posed digital inputs must exhibit tight and objectively measurable correlation.

The first two parallel sub-processes to be discussed are the request user confirmation 20-app-1 sub-process preferably executing as a part of app 20-c-app, or 20-d-app on the asset 20-c, or 20-d, respectively, that interacts with the privately authenticate user 36g-1 sub-process preferably executing on the primary device.

User confirmation requests 20-app-1 are preferably limited to ad-hoc operator 10 presence checks or verifications, as opposed to operator 10 liveness or correlated behavior checks that both require “on-going” measurements of the operator 10. A simple request for example is that the honest broker (thus intermediary app) executing on the primary device 1 capture a current image of the operator 10 to compare to a prior registered image of the operator 10 (all as prior taught) and then to either provide confirmation to the app 20-c-app, or 20-d-app and/or to record on the primary device 1, in each optional case for indication in the work product certificate 36g-wc, thus ensuring that the operator 10 is present at a certain session time within the duration of session operation comprising the creation of the work product 20-c-wp or 20-d-wp, where this “request to confirm operator's presence” is preferably a randomly determined session time that is indicated in the work product certificate and may include any level of confirmation from an “operator present confirmation datum” associated with a session time to even an image of the operator 10 or some derivative of the image of the operator 10 associated with a session time. What is important to understand is that at least the session time of any particular “response challenge” by the app 20-c-app, or 20-d-app via sub-process 20-app-1 cannot reasonably be anticipated by an adversary who must then additionally pose a response to the challenge and then furthermore inject this response as visual information into the data collection path (e.g., a camera) of the primary device 1, all the while pausing the posed operator 10 imagery and the posed asset 20-c, 20-d digital inputs, the demand of which would require an adversary to provide an automated “session observation” process attempting to intercept such requests and then to simulate a sufficient response.

As a further protection against adversaries, the present invention prefers that at least the communications between the privately authenticate user 36g-1 and request user confirmation 20-c-app-1 are secured communications using encryption, such that multiple encrypted requests can be made where some requests are “dummy” requests not to be responded to while then others can include and offset session time, both approaches then further frustrating the adversary. It is further anticipated that the generation of the “request” is determined at least on some occurrences wholly by the honest broker/primary device 1, which then also satisfies the request by authenticating the operator 10's presence through image comparison as prior mentioned, where then the work product app 20-c-app, or 20-d-app is never aware of the “honest broker inserted” presence checks, thus thwarting an adversary attempting to “listen in” on communications between the app 20-c-app, or 20-d-app and the primary device 1. It is also possible that the presence check includes different types, where then type is also encrypted such that the adversary must then also essentially guess which presence check is being required and respond appropriately, even if the time of request is determined by the adversary.

In a preferred system, the camera embedded in the primary device 1 comprises a field-of-view that includes the operator 10's face, upper body, and hands on the keyboard (or otherwise essentially the operator 10's hands in front of and cable of providing tactile inputs to the asset 20-c, or 20-d,) where such technology for mounting a primary device 1-1 smartphone in landscape orientation onto a display of an asset 20-c, 20-d, or off to the side of an asset 20-c, 20-d, is well-known in the marketplace and provides the desired field-of-view.

Thus a request for confirmation of operator 10's presence could check for a facial image match as prior discussed, or even a clothes color match, or a hands present match, where the response is predicated on an analysis of an primary device 1 image that includes imagery for determining each and all possible specific requests, but where the adversary unable to decrypt the secured request would not reasonably be able to predict which one or more types of presence checks have been requested, thereby being unable to properly respond.

Still other presence checks could essentially be what is commonly referred to as a “captcha,” and is designed to present a challenge that requires a level of understanding that is not present in a synthetic system (“bot”/AI), such as an image with multiple objects presented on the display associated with the asset 20-c, or 20-d, asking the operator 10 to pause input and select the proper object from amongst the multiplicity. This captcha style request could also involve presenting or augmenting the request audibly using the asset 20-c, or 20-d's speakers, where then the operator 10 (or a synthetic adversary) must then correctly receive and interpret this audible request, creating even further barriers to the adversary. A careful consideration of the present teachings with respect to the randomly timed prompt-challenge implemented by either the primary device 1 acting alone (such as by capturing an image and confirming any of the operator 10's face, clothes, or hands,) the work product 20-c-app, or 20-d-app, acting alone (such a through a display presented captcha,) or the combination of the primary device 1 acting in combination with the work product 20-c-app, or 20-d-app preferably over secured and encrypted communications has many possible variations, some of which have been discussed or anticipated, others of which will be obvious or become obvious as technology and algorithms advance, such that the present teachings should be considered as exemplary rather than as limiting.

Still referring to FIG. 11B, and now to the process device/app inputs 20-app-2 sub-process preferably executing as a part of app 20-c-app, or 20-d-app on the asset 20-c, or 20-d, working in parallel with the process user movement 36g-2 sub-process preferably executing on the primary device. Process inputs 20-app-2 is at some level already a part of a traditional work product app as substantially any app must translate data input into the asset 20-c, or 20-d as being either or both of app commands or work product content, all as will be well understood by those familiar with computer programming and application development technology.

Such traditional apps processing data input are herein further adapted to be work product apps 20-c-app, or 20-d-app, comprising a sub-process that additionally interprets and processes these same or similar data inputs to asset 20-c, or 20-d as further inputs to or otherwise forms of novel work product metrics, such as where the total inputs are accumulated as percentage interpreted as “app function commands” versus “work product content,” or even more specifically, specific commands such as “insert content” in combination with “work product content” then inserted, etc., each of which can be denoted by associated session times. Hence, the preferred process device/app inputs 20-app-2 process receives limitations that dictate the types and kinds of input data to be provided to a next sub-process 20-app-3 that is responsible for determining the on-going work product metrics, where, as will be clear to those familiar with software architecture, the combination of 20-app-2 and 20-app-3 could be a single combined sub-process, or even divided into more sub-processes, all without departing from the scope and spirit of the present teachings.

What is important to see is that one aspect for determining an organic work product is to characterize the “input nature.” For example, the interpretation of inputs being organic inputs can be based upon any one or more of “complexity of inputs,” “extended duration of time to enter,” “in-part” with respect to the total work product. “being edited” with respect to prior work product. “amount of commands” versus “amount of work product digital content,” as well as other possible interpretations and metrics with respect to any and all data input during a session of operation for creating a work product 20-c-wp or 20-d-wp. Those familiar with the “black box”/prompt-yields-entire-response “output nature” of generative AI will recognize that the organic nature of supplying the inputs to an asset 20-c, or 20-d for use by app 20-c-app, or 20-d-app, respectively, for the creation or modification of a session work product 20-c-wp, or 20-d-wp, respectively, is significantly and objectively measurable and quantifiable as being different from the provision of content by an operator 10 working with a generative AI process, especially as the work product 20-c-wp, or 20-d-wp increases in content size and complexity.

Thus, and adversary desirous of inserting posed data inputs into an app 20-c-app or 20-d-app must then restructure these same data inputs to appear as similarly created by a human operator 10, placing an additional processing burden and challenge for the adversary. Furthermore, one advantage of AI generative content is that it can be generated “single shot” and in a very short session duration as compared to a human session of operation, where this advantage is significantly frustrated if the adversarial synthetic process must then simulate an extended duration of time entry, especially as might be confirmed by any of a primary device 1 or temporal session certifier 26 (see FIG. 10A) that is working to ensure that the received input data is transpiring over extended real-world time, all as a careful consideration and reading of the present teachings will show. It is well-known that the cost to operate such synthetic algorithms can be significant and as such increasing this cost by forcing the synthetic content to mimic organic content entry provides an economic deterrent and is not just technologically problematic to the adversary. What should be understood is that work product metrics as determined by sub-process 20-app-3 by themselves are strongly indicative of an organic input nature versus a synthetic input nature.

However, even if an adversary is sufficiently able and willing to transform otherwise synthetic content to mimic the data inputs representable as organic input for posing and inserting into a work product app 20-c-app, or 20-d-app, the present system further anticipates that the operating system of the asset 20-c or 20-d traditionally tasked with shunting data inputs from the various peripherals (such as a keyboard, trackpad, mouse, touch screen, pen and tablet, etc.) to the work product app 20-c-app, or 20-d-app, respectively, can be further adapted to strengthen these communication paths against the inserting of such synthetically posed data. Still yet further or instead, parallel sub-process 36g-2 for processing user/operator 10's movements, and 36g-3 for determining on-going work-user metrics, creates another significant challenge to the adversary.

Process 36g-2 preferably executing on the primary device 1 is processing data captured exclusively by the primary device 1 and interpretable or indicative of the operator 10's on-going behavioral changes, such as moving the head, changing the eye gaze, moving the hands or even specific fingers, all with respect to a “surface of transduction” or “user interface” provided by the asset 20-c or 20-d for the translation of operator 10 behavior into digital inputs for providing to work product apps 20-c-app, or 20-d-app, respectively. In a preferred configuration, primary device 1 is a smartphone 1-1 or otherwise has access to a camera for capturing on-going images of the operator 10, substantially as operator 10 is providing real-time inputs to the asset 20-c, or 20-d, where these images as prior mentioned have a field-of-view encompassing at least the operator 10's face, but then preferably also the upper body and hands as well as any of the surfaces of transduction, such as a keyboard, mouse, trackpad, etc. Those familiar with the state-of-the-art in image processing will appreciate that existing technologies are already capable of translating video images of a person's face, torso, and hands to create the behavioral information described herein, including but not limited to head movements, eye gaze, lip movements, facial expressions, clothing colors, hand movements, finger movements, finger's touching specific locations (such as a key on a keyboard or a mouse button,) etc. Just as with parallel sub-processes 20-app-2 and 20-app-3, sub-processes 36g-2 and 36g-3, as well as even 36g-1, may alternatively be implemented as a single sub-process or as more than the three preferred and displayed sub-processes, all as will be clear to those familiar with software system and all without departing from the scope, spirit, and teachings of the present invention.

What will be clear upon a careful consideration, is that preferably a primary device 1 executing under its own secured means determines the behaviors of an operator 10 at varying levels of possible detail, ranging from occasional simple head, body, lips, and/or hand motion (thus supporting the indication of operator “liveness” to combine with operator 10's identified “presence” as discussed in relation to sub-process 36g-1,) to more frequent and more detailed head, eye gaze, body, lips, hand, and/or finger motion, all referenced to a surface of transduction with corresponding “input surface area” (such as an eye gaze looking at a portion of a screen altered by way of a challenge-response request made by request user confirmation sub-process 20-app-1, or a particular key on a keyboard being touched by a particular finger of the operator, etc.,) where these more frequent and detailed operator 10 behaviors will exhibit an higher and higher degree of correlation to the on-going work product metrics determined by 20-app-3, or even the processed inputs as determined by 20-app-2 as the frequency and detail are increased, thus providing for various levels of organic work product authentication.

Still referring to FIG. 11B, such work product authentication is preformed by sub-process 36g-4 that receives both work product metrics from sub-process 20-app-3 and work-user metrics from sub-process 36-3 for correlating and determining a corresponding level of organic veracity, where any of the inputs to sub-process 36-4 are preferably kept private and even discarded after the sub-process 36-4 is able to determine a level of organic confirmation to encode into work product certificate 36g-wc. Optionally, work product authenticator sub-process 36-4 provides feedback to app 20-c-app, or 20-d-app to provide the current levels of substantiated organic certification, where then for example app 20-c-app or 20-d-app increases or decreases the requests for user confirmation made by sub-process 20-app-1, or alternatively changes the types of challenge-prompts requests as prior delineated.

In summary with respect to FIGS. 10A, 10B, 11A, and 11B, the careful reader will see that it is possible to determine an organic work product 20-c-wp or 20-d-wp by tracking the data inputs to an asset 20-c or 20-d, respectively, used by an operator 10 to create the work product concurrently with tracking the behaviors of the operator 10 causing the data inputs as transduced by a surface of transduction/user interface, all with varying levels of authentication detail. What the careful reader will also recognize is that as the work product increases in complexity and content size, there is a corresponding increase in the in-part and edited nature of the input content, all further distinguishing the operator 10/organic work product creation from AI generative synthetic work product creation. The reader will also recognize that there are currently a significant number of types and uses of digital work products being generated by human operators 10 that are able to be synthetically created by current or future generative AI and similar algorithmic approaches, where there is a strong market need to differentiate the “organic” work products from the “synthetic” work products. For example, social media platforms such as Twitter and Meta are currently working to provide “verifications” that a work product (e.g., a Twitter “tweet” or Meta posting) is organic including the use of paid services such as Twitter's “Blue Check Mark” or Meta's “Meta Verified,” where again the goal is to help differentiate human social media content from “bots.” As those familiar with these and other such social media services will appreciate, the present teachings provide for a higher degree “organic authentication” than is possible using these currently implemented solutions.

As was also discussed, both the educational market and corporations are challenged by the presentation of synthetic content that is substantially “post-fact” undiscernible from organic content, where the present teachings offer significant technical advantages for the “pre-fact” labeling of all types and manner of digital content as being “organic” in nature. The careful reader will also recognize the broad advantages of the “honest broker” functionality implemented by the use of a primary device 1 as taught in the related applications and herein, where the advantages span the range from an individual proving a sufficient health status to participate in a group function, to an individual proving a sufficient physiological and psychological readiness to operate a physical or digital asset, to yet then also an individual proving that they are the originators of a given work product, where then all of these types of functionality can themselves range from fully anonymous to fully disclosing of the identity and even associated authenticating biometrics of the individual.

As will also be clear with respect to these many and various functions ranging again from health checks to readiness to operate assets, to proof of organic work product, there are significant legal implications that are better adjudicated given the novel information provided by the present invention. It has also been shown that significant histories can be established by first authenticating the individual wishing to enter, wishing to operate, and wishing to demonstrate ownership of work product, where then these histories have been shown to provide significant analytical opportunities to third-parties for better assessing trends in group health, asset operation, and work product creation.

Referring next to FIG. 10C, there is shown a block diagram of system 100-5 with pictorial support for understanding the key concepts for determining any one of or any combination of “certified organic content” work product certificates 36g-1, 36g-2, or 36g-3 that are determined pre-fact of created and/or modified session work product 20-c-wp, 20-d-wp, where certification is based upon any one of or any combination of (i) determinations with respect to the input nature of the work product 20-c-wp, 20-d-wp as determined by analysis of indirect metrics 20-c-m2, or 20-d-m2, (ii) correlations 36g-c determined between the input nature of the work product 20-c-wp, 20-d-wp and the presence, liveness, and/or behaviors of the operator 10 as determined by analysis of direct data/metrics 20-c-m1, 20-d-m1, and (iii) any of user confirmations made in response to preferably session-random user confirmation requests 20-ruc.

System 100-5 preferably comprises an asset system 20-c, 20-d into which an operator 10 provides one or more behavioral movements such as hand and finger movements, eye gaze, lip movements (generating audible voice,) etc., where the operator 10's behaviors are transduced through one or more user interfaces 20-ui such a keyboard, a mouse, a pen tablet, a touch screen, a camera, a microphone, etc., into asset indirect metrics 20-c-m2, 20-d-m2, respectively, indicative of the input nature. Operator 10's behaviors are captured and transduced into asset direct metrics 20-c-m1, 20-d-m1, respectively, indicative of the operator 10's authenticated presence, liveness, and detailed behaviors.

Concurrent and congruent with a session of operation performed by the operator 10 using the asset system 20-c, 20-d to create or modify a session work product 20-c-wp, 20-d-wp, respectively, one or more other independent session observer systems 20-ios (such as authenticating primary device 1 or temporal session certifier 26) are either in (preferably secured) communications with asset system 20-c, 20-d, and/or are observing operator 10 substantially during the session of operation, where the observations include determining any one of or any combination of operator 10's authenticated presence, liveness, and behaviors (such as eye gaze, lip movements, or hand-finger movements). The “pre-fact” indirect metrics 20-c-m2, 20-d-m2 as well as “pre-fact” direct metrics 20-c-m1, 20-d-m1 provide valuable indications of the organic versus synthetic origination of the work product 20-c-wp, 20-d-wp that is then sharable “post-fact” with other persons and systems as certified organic authenticated session work product 36g-awp.

The present system 100-5 teaches the use of any one or more work product authenticators 36g-1, 36g-2, or 36g-3 being executed on an asset 20-c, 20-d, an authenticating primary device 1, or a temporal session certifier 26, respectively. Work product authenticators 36g-1, 36g-2, or 36g-3 determine one or more indications (i), (ii), or (iii) of “organic” origination of the work product 20-c-wp, 20-d-wp through the analysis of the “pre-fact” direct metrics 20-c-m1, 20-d-m1 and indirect metrics 20-c-m2, 20-d-m2. Asset 20-c or 20-d optionally comprises an authenticating imager 20-d-ai for capturing through the transduction of light energy one or more images or video, where imager 20-d-ai embeds one or more authenticating signatures within the image data issued from the imager 20-d-ai.

Referring next to FIG. 11C there is depicted generalized work product app 20g-app, for example comprising any of a system 20-c app (thus, 20-c-app,) or a device 20-d app (thus, 20-d-app). Generalized app 20g-app comprises two highlighted functions, 20g-app-f1 for obtaining/verifying prior work products and 20g-app-f2 for creating new/modifying prior work products, where these functions 20g-app-f1 and 20g-app-f2 are otherwise well-known with respect to software application development and are typically implemented as a single program/app, although herein for teaching purposes separated as functions. Regarding function 20g-app-f1 for obtaining prior work products, it should be understood that this applies to the ingesting by apps 20g-app of any pre-composed data received into the app from an external connected source, thus in a sense data or content pre-existing at least prior to the “moment of insertion” into the app 20g-app's current data maintained during the session-of-operation, if not also typically pre-existing to the session-of-operation itself.

Typically, the traditional basis for an app 20g-app (such as a word processor, 2D/3D drawing tool, picture editor, spreadsheet, programming studio, etc.) provides a user-interface for excepting at least the operator 10's hand motion behaviors (such as typing on a keyboard or moving a mouse,) where then these operator 10 behavior correlated app 20g-app inputs (see asset indirect metrics 20-c-m2 of FIG. 10A and 20-d-m2 of FIG. 10B) are typically interpreted by the app 20g-app as app commands and/or app input data, where at least these indirect metrics (generalized as 20g-m2 but not depicted) can be considered real-time commands and data, if not also real-time operator 10 generated digital content (where a command per se, such as a button press, is distinguished as transient and not being maintained as the content comprising a current session work product 20g-wp).

However, it is noted that the current further adapted app 20g-app's input may for example be a “insert button press” or similar UI interaction caused at least in part by an operator 10's behavior (such as a mouse click,) where then the app 20g-app proceeds to insert a significant amount of pre-existing data/content. The main distinction being taught with respect to the present FIG. 11C is that while pre-existing data/content is often inserted (determinable by analysis of indirect metrics 20g-m2) and in correlation to an operator 10's behavior(s) (determinable by analysis of generalized direct metrics 20g-m1 comprising direct metrics 20-c-m1 of FIG. 10A and 20-d-m1 of FIG. 10B,) non-pre-existing data/content is created minimum-datum-by-minimum-datum responsive to the operator's manipulation of the app 20g-app's UI (determinable by analyzing 20g-m1, and confirmable by analyzing 20-gm2).

It is further noted that “pre-existing” content is not limited to being stored or maintained on the originating system 20-c or device 20-d prior to the commencement of a session of operation for creating or modifying new session work products 20g-wp. For example, a new session of operation of app 20g-wp for creating or modifying a new/updated session work product 20g-wp might ingest or otherwise receive data or content that is transmitted via a communications path where the source of this path is not some form of system 20-c or device 20-d data storage (see FIG. 11D for examples of ingested pre-existing data that is not stored locally on the system or device executing the app 20g-app). It should therefore be understood that pre-existing data is meant to describe any data or content otherwise external to the current dataset comprised by an app 20g-app that is then ingested (inserted) into the current dataset, regardless of the source of this data or content being either of what is often referred to as persistent (e.g., a data storage file) versus transient (e.g., a message generated from the external source that is stored in local memory but otherwise essentially discarded after transmission to the receiving app 20g-app).

It is noted that a social media message such as a text, a “posting,” etc., (often considered as transient) is both typically transmitted from a source for ingestion into an app 20g-app during the session-of-operation, and often also typically stored and maintained post-session as local system 20-c or device 20-d data, all as will be well understood by those familiar with software and networked systems. It is also noted that regardless, for the purposes of the present invention, content composed in response to operator 10's direct 20g-m1 behaviors (such as finger movements on a keyboard) and correlated indirect 20g-m2 transduced inputs (such as data coming from the keyboard in response to the finger movement,) as distinguished from any persistent or transient pre-existing inserted content, is considered as “organically” sourced session work product 20g-wp.

What is important to see is that the data/content was determined to have been composed in its smallest possible units-of-content (e.g. “letter-by-letter” from a keyboard) by the operation 10, as opposed to inserted and typically larger units-of-content (e.g., “ten pages of words”) that are again necessarily pre-existing to the moment of commanded insertion.

Those familiar with various software apps (thus being the traditional basis for 20g-app) will also understand that an app 20g-app's user interface is purposed to allow a user to compose digital content comprising a multiplicity of different “minimal content objects,” for example a single letter in a word processing app, a sized-rectangle in a 2D drawing app, a number associated with a data cell in a spreadsheet app, etc., where each of these minimal content objects are created by the app 20g-app in direct correlated response to an operator's 10's behaviors (most often hand motions interacting with a keyboard or mouse,) but where also these minimal content objects may in fact be represented by significant amounts of “app data.” For example, the traditional data representation of a single letter in a text document is known as an ASCII character comprising 8 bits of data (also referred to as 1 byte,) which is a minimum size of data at least for transductions related to a traditional text-entry keyboard. However, a drawing package representing a rectangle placed in a 2D drawing space requires multiple bytes of data for internal app 20g-app representation. As such, the idea of minimal content is fluid with respect to data size and is more a property of the app 20g-app's allowed (thus “native”) content input functions. Alternatively, “inserted content” that is pre-existing to the moment of insertion (where typically the insertion is at the direction of the operator 10 indicated by a detectable behavior 20g-m1 and/or input data 20g-m2 itself being a “minimal content object” comprising an “insert command”) necessarily or at least typically comprises a multiplicity of minimal content objects to be useful to the operator 10. As a careful consideration will show, inserting a minimal content object (such as a single letter, rectangle, or number) does not leverage the operator 10's behavior, as this same minimal content object could most likely more easily by added to the existing app's 20g-app current content by simply using the app's 20g-app UI. (For example, why insert the letter “A” when it is easier to simply press the “A” on the keyboard?)

What is important to see is that the present teachings with respect to determining a work product certificate 36g-wc in general, is that the operator's final session work product 20g-wp is now additionally described by the certificate 36g-wc whose description preferable includes the work product's 20g-wp sources of content composition (see discussion related to 20g-sc and 20g-rc). One source of content is typically the operator 10 using the app 20g-app's UI to direct the “next insertion of minimal non-pre-existing content,” where other source(s) can provide the “next insertion of non-minimal pre-existing content,” and where these other source(s) of non-minimal pre-existing content are typically accessible to the app 20g-app via programmatic communication (e.g., via the “system clipboard” or an “application programming interface”). A careful consideration will show that this pre-existing other source(s) content could be of either type “organic” (meaning generated by a person) or “synthetic” (meaning generated by AI or at least not by a person,) or some combination thereof.

Still referring to FIG. 11C, in the simplest example operator 10 is using app 20g-app to create new digital content without starting from any pre-existing content or inserting any pre-existing content, and as such function 20g-app-f1 is not invoked. In this simple example, the operator 10 instead uses function 20g-app-f2 to compose. “minimal content object”-by-“minimal content object,” the entirety of the operator's session work product 20g-wp, the culmination of which results in authenticated session work product 36g-awp comprising the combination of substantially the traditional session work product 20g-wp and the herein taught work product certificate 36g-wc representing authentication of operator 10 and verifying operator 10's composition of work product 20g-wp.

As prior stated herein, certificate 36g-wc is minimally indicative of the authenticity of the operator 10 (thus some indication ranging generally from the operator 10 being “a person,” to a specific person identified using one or more personally identifying information, to a person identified by an anonymous ID, all of which being indicative of an “organic agent” causing the work product composition). By implication, certificate 36g-wc is also indicative of this authenticated operator 10 as being at least the compositional-author of the session work product 20g-wp, thus indicating that the authenticated operator 10 (respective of any of the operator 10's tracked presence, liveness, or other concurrent and congruent behaviors) is verified to be the operator of the asset (system 20-c, device 20-d) during the session of operation that generated (“composed”) the work product 20g-wp (even if some or all of the final content comprising the work product 20g-wp is not of the operator 10's own “thought” or “mind creation”). As will be discussed briefly with respect to the present FIG. 11C, but more fully with respect to upcoming figures, app 20g-app preferably but optionally also generates content-authorship view and characterizations 20g-sc with optional reference(s) and optional prompt(s) 20g-rc, where both 20g-sc and 20g-rc comprise a range of possible digital representations.

As a careful consideration will show, an authenticated operator 10 of a session of operation using an app 20g-app to determine a work product 20g-wp may or may not be the content-author of any particular portion (i.e., compositional part) of the entire composition that is the work product 20g-wp, but they are, by virtue of being the “authenticated session operator” 10, the composition-author of the work product 20g-wp. It is then noted that preferably the work product certificate verifies that the authenticated operator 10 is the composition-author (a necessarily “singular” title) of the work product 20g-wp, whereas the authorship view and characterizations 20g-sc is meant to describe at least the authorship of each compositional-part comprising the “singular” work product 20g-wp composition, where there are a potential multiplicity of compositional-parts and thus in a sense content-authorship is a “multi-dimensional” title).

Session authorship view and characterizations 20g-sc are thus meant to describe the origins (“authorship”) of the distinct content parts of the session work product 20g-wp, again, with the understanding that a work product 20g-wp can be of a single-source content-authorship or alternately a sophisticated composition of many possible content-authors, and that these authors may be “organic” (thus people) or “synthetic” (thus algorithms). It is noted that for each session of operation, whether initially creating the session work product 20g-wp or later modifying a prior session work product 20g-pwp, there is always a “current session operator 10” who is then at least the “composer of the composition of the work product,” even if this composition itself comprises little-to-no original content supplied in verifiable correlation with this composing operator 10's own measured behaviors (thus operator 10 is not measured or determinable to also be an author of any appreciable content comprising the composition 20g-wp). It is also noted that it is possible that this composing operator 10 is algorithmic (i.e., “synthetic”,) although currently such algorithms are not devised to operate a traditional app 20g-app via the various traditional input apparatus associated with a system 20-c or device 20-d (e.g., keyboards, mice, digital pens, touch surfaces, microphones, etc.,) and thus also do not have measurable physical behaviors that can be determined and correlated with on-going digital input into an app 20g-app as does a human operator 10 (where then this lacking of measurable physical behaviors is then interpretable as indicating a synthetic compositional author). As taught herein, this human operator 10 effort is determinable by analysis of both the direct metrics 20g-m1 and indirect metrics 20g-m2.

For example, if an operator 10 uses an app 20g-app to create a new session work product 20g-wp, where the work product 20g-wp initially comprises no content but then upon the operator 10's ending a current session of operation of the app 20g-app the work product 20g-wp comprises a non-zero amount of content, the session authorization view and characterizations 20g-sc are purposed to described the resulting non-zero content-composition in terms of the origins (“authorship”) of its one or more compositional digital-parts. If an operator 10 simply uses the app's 20g-app UI to provide the next insertion of minimal non-pre-existing content (e.g., “letter-by-letter”/“character-by-character”/“keystroke-by-keystroke”) than authorship view and characterization 20g-sc preferably indicate that the entire session of operation (regardless of any duration of time) produced a single “session content chunk” as expressed “tracked input by tracked input” by a single author (see also FIG. 11E,) where this single session content chunk could be a single byte or some large multiplicity of bytes of data representing one or many minimal content objects. However, the insertion of pre-existing content is quite typical when conducting a session of operation using an app 20g-app, and in such cases the authorship and characterization 20g-sc preferably indicate each distinct “continuous creation of typically minimal non-pre-existing content” as well as the “substantially immediate insertion of typically non-minimal pre-existing content,” where each type of “created chunk” versus “inserted chunk” of content comprises any of a spatially-contiguous and/or temporally sequential determinable series of minimal content objects. The resulting description 20g-sc of the multiplicity of create or inserted chunks (some created by the operator, some inserted at the command of the operator) also preferably indicates the sources, references, and thus “authorship” of each compositional part (“chunk”) comprising the final composition that is the session work product 20g-wp. Again, it is useful to understand that a session content chunk is a spatially contiguous and/or temporally sequential uninterrupted insertion.

For example, a third-party icon inserted into a current 2D 8.5″×11″ image is “spatially contiguous” and likely comprises a multiplicity of minimal content objects (e.g., rectangles, color fills, lines, etc.) being inserted at the direction of the operator 10 using the app 20g-app UI, where this inserted third-party icon may however also spatially overlap the “interrupted by insertion” prior content (thus no longer being spatially distinct) but will otherwise be temporarily sequential and thus differentiable as a distinct session content chunk, all as a careful consideration will show.

In another example, an operator 10 typing 327 consecutive words into a word processor app 20g-app (typically sporadically over an extended period of session time) before then interrupting this temporally-sequential insertion of 327 consecutive words to request the app 20g-app via the app UI to insert for example a distinct 128 word paragraph copied from a source paper found on the internet has created two session content chunks as herein defined, the first being the “created chunk” of 327 temporally sequential words (perhaps entered via a keyboard over minutes of time) and the second being the “inserted chunk” of the pre-existing 128 words (perhaps inserted programmatically via the system clipboard in milli-seconds,) followed typically by even more session content chucks (created or inserted). The present teachings refer to each session of operation of an app 20g-app by an operator 10 as creating a composition whose net content is the traditional session work product 20g-wp, where the composition of this net content comprises one or more components or “composition parts,” where these components may be any of pre-existing and programmatically inserted as a single inserted content chunk without substantial correlated operator 10 behavior or determined over a duration of sequential session time and comprising a multiplicity of minimal content objects as directed by the correlated behaviors of the operator 10, and where then this composition of net content/session work product 20g-wp is preferably described in the authorship view and characterizations 20g-sc.

Since inserted pre-existing content may be derived from a number of possible sources, original references and prompts content 20g-re is preferably determined for association with any such pre-existing content as described by the authorship view and characterizations 20g-sc, thus recording “original pre-existing” that may later be usefully compared to the work product 20g-wp to ascertain any levels of operator 10 authored “editing” of this pre-existing original content. Those familiar with software and data descriptions will also recognize that artifacts 20g-sc and 20-rc could be combined into a single artifact, or further broken into multiple artifacts. What is preferred is that authorship view and characterizations 20g-sc describe all of the created and inserted content chunks, identifying authorship and identifying the minimal content objects comprising each chunk (all as will be further discussed) along with many possible “input nature” characteristics of each chunk (see prior description and upcoming description). Authorship is then a relatable descriptor to the reference and prompts 20g-rc via preferably a “content chunk unique identifier” used to aggregate and index data in both 20g-sc and 20g-rc. Reference and prompts 20g-rc preferably includes the originally inserted content (such as a picture or text) as well as in the case of “generative AI” optionally what are known in the art as the “engineered prompts” first provided by the operator 10 to the generative AI algorithm, for which the response picture, text, audio, etc., is related, where then any of this originally inserted content being saved as 20g-re is comparable with the final session work product 20g-wp to determine or confirm any amount of post-insertion editing by the operator 10 author.

The careful reader will also understand that the determination and detailed extent of any of authorship view and characterizations 20g-sc as well as reference and prompts 20g-re is controllable via either of the use certificate 35-uc and/or the supervisor certificate 24-sc, where for example an education institution requires via a supervision certificate 24-sc that any and all inserted content into a composition be accompanied by the identification of the source referenced (thus indicative of the original content comprised in 20g-rc,) where as will be discussed this reference may include traditional identifiers (such as an article name, publication source, data of publication, providing web-service, author/creator's name, etc.) or even an associated other work product certificate and/or commercial license, where it is even anticipated that the app 20g-app can prohibit the insertion of any “non-identified” “pre-existing content” as per the directives of the certificates 35-uc or 24-sc.

Still referring to FIG. 11C, in a typical session of operation of an app 20g-app, the session commences with pre-existing session content, where for example an operator 10 “opens the digital document” that is the “operator's prior work product” 20g-pwp, such as a word processor file, an image, a 2D/3D drawing model, an audio file, a computer program, etc. In this case, the session of operation is typically described as “modifying existing work product” as compared to “creating new work product,” where the modification process for the purposes of the present teachings preferably distinguishes between this “opened” session pre-existing work product as opposed to any and all new session content chunks, where these new chunks can be either the result of operator 10's behaviors causing spatially-contiguous or temporally-sequential components comprising one or more minimal content objects, or operator 10's behaviors requesting the insertion of pre-existing typically non-minimal content objects.

Regardless of session of operation insertion timing, whether at the beginning of the session (thus “opening pre-existing content”) or during the session, function 20g-app-f1 teaches the further adaptation of existing traditional apps to additionally determine the prior work product certificate 36g-pwc of any opened/inserted/starting prior work product 20g-pwp, where often this opened/inserted/starting content was originally authored as a composition by the current operator 10 who is then desirous to modify their own prior composition 20g-pwp and to ultimately provide a new certificate (n+1) 36g-wc confirming their “continued compositional authorship.” Thus, in at least some cases, an operator 10 may be intent to modify their own prior work product 20g-pwp in which case function 20g-app-f1 also ingests some or all of the work product certificate (n) 36g-pwp (as herein taught) associated with the prior work product 20g-pwp, such that upon completion of the session of operation a new work product certificate (n+1) 36g-we can be created (or the prior certificate 36g-pwp can be updated) verifying that the authenticated creation-plus-modification cycle is of a single “on-going”/multiple-session compositional authorship, therefore authenticating that operator 10 is the composer of the “on-going” session work product 20g-wp.

It is emphasized that this “on-going” authentication of session work product 20g-wp, created and then subsequently modified, is a key function of the present teachings and goes beyond traditional apps for which a person may self-associate themselves with their work product, but otherwise this self-association (without the presently taught authentication using a primary device 1 (or otherwise “honest broker” app functionality) is not “publicly trustable,” all as a careful reading of the present and related applications will show.

Furthermore, the present teachings now provide objective means for authenticating other inserted session work products 20g-pwp or 20g-owp, which themselves were prior authenticated (via certificate (n) 36g-pwc or 36g-owc, respectively) as having been created by the same operator 10 or some other operator 10, where this “authentication-of-authentication” is accomplished by the verification of prior work product certificates 36g-pwc or 36h-owc, respectively, preferably at the time of work product insertion. Thus the careful reader will see that the present invention not only authenticates created session work products 20g-wp, but also authenticates on-going modified session work products 20g-wp, where furthermore any given work product may comprise components of session content authenticated to have been prior created by the same or other operators 10, thus supporting a virtually limitless number of contributors to any simple (e.g., a single digital document) to any more complex (e.g., compound digital document comprising other compound or simple documents) session work product 20g-wp, each going through any number of editing cycles.

And finally, still referencing FIG. 11C, function 20g-app-f1 preferably supports the insertion of what is often referred to as “common” content that is “free-to-use” and “in the public domain,” where this common content 20g-uwp is often of unknown authorship or authenticity, lacking a work product certificate but perhaps having a verifiable “public-use” license that function 20g-app-f1 preferably acquires for use providing proof-of-common-content authorship. As will be discussed in more detail in reference to upcoming FIG. 11D, typically such other prior work product 20g-owp and/or commons/unknown prior work product 20g-uwp is sourced remotely from system 20-c or device 20-d, such as through a communications link to any of digital information sources 28, where sources 28 are any of remote and not comprised within the computing system 20-c or 20-d executing the app 20g-app or a local and comprised within the computing system 20-c or 20-d executing the app 20g-app, and where regardless each of a remote or local source is “uniquely addressable” and identifiable by the app 20g-app for accessing, and where any information sufficient for representing the unique address/identity of the data source 28 is preferably used as reference information comprising authorship view and characterizations 20g-sc.

Referring next to FIG. 11D, there is shown operator 10 using exemplary system 20-c comprising camera 20-c-cam (optionally including an integrated or separately available microphone) for monitoring any one of or any combination of operator 10's presence, liveness, mood/emotional state, voice, hand movements, eye gaze, or otherwise determinable behaviors. Also depicted are optional further adapted keyboard 20-c-kb and mouse 20-c-ms in communication with system 20-c for providing traditional digital data to system 20-c via traditional “path A” while simultaneously providing substantially the same or similar traditional digital data to an honest broker operating on a primary device 1 (such as 1-1 or 1-2) using a non-traditional “path B.”

Wearable smartwatch primary device 1-2 is shown to determine hand and finger movements to some level of accuracy by at least detecting bio-electrical signals, where the detected movements are provided via “path C” to system 20-c as hand and finger behaviors data. Operator 10 uses system 20-c to create a session work product 20g-wp with authentication certificate 36g-wc (neither 20g-wp nor 36g-we are depicted, see for example FIG. 11C) using an app 20g-app (see also FIG. 11C). During creation and/or modification of a session work product 20g-wp, operator 10 provides directives via distinct behaviors (such as typing on a keyboard or moving and clicking a mouse) that are indicative to the app 20g-app of commands or content being provided by the operator 10 for ultimately providing current session work product 20g-wp, or commands for inserting other content (potentially not authored by operator) into the current session work product 20g-wp.

Content being inserted by app 20g-wp at the direction of operator 10 is depicted as digital content 28 coming from any number of well-known “sources and channels” such as, but not limited to, web content 28-1, AI generated content 28-2, stored documents 28-3, or otherwise data coming from any of connected devices 28-4 (even what are known as “IoT” or “internet of things devices” typically for providing real-time environmental/contextual data). Distinct from this possible “inserted content” 28-1, 28-2, 28-3, and 28-4, there is also digital content 28 that is “operator created content” such entered into system 20-c in direct response to any of operator 10's thoughts 28-5 expressed as possible behaviors (traditionally including typing on a keyboard or moving a mouse,) where then such operator created thought content 28-5 is further differentiated as related to system 20-c displayed information 28-6, printed information 28-7, or otherwise any other information 28-8 visible to the operator 10. What is important to see is that the present invention teaches novel apparatus and methods for substantially distinguishing between “inserted digital content” (a pre-constructed series of one or more minimal content objects) versus “created digital content” (a real-time constructed series of one or more minimal content objects,) where inserted digital content definitively comes from a source that is not currently the operator 10 (although it is possible to be the operator 10's prior work product 20g-pwp or from the operator 10 but non-certified such as 20g-owp, or perhaps known but at least “common” 20g-uwp, see FIG. 11C,) while created digital content may not have originated from the operator 10 (such as 28-6, 28-7, and 28-8) but is at least being processed by the mind of the operator 10 and thus representative of operator thoughts 28-5.

Still referring to FIG. 11D, inserted digital content 28 is well-known in the art where app 20g-app forms a digital communication “channel” to the “source” of this inserted digital content 28, such as again web content 28-1, AI generated content 28-2, stored documents 28-3, or otherwise data coming from any of connected devices 28-4 (where 28-4 may be for example the operator's smartphone comprising digital photographs or a tablet comprising a 2D sketch, or in another example a digital device may be lab/work equipment such as a digital microscope, or otherwise any “internet of things” “IoT” connected device providing information). As those familiar with software systems and computer programming will understand, there are many terminologies for naming a digital “channel” or “source,” but regardless of the terminology what is important to see is that in the realm of digital information, there must exist a unique identifier/name for a given source elsewise this source could not be sufficiently differentiated and accessed by the app 20g-app, and as such regardless of specific software system terminology or current practices, the present invention teaches that any such currently practiced or future practiced digital content source naming system can be implemented using the teachings provided herein to distinguish where (i.e., “what is the source”) of the digital content currently being inserted into the session work product 20g-wp by action of the app 20g-app at the bequest of the operator 10.

It is also important to see that while not required, typically any of inserted digital content will actually comprise two or more minimal content objects as opposed to created digital content which is substantially and traditionally limited to being input one minimal content object at a time by the nature of the system 20-c input apparatus (such as a keyboard or mouse) and the app 20g-app. For example, a word processor app 20g-app accepts letters as a fundamental minimal content object, although this single-letter-at-a-time limitation is primary determined by the use of a traditional “qwerty” keyboard, whereas using what is known as a “stenography keyboard” can create minimal content objects comprising text that is more than a single character/letter of text per input behavior (thus “keystroke”). In another contrary example, many modern word processing apps 20g-app either comprise or can integrate with what is generally known as “speech recognition software” (such as the commercially available Dragon Naturally Speaking,) where then a minimal content object comprising text can be a word (multiple letters) or a phrase (multiple words) at a time.

Regardless, the minimal content object should be understood in the context of at least the input apparatus available to the operator 10 and the app 20g-app being used by the operator 10 to generate the session work product 20g-wp, where this minimum content object could also be a range, for example a single “qwerty” keystroke/letter to a spoken word comprising multiple letters, such that what is important to see is that it is possible for the app 20g-app and/or an work product authenticator 36g (see for example FIGS. 10A and 10B) to determine the possible minimal content objects useable by the operator 10 for forming session work product 20g-wp.

App 20g-app and/or authenticator 36g may use knowledge regarding a minimal content object as a means for recognizing non-minimal content objects (e.g., in relation to a textual document an inserted entire paragraph or page of words is recognized as more than a minimal content object thus being a non-minimal content object,) where then any non-minimal content objects comprise a greater number of minimal content objects than can be reasonable specified by an operator 10 using a “single behavior” (e.g., finger-touch on a keyboard) given the limitations of the currently usable input apparatus and the app 20g-app, all as will be well understood by those familiar with software system and user interfaces and a careful consideration of the present teachings. As prior mentioned, while it is strictly possible that inserted digital content comprise only a single minimal content object (or technically even less digital information down to what is known as a “bit” of information,) to be in practice useful, inserted digital content should provide content-entry advantage to the operator 10, where then this content-entry advantage is typically that the inserted digital content is pre-constructed as a multiplicity of minimal content objects that the operator 10 is desirable of using as a compositional-part without otherwise having to composed this part, minimal-content-object-by-minimal-content-object.

While this semi-distinctive quantity-based determination for inserted versus created content is anticipated and useful for determining for example possibly synthetic (because it is inserted) verses most likely organic (because it is entered via a minimal content object path responsive to the operator 10's behaviors,) it is noted that a stronger indicator of inserted versus created content is to differentiate the “data receiving path” or “communication path”/“data channel” through which the app 20g-app obtains the digital content. In this regard, as will be understood by those familiar with software systems, any such “data receiving path” must by necessity be well identified to and by the app 20g-app and could range from a traditional data input apparatus such as a keyboard, mouse, digital pen, or microphone, to a traditional data connection path such as to a source of web content 28-1, AI generated content 28-2, stored documents 28-3, or otherwise data coming from any of connected devices 28-4. As such this “data receiving path” is very distinctive of inserted versus created content, all as a careful consideration will show.

Still referring to FIG. 11D, what a careful consideration will also show is that there are two circumstances where (a) inserted content may actually be operator 10 authored content, and (b) created content may not actually be operator 10 authored content, where, by the teachings provided herein, it is now possible to further differentiate both (a) and (b) for determining actual authorship, and where it highly useful for the determination of the work product certification 36g-wc and authorship view 20g-sc (thus “organic” versus “synthetic” composition and compositional components). (It is further noted that the apparatus and methods of work product certification being taught herein are composable down to distinct incremental “chunks” or series of minimal content objects, see FIGS. 11E and 11F, whether inserted or created, such that a single session work product 20g-wp may and most often will comprise a multiplicity of distinct inserted versus distinct created digital content, especially as it is first created and then modified or edited over cycles of distinct sessions of operations, see FIG. 11C, where then also the “inserted content” may comprise operator 10 created or non-operator 10 created content and the “created content” may likewise comprise operator 10 created or non-operator 10 created content.)

Regarding (a), inserted content that is actually operator 10 created content, the reader is directed to prior FIG. 11C that provides means for the operator 10 to prove authorship of inserted content that is the operator's prior work product 20g-pwp with proper and sufficient certification 36g-pwc, all as taught herein. It is again mentioned that this certified operator prior work product 20g-pwp may itself comprise one or more chunks of inserted digital content that are not certified to be authored by the operator 10, where then it is noted that the present invention teaches a “content chunk authorship schema” (an alternative name for 20g-sc) showing the content authorship structure comprising a potential multiplicity of differently authored digital content, both created and inserted. This “content-authorship-map” is usable when referencing an operator 10's certified prior work product 20g-pwp to determine if a particular portion (“compositional component”) or all of the prior digital content (the entire composition) being input from this insertion source 20g-pwp is actually certified as at least itself inserted or authored by the operator 10, and then within this further and preferably most likely created by the “mind-work” 28-5 of operator 10 regardless of being inserted or created, where as a careful consideration will show, the nesting of this hierarchy-map can be substantial but can also be sufficiently tracked by the teachings provided herein (see for example FIG. 11F).

It is again noted that the present technical advantages allow for the detailed authorship description 20g-sc of a new session work product 20g-wp comprising possibly significant amounts of inserted digital content that is not definitively certified or otherwise determinable as being authored by the operator 10, but is otherwise authenticated by the present teachings to be at least generated or “composed by” the operator 10, as then recorded in the corresponding work product certificate 36g-wc. Again, it is the on-going determination, tracking, and recording of “chunks” (FIG. 11E, 11F) of inserted verses created digital content forming a novel “content authorship schema” 20g-sc describing the content authorship structure of a work product 20g-wp that uniquely enables during the insertion of a prior work product 20g-pwp the determination of any content that is prior-authored by the operator 10, all as a careful consideration of the teachings herein will make clear. Still referring to FIG. 11D, and now briefly returning to a discussion of minimal content objects, it is noted that some types of apps 20g-app may process visual information as opposed to or in combination with textual information, where then this visual information for example is represented as either of vector based data or bitmap based data, all as will be well understood by those familiar with 2D drawing and digital picture editing (where 3D data typically comprises descriptions of vectors meeting at vertices each with distinct 3D coordinates). As a careful consideration will show, depending upon the particular app 20g-app, visual minimal content objects can also range in complexity from simple dots on a screen, to lines, curves, and other geometric shapes. However, and once again, when inserting content into an app 20g-app that is for example capable of receiving any of minimal visual content objects, it is most advantageous to the operator 10 that the inserted objects are “complex” or non-minimal, thus saving the operator effort in constructing such an inserted object. This same principle for understanding the differentiation of inserted digital content comprising two or more minimal content objects versus created digital content comprising one minimal content object is generalizable across the various types of digital content processed by the virtual limitless number of apps 20g-app available to the operator 10 for composing a session work product. Across this same virtual limitless number of apps 20g-app, the even more definitive determination of a received data path/channel/source of content also remains equally applicable and useful for at least clearly differentiation pre-existing-inserted versus real-time-created digital content.

Regarding (b) above, therefor that created content may not actually be operator 10 content, it is instructive to consider that all digital content being received by app 20g-app via digital input paths available to operator 10 (e.g., using user interface apparatus such as a keyboard, mouse, digital pen, microphone, etc.) must necessarily be information also coming through the mind 28-5 of the operator 10, where then this mindful generation necessarily determines the operator 10's behaviors that are transduced by the particular digital input path apparatus, all as a careful consideration will show. What can also be understood and is depicted in the present figure, operator 10 may be providing original thoughts for digital content or may simply be transducing other digital content such as digital display information 28-6, printed information 28-7, and other visible information 28-8. It is useful to first consider digital display information 28-6 that itself has a known and determinable to app 20g-app digital content source 28 (such as web content 28-1. AI generated content 28-2, stored documents 28-3, or otherwise data coming from any of connected devices 28-4,) where this determination may be directly assessed by the app 20g-app that is providing the displayed information 28-6 or in combination of the app 20g-app in communication with the operating system of system 20-c in the situations where the displayed information is being output by some other app that is not 20g-app currently accepting input from the operator 10 for composing session work product 20g-wp, all as will be understood by those familiar with computers and software applications.

What is also important to see is that either working entirely with its own available information or working for example in combination with the operating system of system 20-c, app 20g-app is able to determine two critical types of information including: 1) what digital content is currently being displayed (and therefore can possibly be viewed by operator 10) on each digital display connected to and being controlled by the system 20-c (such as the depicted laptop computer 20-c screen or a connected “external” display 20-c-cd,) and 2) where such digital content is spatially located with respect to the operator 10 (for example by at least in part using information determined by camera 20-c-cam to spatially determine and calibrate the relative locations of any displays 28-6, any input apparatus (such as a keyboard and mouse,) and the operator 10. Based upon knowing what is the currently displayed content (1), and where is this content being displayed (2), there are many well-known and existing marketplace applications that provide for what is generally referred to as “eye gaze” tracking, and as will be understood by those familiar with eye gaze tracking it is possible to determine at any given moment just before or during operator 10 data input (e.g., via a keyboard as text or a microphone as speech-to-text,) substantially which of any one or more displays 28-6 the operator is “gazing” at and then further also where spatially upon this display is the operator 10 gazing, and thus at what portion of the currently displayed digital content (1) is the operator 10 “looking at.”

By using at least in part any of well-known gaze tracking to determine which of any one or more displays 28-6 are being “gazed-upon” by the operator 10, substantially just before or during the input of minimal content objects, it is possible to determine the “entirety” of displayed digital content possibly being viewed by the operator 10 (thus any content currently being displayed upon the determined operator-gazed-upon display).

Thus, although it is further possible (and available in the marketplace) and somewhat desirable to also determine using gaze tracking what particular “sub-location” of a given gazed-upon display 28-6 is currently being “looked at” by the operator 10, thus translatable to what sub-portion of the entire possible digital content is currently being viewed for stimulating operator 10's mental thoughts 28-5, this is not limiting, as by simply knowing which of any one or more system 20-c controlled displays 28-6 are currently being viewed or where just prior viewed by the operator 10, along with what entirety of digital content being provided by the viewed display 28-6, it is then possible to then compare any current operator 10 inputs to this known provided entirety, where sufficient matching is indicative of an insertion of displayed information 28-6 as opposed to creation of new minimal objects as created by the operator 10 own authorship.

It is also noted, that by making such a correlation between (i) the currently detected spatial eye glance of the operator 10 with respect then to (ii) the head location and orientation of the operator 10 (also available via image processing of the same images captured by a system camera 20-c-cam used for determining eye gaze,) and then to further determine (iii) the current minimal content objects being input by the operator 10 matching to some sub-portion of the known entirety of display content, it is then possible to first use (iii) to determine (iv) an approximate spatial location relative to the display that must be the location of the gazed upon/being entered content (iii), and then to further correlate (iii) to the combination of (i) and (ii), such that the display can be considered as “calibrated” to the operator 10's head location, orientation, and direction of eye gaze such that future determination of (i) and (ii) can be predictive of known (iv) and therefore (iii), all as a careful consideration will show.

Still referring to FIG. 11D, as a careful consideration will then further show, even without a system connected camera 20-c-cam, and therefore any possible eye gaze or head location and orientation information, and even for example considering a system 20-c currently displaying many different concurrent sources of digital content across a multiplicity of displays 28-6 being currently controlled by the system 20-c, the totality of these many different concurrent sources and their particular currently display digital content is limited and well-determinable by the app 20g-app alone or in combination with for example the operating system of system 20-c, such that simply by continuously comparing the inflowing current minimal objects being received by the app 20g-app from the operator 10 through a known user interface (e.g. a keyboard, mouse, digital pen, finger touch, microphone, etc.) to the totality of digital content known to be concurrently displayed, it is then possible to match the input content with one or more displayed content, whereupon consideration of a sufficient number of sequentially input minimal objects is then resolvable into being either definitively matching a significant portion of currently displayed digital content (thus also indicating the known source of the displayed content for use as source, reference, and authorship information 20g-sc) or otherwise not matching any significant portion of displayed digital content and thus indicative of new digital content be generated solely by the operator 10's authorship. As a careful consideration will show, this method of understanding what digital content is concurrently displayed by the system 20-c while the operator 10 is inputting minimal content objects into system 20-c via a user interface, is applicable across the wide variety of types of digital content, most especially including text, symbols, 2D-3D drawings, and digital images.

Conversely, by monitoring the current series of minimal content objects currently being input by the operator 10, app 20g-app and/or work product authenticator 36g may to a high degree of reliability determine that any significant portion of such input operator 10 content is either being “copied” from visually displayed content from known sources or is not being likely copied. As a careful consideration will also show, simply “not being likely copied” from system 20-c displayed digital content such as 28-6-1 or 28-6-2 is not itself fully determinative that the series of minimal content objects being received through the system 20-c user interface into the app 20g-app are of the operator's authorship since it is also possible that the operator 10 is alternatively viewing and “copying from” any of printed information 28-7 or other visible information 28-8 (including a display not being controlled by the system 20-c, such as a second system 20-c being concurrently used by the operator 10 to obtain digital content perhaps for the express reason of surreptitiously copying and presenting as the operator 10's own original content). (It was also prior noted and discussed that beyond visually presented information that can be concurrently presented to the operator 10 during the input of minimal content objects thus available for “copying,” it is also possible for example to present digital content (at least words) audibly to the operator 10, thus where then operator 10 is essentially having content dictated to them from then some other source, with again solutions for detecting this audible-based copying were prior discussed.)

It was also prior mentioned, and is herein stressed, that the preferred system uses a camera such as 20-c-cam or more preferably the camera accessible to the honest broker such as on a primary device 1-1 smartphone positioned to “privately monitor” the operator 10 during the session of operation that is creating or modifying the current session work product 20g-wp, wherein the use of such a camera then preferably supports head location and orientation tracking, eye gaze tracking, and even hand and finger movement tracking. What can then be seen is that using such additional and readily available and well-known technology and resulting information, it is possible to determine at least in many case that the operator 10's just prior or current eye gaze (and associated head location and orientation) are indicative of a concentration that is “away from” the main system 20-c display currently being controlled by the app 20g-app for providing the user interface for entering minimal content objects. Those familiar with typical operator behavior will understand that in most digital content creation situations, an operator 10 that is creating the current minimal content objects from their mind is then also looking or “gazing upon” the system 20-c display that is providing the app 20g-app user interface, and thus tracking eye gaze and/or head location and orientation is largely sufficient for indicating originally authored operator 10 digital content. Hence, when the operator is determined to be substantially looking at the app's 20g-app user interface while also substantially simultaneously entering minimal content objects via any of the system 20-c available input apparatus, it is likely that the series of minimal content objects being currently entered are originally authored operator 10 digital content unless it can be separately determined through analysis that some sufficient series of input minimal content objects sufficiently correlates with any of known and currently displayed digital content (all as prior discussed).

However, it is further noted that it is possible for the operator 10 to move their eye gaze and or head location and orientation back-and-forth between some external digital content source such as 28-7 and 28-8 whose current entirety of digitally presented content is unknown to the app 20g-app, such that when turned away and not looking at the current app 20g-app (which is detectable using eye gaze and head tracking) the operator may be “learning” or mentally copying some next number of minimal content objects, whereafter learning such objects the operator 10 returns their gaze/head orientation to the app 20g-app user interface in order to enter such learned (“copied”) information. There are also many people who are trained to continually gaze at some other source of information such as 28-7 printed material while then also continuing for example to type this 28-7 gazed upon digital into a keyboard being used by the app 20g-app user interface, where then this aforementioned “back-and-forth” eye gaze and head movement is substantially non-existent, but still where the operator 10's current “looking away” while at the same time entering digital content (typically and in practice only an issue regarding keyboard input) is measurable and usable for indicating at least that a series of digital content is “possibly copied.”

Still referring to FIG. 10D, and now also to prior teachings herein regarding the “input nature” of organic digital content, it is further emphasized that in the prior mentioned situation where an operator is copying at least textual information from a printed source 28-7 while not turning their gaze back-and-forth in a determinable manner (thus being more strongly indicative of copying) but rather keeps their gaze substantially fixed upon the printed material 28-7 (or at least not gazing upon the app 20g-app user interface,) thus being somewhat indicative of copying, the present invention still yet teaches the determination of the “being edited” nature as strongly indicative of copying or not copying at least textual digital content.

Specifically, if an operator 10 is in fact copying digital content (text or otherwise) from an external source such as 28-7 or 28-8 for which the app 20g-app is not currently aware of the digital content comprising the visible presentation of 28-7 or 28-8 for “continuous input comparison” as previously discussed, what a careful consideration will show is that such true copying necessarily has the benefit of providing to the operator 10 already “fully edited” or properly composed digital content, thus greatly limiting the “needs to be edited” nature of the currently input digital content. For example, in an extreme case, if a talented operator typed in even a single full page (roughly 500 words) of digital content while solely or primary only looking at a printed material 28-7, without making a single typing mistake, then this resulting series of minimal content objects entered via a valid user interface (such as a keyboard) might otherwise appear to be from the operator's own creative thoughts but is determinable as “likely copied” because of the known unlikelihood of such “perfectly composed” input.

Further concerning the measurable aspects of input nature being taught and discussed herein for the substantiation of “organically authored” work product/digital content, in addition to such content typically comprising intermittent errors causing pausing, position reverting, mouse movements, deletions, insertions, as well as other measurable characteristics of data input entry by an app 26g-app and/or work product authenticator 36g, there are also measurable characteristics of minimal content object entry speed (e.g., the rate of “characters entered per second”) as well as entry cadence (e.g., the variation in the duration between successive entry of each minimal content object,) where as a careful consideration will show, both an increased rate of entry as well as a more regular cadence are characteristics determinable as “likely copied” in that traditional human thought is not just more “error prone” (thus prone to “being edited”) but also is entered at longer rates with a more varied cadence. Before re-discussing how the use of the honest broker operating on a primary device 1 serves as an additional means for verifying the organic nature of input, the present invention also teaches that by using an honest broker/primary app 1 to maintain private information, or otherwise allowing the app 20g-app and/or authenticator 36g to determine and maintain either of private or non-private operator 10 input nature baselines and normals (see work product characteristics history 28g-ch in FIG. 11E,) these determined and stored input nature baselines and normals then serve as data for comparison with characteristics of current data input into system 20-C.

For example, any one of or any combination of the honest broker application executing in private on primary device 1, the app 20g-app, and/or the work product authenticator 36g, or otherwise some application in communication with any of these preferred applications, may determine and store as data associated with operator 10 “input nature characteristics” with regard to, but not limited to the following “content nature characteristics” including (1) specific types of digital content (like word processor documents, 2D/3D drawings, photo editing, computer programs, music, etc.), (2) specific apps 20g-app for entering types of digital content, (3) various computing systems 20-c or 20-d and their various input apparatus (such as keyboards, mice, digital pens, microphones, etc.), (4) times of day and other environmental/contextual conditions, and (5) the nature of the digital content, for example differentiating a “business” verses non-business work product (such as a term paper, legal contract, or patent application as compared to personal notes, emails, etc.).

Where again the input nature characteristics that are measurable or predictable for each of the possible combinations of the content nature characteristics include (but are not limited to) (1) “complexity of inputs,” (2) “extended duration of time to enter,” (3) “in-part,” (4) “being edited,” (5) “amount of commands” verses “amount of work product digital content,” all of (1)-(5) previously having been discussed especially in relation to FIG. 11B, as well as (6) “other possible interpretations and metrics” themselves including operator 10 baselines and normals for any of rates or entry, cadence of entry, frequency or minimal content objects and their combinations (e.g., certain words, phrases, 2D/3D illustration shapes, music notes, etc.) and many other useful metrics determined regarding the actual operator 10 or a type of operator to which the operator 10 is comparable.

As will be discussed in relation to FIG. 11F, based upon the technical advantage of the present system for determining a “series of minimal content objects” comprising a “created content chunk” that has been determinably received through an operator user interface apparatus (such as a keyboard, mouse, digital pen, finger touch, microphone, etc., and as opposed to be “inserted” from a channel/source) as well as determining an “inserted content chunk” as coming from a determined source with known or unknown original authorship (and not therefore from a user interface apparatus,) it is possible to not only maintain a structure and history of the one or more the “created or inserted content chunks” for explaining the authorship of a composite session work product 20g-wp, but it is further possible to assess each content chunk based upon a determined and objective multiplicity of “content nature characteristics” and “input nature characteristics.” The present invention anticipates applying AI tools to analyze this tracked multiplicity of characteristics for best determining informative patterns as current AI technologies have proven so useful in doing, where also the multiplicity of characteristics for a given document's composite content chunks can also be analyzed in virtually any number of larger contexts, for example across multiple documents of the same type or different content types, and/or from the same or different authors, all as will be clear from a careful consideration of the present teachings and from knowledge of how AI algorithms are best supported.

What is important to see is that the present system not only teaches how to identify the one or more distinct content chunks comprising a session work product 20g-wp (see especially FIG. 11E,) but then how to also characterize each distinct content chunk in terms of its one or more nature characteristics, the combination of teachings of which is necessary for allowing any such rich authorship assessment of composed digital content.

Analysis of individual chunks comprising a modern composition of digital content is necessary for the objective determination of the likelihoods of the composite being fluid degrees of organically versus synthetically authored, matching a certain level of work effort, editing effort, etc., where such information is herein referred to as a content chunk “assessment-tag” determined in relation to any given individual content chunk, and then even to particular combinations of content chunks comprising therefore the entire session work product 20g-wp (the reader is directed to FIGS. 11E and 11F for further discussion).

Still referring to FIG. 11D but also generally to the teachings regarding the interplay between the possible components of the system for determining rich authorship of a composite session work product 20g-wp, these software components include a system 20-c or device 20-d operating system, an app 20g-app for providing the user interface to accept operator 10 behaviors directing the formation or modification of the session work product 20g-wp, a companion honest broker preferably executing on a distinct computing element such as a primary device 1, and a work product authenticator 36g executing on either or both of the system executing the app 20g-app or the system executing the honest broker. At least one advantage to the use of a work product authenticator 36g is that the authenticator can maintain an on-going “operator awareness” across multiple possible concurrently or sequentially executed apps 20g-app, where such “multi-tasking” (thus “multi-apping”) is a typical situation for an operator 10 using a system at least such as 20-c. As a careful consideration will show, at any given time, using the same system 20-c, the same operator 10 might be sequentially operating or even concurrently executing multiple applications 20g-app forming an extremely complex content usage and creation environment. The present invention prefers the use of a single “background authenticator” such as work product authenticator 36g that operators in close cooperation with the operating system of 20-c or 20-d and with the one or more sequentially operated or concurrently executing apps 20g-app, where then this separate authenticator 36g for example at least maintains some on-going (thus “cross-app”/“over the separate functioning of two or more apps”) data comprising any of the operator 10's presence, liveness, mood assessment, and other behaviors such as head location, orientation, eye gaze, lip movements, hand and finger movements, etc. As a careful consideration will show, this cross-app continuity of operator 10 “behavior awareness” is highly useful especially in a traditional multi-tasking computing system.

In addition to the components mentioned in the prior paragraph, the present invention anticipates further adapting traditional user interface content entry apparatus, for example tactile input devices such as a keyboard 20-c-akb (of which there are many types such that a keyboard should not be limited to “ascii character”/“letter-symbol” input only) or a mouse 20-c-ams (and otherwise any hand and finger manipulated apparatus,) as well as other forms of operator behavior-to-data transduction means/“input apparatus.”

Regarding the depicted tactile apparatus 20-c-akb and 20-c-ams, as will be well understood by those familiar with such devices, a mechanical motion induced by the operator 10 is transduced into an encoded electrical signal, the signal of which may originate as analog but typically is ultimately transmitted to the attached system 20-c or 20-d as “digitally encoded data” such as but not limited to the familiar “ASCII characters.”

What is important to see is that there is traditionally a single data path depicted as “path A” for communicating from the input device to the system 20-c or 20-d, where then this data is received by what is traditionally referred to as a “data port” with a “software driver” on the system, for then receiving and transmitting to the currently controlling app 20g-app, all as will be understood by those familiar with computing systems and especially devices and operating systems.

As will be clear from a good understanding of such input apparatus and from a consideration of the nefarious desire to insert into this data path originating at the input apparatus and terminating at the app 20g-app some synthetically created data meant to be detected as operator 10 “created”/“organically caused” data, where then this synthetic data posing as organic data could disrupt the desired interpretation of operator content authorship, it is possible to further adapt any input apparatus to comprise a separate and distinct data path such as “path B” depicted, where then path B directs data for example to the honest broker (and/or work product authenticator 36g) preferably executing on a separate computing device such as a primary device 1 or otherwise to some independent session observer system 20-ios (see also FIG. 10C). As a careful understanding will show, any “hacker” wishing to generate posed data would then further need to infiltrate the minimal hardware comprising the input apparatus, minimal hardware which typically does not comprise any such infiltration path and/or is more easily guarded against any such infiltration means or methods. By transmitting original operator 10 created input data along separate paths A and B to separate computing systems, it would then be necessary for any such hacker to infiltrate both/all receiving systems (such as system 20-c or 20-d on path A, or primary device 1 or independent session observer system 20-ios on path B) in an attempt to defeat select features of the present teachings, thus the further adapted “dual-path” input apparatus as taught herein provide further system integrity for the determining of digital content authorship.

This understanding will be especially clear as it is also recognized that the path A receiving system typically executing the app 20g-app may also provide confirmation of received path A input data to the separated path B receiving system such that the path B receiving system has a copy of the original input apparatus data from path A for comparison to the data that has also been received on path B.

Referring next to FIG. 11E, there are shown the mid-level components (some functions, some data) comprising the preferred work product authenticator 36g operating in combination and coordination with one or more concurrently executing work product apps 20g-wp each creating corresponding session work products 20g-wp. As prior stated (see FIG. 11D,) the present system also teaches the creation of session work product certificates 36g-wc, authorship view and characterizations 20g-sc, and original references and prompts 20g-rc, all corresponding to a session work product 20g-wp. From a software deployment perspective, each of work product 20g-wp, certificate 36g-wc, characterizations 20g-sc, and references 20g-rc are distinct digital “artifacts.” The remaining depicted boxes of FIG. 11E represent functions of executable software that may be comprised within a single program (such as a work product authenticator 36g executing on the same system 20-c or device 20-d as the app 20g-app) or distributed over multiple concurrently executing programs (such as a further adapted work product app 20g-app in combination with other functions encapsulated into a work product authenticator 36g and/or an “honest broker” for measuring operator behaviors,) where some programs are executing on system 20-c or device 20-d and whereas others are executing on other systems such as but not limited to a primary device 1 or an independent session observer system 20-ios. Thus, the present FIG. 11E should be considered as teaching a preferred set of technical functions and data combining to provide the novel technology described herein for enabling the benefits of “digital content labeling,” rather than limiting the preferred deployment as one or more executable programs/system “artifacts.”

As will be clear from the consideration of multiple apps 20g-app being available in the marketplace for use in creating any particular given type of digital content, such as multiple different word processing applications, or multiple different “photographic” image editors or 2D/3D vector image creators, etc., since all apps in a category for generating similar digital “minimal content objects” are typically adapted to receive the same common inputs (such as from a keyboard, mouse, microphone, graphics tablet, etc.) but may still internally process such common inputs in different ways while still forming and outputting commonly exchangeable minimal content objects (such as “letters,” bitmap images, rectangles, etc.,) it is desirable to maintain standard codes 36g-sc for representing actionable inputs that are subsequently “universally analyzed” for the identification of “content chunks.” For example, Microsoft supplies the well-known word processor called “Word” while Apple offers a different word processor known as “Pages.” Generally speaking, both are examples of work product apps 20g-app that create at least text based session work products that may also be composed with pictures, spreadsheets, or similar data tables, 2D images or primitive shapes, etc. Both produce session work products 20g-wp that may be interpreted by other apps 20g-app (including each interpreting the other's output 20g-wp or at least some “converted” version of this output 20g-wp). Both Word and Pages are designed to accept operator 10 input from similar apparats such as keyboards, mice, microphones, graphics tablets, etc.

However, as will also be clear, each of the various available marketplace applications offer different user interfaces with many similar but at least some different commands, and where the similar UI commands may be invoked by different inputs (e.g., different keyboard shortcuts or different UI button presses or sequences of selections). Given these considerations, it will also be clear that the internal representation of inputs received by each of these representative programs may be and are likely in at least some cases to be different (e.g., the “copy command” or a “character” such as a particular letter or punctuation, or a geometric graphic like a rectangle) where one of the goals of continuous input segmentation function 20g-is is to translate program-unique representations of internal commands and data into a universal/“standard” set of codes which may then be processed generally without pre-knowledge of the originating program and without concern for the internal data representations used in any given market application 20g-app, all as well be well understood by those familiar with software applications.

Still referring to FIG. 11E, what is desirable is that continuous input segmentation function 20g-is preferably executing within a specific app 20g-app in order to translate specific internal app 20g-app inputs (minimal commands or data) at their time of occurrence into standardized/“generalized” “minimal content objects” output to a generalized work product authenticator function 36g-a. Thus, the outputs of function 20g-is are abstracted from the specifics of a given specific marketplace app 20g-app (such as Word, Pages, Illustrator, etc.) and can themselves be interpreted and processed by a common or what is often referred to as a “generalized” algorithm that is otherwise unaware of the details of each specific programs internal representations of commands and data. Standardized representation codes 36g-sc for types of commands or data input and processed by a given type of app 20g-app (like a word processor) or at least for a given type of digital content (like a page of text) are now able to be commonly processed by a continuous input-behavior correlation and authentication function 36g-3a without regard or need to understand internal information related to any specific work product app 20g-app, all as will be well understood by those familiar with software systems and programming.

It is noted that a typical market application (like Word or Pages) processes any such operator 10 inputs (e.g., coming from keyboards, a mouse, graphics tablet, microphone, etc.) in order to incrementally create or edit the session work product 20g-wp, where any such processing is performed “input-by-input” typically sporadically over time, and where each of these incremental operator-behavior-caused inputs (commands or data) are “lost” as representations of individual pieces of information comprising characteristics such as “at this time, this input was processed” in favor of the representations of the resulting effects on the building and changing session work product 20g-wp. Thus, after a typical session of operation of a traditional application, it is possible to review the resulting work product 20g-wp but not the individually received and time correlated, step-by-step, input-by-input, commands and data received via input apparatus in direct response to operator behaviors, thus any correlation between operator behaviors and these various inputs are themselves lost and cannot be characterized, the characterizations of which are taught herein to comprise the “input nature” of the session work product 20g-wp apart from the resulting final work product 20g-wp itself, where this input nature is then highly useful and beneficial for determining at least content authorship and organic vs. synthetic content composition.

Apart from maintaining all individual app 20g-app inputs (commands or data) as standardized minimal content objects for output to input-behavior correlator 36g-3a and useful for revealing the input nature of a session work product 20g-wp, with respect to “command” inputs translated into standard minimal content objects there are typically many various and possible “keyboard shortcuts,” “menu options,” “voice inputs,” etc., for allowing an operator using an app 20g-app to indicate a given desired command in any of a multiplicity of ways, even though a multiplicity of different input apparatus, where for the purposes and efficient utility of function 36g-3a any such “multiplicity of ways” always results in the same standard minimal content object for representing the command. Thus an app 20g-wp might have 6 different ways for an operator 10 to indicate via an input apparatus that a “copy” command is to be executed, where for each of these 6 different ways the continuous input segmentation function 20g-is generates the same standard codification for minimal content object representing a “copy command.” (Again, the present teachings allow for such a “copy command” to have a single universal/standard codification across virtually all applications 20g-app, regardless of the digital content type (since in this case a “copy command” is broadly applicable across all manner of digital content). Other broadly used command notions include “select,” “paste,” “insert,” “delete,” “open,” etc., all of which benefit from the present teachings to not only encode as individual minimal objects for subsequent analysis revealing input nature, but also to encode using a standard code supporting a generalized input-behavior correlation and authentication function 36g-3a, and therefore also a generalized work product authenticator 36g, where the many advantages of such generalization will be well understood by those familiar with software development and maintenance.

In recap, the careful reader will note at least two distinct advantages to the standardization of app inputs prior to attempting to universally segment various different digital content across any and all possible apps. First, each of different apps 20g-app will provide a “universally recognized” or standard indication of each various typical and often common commands and/or data (such as “paste,” “bold,” “group,” “change line size,” “letter X,” “rectangle,” “circle,” etc.) made available at least across various apps for creating similar digital content. Second, even within a single application 20g-app, it is not untypical to provide the operator 10 with multiple means for indicating through the app UI that a given command should be executed, such as “copying” data from the app to the clipboard and then “pasting” data from the clipboard into the app.

Thus, for a single given app 20g-app that may present hundreds of possible commands through the use of a UI, where at least some are “keyboard shortcuts” indicated via a combination of keys, and others are “UI components” such as “clickable” buttons or sliders, it is possible to reduce this multiplicity of possible commands to a much simpler and useful limited set of standard commands relevant to the determination of authorship and input nature. The present invention also anticipates that for example a word processor app uses a single codification such as “text formatting” to represent any and all possible text formatting such as bolding, italicizing, changing a font size, etc., since as a careful consideration will show, such any formatting changes are typically irrelevant to a determination of authorship and are commonly indicative of input nature.

In other types of “layout design” application, where for example of digital artist is creating web or print copy and design and layout are issues of authorship and not just input nature, it is possible then to for example use different standard codes for the different types of “layout design formatting,” where the actual standard code sets to use can be dictated by the type of digital content or digital content processing to be authorized, which may also be a setting in any use certificate 35-uc and/or supervisor certificate 24-sc).

Still referring to FIG. 11E and the purpose and use of standard codes 36g-sc by any app 20g-app, a careful consideration will also show that for a given digital content type, a significant number of minimal content objects that are content (data) versus commands can be generalized into a single code for continuous input segmentation 20g-is. For example, in a word processing app 20g-app accepting an on-going multiplicity keystrokes (each being a single minimal content object,) rather than passing an indication of each distinct received and processed keystroke (such as an “L”, “V”, “+” or “;”, etc.) it is preferable to generalize such keystrokes into a single standard code for “text” or perhaps a single code for “a letter” versus a single code for “punctuation,” etc. What is important to see is that by using standard codes for various input being processed as data by any given app 20g-app for creating various types of content, while a burden is placed on the app provider to add additional programming or otherwise integrate function 20g-is to translate all possible inputs into the various standard codes 36g-sc, a significant advantage is created by supporting a universal work product authenticator 36g predicated on the processing of standard codes rather than needing to understand all possible input representations coming from all possible programs.

Still referring to FIG. 11E, and now discussing continuous behavior monitoring function 36g-2, the reader is reminded that operator 10 behaviors ranging from simple “presence” (i.e., “the operator is in view”) to more complex “liveness” (i.e., “the operator is in view and moving”,) all the way to sophisticated determinations of detailed behaviors such as the current operator 10 “eye gaze,” “hand or finger movements,” “lip movements,” “facial expressions,” etc., can all be determined using existing and well-known technology. The present application teaches the use of any such available (or future) operator 10 behavior sensing and tracking technology further adapted to use standard codes for inputs and behaviors 36g-sc for accomplishing the novel standardization of the codification of such operator 10 behaviors. Traditional behavior monitoring will be understood to be more “continuous” and less “segmented,” where segmentation into individual “minimal behavior objects” (such as a significant change in head posture, eye gaze, finger movement, etc.) is preferably predicated on any one of or any combination of a) exceeding a delta change condition with respect to a current or prior pose/position of behavior (e.g., the operator is looking straight ahead and then turns/rotates their head more than a “delta change” of “x degrees” to the right or left,) b) the determination of significant app 20g-app or input apparatus “engagement” (e.g., the finger of the operator is determined to strike a keyboard key surface or the hand of the operator is determined to be holding the mouse and then moves exceeding a delta change,) or c) triggered by a received signal from the continuous input monitoring function 36g-3a (e.g., where function 36g-3a receives a substantially real-time input minimal object from segmentation function 20g-is and then at least in part uses the timing of this receipt to initiate the transmission of a “capture current operator pose” or similar signal to continuous behavior monitoring function 36g-2.

Still referring to FIG. 11E, as a careful consideration will show especially to those familiar with the art of image processing in general, and body motion tracking in particular, that it is optimally necessary to calibrate the tracking system using some form of preferably 3D coordinate system, either absolute or relative such as input apparatus special coordinates system 20-scs. It is not the purpose of the present invention to teach a preferred means and method for the calibration of for example systems using a fixed camera to image a person for translation into 3D body poses as considerable teaching is available in the art and many approaches are consider sufficient for the present purposes. During calibration, function 20-scs preferably determines a substantially fixed set of spatial relationships (again, in any of an absolute or relative coordinate system, all as is well-known) between the one or more tracking cameras and preferably all of the various system apparatus available for use by the operator 10, especially those used for accepting operator 10 inputs such as a keyboard and mouse, as well as apparatus for providing information to the operator 10 such as one or more system displays (28-6).

Once these preferably 3D spatial relationships have been determined, it is then possible to track the relative and “free” motion of the operator 10's body (e.g., head, eyes, lips, torso, hands, fingers, etc.) as the operator 10 physically interacts with the various input apparatus, even when this interaction does not involve physical contact (e.g., interpreting hand gestures and lip movements). What is desirable is that continuous behavior monitoring function 36g-2, based upon some “segmentation trigger” (a), (b), or (c) (see the preceding paragraph) provides minimal behavior objects at these times of triggering to continuous input-behavior correlation and authentication function 36g-3a, where each provided minimal behavior object preferably comprises a description sufficient to identify the “body object” (e.g., head, eye, hand, finger, etc.), a current or change in body object “pose,” as well as an indication of which system apparatus (keyboard, key on keyboard, mouse, mouse button, digital pen, microphone) is the suspected or confirmed transducer of the body object pose (assuming a moment in time representing possible motion for transducing).

Still referring to FIG. 11E and now to the continuous input-behavior correlation and authentication function 36g-3a, it is first noted that the “operator authentication” has its own separate value for assuring that a given session of work (resulting in a new of modified session work product 20g-wp) was conducted based upon the behavioral inputs of a given operator 10, where the identity of the operator 10 is preferably only known to the “authenticating app” such as an honest broker app executing on the primary device 1 being used in combination with the app 20g-app and work product authenticator 36g (see also especially FIG. 11B).

Minimal assurance is established for example by the primary device 1-1 being temporarily mounted to the system 20-c (or being system 20-d) and consistently viewing the operator 10 during the session of operation thereby capturing preferably a multiplicity of images of the operator 10 while the operator 10 is positioned at the system 20-c or device 20-d for providing system and app inputs to app 20g-app, all as prior discussed.

Several variations have been described herein for using each currently captured image of the operator 10 to for example compare with a prior confirmed image or with a prior registered image (see also FIGS. 3A, 10A, 10B, and 10C) to authenticate the operator.

As also prior discussed, the proximity of an operator 10 wearing a primary device 1 such as smartwatch 1-2 can also provide assurance that the operator is “present” at the system 20-c or device 20-d during the session of operation (even without camera based authentication) and thus also at least the author of the composition of the created and/or modified session work product 20g-wp, although not yet confirming which parts of this authored composition are “organic” representing the authors own efforts or “synthetic” representing the efforts of some algorithm such as a generative AI algorithm, or even “inserted” from other organic or synthetic sources, all of which is possible to determine using the present teachings as prior discussed and also as now being described in relation to the various functions of work product authenticator 36g. It is also again noted that the authenticated identity of operator 10 may be shared by the honest broker primary device 1 with the work product authenticator 36g for encoding with the work product certificate 36g-wc, or replaced by an “anonymous operator ID,” or simply an indication that the operator is known and thus the composition is “organic” for use in the certificate 36g-wc or similar artifact. And finally the reader is reminded that the level of authentication and preferred means of authentication, as well as the sharing of various levels of the operator 10 identity are preferably specified in either of the use certificate 35-uc or supervisor certificate 24-sc (see especially FIGS. 10A, 10B, 11A, and 11B).

Beyond authentication of the current operator 10 of an asset (such as system 20-c or device 20-d) currently being used in a session of operation resulting in a session work product 20g-wp and corresponding work product certificate 36g-wc, where the authentication again provides a form of “proof-of-authorship” at least of the composition of work product 20g-wp if not then also the component parts of 20g-wp, the function 36g-3a also and importantly determines correlations between the on-going and presumably sporadic inputs being processed by the work product app 20g-app and then output via input segmentor 20g-is as current minimal content objects (commands or data) in comparison to the presumably sporadic body behaviors being processed by behavior monitor 36g-2 and then output as current minimal behavior objects. As a careful reading of the present application will show, there are various ways of determining input object verses behavior object correlations amounting to various possible “levels of assurance.” In at least one method, function 36g-3a determines such correlations by comparing the timing of the received objects, where a careful consideration will show that keyboard inputs forming minimal content objects (either commands or data) should align temporally with hand movements at a “gross” level and finger movements as a “detailed” level. (However and again, even the measured and determined continuous presence (identity) and preferably liveness (motion) of the operator positioned “at” the system 20-c or device 20-d can be sufficient for correlating a series of many minimal content objects extending over a duration of session operation time bounded by only a first and a last minimal behavior object (e.g., that is confirming the presence and possibly also the liveness of the operator 10).

Beyond time correlation, there is also expected to be “input apparatus correlation,” where a minimal content object is described as being received via a specific input apparatus and the corresponding minimal behavior object is described as locating a portion of the operator 10's body (such as their hand) substantially in spatial contact or proximity with the specific input apparatus, or in another example the specific input apparatus is a microphone and the correlated minimal behavior object is operator 10 lip movement at substantially or at least roughly the same session time. (It is also noted that the use of voice recognition for determining content inputs can also be used to determine and authenticate the operator 10's voice patterns, often called “voice recognition,” and that otherwise the behavior monitor 36g-2 may comprise any combination of a camera for determining correlated operator 10 lip movement or a microphone for determining correlated operator 10 voice inputs.)

Still referring to FIG. 11E, function 36g-3a preferably modifies any of the processed minimal content objects to for example indicate a degree of correlation with any zero or more minimal behavior objects, where the degree of correlation ranges for example from “no correlating behavior object found” to “a sufficiently correlating behavior object found” (like a body presence behavior object) to “multiple highly correlating behavior objects found” (like body presence, liveness, hand, and finger movement behavior objects). What is desirable and preferred is that function 36g-3a first receives and second attempts to correlate all received minimal content objects with zero or more received minimal behavior objects (if such objects are being determined by a monitor 36g-2). After performing such content-to-behavior correlation, where again “content” can be a command or data, function 36g-3a then also at least provides the incoming processed stream of minimal content objects to work product characterization and chunking function 36g-4a and preferably also provides the incoming processed stream of minimal behavior objects to detect mind-copying function 36g-4b (via function 36g-4a).

Work product characterization and chunking function 36g-4a is primarily responsible for receiving the stream of standardized and correlated minimal content objects from function 36g-3a (possibly and typically originating from a multiplicity of concurrently operating and different active apps 20g-app (where each minimal content object preferably comprises data identifying its originating app 20g-app) and for processing this stream, preferably app-chunk-by-app-chunk into one or more chunk-metrics regarding the “input nature” of the chunk (where input nature has been prior discussed herein, see especially FIG. 11B and sub-process 20-app-3). It is important to see that in a traditional work product app all inputs are processed substantially immediately upon receiving, where this processing results in changes to the state of the application and/or changes to the state of the session work product, and that after processing the originally received command and/or data is no longer represented or otherwise no longer represented as a “distinct” input (e.g., a minimal content object as herein taught). Those familiar with software applications will recognize that there has traditionally been no need or utility for both immediately processing incoming inputs (commands or data) to determine an app-state or work product change and then, additionally, essentially “reformatting” or otherwise translating these same inputs (commands or data) into representative minimal content objects (let alone “standardized” minimal content objects,) where the present invention teaches novel apparatus and methods as well as the needs and uses for no longer “throwing away” such valuable input information.

Function 36g-4a preferably has access to a persistent work product characteristics history 20g-ch that may for example be stored locally with operator 10 only characteristic information or may be stored remotely and comprise valuable session characteristics related to a multiplicity of other operators. Regardless, characteristics history 20g-ch maintains for example the input-nature (various metrics) related any number of different types of digital content (e.g., documents, drawings, spreadsheets, computer programs, etc.) and any number of operator related features (e.g., experience level, entity association, baseline metrics, etc.,) and where function 36g-4a access includes updating history 20g-ch as well as receiving prior information from 20g-ch for use in determining current characteristics and metrics.

Still referring to FIG. 11E and function 36g-4a for characterizing and chunking work product content, it is important to recognize that the operator 10 from the highest perspective is a “content composer” that uses an app 20g-app as a means to compose one or more “chunks” of content (i.e., “content parts”) into a final composition. A content chunk is either (a) “created current content,” or (b) “inserted prior content.” Created current content is defined by any uninterrupted series of one or more operator 10 standard minimal content objects (commands or content) that do not comprise a command request to “insert prior content.” Insertion commands are well-known in the art of applications and include the “paste (content) command,” where “inserting” or “pasting” requires that the inserted or pasted content is “prior content” and thus already pre-existing and typically comprising two or more minimal content objects (typically comprising data and not commands). Hence, the natural transition from created current content (a) into inserted prior content (b) is determinable based upon the occurrence of minimal content object comprising a command with a standardized “insert” code. Inserted. “pre-existing” content is often substantial (e.g., an entire paragraph of words or even pages of words, or a picture) and its “insertion timing” is essentially instantaneous, and thus the insertion command marks a transition from (a) to (b) and the directly back to (a) as the inserted content is always a single complete content chunk. While not typical, it is possible for an operator to specify two or more consecutive insert commands, where then it is preferred to treat each insert command as generating a distinct content chunk (b), i.e., rather than joining all consecutive inserted content chunks (such as b1, b2, b3) that are not interrupted by current created content (a) as a single chunk (b).

The following are provided as example pseudo representations of content chunking: [created current content], {insert command}, [inserted prior content], [created current content], {insert command}, [inserted prior content], {insert command}. [inserted prior content], [created current content] Replacing “insert command” with “ic”, the preceding pseudo representation becomes: [a1], {ic}, [b1], [a2], {ic}, [b2], {ic}, [b3]. [a3] . . .

Removing the insertion command symbols “{ic}” that are not content chunks becomes: [a1], [b1], [a2], [b2], [b3], [a3] . . .

As a careful consideration will show, each created content block a1, a2, a3 . . . etc., represents a series of one or more command or data inputs received and processed by app 20g-app and then output via function 20g-is as a series of one or more corresponding minimal content objects, themselves received and correlated with behaviors by input-behavior correlation function 36g-3a and then output to chunking function 36g-4a for the determination of the blocks a1, a2, a3 . . . etc, based primarily upon detected transitions from creating to inserting content as just discussed. As a careful consideration will also show, “created content” blocks a1, a2, a3 . . . etc. are substantially provable by the teachings herein to have been caused by the actions (behaviors) and therefore the thoughts 28-5 of the operator 10. (What is not yet clear and may be still doubted, is whether these actions caused by operator 10's thoughts 28-5 represent “original” operator 10 thoughts, or otherwise represent non-original operator 10 thoughts and thus a form of “mind-copying” where the operator 10 is first looking at other content that is not of the operator 10's origin/authorship, or at least not the operator 10's “current origin” as it is also possible that the operator 10 is looking at and “mind-copying” their own prior content, see upcoming function for detecting “mind-copying” 36g-3b.)

Likewise, a careful consideration will show that each “inserted content” block b1, b2, b3 . . . etc., represents a single “insert content” command received and processed by app 20g-app and then output via function 20g-is as a corresponding minimal content objects (including the actual inserted content or preferably a reference to the inserted content,) themselves received and correlated with behaviors by input-behavior correlation function 36g-3a and then output to chunking function 36g-4a for the determination of the blocks b1, b2, b3 . . . etc., based primarily upon detected transitions from creating to inserting content as just discussed. As a careful consideration will also show, “inserted content” blocks b1, b2, b3 . . . etc. are substantially provable by the teachings herein to have been caused by the actions (behaviors) and therefore the thoughts of the operator 10. (What is not yet clear and may be still doubted, is whether the inserted content is verifiable as to its source and authorship, and whether or not the authorship is of the operator 10 or not of the operator 10, see upcoming inserted content identification function 36g-4c and determine insertion source function 36g-4d.)

Again, regarding chunking function 36g-4a, what is important to see is that the operator 10 as the composer of the session work product 20g-wp, substantially always provides any of zero or more “insert content” commands or directives to app 20g-app via an authenticatable input in order to at least in part cause app 20g-app to pause any current created content entry and to then insert some pre-existing content into the current state of the work product 20g-wp, thus the transition form created content entry (type “a”) to inserted content entry (type “b”) is determinable by first maintaining a description (herein the “minimal content objects”) of all app 20g-app inputs (commands and data) after initial processing into app and work product state changes, where then upon subsequent analysis these same minimal content objects comprising (preferably standardized) insert commands may be used to differentiate the end of a current “a” or “b” content chunk, the beginning of the now being inserted “b” content chunk, where this now being inserted “b” chunk is also “closed out” and any next data (whether created “a” or inserted “b”) will begin the next sequential chunk.

Still referring to FIG. 11E, upon determining the beginning of any of chunks “a” or “b,” chunking function 36g-4a then preferably determines a unique identifier/codification for the determined just beginning chunk and then communicates this chunk-ID or similar to content marking function 20g-cm (where at the beginning of a new session of operation of app 20g-app for creating or modifying a session work product 20g-wp it is assumed that a new chunk “a” or “b” is about to begin and thus function 36g-4a supplies a first chunk-ID to function 20g-cm substantially at this beginning of session time). Content marking as a general principal is well-known in the art, even the content marking of what is herein referred to as a minimal content object (that is data,) such as a letter of text, a geometric object in a vector drawing, a cell number in a spreadsheet, etc., where as those familiar with various traditional digital content apps such as word processor, drawing packages, and spreadsheets will understand that for example, individual minimal data (letter, geometric object, cell number) can for example be grouped (using a group code/mark) with other minimal data, assigned a formatting attribute, tagged with a comment, etc., all of which are herein recognized as types of content marking.

The present invention teaches a new type of content mark preferably applied to all one or more minimal content objects comprising a determined content chunk. “a” or “b,” where again, these “one or more minimal content objects” to be marked are data, as opposed to commands, and where these minimal data are typically whole (cannot be sub-divided) constituent pieces of a presumed much larger composition of data that is the session work product 20g-wp. This new content chunk mark comprises preferably the minimum of a chunk ID or equivalent, where then this chunk ID is associated with the authorship view and characteristics artifact 20g-sc that comprises additional authorship and characteristics specifications (such as input nature metrics) associated with the chunk ID and therefore the marked data comprising presumably a portion of the session work product 20g-wp. As a careful consideration will show, once marked with what is essentially an originating chunk ID, each minimal content object may then as a result of future operator 10 commands and data entry be any of compositionally moved, transformed, deleted, or otherwise edited, all while maintaining the original chunk ID, thus forming a highly useful, resilient, and permanent content authorship “fingerprint.”

For example, an inserted chunk “b” might comprise pages of “synthetic” words provided by an AI algorithm such as what is currently known in the marketplace as Chat GBT, where then all of the individual letters of the pages of inserted (“synthetic”) words are individually marked with a minimum of a chunk ID. Subsequent to this first insertion and marking, the operator 10 might specify commands for moving portions of this inserted synthetic chunk “b” to then be mixed into other created or inserted chunks, thus beginning a blending of content, where by maintaining the original inserted content (see the discussion related to original references and prompts 20g-rc,) having the authorship view and characterizations 20g-sc with chunk ID related characteristics, and then also allowing for a the permanent association of an original chunk ID with the comprising individual minimal content objects, it is now possible to provide a sophisticated assessment of composed, mutli-sourced blended content compositions of virtually and type of digital content.

Furthermore, as the careful reader will understand, it is possible to have a first inserted chunk (again and e.g., a large text inserted comprising hundreds to thousands of words or a complex vector based image comprising tens to hundreds of geometric vector shapes,) where after insertion each of the member minimal objects are permanently marked with a effectively unique chunk ID, and then a series of new created minimal objects (themselves forming a new created “organic” chunk “a”) are interposed into the prior inserted “synthetic” chunk “b,” thus instantly forming mixed or blended organic-synthetic content where each component minimal object, created or inserted, organic or synthetic, is discernable at least by its chunk ID, and where then valuable session characteristics and metrics regarding the created chunk modifying the inserted chunk are determinable and useful for evaluating what is herein referred to as “editorial effort.” It is noted that each app developer can consider and define its own minimal content objects such as a “letter” verses a “word,” or this can be a setting that is adjustable based upon any of the use certificate 35-uc or supervisor certificate 24-sc. It is further noted that once a content authorship assessment (see upcoming 36g-4g) is determined, preferably using the original chunk IDs distinguishing a potential multiplicity of mixed and blended minimal created and inserted content objects, it is possible to reassign to any portions of the session work product new (e.g., “mixed authorship”) chunk IDs, where these new meta-chunk IDs preferably are additionally added to each minimal content object (thus maintaining the original chunk IDs,) but could also serve to replace the original chunk ID thus making the original ID not permanent.

Still referring to FIG. 11E, those familiar with software apps and architecture will note that many variations of the presently depicted functions, including their communication paths as well as their deployment in any of various possible executable programs including app 20g-app, work product authenticator 36g, honest broker 1, or independent session observer 20-ios, are possible, and as such the present depiction should be considered as exemplary and for the purposes of teaching key processing and data transformations all leading from the traditional app simply outputting a work product to the herein taught app 20g-app outputting an authenticated certification 36g-wc and authorship characterization 20g-sc of and along with a work product 20g-wp. In one example variation from the present depiction, continuous input segmentation function 20g-is alternatively communicates with content marking function 20g-cm when an input command designating an “insert content” request is first processed, where then function 20g-cm assigns its own “next chunk ID” and communicates such information back to function 20g-is for association with the minimal content object that represents the insert content request/command (thus then being eventually provided to characterization function 36g-4a that is no longer required to perform this chunk ID determination for communication to content marking function 20g-cm). What is further noted is that app 20g-app typically has valuable knowledge usable for determining both the type of source of the inserted content, e.g., an API interface, a disc file, the system clipboard, etc., as well as the reference/identification of that actual source (e.g., a web-service name, a filename, etc.,) where then at least this source identification and possibly also reference identification of inserted content, collectively “inserted content identification” data may be provided through any number of communication paths to the work product chunking function 36g-4a and thereby subsequent work product authenticator functions such as 36g-4b and 36g-4c.

Regarding an app 20g-app knowing the identity or specific reference of app-inserted content (as opposed to the type of the source of inserted content, i.e., “coming from the system clipboard,” “coming from a drag-and-drop request,” “coming from a disk file access,” “coming from a web-service API,” etc.) it is often the case in modern computing that an operator 10 is executing multiple concurrent apps 20g-app, e.g., “app 1” and “app 2.” In many such cases the operator 10 might first use the interface of “app 1” to locate specific content (e.g., where “app 1” is a web browser, or where “app 1” uses an API to access a web-service for generative AI, etc.,) and after locating may the select some of this found count for “clipping” or “copying” to the system clipboard, in which case the identity of the reference content as knowable to “app 1” is then traditionally not transferred to the traditional clipboard, thus becoming “lost” information to any subsequent “app 2” that then “pastes” this copied reference information into “app 2”'s current work product 20g-wp. Hence, even though “app 2” can identify that the type of the source, hence the source of the inserted content is the system clipboard. “app 2” cannot otherwise also determined the identity of the originally referenced content that was copied by “app 1” prior to pasting by “app 2,” all as will be well understood by those familiar with traditional application software and systems.

The present application teaches further adapting the traditional system clipboard to provide clipboard monitoring function 28-9m, where function 28-9m preferably further maintains the original “copying” app 20g-app's identity (in this present example, the identity of “app 1”,) the type of source the copying app drew the copied information from (where it is noted that this source may also simply be the “app 1”'s own currently displayed work product 20g-wp as well as other sources already discussed or otherwise well-known in the art,) and the identity of the reference of the copied information (such as “Wikipedia article xyz,” “AI web service Chat GBT4,” “person X that is the authenticated operator 10 of app 1 and originally created the copied information,” “Google Image Search-Getty sample image xyz.” “website xyz, page 999,” “image captured by person NNNN,” etc.).

Still referring to FIG. 11E and function 36g-4a, it is important to see that chunk transitions are determinable by detecting at least any insert data commands received by the current app 20g-app, where then this inserted data must have a known source type to the app 20g-app, but traditionally may not have a known reference identity (e.g., if being inserted from the system clipboard). It is also important to see that an insert of content into a current session work product 20g-wp is a “complete operation” (occurring in perhaps milliseconds of compute time and thus minimal session time duration) as opposed to created content being entered via the app 20g-app's user interface including input apparatus in a minimal-data-object-by-minimal-data-object “uninterrupted” (i.e., by an insert request) manner, and typically occurring over minutes and longer of session time duration (even if still only milliseconds of aggregate compute time). Thus, the careful reader will see that an insert content request has a determinable “beginning time” substantially equivalent to the time of receipt of the operator 10's inputted command, and that at this point a new chunk ID can be generated (either internally and alternatively by the app 20g-app as prior discussed, or preferably by function 36g-4a) and provided to the content marking function 20g-cm. The content marking function then substantially and immediately has all of the inserted content available for marking. At substantially this same “beginning time,” any insert command has a commensurate “ending time,” where then this ending time also timewise delineates the ending of the work product “b” chunk and the establishing the “beginning time” of the next work product chunk (possibly “a” or “b”). While this next work product chunk could then be another amount of inserted content (thus back-to-back “b” chunks,) it is more typical that the next chunk beginning is a created chunk “a” comprising a sequence of operator 10 inputs (commands or data).

Regardless, it is also important to see that certain functions are best triggered at the begging time as opposed to the ending time of a determined chunk “a” or “b,” even knowing that in practice the beginning and ending times of an inserted chunk “b” are substantially equivalent. This understanding helps the reader to recognize the purposeful and preferred sequence of functions being presently depicted. First, detect that transition from a chunk “a” to “b,” from “b1” to “b2,” or from “b” to “a”. Second, assign a chunk ID and mark all inserted chunk “b” minimal content objects “immediately” or mark all being created chunk “a” minimal content objects as they are being created/accepted as UI inputs over some duration of session time. Third, upon a subsequent detected chunk transition. “close” the current chunk by performing the determine/identify exchanged content function 36g-4b. The overall purpose of function 36g-4b is to determine the original “authorship” of the current chunk now being “closed,” This authorship, as a careful consideration will show, can be determined as the operator 10 (for created chunks “a”) or some other known or unknown author for inserted chunks “b”, where, as prior discussed the author of an inserted chunk “b” may be “organic” (a person, either identified or not identified but still known to be a person and not an algorithm) or may be “synthetic” (e.g., an AI or other type of algorithm or computing function). Also as prior discussed, even the created chunk “a” may be of some other authorship than the operator 10, if the operator 10 is doing what is herein referred to as “mind-copying,” hence looking at a source of visually presented content and then using that as the prompt to enter their created content, and in the simplest example reading text off a book 28-7, from display screen 28-6, or from some other visible information 28-8, and then substantially typing into a keyboard or speaking into a microphone the text that is being currently read. Hence and again, the purpose of determine/identify exchanged content function 36g-4b includes determining the authorship of content being created or inserted into the current session work product 20g-wp via the app 20g-app.

Still referring to FIG. 11E, function 36g-4b is depicted as comprising three sub-functions, namely detect “mind-copying” 36g-4b1, determine inserted source/reference 36g-4b2, and determine exported prompts 36g-4b3. Regarding detecting mind-copying function 36g-4b1, it is important to see that it is possible to objectively determine that a current content chunk is of type “a,” and thus being “created” by the operator, simply by the app 20g-app internally receiving one or more minimal content objects (commands or data) via a connected input apparatus (such as a keyboard, mouse, microphone, digital tablet, etc.) Hence, the initial assumption is that created content is being authored by the operator 10. The present invention teaches several ways for verifying this assumption including, 1) detecting operator 10 behaviors such as eye gaze that are indicative of copying, 2) detecting a correspondence between known displayed content and a significant multiplicity of minimal content objects comprising the created chunk, and 3) detecting a chunk input nature that is indicative of copying rather than original thought, where then it is also shown herein that the combination of (1), (2), and (3) can provide strong evidence of created chunk operator authorship, or other source authorship, even in some cases such as (2) identifying the other authorship.

As a careful reading will show, each created minimal content object (command or data) having been processed by continuous input-behavior function 36g-3a may comprise related “eye gaze” information (see FIG. 11D for a portrayal of exemplary possible operator 10 eye gaze content localities,) where this optional “may comprise” depends upon the type of continuous behavior monitoring 36g-2 being implemented by a system following the teachings provided herein. Assuming that eye gaze is being tracked, a well-known technology, detect mind-copying function 36g-4b1 may look at the current eye gaze content location per each created minimal content object, were an eye gaze for example focused on the screen presenting the user interface of the app 20g-app and immediately displaying either the input minimal content object or some effect based upon the object, is a traditional indicator that the content is of the operator 10's own authorship.

However, as the careful reader will also note, continuous behavior monitoring 36g-2 may additionally generate minimal behavior objects for processing by correlation function 36g-3a that are not (c) triggered to substantially correspond to the session time when any given minimal content object is being received and processed, but rather are (a) determined based upon exceeding a delta change condition with respect to a current or prior determined pose. Thus, detect mind-copying function 36g-4b1 may determine that the operator 10 is consistently and substantially “looking away” from the user interface of the app 20g-app during some duration of session time when created content is being received and processed, therefore being more indicative of copying.

To support the assessment of potential mind-copying, the present invention teaches a new function similar to clipboard monitoring 28-9m and herein referred to as displayed content monitoring 28-6m. The careful reader will understand that any of one or more system displays such as 28-6-1 or 28-6-2 (see FIG. 11D) act to present information for potential copying by the operator 10, and in that sense are also a form of a “mind-clipboard,” Displayed content monitoring 28-6m preferably maintains (or has access to) any and all information being presented to the operator 10 via a visual output, such as a system display, or even an audio output, such as speakers or headphones. Along with this displayed (or audio) information, function 28-6m preferably also monitors or has means for determining any of the identity of the app 20g-app providing the presented information and more preferred the source type and reference identity of the presented information (terms already defined above). It is preferred that sub-function 36g-4b1, upon notification by function 36g-4a of the termination/“ending” of a chunk “a” of created content, then determines the minimal content objects comprising this created chunk “a,” where this determination can for example be accomplished by accessing the session work product 20g-wp for all content marked with the currently terminated chunk's ID. Once the currently terminated chunk “a” minimal content objects are determined by sub-function 36g-4b1, it is then possible for the sub-function to compare this determined chunk “a” content to all content currently being maintained by display content monitor 28-6m, thus content available for visual (or audible) copying by the operator 10. Upon a sufficient match, sub-function 36g-4b1 may assign another authorship to some or all of the minimal content objects comprising the currently terminated chunk “a,” where then sub-function 36g-4b1 provides to content marking function 20g-cm authorship information (such as preferably the reference identity,) and where the marking function 20g-cm then marks the appropriate “matching”/“copied” minimal content objects comprising the currently terminated chunk “a” with the authorship information determined from the content monitor 28-6m, and the also marks any and all other remaining currently terminated chunk “a” minimal content objects as being authored by the operator 10.

A careful consideration will show that a correspondence between known currently terminated created chunk “a” content and displayed monitored content is the strongest indication of “mind-copying,” What is preferred is that any eye gaze behaviors are interpreted as “supporting” or “not supporting” mind-copying and are used as an additional feature for marking authorship within session work product 20g-wp by function 20g-cm of a and within a created chunk of content. Hence, created content might be marked as “operator 10 authored, supported by eye gaze tracking,” verses “operator 10 authored, questioned by eye gaze tracking,” verses “operator 10 confirmed copied from displayed or audible information sourced from referenced author xyz” “supported or not supported” by “eye gaze tracking” (for keyboard entered operator 10 minimal objects) or “lip syncing” (for microphone entered operator 10 minimal objects). In addition to the supportive nature of eye gaze tracking, the input nature associated with the minimal content objects determined to comprise a just terminated chunk “a”/created content may also be used as an indication of mind-copying. As a careful reading and consideration of the teachings provided herein will show (see for example the discussion of input nature related to FIG. 11B,) input nature that exhibits less editing and a faster input cadence is for example indicative or mind-copying, whereas a normal operator 10 entering a larger amount of minimal content objects comprising a created chunk is more likely to “make mistakes,” pause to think. “go back to prior locations in the flow of objects,” change objects, etc. This input nature characterization is portrayed in the present figure as preferably implemented by work product chunking and characterization function 36g-4a that also preferably has access to work product characterization history 20g-ch, where history 20g-ch is preferably indicative of the input natures across many types of operators, many types of apps, and many types of digital content, as well as the specific “baseline” input natures for the operator 10 these same apps and types of digital content.

What can be seen is that “mind-copying” by an operator 10 can be both definitively determined based upon sufficient correspondence with any of displayed (or audible) content as monitored 28-6m, and then further supported by correlated and uncorrelated operator 10 behaviors as well as the determined input nature of the created chunk of content under consideration. Thus, the present system teaches the objective marking of chunks “a” of created content (known to be input by the operator 10,) along with additional marking of the minimal objects comprising the created chunk “a” with known authorship (the operator 10 or not) as well as indications supporting or not supporting the determination of authorship, where the careful reader will note that for any given chunk “a” of created content, some minimal content objects may be marked with a different authorship than objects in the same chunk, even if all the minimal content objects are marked as “created by” (i.e., “entered by”) the operator 10.

Still referring to FIG. 11E, for inserted content chunks “b,” determine identify exchanged content function 36g-4b may invoke sub-function determine inserted source/reference identity 36g-4b2, where sub-function 36g-4b2 has access to clipboard monitoring 28-9m, all as prior discussed. Also as discussed, inserted content chunks “b” are substantially a single “content event,” with a begging and ending time bounding a minimal session time duration itself indicative of an insert operation versus a created object-by-object operator 10 entry operation. As inserted chunks “b” are detected and determined they can be assigned a specific chunk ID and marked by content marking function 20g-cm. Upon or after so marking, it is desirable to also identify the authorship of the potential multiplicity of minimal content objects comprising the just inserted content chunk “b”. As prior discussed, any content prior copied to the system clipboard is preferably also associated with any of the copying app 20g-app, and then preferably the copying app 20g-app's determinable source type and reference identity such that any copied content thereafter pasted from the clipboard comprises such authorship information which may then be transmitted to content marking function 20g-cm for appropriately marking all minimal content objects comprising the just terminated inserted chunk “b.” The careful reader will note that it is possible that the content being contained and tracked by the clipboard monitoring function 28-9m may be of multiple authorships, where for example an operator 10 working in a “first app” 20g-app creates a mix of operator 10 authored and referenced copied content (thus being at least two content chunks with respect to the “first app” current session work product 20g-wp,) which is then copied as a “single copied chunk of mixed authorship content” to the system clipboard, where this mixed authorship is tracked by the clipboard monitor 28-9m and made available to the determine inserted source/reference identity sub-function 36g-4b2 for ultimate provision to content marking 20g-cm. And finally, it is noted that when marking authorship for both mind-copied created minimal content objects and inserted minimal content objects, it is also possible to mark the objects with an app ID representing the original work product app 20g-app used to create the mind-copied or inserted content, along with even a chunk ID associated with the one or more chunks of authored content from the session work product associated with the work product app ID, all as a careful consideration will show.

Still referring to FIG. 11E and now with respect to sub-function 36g-4b3 for determining exported prompts, as will be understood by those familiar at least with what is known as “AI generative content” but otherwise in general what can be understood as “prompted content for insertion,” it is typical that an operator 10 using for example a “first app” generates a created chunk “a” of content (typically but not mandatorily authored by the operator 10) and presents this created chunk “a” through an interface to an application service as a “prompt” for inducing a response from the service. Regarding what is referred to as “generative AI,” the crafting of such input prompts by the operator 10 is known to cause possibly significant differences in the response from the service, and that therefor such operator 10 crafting of the prompt is a “creative act” in and of itself. This crafting is often referred to as “prompt engineering” and is even becoming a studied art. Prompt engineering is especially important for generative AI with respect to many forms of synthesized content including text, bit map images, video and/or audio, computer programs, etc. The present teachings recognize the value of maintaining the original prompts created by the operator 10 that at least in part caused any of what eventually may become inserted content (whether inserted through mind-copying or the clipboard, or even via an API interface,) since again the determination of the prompt itself as a created chunk “a” is an act of important authorship that is ideally associated with the final work product 20g-wp.

In this regard and for example, a “first app” 20g-app through which the operator 10 provides at least one presumably (but not necessarily or restrictively) created content chunk “a,” is in communication with the “generative AI” app via for example an API, such that the “first app” 20g-app typically provides a user interface means for the operator 10 to indicate that for example the current prompt content (that the operator is looking at as identified as a prompt by the “first-app” user interface) is to be now transmitted to (or otherwise used by) the “generative AI” app. This user interface means is a type of command (such as a “button” or the “return key”) to be activated by the operator 10 and as such becomes a determinable minimal content object (command) that is made available to sub-function 36g-4b3 via the succession of described and portrayed functions, where then sub-function 36g-4b3 is triggered at least in part by this minimal object command to determine the content of the exported prompt. Upon receiving a “prompt provided” or similar minimal object command, it is then possible and preferable that sub-function 36g-4b3 determines the contents of this prompt which may be as simple as referring to the currently closed chunk ID, but otherwise is determinable by interacting with the work product app 20g-app that has presumably just transmitted this same content via the generative AI “first app” API (in this example). What is important to see is that the present system teaches the objective determination of “prompt content” for recordation in reference to the session work product 20-wp and authorship assessment 20g-sc, where the prompt is most often but not necessarily authored by the operator 10 but otherwise has a determinable content authorship all as herein described, and where, as will be understood by those familiar with at least generative AI this prompt content will be at least in part a “creative cause” of any “returned content” provided in response by the “first app,” this “returned content” of which may then itself in part or in full become inserted work product content “b” either via the “first app” or some “second app,” all as a careful reading and understanding of the present teachings will show. It will also be clear that a prompt engineered and used in a “first app” and then causing returned content used in a “second app” can then itself using the teachings provided herein be transmitted via a number of means for storage in the original references and prompts 20g-rc associated with the work product 20g-wp being created by the “second app.”

Still referring to FIG. 11E, after function 36g-4b has determined the final authorships (operator or non-operator) of any created chunk “a” or inserted chunk “b,” and has determined any prompts for causing any of response content used for mind-copied chunks “a” or inserted chunks “b,” function 36g-4c then preferably records in original references and prompts artifact 20g-re all original inserted reference content and all original prompts causing at least in part some response content comprising any of a chunk “a” or “b.”

Following the completion of functions as described so far in relation to FIG. 11E, at some point app 20g-app will seek to close out the current session and therefore the work product 20g-wp. While it is possible and traditional for app 20g-app to substantially continually maintain the “thus far completed” work product 20g-wp, it is optional that the function 36g-4d for providing content authorship and assessment is only invoked by the app 20g-app or the work product authenticator 36g at a determined “end-of-current-session,” although this function 36g-4d can also be for example executed at least “chunk-by-chunk,” and or otherwise at multiple points in session time during a current session, or even at the command of the operator 10 as expressed through for example the app 20g-app user interface for providing the operator 10 with a current “work product content authorship assessment.”

There are many possible determinations that can be provided by content assessment function 36g-4d, all optionally under the control or input of either the use certificate 35-uc and/or the supervisor certificate 24-sc, where such assessments can include for example: 1) the total percentage of work product 20g-wp content that was determined to be originally provided through the mind of the operator 10 without discernable mind copying, as opposed to the amount of work product content that is otherwise determined to be not of operator 10 original authorship, whether of chunk type “a” or “b,” 2) the amount of operator determined editing (thus chunk “a”) with respect to any of non-operator authored content (whether of chunk type “a” or “b,”) and 3) the objective “quality” or other measures of quality and or effort devised for evaluating the operator 10's authored content at least in terms of the operator 10's known baselines and history, or with respect to other person's baselines or histories. As a careful reading of the present teachings will show, it is now possible to allow an operator using virtually any traditional app that is further adapted as described herein for creating or modifying virtually any type of digital work product to objectively demonstrate via a digital certificate that the work product that is output from the app is of their own “authenticated composition,” with then further proof of the sources and references defining the authorship for the various possible one or more parts of the authenticated composition (including their own authorship or some other person or non-person/algorithm's authorship,) with then still further proof of any editing and re-composition by the operator of parts of the composition that were not originally authored by the operator, with still then other verification regarding the input natures of the various compositional parts supporting significant new types of authorship and work product effort type metrics.

In a current marketplace example of the urgent need for a system for providing the authentication and authorship of digital work products as described herein, there is a well-known modern artist by the name of Greg Rutkowski whose artworks have been used in what are known as the “training sets” of a generative AI engine marketed as Stable Diffusion. The net result is that Stable Diffusion is able to generate digital artwork of a very similar style to Mr. Rutkowski to the point where according to a Decrypt publication article dated Jul. 29, 2023 and entitled “Greg Rutkowski Was Removed From Stable Diffusion. But AI Artists Brought Him Back,” “Rutkowski himself struggled to differentiate between AI-generated pieces and his genuine works.” (It is note that at least some of these “mimicked” AI digital creations are generated in response to engineered prompts comprising text only.) The article goes on to state that Mr. Rutkowski requested that Stable Diffusion remove his artwork from their training set, where although Stable Diffusion complied with Mr. Rutkowski's request other persons using what are known as “AI open-source learning models” such as “LoRa,” proceeded to train these models on the Rutkowski portfolio resulting in still more synthetic mimicry of organic patterns.

Those familiar with the many legal and ethical questions surrounding the unauthorized use of organic (people-based) work products (of virtually any sort that can effectively be digitized) will also recognize the value of the teachings provided herein for digital work product labeling. President Biden, the current US President as of the time of the application of this provisional, recently announced an intention being formed between the US Government and the “AI tools industry” to provide for what is being called “AI labeling.” This AI labeling then is presumed to be a provision such as a “hidden watermark” or similar that is originally provided by the individual AI generative tools and for example web-services such as Stable Diffusion or Chat GBT.

Upon a careful consideration, and as pointed out in the Decrypt article referenced above, unfortunately, single individuals using open source AI now have the ability to train on virtually any digital content available in the marketplace, whether properly licensed or not, where these open source AI algorithms (unlike Stable Diffusion as of the time of this writing) are even executed “locally” on the host system (i.e., 20-c or 20-d) making it virtually impossible for the AI tool providers to comply with this stated government-industry “AI labeling” goal (e.g., its not their tool and its not running on their systems).

Regarding the use of watermarks for tracking generated AI content, in August of 2023, Futurism.com published an article entitled “When AI Is Trained on AI-Generated Data. Strange Things Start to Happen.” A first point being made is that as more unlabeled AI synthetic data is distributed on the internet, this same synthetic data is “scrapped up” with organic data (because of the lack of a label for excluding). It was pointed out that as the percentage of synthetic data begins to approach organic data in an AI training set this is when unwanted generative results are occurring and that “pretty soon, there's going to be more synthetic images on the internet than real images . . . there are going to be more synthetic websites than real websites, and there's going to be more fake text than real text.” The article goes on to state that “your best shot is using something like watermarking to be able to detect synthetic data and maybe remove it.” However, as the article then points out a watermark “intentionally introduces an artifact” into the generative AI content, where then this (watermark) artifact “can or will be amplified” leading to other unwanted results. Thus and considering the many drawbacks of the present state-of-the-art solutions for labeling AI content, it will be seen that the present invention does effectively label all organic and synthetic content, even as it intermixes, maintaining authorship and hence identifying for example which AI algorithms created which content, all without watermarks and all without easily being defeated by the use of open source programs, where for example even a highly industrious forger is unlikely to create their own apps for creating various compositions of digital content and where not having a certificate for any digital content uploaded to the internet is itself a label sufficient for ignoring the content in an AI training dataset.

As a careful consideration will show, relying upon the self-motivated user of at least “locally executed” synthetic AI tools (typically “open source”) to willingly provide verification that their works are “not originals,” “not organic,” or otherwise “synthetic,” even when passing their synthetic work products off as organic originals may provide significant marketplace advantages and benefits, is unrealistic and also beyond the control of the mimicked authors. The present invention refers to this non-authored/mimicked work product labeling as “negative labeling,” wherein products that are “not organic” are so labeled by their human “prompters” or even “AI systems” prompting other AI generative tools. In contrast, the present invention refers to the teachings provided herein for authenticated authored work product labeling as “positive labeling,” where the operator seeks to show positive-proof of their own authenticity thus by implication any otherwise non-discernable mimicked digital work product lacking such positive-proof/certification can be assumed to non-original even synthetic, even if the mimicked work product lacks a “label.” Rather than an AI “synthetic-centric” labeling system, the present invention calls for virtually the opposite approach of an operator “organic-centric” labeling system, so that artists such as Mr. Rutkowski can using their own system (i.e., 20-c or 20-d) authenticate that their own digitally created work products where of their own creation and composition, and even going so far as to provide information on any digital work product components that are not of their own authorship but that we incorporated into their own work product composition, as well as how these inserted work products may then have been edited.

Still referring to FIG. 11E, and now finally to the last generate certificate function 36g-4a, as has been prior discussed an important new artifact is taught herein for accompanying with a session work product 20g-wp, namely a session work product certificate 36g-wc (see also FIGS. 10A, 10B, 11B, and 11C, as well as upcoming FIGS. 11F and 11G for further detailed discussion regarding certificate 36g-wc). Among the purposes for the certificate 36g-wc is to provide the work product 20g-wp compositional author, operator 10, with a means for positively verifying that the work product 20g-wp was composed by them, where this proof-of-organic composition preferably includes at least some encrypted information that works to ensure a tamper-proof certificate and a tamper-proof work product (see FIG. 11F). Towards these ends for ensuring that a work product has not been tampered with, certificate 36g-wc preferably comprises some information quantifying the session work product 20g-wp, such as a well-known hash code, where one or more hash codes quantifying one or more of the entire work product 20g-wp or any of its constituent parts are for example encrypted by the honest broker executing on a primary device 1 representing the operator 10 and possibly in coordination with the work product app 20g-app representing the app, or the work product authenticator 36g representing a third party, where this encryption otherwise remains non-discernable to even the operator 10, and where preferably the “public key” or similar for enabling this secured encryption has been pre-obtained through a secured process by the honest broker 1 (work product app 20g-app, or work product authenticator 36g) from for example a supervisor app 24-app (see FIG. 11A) and/or from a registrar (see upcoming FIG. 11G).

As a careful consideration will show, once the work product 20g-wp has been “closed” by the work product app 20g-app, it is then preferred that the work product 20g-wp is substantially undiscernible (not including what is known as “meta-data” for providing additional work product descriptions) from a traditional work product so that any other traditional work product viewer, processor, service, etc., might then process the work product 20g-wp without requiring any further or substantial adaptation. However, upon the need, any such other traditional work product viewer, processor, service, etc., might also request or require a work product certificate 36g-wc to either allow normal processing and/or to allow additional “identification” for publication and limitation of transformations and distribution of the work product 20g-wp. Upon receiving the requested and encrypted certification 36g-wc corresponding to the work product 20g-wp, if the processor for example is a supervising entity 24 (see especially FIGS. 10A and 10B) then preferably the supervising entity using their own corresponding “private key” (all as well-known in the art of digital asymmetric encryption) may decrypt at least a sufficient part of the certificate 36g-we for then receiving at least some information such as one or more hash codes that can then be used to confirm that the corresponding work product 20g-wp has not been tampered with (thus causing a newly generated hash code to not match the decrypted hash code, all as will be understood by those familiar with the art of hash code usage).

It is also possible, for example, that some other desirous processor of the work product app 20g-app that is not the supervising entity 24 or otherwise has no means for decrypting any sufficient portion of the certificate 36g-wc, to then provide the certificate 36g-wc to any of a valid supervising entity 24 or a registrar used by for example the honest broker 1, work product app 20g-app, or work product authenticator 36g for registering the work product and/or otherwise encrypting the certificate 36g-wc, where then this supervising entity 24 and/or registrar (see especially upcoming FIG. 11G) decrypts the certificate 36g-wc on behalf of the other desirous processor and then verifies for example that one or more current hash codes of the corresponding work product 20g-wp (either determined by the other desirous processor and provided to the entity 24 or registrar, or determined by the entity 24 or the registrar) match or do not match the decrypted and corresponding one or more certificate included hash codes.

And finally with regard to both FIG. 11E and generate certificate function 36g-4c, it is noted that an author (the operator 10) may have one or more supervisors, e.g., such as an educational supervisor, work supervisor, publication supervisor, social media supervisor, etc., each of which who may have issued distinct supervisor certificates applicable to any given single work product 20g-wp. Furthermore, the author (operator 10) may wish to use one or more registrars (see FIG. 11G) for registering their work products 20g-wc as a means of enabling a form of public verification of the authenticity and authorship of a given work product 20g-wc by way of a specific one or more registrars who can provide certificate and work product authenticity affirmation and confirmation of the lack of work product tampering. Based upon this potential multiplicity of supervisor certificates and possible registrar certificates, generate certificate function 36g-4e can be configured to generate one or more certificates addressable and usable by any one or more supervisors and/or registrars, even where this addressability includes information within a “shared certificate” that is individually encrypted using different public keys from different supervisors and/or registrars. It is further preferred that for each work product there is always at least one certificate generated as, or serving as, a “generic” certificate comprising all or a significant portion of the multiplicity of information determinable using the present teachings or future modifications of the present teachings, especially including corresponding creation of the authorship view and characteristics 20g-sc that can be controlled or influenced by a use certificate 35-uc or supervisor certificate 24-sc, such that this “generic” certificate and corresponding “generic” authorship view may also be used to generate most if not all of a future required supervisor certificate or registration certificate at some later time after the work product 20g-wp has been “closed.”

Referring next to FIG. 11F there is shown a block diagram of the key artifacts comprising authenticated work product 36g-awp and produced by the novel apparatus and operation of the present teachings for determining the authenticity and authorship of a digital work product 20g-wp, where work product 20g-wp has been further adapted to comprise content marks having correspondence with content authorship view and characterizations 20g-sc, where then 20g-wp and 20g-sc have correspondence with original refences and prompts 20g-rc, all certified by any one or more work product certificates 36g-wc. The traditional session work product often comprises what is known as an assembly, where such assembly has at least two parts comprising the actual content, often referred to as “raw” content, as well what is often referred to as “meta-data” or “header-data” describing for example date of origination, last modified date, a self-described author (meaning simply that the operator chooses to indicate some authorship without any means for authentication,) etc. It is also often the case that the traditional work product includes “linkage information” for identifying standard transformation functions to be applied to the raw content, such as “embedded fonts,” color schemes, etc. The present invention anticipates that any such traditional work product information is simply then further augmented for example such that preferably the “raw content” also includes content marks indicative of discreate content “a” or “b” chunks comprising the raw content, all as discussed in relation to content marking function 20g-cm of FIG. 11E. As will also be clear, while there are anticipated advantages to implementing the newly taught content authorship view and characterizations 20g-sc, the original references and prompts 20g-rc, and the work product certificate 36g-wc as distinct artifacts from the traditional work product assembly, this should not be considered as limiting, as those familiar with data processing will understand that the traditional digital work product assembly can be further adapted to comprise any one of or any combination of such newly taught information 20g-sc, 20f-rc, and 36g-wc while still accomplishing the many uses and benefits taught herein.

With at least some work products, such as images comprising some form of pixel related representation, the preferred new work product embodiments additionally comprise one or more embedded signatures (see FIG. 10B). It is noted that unlike the current watermarks being suggested and implemented for distinguishing at least AI generated images and video, the presently taught embedded signatures are preferably implemented within the traditionally known “noise levels” of the data comprising the images or video, as opposed to traditional and presently used AI image watermarks that are so differentiated from the surrounding pixels as to become what in engineering is known as signal verses noise, and in data science is often referred to as a feature or “image artifact.” Those familiar with engineering in general and data science in particular, will understand that it is necessary that the traditional watermark be clearly distinguishable signal-data (as opposed to a “noise-data watermark” as described herein) in order for a generalized algorithm searching images without any other pre-known information (such as the seeds and encoding parameters discussed herein in relation to FIG. 10D and the authenticating imager 20-d-ai) to be able to recognize the pattern of the traditional watermark irrespective to any other possible patterns of pixels in any possible image or video.

As will also be clear, this must-be distinguishable across virtually all possible pixel arrangements requirement of a traditional watermark will at least 1) make it easier for a nefarious actor to also identify and then remove the same watermark thus defeating the AI content label, and 2) must be first removed and replaced as data from the original traditionally watermarked AI generated image by the consuming AI training mechanism so that this traditional watermark isn't itself then learned and repeated by the subsequent AI algorithm. Unlike traditional watermarks that are intentionally designed as clear signal-data to be easily detectable “above the noise,” the present teachings show how to provide an embedded signature “within the noise” inherent within virtually any “digitally sampled sensor data” such as data provided by image sensors creating a multiplicity of “sampled pixels” (as discussed herein) or by microphones/audio sensors creating a multiplicity of “sampled sounds” (not discussed, but for which the teachings are directly applicable and anticipated).

Unlike traditional watermarks that can therefore be universally discovered by any algorithm using well-known data filtering techniques, the presently taught noise-embedded signatures are virtually undetectable without also knowing the pre-requisite information of for example where to look in the multiplicity of noise and what to look for, where by encrypting this pre-requisite information or otherwise keeping this information secured to a “verifier” service (such as provided by the sensor manufacturer or even an individual honest broker, all as taught in relation to FIG. 10D) such noise-embedded and “secured watermarks” as presently taught serve the dual purpose of (a) allowing any interested party to inquire with the “verifier” service to determine if the image or video is based upon an original transduction of the light or audio being represented as opposed to a digital mimic of such original light or audio (thus an AI generative “synthetic”,) and (b) allowing for the direct processing of the noise-embedded watermarked images without any need to remove this noise-embedded signature data over a concern that the embedded signature (“secured watermark”) might be learned by the consuming AI algorithm and in some form become undesirably repeated data within new generative images, all as a careful consideration will show. Thus, the presently taught noise-embedded “secured watermarks” have clear advantages over the traditional signal-embedded “unsecured watermarks” for use in “positively” marking “organic” original light and sound transduced image and audio data as opposed to “negatively” marking “synthetic” (i.e., “not organic”) mimicked light and sound image and audio, where images and audio not positively marked and verifiable as organic can be suspected as, or otherwise treated as synthetic without verification as to otherwise.

Although not discussed specifically herein, other forms of embedded content signatures for other non-naturally transduced data (where naturally transduced data refers at least to image and audio sensors) will be obvious to those skilled in the various types of digital content, and thus these forms (beyond images and audio) may also comprise embedded signatures used for substantially the same purpose of providing means for determining if the raw content of a work product has been altered without the use of a work product authenticator, in any of the many deployments discussed herein, where such alteration conceptually serves to invalidate the certificate 36g-wc at least to some degree, all as a careful consideration will show.

Also as prior discussed, virtually all digital work products can be “hashed” to determine a unique code that represents the exact composition of data, such that any changes to this exact composition results in a different hash code. Regardless of signatures, the present invention prefers that a hash code of at least the “raw digital content” comprising the work product assembly is determined at the time the work product is “closed” by the work product app 20g-app at the termination of a session of operation. It is herein taught that this at least one hash code is encrypted and stored preferably in the work product certificate 36g-wc, and for example can only be decrypted by any one of or any combination of a supervisor 24, a registrar (see FIG. 11G,) or even by the operator 10's honest broker app executing on a primary device 1, again all as prior discussed. Such an encrypted hash code is then available for comparison by any third-party reviewing a session work product 20g-wp along with its corresponding work product certificate 36g-wc, where the third-party can determine a “current hash code” of the work product 20g-wp and then provide this current hash along with the certificate to any a supervisor 24 or registrar digital service, where then this service itself never has possession of the work product 20g-wp but simply decrypts at least a portion of the certificate for which the service's private key (for example) is applicable as the asymmetric key (all well-known,) and then compares the current and decrypted hash codes providing back to the third-party preferably only a “pass-fail” indication and not for example also providing the original now decrypted hash code.

Work product certificate 36g-wc preferably further comprises data of at least three types including 1) identifying data, 2) tamper detection data, and 3) assessment data. Identifying data (1) is often found, or at least available as “fields of data” that can traditionally and optionally be provided by the operator or the app to specify their own identifying information along with the digital work product, albeit not the authenticated identifying information as herein taught, where the present invention prefers that any such traditional identifying data populating the work product certificate 36g-wc is replaced by or accompanied with the herein taught “automatically authenticated identifying information” (again, not identifying information as optionally entered by the choice of the operator 10, but rather identifying information automatically determined by any one of or any combination of the app 20g-wp, work product authenticator 36g, and/or honest broker executing on a primary device 1 about any one of or any combination of the operator 10's identity, the operator 10's honest broker primary device identity, the work app 20g-app's identity, the work product authenticator 36g's identity, the asset (system 20-c, device 20-d)'s identity, or even the further adapted input apparatus identity (see 20-c-akb and 20-c-ams in FIG. 11D,) thus being “automatic identities”).

As stated, this identifying data also preferably comprises data describing the system used to generate the work product 20g-wp, again automatically determined so that this data cannot simply be set at the choosing of the operator (thus possibly “tamped with,”) where such system identifying data might comprise for example a unique identifier for any one of or any combination of the work product app 20-g, the work product authenticator 36g, the honest broker running on the primary device 1, the primary device 1 itself, the system 20-c, 20-d, or even one or more input apparatus such as further adapted keyboard 20-c-akb and further adapted mouse 20-c-ams. As a careful consideration will show, this “automatically” determined system identifying data provides another means for authenticating the work product 20g-wp, where for example the operator 10 may choose to register any such “automatically identifying” data with the honest broker 1 or a registrar (see registrar 29 in FIG. 11G,) with the intention of describing the only valid system environments for providing a work product 20g-wp originating from the operator 10, and thus any nefarious agent attempting to forge a work product 20g-wp to appear to be from an operator 10 must then also know or discover additional identifying information as described, all of which increases the difficulty for the nefarious agent and helps to protect the operator 10's intellectual property (i.e., the digital work product 20g-wp,) where in this way identifying data (1) is similar to tamper data (2).

However, tamper data (2) is also directed toward identifying characteristics of a completed “closed” work product 20g-wp, such as what is known in the art as a hash code, where any such identifying characteristics are usable for performing a verification to ensure that a closed work product 20g-wp is then not later “tampered with” or otherwise altered without the used of the herein taught system (which then by the teachings herein would confirm a prior work product 20g-pwp with prior work certificate 36g-pwc, allow app 20g-app modifications to create a new modified work product 20g-wp, with a corresponding new certificate 36g-wc (n+1) (see FIG. 11C). The present invention is not intended to be limited to the use of the existing art hash code, as other measurements of a closed work product 20g-wp can be devised, for example an algorithm that totals the byte count of every “Xth” byte found in the work product, where X is determined by a formula with variables only known to the encryptor/decryptor. Any one or more solutions are considered as minimal acceptable for the purposes of warding against work product 20g-wp tampering.

Still referring to FIG. 11F, the third preferred type of certificate 36g-wc data is assessment data, for example describing the percentage of the work product 20g-wp determined to be “organic” (thus authored by the authenticated operator 10, which may include any or both of created chunks “a” and inserted chunks “b” all as a careful reading of the present invention will show,) the percentage of “sourced” or “inserted” content (i.e., chunks “b”) as well as the percentage of identified versus unknown references, the percentage of this same sourced content that has been edited by the operator 10, a measure of the percentage of “organic” operator 10 applied effort, for which multiple formulations are possible with the goal for example of providing a quantification regarding any of the total content that is determined as authored by the operator combined with the level of complexity of this self-authored content verses any additionally present sourced/inserted content, as well as some measurement of content cost, for example including a time element and/or a comparison to baseline or historical performances, etc. The present description and depiction of assessment data is meant to be exemplary, as many formulations and valuable insights using new “work product assessment metrics” can be determined based upon the new functions and data taught by the present invention, and thus the teachings should not be limited to comprising only the assessment data identified herein, or even using all of the herein described assessment data, as many variations will be clear to those skilled in the art and also based upon the needs related to a specific type of digital content.

Those familiar with the challenge of maintaining corporate and government data and digital content secrecy will understand that many of the herein taught certificate 36g-wc information are usable as a means of for example in tracking the original and various “identities” (such as the operator 10, system 20-c, device 20-d, work product app 20g-wp, work product authenticator 36g, honest broker and primary device 1, as well as input devices such as 20-c-akb and 20-c-ams) of any digital content that is found to be generally distributed beyond the corporate or government internal eco-system, where such tracking then allows for a systematic review of internal processes. Conversely, as a careful consideration will show and as will be made clearer in FIG. 11G especially with respect to encrypted private content 20g-wp-prv and the authenticated work product viewer/browser 37, it is also possible to prohibit any non-officially released digital content from even being decrypted for access or viewing, by for example assuring that any one of or any combination of the attempting accessor-viewer is the original operator-composer 10 or supervisor 24, that the app 20g-app attempting to access-view the digital content is the originating app 20g-app, or similarly that the access-viewing of the digital content is being attempted on the original system 20-c or device 20-d using the original input devices (such as 20-c-akb and 20-c-ams). The present invention further anticipates that for example at least any of the system 20-c, device 20-d, or primary device 1, can determine a current geolocation (such as using any of a GPS or LPS system that is all well-known in the art,) where then for example any of the work product app 20g-app, work product authenticator 36g, or honest broker and primary device 1, works to prohibit access and or viewing of the work product 20g-wp based upon the current geolocation. As will also be made clear by the teachings of FIG. 11G, any desirous third-party having possession of any encrypted private work product content (again, see 20g-wp-prv of FIG. 11G) and an associated certificate such as 36g-wc (or 29-rc) may use a registrar 29 to gain access or viewing, where registrar 29 for example provides author services 29-srv in order to substantiate and allow the desirous third-party to gain access or viewing of the private and encrypted digital content, all supporting the many benefits of certificates such as 36g-wc taught herein.

An important new artifact, the content authorship view and assessments 20g-sc is enabled by the presently taught work product functions (see especially FIG. 11E) and provides for viewing any otherwise traditional digital work product as comprising one or more compositional parts, where each part comprises both origin and authorship information. As depicted and prior described in FIG. 11E, the herein taught further adapted processing of a work product app 20g-app serves to break the work product 20g-wpp into one or more discrete chunks of content, such as “created content” chunks “a” portrayed as 20g-wp-a1 and 20g-wp-a2, and “inserted content” chunks “b” portrayed as 20g-wp-b1, where a work product 20g-wp must comprise at least one chunk “a” or “b” but is otherwise not limited in the number of chunks “a” or “b,” all as a careful consideration will show. As discussed, the portrayed chunks comprising view 20g-sc are preferably separate and distinct data from the work product 20g-wp, although those familiar with software will understand that a further adapted work product 20g-wp could also directly comprise this view 20g-sc information, thus being a single “document” or even “document assembly.” Each chunk “a” or “b” preferably comprises identifying information for example including a chunk ID that may then be used by work product 20g-app content marking function 20-cm (see FIG. 11E) to tag or associate with each minimal object comprising a given chunk (keeping in mind that although a typical inserted chunk “b” is received by the app 20g-app as a single multiplicity of one or more minimal content objects, each of the minimal content objects within this inserted multiplicity is preferably marked with the chunk ID as a means for supporting original chunk identification as any of the individual minimal content objects comprising the inserted chunk “b” may later be “moved” or repositioned with the work product 20g-wp, for example no longer even being perceptually associated with the original inserted multiplicity, all as will be understood by a careful consideration and knowledge of apps and digital content). Per chunk information may also include the “beginning” or “starting” time of the chunk with respect to for example a “session time” as well as an “ending” or “stopping” time.

While other types of data for “storage” with each chunk “a” or “b” will be obvious based upon a careful reading of the purposes described herein, what should also be recognized is that each chunk “a” or “b” preferably comprises its own associated “assessment tag” comprising a multiplicity of possible information or “characteristics” such as metrics or the “input nature” (see especially FIG. 11B) of the chunk, where then even this proposed starting and ending time of a chunk could alternatively be provided within the assessment tag data. Those familiar with software and data structures will also recognize that what is portrayed as two objects, such as a chunk “a” 20g-wp-a1 and an associated assessment tag 20g-wp-alt, could likewise be implemented as a single data object comprising the chunk ID and all related chunk assessments and characteristics, or even as more than two data objects, and thus the present depiction should be considered as exemplary and for teaching purposes, but not as a limitation of the present teachings. Assessments and characterizations are preferably directed toward at least describing the input nature of the multiplicity of minimal content objects uniquely comprising the given associated chunk (and otherwise no objects from other chunks,) as well as optionally comprising information derived by comparison of such chunk characteristics with baseline and historical characteristics data 20g-ch (see FIG. 11E). Thus an assessment tag such as 20g-wp-alt or 20g-wp-blt are meant for describing preferably at least one or more of the novel input nature characteristics taught herein regarding a single chunk/part/portion of what may be a complex composition comprising the work product 20g-app.

Assessment preferably also includes indications of authorship, source types, references, and even in the case of created content input apparatus types (e.g., keyboard, mouse, digital tablet) and then even unique apparatus IDs (see the teachings related to FIG. 11D related to keyboard 20-c-akb and mouse 20-c-ams,) where the careful reader will understand that associating an input apparatus ID with a given chunk is another useful way to support the assessment of authorship while also thwarting forgeries, all as prior discussed. It is noted that any and all of the data comprising an assessment tag could also be encrypted, for example using any of a supervisor, registrar, honest broker, or even and “app” public key, thus helping to ensure that potentially private information such as an input apparatus ID is not vulnerable to being stolen by the simple reading through of the view and characterizations data 20g-sc. What will also be understood is that in addition to the valuable input nature characteristics taught herein, there is a growing body of art for using AI algorithms to assess content in terms of some qualification if not also a quantification. For example, AI algorithms are now being developed to help assess if some content is likely organic or synthetic, thus “AI detecting AI,” AI algorithms are also anticipated herein to be able to learn a given operator's “data/content styles” such as word choices, sentence length, sentence complexity, etc., for text or drawing objects, object group complexities, etc., for 2D/3D content, where then this learning of operator 10 style, especially using historical characteristics 20g-ch (related to the operator 10 or a type of operator 10) also becomes a valuable supportive indicator or authorship and possible “applied effort” and “content cost.”

Still regarding FIG. 11F and view and characterizations 20g-sc, along with preferably distinct assessments “per chunk,” it should be recognized that at least some chunks of “a” created content (such as 20g-wp-a2) may comprise “edit content” for example related to an inserted chunk “b” (such as 20g-wp-b1). This depiction should be seen as exemplary as many possible “nestings” of chunks are possible, where, as will be clear, a given chunk “a” of created content can represents edits crossing over one or more other “a” or “b” prior chunks. What is important to see is that the view 20g-sc preferably tracks chunk-by-chunk the building of what might be simple a single-chunk/single-author/single-session work product 20g-wp to very complex multi-nested-chunk/multi-author/multi-sessions of edit-cycle work product 20g-wp, where again the minimal objects comprising each next chunk are themselves preferably tagged with the next chunk ID after which the minimal objects may then be deleted, moved, modified, etc., but all still subject to be “viewed” by chunk/author/reference, etc., as taught herein. It is also important to see that each distinct chunk preferably has its own assessment, including “nested chunks” that are inserted into or otherwise editing prior chunks.

And finally, the present depiction shows that other assessment tags such as 20g-wp-mt1 may be generated and limited to some or all of the work product 20g-wp, including only a portion of a single chunk, or multiple portions or entire chunks, thus providing with a sophisticated tool for algorithmically assessing virtually any complexity of composition/work product 20g-wp.

And finally with reference to FIG. 11F, there is portrayed in the lower right corner a digital work product 20g-wp whose content is viewable “as normal” but then augmented/overlaid/relatable to the authorship view and characterizations 20g-sc, where many means for implementing such augmentation, overlay, or relating of information are possible and valuable, all as will be clear and well understood by both a careful reading of the types and purposes of the newly herein taught work product 20g-wp data and by those familiar with user interfaces. The present depiction also portrays that anticipated linkage from the view and characterizations 20g-sc, or from the work product 20g-wp itself, to any and all original references and prompts 20g-rc (see FIG. 11E).

Referring now to FIG. 11G, there is shown the authenticated operator 10 using any variation of work product app 20g-app with work product authenticator 36g to create/modify a session work product 20g-wp validated by a work product certificate 36g-wc, all as prior discussed herein. Such prior discussions were primarily focused on the apparatus and methods for determining what was collectively referred to as an “authenticated session work product” 36g-awp (see for example FIGS. 10A, 10B, 11B, 11C, and 11D) comprising at least work product 20g-wp and certificate 36g-wc, and then also preferably comprising authorship view and characteristics 20g-sc and original references and prompts 20g-rc (both not depicted in the present figure,) whereas the present figure is focused on the subsequent generalized distribution and authenticated consumption of such authenticated session work product 36g-awp.

Regarding distribution, authenticated work product 36g-awp is shown to be both registered (with a registrar 29) and divided into public summary content 20g-wp-sum verses private content 20g-wp-prv, where a public summary 20g-wp-sum is in general able to be consumed substantially similarly to today's current “not-authenticated” or traditional work product, such as viewing the summary via a traditional web-browser, while the private content 20g-wp-prv is restricted to viewing through the authenticated work product viewer/browser 37 that amongst other benefits ensures that the operator 10 of the viewer/browser 37 consuming any of private content 20g-wp-prv is authenticated to be organic (thus a person) and otherwise not a synthetic mimic or “bot” (thus, for example, a synthetic algorithm that is attempting what is referred to in the art as “scrapping” digital content for use in training algorithms, as will be well understood by those familiar with today's “LLMs” or large language learning models/AI tools).

Besides the many benefits that are being provided to the authenticated work product 36g-awp creator-modifier 10, a careful consideration of the present figure will also show the many benefits being provided to the consumers of 36g-awp including receiving heretofore unavailable proof-of composer (creator-modifier) 10 authentication, identification of single or multi-authorship within the composition 20g-wp, and “tamper-proof” knowledge ensuring that the composition 20g-wp has not been further modified by unauthorized persons or algorithms. Such many benefits are preferably provided at least in part through the use of author services 29-srv provided by a registrar 29. Registrar 29 is preferably a web-service available to the creator-modifier (session operator) 10 of the product 36g-wp, where registrar 29 acts as an intermediary to verify the work product 20g-wp's authenticity and proper certification 36g-wc. For example, in at least one embodiment, any of the work product app 20g-app or work product authenticator 36g (or even the honest broker app executing on a primary device 1) acts as the “registering agent” and establishes preferably secured communication with the registrar 29 for providing unique identity information (such as any one of or any combination of the honest broker ID associated with creator-modifier 10, an ID associated with the work product authenticator 36g ID, or even an ID associated with a work product app 20g-app) along with the work product certificate 36g-wc to the registrar 29. The registrar 29 then responds to the registering agent providing a registrar certificate 29-rc for distribution along with any of the authenticated session work product 36g-awp.

Preferably summary (public) work product content 20g-wp-sum is then distributed along with the registrar certificate 29-rc using traditional digital content distribution means available to the creator-modifier 10, where for example traditional means comprise the internet and web-sites, where then for example the traditional web-browser (that is for example implemented using a version of HTML) is further adapted to process the registration certificate 29-rc. Thus, summary content 20g-wp-sum is made available via the widest distribution means for review and interaction by any desirous content “consumer,” (where this consumer may notably by “organic” and therefore a person or “synthetic” and therefore an algorithm/“bot”).

Registration certificate 29-rc also preferably includes sufficient unique registrar 29 identification information whereby the consuming party's traditional internet access means (such as a browser) may determine or otherwise ascertain how to connect or engage the consumer with the registrar 29's web-services including author services 29-srv. The registrar 29's web-services are minimally able to verify the consumer that the authenticated session work product 36g-awp as represented by the public summary 20g-wp-sum is registered and by implication at least composed by an authenticated “organic” creator 10. Registrar certificate 29-rc also further comprises some work product unique identifier for use by the registrar 29 to identify the registered authenticated work product 36g-awp, where upon being presented by the consumer's internet access means (or similar) with this unique work product identifier, the registrar services 29-srv are then able to retrieve the associated work product certificate 36g-we in order to provide additional certificate 36g-wc available information. Alternatively or additionally, any of work product characterization and assessment information (see for example FIG. 11F) comprised in the work product certificate 36g-we may be copied into the registrar certificate 29-rc and thus made available to the consumers simply by processing the registrar certificate 29-rc obviating the need to engage the author services 29-srv except for additional information or other services as will be described or apparent to those skilled in the necessary arts.

In the preferred operation, the registrar 29's author services 29-srv also confirms for the consumer that the work product unique identifier is registered with the registrar 29 (and thus the registrar 29 has access to additional private content 20g-wp-prv, etc.) but then also allows the consumer to engage with additional services. Additional services are preferably approved or even feature limited by the creator-modifier 10 of the work product 36g-awp and for example include providing to the consumer any one of or any combination of: 1) creator vetting and contact, 2) work product verification, 3) means for obtaining access to private content 20g-wp-prv including optional payments and licenses, and 4) means for obtaining “content ownership” associated with the private content 20g-wp-prv such as receiving what is known in the art as an “NFT” or “nonfungible token.” Thus, as the careful reader will observe, registrar 29 preforms many valuable and new services 29-srv (to be further discussed) for an otherwise traditional content creator-modifier 10, enabling by analogy an “organic market” for digital content, where consumers and creators meet in virtual to buy, sell, and exchange authenticated “organic” digital content (where it is then also understood that creators may wish to distribute some forms of synthetic content or organic-synthetic content mixes, but where it will also be clear that the consumer is made fully aware of the composition of any work product 20g-wp, as described herein).

Still referring to FIG. 11G, creator vetting and contact services (1) for example comprise means for the consumer to obtain and verify any of credentials maintained by the registrar 29 concerning the creator-modifier 10, where even in providing any such credentials the creator-modifier 10 may remain anonymous if so desired (where credentials may for example include professional or educational accreditation, third-party certifications, etc.). Communication/contact services may also allow for an exchange of information between the consumer and the creator-modifier 10 over some communication means or some communication time frame, where presumably then the consumer may engage the creator-modifier 10 with questions, requests, negotiations, etc. The vetting services may also provide ratings and prior performance or review information regarding any of the creator-modifier 10, the work product 20g-wp, or any other creator-modifier 10 work products, where such ratings and reviews are traditionally available on other types of e-commerce platforms and will be well understood by those familiar with the art web marketing and social media.

Work product verification (2) for example comprises providing any of work product certificate 36g-wc information (see FIG. 11F for examples) and otherwise confirming the “non-tampered” state of either or both of the public summary 20g-wp-sum or the private content 20g-wp-prv (where the private content 20g-wp-prv then is known to the consumer as per any of means provided or implied herein). One preferred means for confirming non-tampered states of either the public summary 20g-wp-sum or the private content 20g-wp-prv is for the consumer to use a known method for determining a hash code of either of 20g-wp-sum or 20g-wp-prv and presenting such hash code to the author services 29-srv, where upon receipt the services 29-srv accesses the work product certificate 36g-wc to confirm that the original hash codes as determined by either of the work product authenticator 36g or the work product app 20g-app at the time of work product “closing” and stored in the certificate 36g-we are sufficiently matching, all as will be understood by those familiar with the use of hash codes.

Author services means for allowing the consumer access to private content 20g-wp-prv (3) may also include taking forms of optional payments (using any of well-known e-commerce means) and providing any of well-known use licenses. When engaging services (3), the consumer has means for receiving or accessing private content 20g-wp-prv, but then is preferably limited to accessing any such private content by only using the authenticated work product viewer/browser 37, to be discussed in greater detail shortly. As is depicted, when the creator-modifier 10 is registering a work product 20g-wp preferably represented as a public summary 20g-wp-sum as well as a more detailed or “full” private content 20g-wp-prv, the registrar provides the work product authenticator 36g or the work product app 20g-app with a registrar public key 29-k1, where then this public key 29-k1 is used to encrypt either or both of the private content 20g-wp-prv and the work product certificate 36g-wc, thereby providing even further security as only the registrar using the registrar private key 29-k2 may then perform decryption, as will be understood by those familiar with asymetric data encryption services. It is noted that the creator-modifier 10 may elect to release the entire session work product 20g-wp as the public summary 20g-wp-sum, thus allowing any consumer (organic or synthetic) to freely review the entire work product 20g-wp, where then the author services 29-srv are not used to obtain the full work product 20g-wp (and thus any private content 20g-wp-prv,) but may still be used for all other described services, especially including the authentication and “tamper-proof” verification of the summary 20g-wp-sum (and thus the entire work product 20g-wp). Conversely, the public summary 20g-wp-sum might comprise minimal to even no information other than perhaps some work product 20g-wp name, where then the entire session work product 20g-wp is only available as private content 20g-wp-prv.

Author services 29-srv (4), means for obtaining “content ownership” associated with the private content 20g-wp-prv such as receiving what is known in the art as an “NFT” or “nonfungible token,” are meant to use any of well-known or to become known digital content ownership exchange mechanisms and means, and thus as the careful reader will understand provide a service to the creator-editor 10 and the consumer by encapsulating any such services via the author services 29-srv user interface.

Still referring to FIG. 11G, any such registrar services 29-srv are again preferably web-services made available through the internet or “cloud” 26 for use by the operator 10 using a system 20-c (or device 20-d). Up to this point in the teachings regarding the present figure, the operator 10 was characterized as the creator-modifier 10 of the authenticated session work product 36g-awp that they were desirous of distributing to the larger market through the internet, where the presently taught work product registrar 29 provided a valuable service to this creator-modifier 10 by “hiding” the complexity of various services such as vetting, contact management, work product verification, collection of payments, issuing of NFT's etc. From this point in the description of the present figure forward operator 10 will be referred to as a work product “consumer” 10 that is desirous of interacting via the internet available registrar services 29-srv in combination with traditional web search engines that have been further adapted to be authenticated search engines 28-sr providing authenticated search results. It is noted then that the creator-modifier 10 and the consumer 10 are both authenticated operators 10 of a system 20-c or device 20-d, where this “authentication” proving identity when operating an asset such as 20-c or 20-d is important for fully appreciating the type of services 29-srv that are essentially reserved for and limited to both the provision of content that is at least “organically composed” (even if comprising some portion of synthetic content) and the “organic” consumption of any such “organic” content, where organic consumption is limited to rates and types of consumption traditionally possible for people but often significantly exceeded by algorithms that are for example “scrapping” content.

Just as new authentication tools were required on the production-side of the work product 20g-wp, so also the consumption-side also requires a new tool, which is the authenticated work product viewer/browser 37. Thus the careful reader will see the succession of tools that herein form the “authenticated digital content work flow” starting with the work product authenticator 36g and work product app 20g-app (as well as any of the other related teachings herein such as the honest broker app executing on a primary device 1,) proceeding to the author services 29-srv, through the authenticated search engine 28-sr, and ending in the authenticated work product viewer/browser 37, together collectively enabling what is herein referred to as the “organic market” for digital content, where such a market can be exclusively restricted to participation between only persons as content creator-editor's 10 and only persons as content consumers 10.

Those familiar with software systems and architecture will understand that there are may alternatives for implementing this succession of tools providing the authenticated digital content workflow, and as such that described embodiments should be considered as preferred but not limiting as many alternatives may offer unique benefits. What is important to see is that the workflow taught herein collectively provides an assurance that “people-are-exchanging-with-people” various digital content as opposed to the current environment where the sudden marketplace rise of LLMs (large language models) (or other algorithms) has presented an uncertainty that people may be unwittingly interacting with “bots” posing as people or consuming digital content of uncertain origins including being of a significantly or entirely synthetic nature.

As will also be appreciated from a careful consideration, although the present depiction is of digital content that is created or modified, and then “closed” and distributed, thus in a non-real-time connection between the creator-editor 10 and consumer 10 (allowing the creator-editor 10 to scale a single work product 20g-wp into distribution with a multiplicity of possible consumers 10 over a longer duration of time,) it is also possible that a real-time connection can be formed for example via an internet social media or similar service, where the two endpoints in the real-time connection are each being operated by persons using any one of or any combination of the herein taught authenticating work product app 20g-app, work product authenticator 36g, honest broker and primary device 1, or authenticating work product viewer/browser 37. As will be understood, this enablement of assuring that “people-are-exchanging-with-people” is then extensible into real-time connected two-party or even multi-party social exchange platforms, including “on-line social services,” medical or life advice services, shopping assistance, dating or meetup services, or even internet video gaming, etc., where then all parties are assured that each end point in the real-time conversation/exchange/interaction is operated by an authenticated person using an authenticating app 20g-wp. With the rise of internet available synthetic on-line social services, etc., and the ability of such synthetic “end-points” in a real-time conversation to convincingly mimic a person, such as through text, voice, and perhaps soon even audio-visual representations, there is a growing societal concern, especially with respect to the more vulnerable persons (such as youth, the elderly, the mentally disabled, and even simple less sophisticated and aware consumers,) that these vulnerable persons might be unaware that they are engaging with synthetic service end-points and thus perhaps more disadvantaged. As implied by the present figure, any real-time connection between one or more parties (each concurrently acting as both content creator-editors 10 and consumers 10) can be implemented using the end-point authentication teachings provided herein, even to the point where a particular end-point is authenticated to be operated by a person 10 (with shared or not shared identity,) but where this person 10 is inserting content into the “real time conversation” that is not authored by the person, including synthetic content.

Still referring to FIG. 11G, as a careful consideration will show, the present system allows for at least some “public” content 20g-wp being provided via the authenticated workflow described herein that is otherwise distributed and consumed in a traditional manner using traditional and widely available tools, with the crucial exception that this public content 20g-wp comprises an associated registrar certificate 29-rc (or in alternative embodiments the work product certificate 36g-wc itself). Regarding this new certificate 29-rc comprising information for providing a digital content label and/or related work product 20g-wp information, a first marketplace benefit is provided by further adapting a traditional web browser (such as Chrome, Edge, Firefox. Vivaldi, Opera, and Safari) to recognize this association between public content 20g-wp-sum and the certificate 29-rc, and then to use preferably public information comprised in the certificate (such as the registrar 29 service name, or any of the characterizations and assessments associated with the work product 20g-wp for which the summary is applicable, see especially FIG. 11F,) or otherwise to communicate with the registration service 20-srv to obtain and provide such information for viewing and use by the consumer 10 operating the further adapted traditional web browser on their system 20-c or device 20-d. Those familiar with HTML protocols will also recognize other ways for implementing these net effects, where at least one way is to transfer any of such registrar certificate 29-rc information into meta-data associated with the digital content, where the traditional web-browser already comprises means for processing such meta-data and thus the further adaptation is removed from the web-browser.

In yet another beneficial improvement enabled by the present teachings, the traditional search engine (such as Google, Bing, Baidu, Yahoo, and AOL search) are further adapted to process any of such registrar certificate 29-rc information, in any form or implementation including comprised within or without certificate 29-rc, where then such processing effects the search results for the consumer 10, such as by limiting, filtering, and/or sorting search results based upon indications of “authenticated organic” composition, and then even some processing effect based on the percentage of synthetic content within the composition, as well as other possible processing effects that will be apparent to those providing such search engines based upon a careful reading of the many new and valuable metrics, measurements, characteristics, assessments, etc., taught herein with respect to an authenticated work product 36g-awp. While such and similar processing effects as enabled herein are provided through further adapted traditional search engines and traditional browsers for accomplishing any of possible authenticated search results 28-sr, what is then preferred is that unlike traditional solutions where a consumer of digital content can “sec,” “peruse,” and even “download”/“obtain a copy of” the entire digital content/work product (whether through a separate viewer or through a web browser) the consumer 10 is limited to any accessing and processing of any of private content 20g-wp-prv to using the herein taught authenticated work product viewer/browser 37.

It should be first recognized that viewer/browser 37 uses any of the apparatus and methods taught herein to authenticate consumer 10, for example where viewer/browser 37 works with an honest broker executing on a primary device 1 in communication with the system 20-c or device 20-d such that the honest broker 1 provides preferably real-time authentication that the current operator 10 of the asset 20-c or 20-d is for example known and a person, even if the operator 10 remains anonymous to the asset 20-c, 20-d or the viewer/browser 37, all according to the many teaching provided herein. In comparison, a traditional document viewer such as the Adobe Reader or any of the aforementioned traditional web browsers reasonably assume but does not verify that the operator 10 of the traditional viewer or browser is a person, let alone an authenticated person. What should also be recognized is that the newly taught private content 20g-wp-prv is encrypted preferably using the registrar public key 29-k1, and thus a traditional viewer or browser cannot otherwise decrypt the private content 20g-wp-prv. This encrypted private content 20g-wp-prv, as decided by the original creator-modifier 10, is then precluded from any access without both consumer 10 authentication and use of author services 29-srv. The careful reader will observe a first benefit in that any such private content 20g-wp-prv, although distributed “on-line” and through the world-wide-web/internet, cannot be “scrapped” and thus download used by algorithms (for example to train LLMs) thus serving as an enforcement of use and licensing for the people that are creator-modifiers 10, which is a presently an urgent issue in the marketplace.

Still referring to FIG. 11G, what will also be apparent is that the present teachings provide for the assurance that only a person (as only a person can be authenticated) is attempting to consume private content 20g-wp-prv, where then after authentication and using viewer/browser 37 the authenticated consumer 10 may communicate with author services 29-srv respective of private content 20g-wp-prv to then decrypt such content (using a provided registrar decryption key such as 29-k2) or were the registrar services 29-srv alternatively upon request provides via a secured communication path the decrypted private content 20g-wp-prv. What should also be recognized is that preferably the viewer/browser 37 maintains encrypted communications with author services 29-srv, all well-known in the art, such that even upon receiving of a private key 29-k2, the key 29-k2 remains effectively secure even when being used on the consumer 10's local system 20-c or device 20-d. What will also be understood is that key itself can be used and immediately destroyed as data by the view/browser 37, providing another means for securing the key 29-k2 from “copying” and “sharing.”

In still yet another variation, after viewing public summary content 20g-wp-sum using viewer/browser 37, and thus being already authenticated, consumer 10 may then request from the services 29-srv the transmission of the encrypted private content 20g-wp-prv, where then this encryption is accomplished by the services 29-srv just prior to transmission and using a unique asymmetric key pair 29-k1/29-k2 that is only useable by the consumer 10 as it is locked to the authenticated consumer 10 and for example the honest broker ID associated with the consumer 10 (all as a careful reading will show as possible). Thus, the consumer 10 can retain a copy of the “personally encrypted” private content 20g-wp-prv on their system but can still only access this content 20g-wp-prv by first proving their authenticity to for example the honest broker 1 in communication with the viewer/browser 37, after which the unique private key 29-k2 is allowed to be used by the viewer/browser 37 repeatedly, and will otherwise not work for any other desirous operator-consumer 10. What is important to see is that various means are already known and/or are herein discussed that ensure that encrypted private content 20g-wp-prv cannot be consumed accept in the “authenticated presence” of a consumer 10 that is a person, thus again precluding unauthorized access and processing by algorithms and “bots.”

Still referring to FIG. 11G and viewer/browser 37, a careful consideration will also show that upon viewing of any decrypted private content 20g-wp-prv it is normally possible to cause system 20-c or even device 20-d to perform a copy-to-clipboard function, thus beginning a process of at least copying in some format the currently viewable private content 20g-wp-prv. The present invention anticipates that viewer/browser 37 is only usable by the consumer 10 when also the herein taught clipboard monitoring (see function 28-9m of FIG. 11E) is being used, where such presently taught clipboard monitoring then also records the source/reference/authorship of the private content 20g-wp-prv along with any copied content. It is also noted that the author services 29-srv preferably provides authorship view and characterizations artifact 20g-sc to viewer/browser 37, such that when operator 10 is reviewing (or copying to the clipboard) any of private content 20g-wp-prv the authorship of each create chunk “a” or inserted chunk “b” of the private content is accessible for review (or copying to the clipboard,) all as a careful reading of the present invention will show.

Furthermore, it is preferred that work product viewer/browser 37 may only be invoked and used for accessing private content 37 when the viewer/browser 37 is able to determine via the operating system that only work product apps 20g-app using an authenticator 36g are in concurrent operation, such that any subsequent pasting from the clipboard results in the forwarding of the source/reference/authorship into the app 20g-app for associating with that apps currently inserted chunk “b” (again, as a careful reading will show).

It is further anticipated that viewer/browser 37 may also allow viewing of private content 20g-wp-prv even while non-compliant apps are concurrently being used by the operator 10, (such as apps that paste from the clipboard as traditionally practiced without concern for retrieving and annotating the pasted content with any of the source/reference/authorship information,) where then the clipboard monitoring process as taught herein and assured by the viewer/browser 37 to be running blocks or otherwise interacts with the operating system to prohibit any such copying to the clipboard, thus also blocking any pasting into a non-compliant app.

It is also possible that some motivated individual can develop a computer program for scrapping memory or similarly accessing any private content 20g-wp-prv being currently displayed by the viewer/browser 37. As those familiar with the human vision system will understand, human operators integrate visual information over for example 1/30^{th} of a second, whereas a traditional display outputs information at 2 to 4 times this rate.

As a precautionary measure working against memory scrapping programs, the present invention anticipates that viewer/browser 37 will present the same next “ 1/30^{th} of a second” of private content 20g-wp-prv in for example 2 to 4 different data representations, where then the human eye of the operator 10 integrates these 2 to 4 different representation into a single integrated view that is substantially the private content 20g-wp-prv, but where then the scrapping program picks up only a single of the 2 to 4 different representation per individual memory scrape. The type of differentiation per each 1/30 of a second can then also be randomly alternated, were sometimes the 2 to 4 different representations randomly omit different areas of content, or always present all content but provide this in inverted colors or intensities, where other differences are possible and thus the scraping program even if copying all 2 to 4 different representations must then provide additional effort to combine the information into a sensible representation of private content 20g-wp-prv.

What should be understood is that the authenticated viewer/browser 37 working with the operating system of the system 20-c or device 20-d can effectively and substantially block currently viewed private content 20g-wp-prv from being copied to the clipboard and shared in an unauthorized way with any non-compliant apps, thus making unauthorized copying of the creator-modifier 10's work product 20g-wp much more difficult if not fully prohibited. It should also be understood that private content 20g-wp-prv may be restricted to only being accessible via a herein taught work product app 20g-app (working in cooperation with a work product authenticator 36g) such that the viewer 37 is a function invoked by and within the control of the app 20g-app (as opposed to a distinctly operating app, all as will be understood by those familiar with computing programming and software systems,) where using such internal control by the app 20g-wp private content 20g-wp-prv is then inserted using more traditional methods, like a web-service/“API” interface with the author services 29-srv and thus not going through the clipboard thereby allowing that the clipboard may then be disabled by the system 20-c/20-d during such private content 20g-wp-prv viewing and accessing.

As will also be understood by those familiar with algorithmic scrapping of internet data sources, such scrapping of content seeks to operate at very high rates of data transmission, whereas human consumers 10 cannot consume content at nearly these same rates, and thus any of the author services 29-srv, the further adapted traditional search engines or browsers 28-sr, and/or the viewer/browser 37 are also preferably further adapted to throttle or limit the rate and amount of authenticated private content 20g-wp-prv being decrypted and otherwise provided to match a “person's” verses a “bot's” consumption rate. And finally, it is also anticipated that viewer/browser 37 ceases to display any private content 20g-wp-prv when the viewer/browser 37 looses what is known in the art as “the current focus,” and thus the consumer 10 has indicated for example by clicking on the user interface of another application that they are switching away from the present use of the viewer/browser 37, in which case the viewer/browser 37 is invoked a final time before the switching of focus and during this last invoking clears the private data 20g-wp-prv being currently displayed, all of which acts to thwart “mind-copying” from the viewer/browser 37 displayed information as a careful reading of the present invention will show.

Regarding all of the figures in the present invention that include blocks with arrows between blocks including arrow heads typically interpreted as indicating a direction of the flow of information, while such arrows heads are being used to best support the teaching of concepts, they should not be considered as limiting to the single direction of flow. Those familiar with software will understand that data is typically passed (by “value” or “by “reference”) but otherwise transmitted from a first component to a next component but that this communication between components may include for example “hand shaking” or otherwise some backflow of information that is not explicitly herein discussed or portrayed with a corresponding arrowhead. Thus, it should be recognized that it is also possible that some components/functions provide or receive inputs from other components/functions, depicted and not depicted, even if connecting flow arrows are not provided or discussed.

### CONCLUSION AND RAMIFICATIONS

As will be apparent from a careful consideration of the teachings herein, and from a familiarity with a domain of assets (such as automobiles and driving, private homes and private rentals, heavy industrial equipment and operations with repetitive or creative aspects) wherein operation of such assets includes significant liabilities (such as loss of life, damage to asset or environment, business loss due to poor operation even leading to halted operation, etc.), it is desirable for any of the asset owner or otherwise responsible managing party, operator(s), asset or operation insurers, or regulators and operation enforcers to have increased assurance of operator and asset “readiness” for the intended operation.

Those familiar with the rapid pace of enhancement in personal wellness devices and their increasingly varied and more accurate measurements of bodily characteristics, functions, states, “health metrics,” etc. will also appreciate that creating meaningful physiological assessments of “physical readiness for task” is now possible. As will be familiar to those with an understanding of psychology, especially in combination with technology for measuring human behaviors and expressions indicative of internal mental states such as indications of emotion and stress via the use of image analysis, it is now also possible to combine such physiological and psychological assessment to create an even more detailed and accurate “physical and mental readiness for task.”

Those familiar with the many ways to exchange digital information across the vast array of marketplace computing elements such as smartphones, wearables, laptops, desktops, remote services, etc., as well as those familiar with the vast and growing resources of on-line asset and task-instructional media, will also understand that it is now possible to provide distributed informal learning systems to augment formal learning systems, and that in either case it is now possible to use digital certifications of sufficient formal or informal asset and/or task-instruction to determine an even more detailed and accurate “physical, mental, and experience readiness for task,” where experience is being used broadly to include both actual operation experiences (e.g., “flight hours”,) as well as time spent receiving formal and informal training possibly resulting in certificates of training and education, or otherwise confirmation of any other completed “competency enhancement” task anticipated to affect operation performance.

Those familiar with sensors and automation, the “Internet of Things” (“IoT”), and the rapid pace of engineering to create “smart assets” (e.g., “smart cars”) will understand that significant new “asset self-measurable” performance data is now available per each operation of the asset (e.g., “driving a car,” “occupying a building,” “overseeing an industrial process,”) where this information is usable to increase real-time performance feedback to the operator facilitating real-time performance assistance and also objective post evaluation of the performance a specific task (applicable to the asset, the operator, and the particulars of the session of operation).

What will also be apparent to those familiar with wellness devices determining health assessments related to an operator is that such assessments may be determined pre-asset-operation (as well as during and after asset operation,) whereas “asset self-measurement” by “smart assets” is typically during asset operation. The present invention as well as the prior related applications have shown that it is now possible to further adapt personal mobile computing devices such as smartphones and wearables including smartwatches to not only create wellness measurements but to also allow the authentication of these measurements in private, where such privately authenticated wellness measurements are combinable using public regiments into a publicly trustable “current wellness state” of an individual, where then this wellness state may be made public at the discretion of the individual without requiring that the underlying wellness measurements be shared with the public. These personal mobile devices are referred to in the related applications and herein as “primary devices” and where shown to communicate with “companion devices” such that the measurements from the companions could themselves be authenticated by the primary device, thus greatly expanding the health metrics for use in determining an operator's authenticated health state.

What will be apparent to those familiar with smart assets self-capturing session related operation metrics is that while such assets may relate their unique asset ID to such captured operation metrics, especially in the larger commercial markets where also specific assets might be shared by more than one operator, no effective means are available for authenticating that a smart asset's self-measurements are relatable to a specific operation performed by specific operator.

Amongst the many technical advantages taught herein, and the many market benefits derived from these technical improvements, the careful reader will see that it is now possible to determine a “physical, mental, and experience readiness for task” relating to an operator that is combinable with a “readiness for performance” related to an asset, where even the operator's and asset's readiness can be dependent upon the planned session of operation, the plan of which itself may have variable features contributing for example to different operator skill and preparedness requirements, or different asset functioning requirements. The careful reader will also recognize that it is now possible to authenticate “smart-asset” gathered data per a specific session of operation as being “about” or “of” a specific person, whether this person is identified or remains anonymous for example via an anonymous operator ID. It can be seen that this new authenticated operator-asset-session data advantageously covers pre-session, during-session, and post-session (where pre-session and post-session are together non-session,) and further that such non-session and session related and authenticated data, whether anonymized or not, can be accumulated for significant marketplace advantages especially including an increase in operator and asset “safety.”

It will also be clear that the present systems allows for the determination of personal operator histories and baselines with respect to measures of readiness, where some operators might be more susceptible to operation failures at approximately the same underlying measurements of physiological and/or psychological state. For example, one operator might be more susceptible to operation failure with a “tiredness” measure of “6” or a “impaired” measure of “3” (these are arbitrary scalers selected for illustration only) versus a different operator with the same measures but a different baseline determined by past (i.e., historical) performances, where it is possible using the present system to find statistical correlations between this difference in operator-task susceptibility to failure and the accumulated historical measures relating to for example task familiarity, task difficulty, operator group associated (such as age group, sex group, etc.,) etc., and thus the present system and methods teach the accommodation of individual differences within group norms. Based upon this understanding, it will also be clear that asset “owners” and other third-party's with a vested interest in the proper operation of an asset by an operator, might then create more “individualized” policies and restrictions, thus better fitting the broader marketplace made complex by individuality in operators, assets, and planned sessions of operation.

A consideration of the broad meaning of asset with respect to the present invention will show that assets should not be limited to material/physical objects, where for example in the emerging metaverse operators via virtual reality “VR” equipment may “operate” “digital assets,” where then the operation of these digital assets has a meaningful affect on at least other persons also participating in the metaverse experience. There is a known concern that individual persons might enter the metaverse experience under and alias with an intention to interact with other persons (generally here referred to as a performance/operation) so as to cause possible harm. One suggested remedy proposed in the market is to verify the identity of, thus “authenticate” and track each person operating in a metaverse experience. This in turn raises privacy concerns and introduces the traditional solution of requiring persons wishing to operate the asset (hear broadly the metaverse, including specifically “VR” apparatus such as a VR headset) to enroll with the metaverse (asset) provider before use (operation,) and then to undergo a process of verification of identity based upon enrollment prior to each metaverse use.

What can be seen by careful consideration of the teachings herein is that the VR headset is a localized controller dedicated to a “driver”/operator, akin to the front seat of an automobile wherein the occupant of the this “front-seat” location has access to operational control of the asset (metaverse/automobile). What can be further recognized is that this localized controller (i.e., VR headset like a driver seat with automobile controls) can include the equivalent of “occupancy detection” (e.g., the VR headset is determined to be in contact with a person's face by any of several well-known methods including cameras and contact sensors) and that upon detection this localized controller may then capture one or more “confirmation biometrics” (such as a facial image or a pulse) to provide to a primary device for authentication (similar to the descriptions herein of a vehicle interacting with a primary device to confirm an vehicle occupant prior to providing access to asset operational controls).

As will be understood by a careful reading of the present teachings, this asset to primary device method allows an operator to “enroll” with the intermediary app running on the primary device and to exclusively provide personal information such as name and “registration biometrics” to the intermediary app (as opposed to the metaverse/asset provider,) where then the intermediary app receives the confirmation biometric from the VR headset and confirms to the metaverse (asset) service regulator that the person occupying the localized controller is known to the intermediary app, where then the intermediary app may supply personal identifying information (thus not anonymous) or preferably an anonymous operator ID (that remains consistent across multiple uses/operations of the asset,) such that the asset (e.g., metaverse implementation) can then begin to accumulate “metaverse operation data” (like “vehicle operation data” described herein). This provides the same significant benefits in the digital world of the metaverse as are herein described for the physical world, where the benefits allow for safer operation, the ability to fit individualized operation limits based upon operator readiness (measured for example by physiological, psychological, experiential, competency/training preparedness, etc.,) the ability to create operation assessment including baselines and histories, as well as the ability for third parties to determine and implement restriction policies.

It will also be understood by those familiar especially with the training potential of the metaverse, that individuals (anonymous or not) could participate in realistic simulations (e.g., driving a car, flying a plane, operating complex equipment, performing a medical procedure) and where the individuals are intentionally subjected to a range of “readiness” states (by any combination of measures discussed herein or reasonably recognized as contributing to potential performance impairment,) such that the digital world “real-life” training system accumulates valuable information for predicting real-life performance in a physical situation modeled and simulated in the metaverse. As will be understood especially by the interested third parties, this ability to create “authenticated digital asset” operation data across a range of real-life measured “authenticated operator readiness” that can then be applied to parallel “real-life asset” authenticated operation provides significant marketplace advantages for developing better assets, operational features and limits, as well as operation policies and restrictions.

Thus the reader should see that the many teachings of novel technical apparatus and methods provided herein should not be limited to the chosen exemplary use cases but should rather be understood as generalizable across a virtually unlimited set of “operation domains” comprising operators in many possible and objectively measurable and authenticatable states of readiness and comprising assets with many possible features of operation, where again the assets are physical or digital.

As a careful reader will also see, the present invention provides many apparatus and methods, some of which are novel and useful by themselves or in various combinations, as will be obvious to those skilled in the various arts. Those skilled in the various art herein described will also understand that newer technologies useful for determining newer operator health measurements or readiness formulations as well as newer asset readiness formulations will become available for providing the same or anticipated functions as herein described. As such, the present invention and all of its preferred and alternative embodiments should be considered both as apparatus and methods that are exemplary and not limitations of the present invention, as these same apparatus and methods may be better implemented in the future using newly developed technologies not available at the time of the present invention. Furthermore, while the present invention has uses covering assets such as vehicles, equipment, buildings, structures, or processes, physical or digital, this list and any other types of assets mentioned herein or within the art referenced herein should not be considered as limiting, as virtually any asset capable of operation by an operator(s) for example including sports teams, stores, restaurants, schools, healthcare facilities, etc. are able to benefit from the present teachings.

Furthermore, the various embodiments described above are provided by way of illustration only and should not be construed to limit the scope of the disclosure. Various modifications and changes may be made to the principles described herein without following the example embodiments and applications illustrated and described herein, and without departing from the sprit and scope of the invention.

1. (canceled)

2. (canceled)

3. (canceled)

4. (canceled)

5. (canceled)

6. A system for determining and labeling the informational contribution of a person to any one of or any combination of the input, modification, or insertion of digital content as determined by information processing, comprising:
one or more devices or systems for executing one or more information processing algorithms, where at least one of the one or more devices or systems comprises or otherwise communicates with one or more sensing or otherwise input means for determining one or more inputs of contributing information by the person to the one or more information processing algorithms, and
where at least one of the one or more information processing algorithms determines a likelihood of correlation between any of the one or more inputs of contributing information made by the person and any of the input, modified, or inserted digital content determined by the information processing responsive to the one or more person inputs, and where at least one of the one or more information processing algorithms determines and provides one or more labels based at least in part upon the determined likelihood of correlation, where the one or more labels associate any of the specific correlated digital content input, modified, or inserted during the information processing with one or more indications representative of any one of or any combination of the person, the one or more devices, the one or more information processing algorithms, any of one or more determined sources of the specific correlated digital content, or otherwise any information derivable from one or more characterizations of the correlated one or more minimal content objects.

7. The system of claim 6, wherein:
the one or more inputs of contributing information comprise any one of or any combination of presence, identity, liveness, prompted responsiveness, or otherwise behaviors of the person, where the behaviors of the person comprise movements, gestures, and sounds caused or otherwise emitted by the person, and where any one of or any combination of the behaviors of the person are transduced by the one or more sensing or otherwise input means into the inputs of the person comprising any of application minimal content objects or application user interface commands, and
the one or more labels comprise any one of or any combination of personally identifying information, non-personally identifying information, information identifying or otherwise regarding to any of the one or more devices or systems used by the person during the information processing, information regarding to or otherwise identifying any of the one or more information processing algorithms, information relating to or otherwise identifying any of the time of occurrence or the duration of any of the one or more person inputs, and information relating to a geolocation regarding to or otherwise identifying where the person was physically located while using the one or more devices or systems for executing the information processing.

8. The system of claim 7 comprising two or more devices or systems for increasing the privacy of the person, wherein:
at least one of the two or more devices or systems is limited to functioning as a first authenticating device or system for exclusively executing one or more authenticating algorithms and at least one of the two or more devices or systems is limited to being a second development device or system for executing the one or more information processing algorithms;
at least any of the one or more inputs of contributing information used for determining the presence, the identity, and the liveness of the person are determined, received, or otherwise processed exclusively on the at least one first authenticating device or system by the one or more authenticating algorithms;
the at least one first authenticating device or system receives information from the at least one second development device or system sufficient for use in determining the likelihood of correlation, and
the at least one second development device or system determines and otherwise provides a session work product substantially representative of the digital content determined by the one or more information processing algorithms, and the at least one first authentication device or system determines and otherwise provides the one or more labels associated with the work product.

9. The system of claim 7 wherein the labeling of the informational contribution of the person is representative of the one or more information contributions made by the person during at least one distinct session of operation of the one or more devices or systems, where the digital content input, modified, or inserted during the session of operation is session work product, where the application user interface commands comprise any one of or any combination of modify existing content commands to alter or otherwise modify any of the existing session work product or insert new content commands to insert into the session work product one or more other pre-existing digital content objects each comprising one or more minimal content objects, where the session work product comprises any one of or any combination of the minimal content objects determined or otherwise input, modified, or inserted based at least in part upon any one of or any combination of the person input application minimal content objects and the person input application user interface commands, and where the one or more labels are associated with any one or more of the session work product minimal content objects.

10. The system of claim 9 wherein the determined likelihood of correlation between any of the determined inputs of contributing information made by the person, and any one of or any combination of the minimal content objects comprising the session work product is based at least in part upon any one of or any combination of a time and duration of occurrence during the session of operation associated with any one or more of the contributing information, and any one of or any combination of a time and duration of occurrence during the session of operation associated with the input, modification, or insertion of a chunk of content comprising any one of or any combination of the minimal content objects comprising the session work product.

11. The system of claim 6 wherein the determined likelihood of correlation is based at least in part on any one of or any combination of types of authentication comprising:
(1) authenticating the presence of the person substantially at the commencement of or during the information processing;
(2) authenticating the liveness of the person substantially during the information processing;
(3) authenticating one or more behaviors of the person that are contemporaneous or congruent with one or more inputs received during the information processing;
(4) authenticating an input nature of one or more inputs with respect to any one of or any combination of a complexity of the inputs, an extended duration and time statistics of the inputs, a size of content input, an in-part metric of content input, a ratio or otherwise indication of the minimal content objects input verses the commands input, or an indication of editing of content, and
(5) authenticating a sufficient response by the person to a system challenge.

12. The system of claim 6 further comprising:
a supervisor or otherwise use certificate specifying any one of or any combination of limitations comprising (a) operating limits, (b) application limits, (c) types of authentication limits, and (d) extents of authentication limits, where the certificate is received or otherwise made available for use by any one of or any combination of the one or more devices or systems and the one or more information processing algorithms in order to determine the limits to be implemented by the one or more devices or systems and the one or more information processing algorithms, where the limits are implemented at any one of or any combination of before or during the input, modification, or insertion of the digital content.

13. The system of claim 6 wherein the digital content is a compositional work product composed over one or more sessions of operation, where each of the one or more sessions is conducted by the same or a different authoring person, where the one or more labels indicate the informational contributions to the compositional work product made during each of the one or more session of operation by the same or the different authoring person, and where access to the compositional work product by a consumer person is restricted, further comprising:
a work product access program executing on one or more consumer devices and systems for use by the consumer person to view, browse, or otherwise access the compositional work product, where the work product access program comprises one or more authenticating algorithms for authenticating any of the presence, identity, or liveness of the consumer person, and
a registrar service for receiving and at least in part providing the compositional work product to the work product access program, where any one of or any combination of the registrar service or the work product access program restricts access to the compositional work product based at least in part upon the authentication of the consumer person and at least in part upon the information comprising the one or more labels.

14. A method for determining one or more content sources and one or more most likely causal agents relating to digital content for use in labeling the digital content, comprising the steps of:
using one or more computational means for executing one or more content algorithms for (1) composing or otherwise determining the digital content, and
providing one or more authenticator algorithms augmenting or otherwise communicating with the one or more content algorithms, where the one or more authenticator algorithms are used at least in part to (2) determine the one or more sources of the digital content substantially at the time the digital content is received, composed or determined by the one or more content algorithms, (3) determine the one or more most likely causal agents in association with the one or more sources of the digital content, and (4) determine at least one label for the digital content (1) comprising a combination of representations of (2) and (3).

15. The method of claim 14 wherein:
each of the one or more determined content sources (2) provide either a part of or all of the digital content and are any one of or any combination of (2a) one or more input apparatus or otherwise interfaces for use by a person to input any of the digital content or application commands to the one or more computational means, (2b) one or more pre-existing documents or otherwise digital content locally or remotely accessible to the one or more computational means, and (2c) one or more content generative algorithms, and
the one or more causal agents (3) of the digital content comprises any one of or any combination of (3a) a person, or (3b) a specific algorithm.

16. The method of claim 14 wherein determining the one or more most likely causal agents (3) further comprises any one of or any combination of the sub-steps of:
(3i) monitoring a person currently in-use of the one or more computational means substantially while the person is providing inputs to the one or more computational means, where monitoring determines one or more person characteristics comprising any one of or any combination of the presence of the person, the identity of the person, the liveness of the person, and otherwise any behaviors and movements of the person, and where the extent of the person (3a) or the specific algorithm (3b) being determined as a likely causal agent is based at least in part upon the extent of correlation between any of the one or more monitored person characteristics and any of the congruent or concurrently composed or otherwise determined content;
(3ii) assessing any one of or any combination of the digital content and the person-inputs to determine one or more input nature metrics comprising any one of or any combination of a measure of complexity, a measure of duration or otherwise time statistics, a measure of content size, a measure of in-part content, a measure of content to associated commands, or a measure of content editing, and where the extent of the person (3a) or the specific algorithm (3b) being determined as a likely causal agent is based at least in part upon the one or more input nature metrics, and
(3iii) presenting the person with a system challenge and subsequently determining if a sufficient person response was received in conjunction with any one of or any combination of the monitoring of the person (3i) or the inputs of the person (3ii), and where the extent of the person (3a) or the specific algorithm (3b) being determined as a likely causal agent is based at least in part upon any one of or any combination of the system challenge and the sufficiency of the person response.

17. The method of claim 16 for protecting the privacy of a person determined to be a causal agent (3a) further comprising the step of:
using one or more additional computational means for executing at least the one or more authenticator algorithms implementing (3i) and (4), where the additional computational means are in communication with the one or more computational means, and
(5) encrypting the at least one label determined in (4) such that the at least one encrypted label can only be decrypted by securely exchanging encryption information with an intermediary algorithm executing on or in communication with the one or more additional computational means.

18. The method of claim 17 for restricting access by a content consumer person to the labeled digital content, further comprising the steps of:
(6) using one or more computational means for executing a content access program for use by the content consumer person to access the labeled digital content, and
(6i) monitoring the content consumer person currently interacting with the content access program, where monitoring determines one or more person characteristics of the content consumer person, and where the content access program restricts access to the digital content by the content consumer person based at least in part by any one of or any combination of the person characteristics determined of the content consumer person and any of information comprising the at least one label.

19. The method of claim 14 for restricting or otherwise specifying one or more limitations for any one of or any combination of (1), (2), (3), or (4) further comprising the step of:
(7) providing one or more certificates for use by any one of or any combination of the content algorithms or the authenticator algorithms substantially before or otherwise at the time the digital content is being composed or otherwise determined (1), where the one or more certificates comprise information specifying any one of or any combination of (a) operating limits, (b) application limits, (c) types of authentication limits, and (d) extents of authentication limits.

20. A system for determining and labeling the informational contribution of a person to any one of or any combination of the input, modification, or insertion of digital content comprising a work product as determined by information processing during a session of operation conducted on one or more computational means, comprising:
one or more computational means for use by the person during the session of operation, where the one or more computational means executes the information processing for any one of or any combination of the inputting of new content into the work product, the modifying of existing content comprising the work product, and the determination and otherwise insertion of pre-existing content into the work product;
one or more interface means in communications with the one or more computational means for use by the person to specify person-inputs comprising any one of or any combination of minimal content objects or application commands, where at least one of the one or more interface means provides or otherwise determines any one of or any combination of person characteristics, where person characteristics comprise indications of a presence of the person, an identity of the person, an indication of the liveness of the person, responses of the person to challenges, and otherwise behaviors of the person, where the behaviors of the person comprise movements, gestures, and sounds caused or otherwise emitted by the person;
one or more content algorithms comprising the information processing for receiving the person-inputs comprising any one of or any combination of one or more of the minimal content objects for use as new digital content and one or more of the application commands for use in directing the modification of existing content or the determination and otherwise insertion of pre-existing content into the work product, and
one or more authenticating algorithms comprising the information processing and either augmenting or otherwise communicating with the content algorithms, where the one or more authenticating algorithms determine an extent of the informational contribution of the person to the creation, modification, insertion, or otherwise alteration of the work product during the session of operation based at least in part upon a determined sufficient correlation between any one or more of the person characteristics and any one or more of the person-inputs, and where based at least in part upon the determined extent of the informational contribution of the person the one or more authenticating algorithms determine one or more descriptions of the informational contribution of the person to the work product for association with the work product.

21. The system of claim 20 wherein the sufficient correlation as determined by the one or more authenticating algorithms is based upon any one of or any combination of:
one or more timing-comparisons of an input session time during the session of operation when any one or more of the person-inputs was received and otherwise processed by any of the one or more content algorithms and a content session time when any of the digital content comprising the work product was added to or otherwise amended by any of the one or more content algorithms;
one or more command-comparisons identifying pre-existing content being added to or otherwise amending the work product based at least in part upon person-inputs specifying application commands to insert pre-existing content;
one or more copy-comparisons of content being added to or otherwise amending the work product substantially matching at least part of or all of any one or more of external content known or otherwise available to any one of or any combination of the one or more content algorithms, the one or more authenticating algorithms, and otherwise any other algorithm executing on or otherwise communicating with the one or more computational means, where external content is any content not comprising the work product, and where copying of external content comprises algorithmic copying or person mind-copying;
one or more input-nature assessments of given content being added to or otherwise amending the work product, where the one or more input-nature assessments are determined at least in part by analyzing any one of or any combination of the person-inputs and the given content, where the one or more input-nature assessments comprise any of indications of a complexity of the person-inputs in relation to the given content, a complexity of the given content, a duration of the session time over which the given content is added or otherwise amended to the work product in comparison to an amount of or size of minimal content objects comprising the given content, a statistical measure of the duration of the session time between minimal content objects comprising the given content being added or otherwise amending the work product, an edit-measure indicative of any of the temporal or spatial sequencing over a given duration of session time of a multiplicity of given minimal content objects comprising the given content and any interposing person-inputs not specifying the given minimal content objects, and an edit-measure indicative of the relative spatial or temporal locations of the given minimal content objects comprising the given content being added with respect to existing work product content over a duration of session time;
one or more person-characterizations determined as spatially occurring in a sufficiently congruent location with respect to any one of or any combination of the one or more interface means and any one or more components of the one or more computational means;
one or more person-characterizations determined as temporally occurring over a sufficiently concurrent duration of session time with respect to any of the person-inputs, and
one or more challenge-response exchanges resulting in a sufficient response from the person, where a challenge is any interrupting information provided to the person during the session of operation by any one of or any combination of the one or more authenticating algorithms and the one or more content algorithms, and where a sufficient response is based at least in part upon any one of or any combination of one or more of the person characteristics and one or more of the person-inputs.

22. The system of claim 20 for increasing the privacy of the person wherein:
at least the determination of the person characteristics is restricted to execute on one or more second computational means distinct from the one or more computational means.

23. The system of claim 20 where the operation of any one of or any combination of the one or more computational means, the one or more user interfaces means, the one or more content algorithms, and the one or more authenticating algorithms are limited, further comprising:
one or more digital certificates comprising any of (a) operating limits, (b) application limits, (c) types of authentication limits, and (d) extents of authentication limits, where the one or more digital certificates are received or otherwise made available for use by any one of or any combination of the computational means and the algorithms in order to determine and implement any one of or any combination of the limits of operation (a), (b), (c), or (d).

24. The system of claim 20 for restricting access to the work product to a content consumer that is an authenticated second person, further comprising:
a work product access program for use by the content consumer for accessing the work product, where the work product access program is executed on a consumer computing means that receives or otherwise accesses the work product, where the work product access program comprises one or more interface means for determining any one of or any combination of person characteristics of the second person, and where the work product access program either allows, denies, or otherwise restricts access to the work product based at least in part upon any one of or any combination of the second person characteristics.

25. The system of claim 20 wherein determining the one or more descriptions of the informational contribution of the person to the work product for association with the work product, further comprises any one of or any combination of:
determining a content source of any of a part of the work product or all of the work product, where the content source comprises one or more identifications of any one of or any combination of a person, a website, a generative AI algorithm, a pre-existing document, a connected device, and an input apparatus used by or functioning as any of the one or more interface means for receiving any of the person-inputs;
determining any one of or any combination of authenticated identifying information, automatically identifying information, tamper detection information, and assessment information;
determining one or more content marks for association with one or more content chunks or otherwise components of the work product, where the content marks comprise information indicating any one of or any combination of the content source, references information, generative AI prompt information, embedded signatures, or authorship information;
determining a content authorship view and characterizations of the work product based at least in part upon any one of or any combination of the content marks and further comprising any one of or any combination of the authenticated identifying information, the automatically identifying information, the tamper detection information, and the assessment information;
determining restriction information indicating any one of or any combination of any of a part of the work product or all of the work product that are to receive restricted access, and any combination of limits to systems, devices, or geolocations using or within which the restricted access to the parts of the work product or all of the work product is allowed, disallowed, or otherwise restricted, and where restricted access comprises access limited to or by an authenticating work product viewer or browser, where the authenticating work product viewer or browser restricts access based at least in part upon any one of or any combination of the restriction information and a confirmation of one or more person characteristics determined by the authenticating viewer or browser about a content consumer person currently using the authenticating viewer or browser;
determining or otherwise processing payments based at least in part upon any of the restriction information, and
determining one or more work product labels for association with the work product, where the one or more work product labels comprise any one of or any combination of one or more of the extents of the informational contribution of one or more persons, one or more of the sources of information associated with the content of the work product, any of the authenticated identifying information, any of the automatically identifying information, any of the tamper detection information, any of the assessment information, any of restriction information, and any of payment related information.
